(19) United States
(12) Patent Application Publication
(10) Pub. No.: US 2018/0355043 A1
(43) Pub. Date: Dec. 13, 2018

(54) Antibodies Specifically Binding HLA-DR and Their Uses

(71) Applicant: Janssen Biotech, Inc., Horsham, PA (US)

(72) Inventors: Christian Martinez, San Diego, CA (US); Qiang Chen, San Diego, CA (US); Melissa Swiecki, Blue Bell, PA (US); Robert Kuhn, North Wales, PA (US); Hong Zhou, San Diego, CA (US); Karen Duffy, Trappe, PA (US); Stephane Becart, San Diego, CA (US); Chichi Huang, Berwyn, PA (US); Xiefan Lin-Schmidt, Ambler, PA (US); Sheng-Jiun Wu, Broomall, PA (US); Jeffrey Luo, Malvern, PA (US); Galina Obmolova, Phoenixville, PA (US); Robin Ernst, Kintnersville, PA (US)

(21) Appl. No.: 16/062,255

(22) PCT Filed: Dec. 16, 2016
(86) PCT No.: PCT/US16/67235

§ 371 Date: Jun. 14, 2018

Related U.S. Application Data
(60) Provisional application No. 62/268,570, filed on Dec. 17, 2015.

(51) Int. Cl. C07K 16/28

(52) U.S. Cl. CPC C07K 16/2833; C07K 2317/92

(57) ABSTRACT

The present invention relates antibodies or antigen-binding fragments thereof specifically binding HLA-DR, polynucleotides encoding the antibodies or fragments, and methods of making and using the foregoing.

### SEQUENCE LISTING

This application contains a Sequence Listing submitted via EFS-Web, the entire content of which is incorporated herein by reference. The ASCII text file, created on 15 Dec. 2016, is named JBI5078WOPCT_ST25.xt and is 254 kilobytes in size.

### FIELD OF THE INVENTION

The present invention relates to antibodies and antigen-binding fragments thereof specifically binding HLA-DR, polynucleotides encoding the antibodies or fragments, and methods of making and using the foregoing.

### BACKGROUND OF THE INVENTION

Major Histoconmpatibility Complex (MHC) Class II molecules are used to present antigen-derived peptides to CD4⁺ T cells. Humans have three MHC Class II molecules: HLA-DP, HLA-DQ, and HLA-DR, each consisting of an alpha/beta (α/β) chain heterodimer that binds a peptide inside the cell and carries it to the cell surface for presentation. MHC Class 11 molecules are expressed on the surface of antigen-presenting cells (APCs) that include B cells, macrophages, and dendritic cells.

HLA-DR α chain, encoded by HLA-DRA1, is highly conserved. HLA-DR β chain, encoded by HLA-DRB1 or one of its paralogues HLA-DRB3, HLA-DRB4 or HLA-DRB5, is hyperpolymorphic. Antigen-presenting cells from all individuals express an alpha chain encoded by HLA-DRA1 and a beta chain encoded by HLA-DRB1, but can additionally express an alpha chain that pairs with one or two HLA-DRB3, HLA-DRB4, and HLA-DRB5-encoded chains. Therefore, an individual can express two to four HLA-DR isoforms depending on the maternal and paternal alleles inherited.

HLA-DRB1 in particular is associated with many human autoimmune diseases. Variations in the HLA-DRB1 gene can affect the specific peptides presented by HLA-DR, which in turn affects which antigen-specific CD4⁺ T cells will recognize and respond to that HLA-DR/peptide complex. The genetic association of HLA-DRB1 with autoimmune disease implicates the presentation of peptides to helper T cells in disease initiation and/or progression. T cell activation appears to be an early step in autoimmune disease, representing the initial recognition of a self-peptide as foreign. Pathogenic CD4⁺ T cells can directly cause tissue damage, but can also trigger B cell activation leading to the production of autoantibodies.

Polymorphisms in HLA-DRB1 have been found to be associated with diseases including rheumatoid arthritis (RA), systemic juvenile idiopathic arthritis, Grave's Disease, Hashimoto's thyroiditis, myasthenia gravis, multiple sclerosis, systemic lupus erythematosus, and type 1 diabetes (reviewed by Gough and Simmonds, Curr Genomics 2007; 8(7): 453-465 and Shiina et al., J Human Genetics 2009; 54: 15-19). Amino acids 70-74 on the side of the peptide binding pocket of the beta chain have been called the “Shared Epitope” and include positively charged residues (QKRAA, QRRAA, or RRRAA). The Shared Epitope is present in HLA-DRB1 alleles HLA-DRB1*01:01, *01:02, *04:01, *04:04, *04:05, *04:08, and *10:01, which are thought to preferentially accommodate citrullinated peptides, peptides in which the amino acid arginine has been modified to citrulline. About two thirds of RA patients have autoantibodies called ACPA (anti-citrullinated protein antibodies) present in their serum, hypothesized to arise as a result of citrullinated peptide recognition after presentation by “Shared Epitope” HLA-DR molecules.

HLA-DR is also expressed on a variety of hematologic malignancies as well as solid tumors and has been pursued for antibody-based therapy in these indications (Schweighofer et al., Cancer Immunol Immunotherap 61(12) 2367-73, 2012; Stein et al., 2006. Blood 108:2736-44; Altamonte et al., Oncogene 2003 22:6564-6569) although safety concerns exist with this approach.

Thus, there is a need for therapeutics to treat HLA-DR-mediated diseases such as autoimmune diseases and HLA-DR positive tumors.

### BRIEF SUMMARY OF THE INVENTION

The invention provides for an isolated antibody or an antigen-binding fragment thereof specifically binding HLA-DR, wherein the antibody or the antigen-binding fragment thereof competes for binding to HLA-DR with an antibody comprising
a heavy chain variable domain (VH) of SEQ ID NO: 58 and a light chain variable domain (VL) of SEQ ID NO: 61;
the VH of SEQ ID NO: 56 and the VL of SEQ ID NO: 60;
the VH of SEQ ID NO: 57 and the VL of SEQ ID NO: 61;
the VH of SEQ ID NO: 137 and the VL of SEQ ID NO: 61;
the VH of SEQ ID NO: 138 and the VL of SEQ ID NO: 61;
the VH of SEQ ID NO: 139 and the VL of SEQ ID NO: 61;
the VH of SEQ ID NO: 140 and the VL of SEQ ID NO: 142; or
the VH of SEQ ID NO: 141 and the VL of SEQ ID NO: 61.

The invention also provides for an isolated antibody or an antigen-binding fragment thereof specifically binding HLA-DR, wherein the antibody or the antigen-binding fragment thereof comprises
a heavy chain complementarity determining region 1, 2 and 3 (a HCDR1, a HCDR2 and a HCDR3) of SEQ ID NOs: 73, 74 and 75, respectively, and a light chain complementarity determining region 1, 2 and 3 (a LCDR1, a LCDR2 and a LCDR3) of SEQ ID NOs: 76, 77 and 78, respectively;
the HCDR1, the HCDR2, the HCDR3, LCDR1, the LCDR2 and the LCDR3 of SEQ ID NOs: 39, 42, 46, 50, 52 and 54, respectively;
the HCDR1, the HCDR2, the HCDR3, the LCDR1, the LCDR2 and the LCDR3 of SEQ ID NOs: 40, 43, 47, 51, 53 and 55, respectively;
the HCDR1, the HCDR2, the HCDR3, the LCDR1, the LCDR2 and the LCDR3 of SEQ ID NOs: 41, 44, 48, 51, 53 and 55, respectively;
the HCDR1, the HCDR2, the HCDR3, the LCDR1, the LCDR2 and the LCDR3 of SEQ ID NOs: 41, 45, 49, 51, 53 and 55, respectively;
the HCDR1, the HCDR2, the HCDR3, the LCDR1, the LCDR2 and the LCDR3 of SEQ ID NOs: 123, 126, 129, 51, 53 and 55, respectively;
the HCDR1, the HCDR2, the HCDR3, the LCDR1, the LCDR2 and the LCDR3 of SEQ ID NOs: 123, 126, 130, 51, 53 and 55, respectively;
the HCDR1, the HCDR2, the HCDR3, the LCDR1, the LCDR2 and the LCDR3 of SEQ ID NOs: 123, 126, 131, 51, 53 and 55, respectively;
the HCDR1, the HCDR2, the HCDR3, the LCDR1 the LCDR2 and the LCDR3 of SEQ ID NOs: 124, 127, 132, 134, 135 and 136, respectively; or
the HCDR1, the HCDR2, the HCDR3, the LCDR1, the LCDR2 and the LCDR3 of SEQ ID NOs: 125, 128, 133, 51, 53 and 55, respectively.

The invention also provides for an isolated antibody or an antigen-binding fragment thereof specifically binding HLA-DR, wherein the antibody or the antigen-binding fragment thereof comprises certain VH, VL. HC and LC amino acid sequences as described herein.

The invention also provides for an isolated antibody or an antigen-binding fragment thereof that specifically binds HLA-DR comprising
the HCDR1, the HCDR2, the HCDR3, LCDR1, the LCDR2 and the LCDR3 of SEQ ID NOs: 39, 42, 46, 50, 52 and 54, respectively;
the VH of SEQ ID NO: 56 and the VL of SEQ ID NO: 60; and/or
the HC of SEQ ID NO: 84 or 96 and the LC of SEQ ID NO: 88.

The invention also provides for an isolated antibody or an antigen-binding fragment thereof that specifically binds HLA-DR comprising
the HCDR1, the HCDR2, the HCDR3, the LCDR1, the LCDR2 and the LCDR3 of SEQ ID NOs: 40, 43, 47, 51, 53 and 55, respectively;
the VH of SEQ ID NO: 57 and the VL of SEQ ID NO: 61; and/or
the HC of SEQ ID NO: 85 or 97 and the LC of SEQ ID NO: 89.

The invention also provides for an isolated antibody or an antigen-binding fragment thereof that specifically binds HLA-DR comprising
the HCDR1, the HCDR2, the HCDR3, the LCDR1, the LCDR2 and the LCDR3 of SEQ ID NOs: 41, 44, 48, 51, 53 and 55, respectively;
the VH of SEQ ID NO: 58 and the VL of SEQ ID NO: 61; and/or
the HC of SEQ ID NO: 86 or 98 and the LC of SEQ ID NO: 89.

The invention also provides for an isolated antibody or an antigen-binding fragment thereof that specifically binds HLA-DR comprising
the HCDR1, the HCDR2, the HCDR3, the LCDR1, the LCDR2 and the LCDR3 of SEQ ID NOs: 41, 45, 49, 51, 53 and 55, respectively;
the VL of SEQ ID NO: 59 and the VL of SEQ ID NO: 61; and/or
the HC of SEQ ID NO: 87 or 99 and the LC of SEQ ID NO: 89.

The invention also provides for an antibody or an antigen-binding fragment thereof specifically binding HLA-DR of the invention conjugated to a heterologous molecule.

The invention also provides for a pharmaceutical composition comprising the antibody or the antigen-binding fragment thereof of the invention and a pharmaceutically accepted carrier.

The invention also provides for a polynucleotide encoding the VH, the VL, the VH and the VL, the HC, the LC or the HC and the LC of SEQ ID NOs: 56, 57, 58, 59, 60, 61, 84, 85, 86, 87, 96, 97, 98, 99, 137, 138, 139, 140, 141, 142, 149, 150, 151, 152, 154 or 154; or comprising the polynucleotide sequence of SEQ ID NOs: 79, 80, 81, 82, 83, 90, 91, 92, 93, 94, 95, 100, 101, 102, 103, 121, 143, 144, 145, 146, 147, 148, 155, 156, 157, 158, 159 or 160.

The invention also provides for a vector comprising the polynucleotide of the invention.

The invention also provides for a host cell comprising the vector of the invention.

The invention also provides for a method of producing the antibody or the antigen-binding fragment thereof of the invention, comprising culturing the host cell of the invention in conditions that the antibody is expressed, and recovering the antibody produced by the host cell.

The invention also provides for a method of treating or preventing HLA-DR-mediated disease, comprising administering to a subject in need thereof a therapeutically effective amount of the antibody or the antigen-binding fragment thereof of the invention for a time sufficient to treat HLA-DR-mediated disease.

The invention also provides for a method of suppressing an immune response towards a self-antigen, comprising administering to a subject in need thereof the antibody or the antigen-binding fragment thereof of the invention for a time sufficient to suppress the immune response towards a self-antigen.

The invention also provides for an method of treating HLA-DR expressing tumor, comprising administering to a subject in need thereof a therapeutically effective amount of the antibody or the antigen-binding fragment thereof of the invention conjugated to a cytotoxic agent for a time sufficient to treat HLA-DR expressing tumor.

The invention also provides for an anti-idiotypic antibody binding to the antibody or the antigen-binding fragment thereof of the invention.

The invention also provides for a kit comprising the antibody or the antigen-binding fragment of the invention.

The invention also provides the antibody of the invention for use in therapy.

### BRIEF DESCRIPTION OF THE DRAWINGS

FIG. 1 shows the HCDR1 amino acid sequences and the HCDR1 genus sequence of select antibodies. The genus sequence was determined by generating molecular models for all Fv (VH/VL pairs) in MOE (CCG, Montreal) using a default protocol for antibody modeling. For CDRs that have different lengths, these structural models were aligned based upon the structurally conserved regions and the structurally equivalent CDRs positions were identified.

FIG. 2 shows the HCDR2 amino acid sequences and the HCDR2 genus sequence of select antibodies. The HCDR2 genus sequence was generated as described in FIG. 1.

FIG. 3 shows the HCDR3 amino acid sequences and the HCDR3 genus sequence of select antibodies. The HCDR3 genus sequence was generated as described in FIG. 1.

FIG. 4 shows the LCDR1 amino acid sequences and the LCDR1 genus sequence of select antibodies. The LCDR1 genus sequence was generated as described in FIG. 1.

FIG. 5 shows the LCDR2 amino acid sequences and the LCDR2 genus sequence of select antibodies. The LCDR2 genus sequence was generated as described in FIG. 1.

FIG. 6 shows the LCDR3 amino acid sequences and the LCDR3 genus sequence of select antibodies. The LCDR3 genus sequence was generated as described in FIG. 1.

FIG. 7 shows the alignment of the amino acid sequences of the heavy chain variable regions (VH) of select antibodies specifically binding HLA-DR. The VH domains are identified by their SEQ ID NO: at the beginning of each row. CDR sequences (defined by Kabat) are underlined.

FIG. 8 shows the alignment of the amino acid sequences of the light chain variable domains (VL) of select antibodies specifically binding HLA-DR. The VL domains are identified by their SEQ ID NO: at the beginning of each row. CDR sequences (defined by Kabat) are underlined.

FIG. 9 shows the binding of the indicated antibodies to DR4G89 (HLA-DR4 in complex with hemagglutinin peptide HA_304-318) measured using Meso Scale Discovery (MSD) technology. ECL: electrochemiluminescence signal.

FIG. 10 shows the binding of the indicated antibodies to DR4G93 (HLA-DR1 in complex with hemagglutinin peptide HA_304-318) measured using MSD technology. ECL: electrochemiluminescence signal.

FIG. 11 shows the binding of the indicated antibodies to DR4G90 (HLA-DR4 in complex with collagen II peptide CII_1236-1249) measured using MSD technology. ECL: electrochemiluminescence signal.

FIG. 12 shows the binding of the indicated antibodies to DR4G99 (HLA-DR1 in complex with collagen II peptide CII_236-1249) measured using MSD technology. ECL: electrochemiluminescence signal.

FIG. 13 shows the frequency of dead B cells (% Annexin V⁺ Live/Dead⁺ CD3⁻ CD20⁺) in human PBMCs after 20 hours in culture with 2 pig/ml anti-HLA-DR antibodies as compared to an isotype control.

FIG. 14 shows the frequency of apoptotic B cells (% Annexin V⁺ Live/Dead⁺ CD3⁻ CD20⁺) in human PBMCs after 20 hours in culture with 2 μg/ml anti-HLA-DR antibodies as compared to an isotype control.

FIG. 15A shows the structure of HLA-DR4 (DR4G86) in complex with DR4B117.

FIG. 15B shows the structure of HLA-DR4 (DR4G86) in complex with DR4B127.

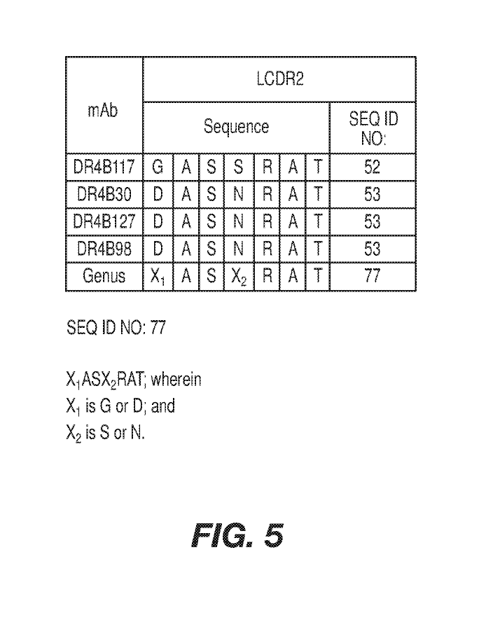

FIG. 5I shows the structure of HLA-DR4 in complex with T-cell receptor (TCR).

FIG. 16A shows that DR4B117 and DR4B127 do not block HLA-DR interaction with cognate TCR, whereas DR4B4, DR4B5 and DR4B6 do.

FIG. 16B shows that DR4B22. DR4B30 and DR4B33 do not block HLA-DR interaction with cognate TCR, whereas DR4B6 does.

### DETAILED DESCRIPTION OF THE INVENTION

All publications, including but not limited to patents and patent applications, cited in this specification are herein incorporated by reference as though fully set forth.

It is to be understood that the terminology used herein is for the purpose of describing particular embodiments only and is not intended to be limiting. Unless defined otherwise, all technical and scientific terms used herein have the same meaning as commonly understood by one of ordinary skill in the art to which the invention pertains.

Although any methods and materials similar or equivalent to those described herein may be used in the practice for testing of the present invention, exemplary materials and methods are described herein. In describing and claiming the present invention, the following terminology will be used.

As used in this specification and the appended claims, the singular forms “a.” “an,” and “the” include plural referents unless the content clearly dictates otherwise. Thus, for example, reference to “a cell” includes a combination of two or more cells, and the like.

“Specific binding”, “specifically binds”, “specifically binding” or “binds” refers to an antibody binding to an antigen or an epitope within the antigen with greater affinity than for other antigens. Typically, the antibody binds to the antigen or the epitope within the antigen with an equilibrium dissociation constant (K_{D}) of about 1×10⁻⁷ M or less, for example about 5×10⁻⁸ M or less, about 1×10⁻⁸ M or less, about 1×10⁻⁹ M or less, about 1×10⁻¹⁰ M or less, about 1×10⁻¹¹ M or less, or about 1×10⁻¹² M or less, typically with the K_{D} that is at least one hundred fold less than its K_{D} for binding to a non-specific antigen (e.g., BSA, casein). The dissociation constant may be measured using standard procedures. Antibodies that specifically bind to the antigen or the epitope within the antigen may, however, have cross-reactivity to other related antigens, for example to the same antigen from other species (homologs), such as human or monkey, for example Macaca fascicularis (cynomolgus, cyno), Pan troglodytes (chimpanzee, chimp) or Callithrix jacchus (common marmoset, marmoset). While a monospecific antibody specifically binds one antigen or one epitope, a bispecific antibody specifically binds two distinct antigens or two distinct epitopes. “Antibody specifically binding HLA-DR” or “an anti-HLA-DR antibody” refers to an antibody specifically binding at least HLA-DR4 composed of HLA-DRA1*01:02 α chain and a HLA-DRB1*04:01 β chain having amino acids sequences shown in SEQ ID NOs: 13 and 14, respectively. As various HLA-DR proteins are encoded by allelic variants of the genes encoding the HLA-DR α and HLA-DR β chains, the antibodies specifically binding HLA-DR may also specifically bind other HLA-DR proteins, such as HLA-DR1, HLA-DR3, HLA-DR10 and HLA-DR15.

“Antibodies” is meant in a broad sense and includes immunoglobulin molecules including monoclonal antibodies including murine, human, humanized and chimeric monoclonal antibodies, antigen-binding fragments, bispecific or multispecific antibodies, dimeric, tetrameric or multimeric antibodies, single chain antibodies, domain antibodies and any other modified configuration of the immunoglobulin molecule that comprises an antigen binding site of the required specificity. “Full length antibody molecules” are comprised of two heavy chains (HC) and two light chains (LC) inter-connected by disulfide bonds as well as multimers thereof (e.g. IgM). Each heavy chain is comprised of a heavy chain variable domain (VH) and a heavy chain constant domain, the heavy chain constant domain comprised of subdomains CH1, hinge, CH2 and CH3. Each light chain is comprised of a light chain variable domain (VL) and a light chain constant domain (CL). The VH and the VL may be further subdivided into regions of hypervariability, termed complementarity determining regions (CDR), interspersed with framework regions (FR). Each VH and VL is composed of three CDRs and four FR segments, arranged from amino-to-carboxy-terminus in the following order FR1, CDR1, FR2. CDR2. FR3, CDR3 and FR4.

“Complementarity determining regions (CDR)” are “antigen binding sites” in an antibody. CDRs may be defined using various terms: (i) Complementarity Determining Regions (CDRs), three in the VH (HCDR1, HCDR2, HCDR3) and three in the VL (LCDR1, LCDR2, LCDR3) are based on sequence variability (Wu and Kabat, (1970) J Exp Med 132:211-50; Kabat et al., Sequences of Proteins of Immunological Interest 5th Ed. Public Health Service, National Institutes of Health, Bethesda, Md., 1991). (ii) “Hypervariable regions”, “HVR”, or “HV”, three in the VH (H1, H2, H3) and three in the VL (L1, L2, L3) refer to the regions of an antibody variable domains which are hypervariable in structure as defined by Chothia and Lesk (Chothia and Lesk, (1987) Mol Biol 196:901-17). The International ImMunoGeneTics (IMGT) database (http://www_imgt_org) provides a standardized numbering and definition of antigen-binding sites. The correspondence between CDRs. HVs and IMGT delineations is described in Lefranc et al., (2003) Dev Comparat Immunol 27:55-77. The term “CDR”, “HCDR1”. “HCDR2”, “HCDR3”, “LCDR1”, “LCDR2” and “LCDR3” as used herein includes CDRs defined by any of the methods described supra. Kabat Chothia or IMGT, unless otherwise explicitly stated in the specification.

Immunoglobulins may be assigned to five major classes, IgA. IgD, IgE. IgG and IgM, depending on the heavy chain constant region amino acid sequence. IgA and IgG are further sub-classified as isotypes IgA1, IgA2, IgG1, IgG2, IgG3 and IgG4. Antibody light chains of any vertebrate species may be assigned to one of two clearly distinct types, namely kappa (κ) and lambda (λ), based on the amino acid sequences of their constant domains.

“Antigen-binding fragment” refers to a portion of an immunoglobulin molecule that retains the antigen binding properties of the parental full length antibody. Exemplary antigen-binding fragments are heavy chain complementarity determining regions (HCDR) 1, 2 and/or 3, light chain complementarity determining regions (LCDR) 1, 2 and/or 3, the VH, the VL, the VH and the VL, Fab, F(ab′)2, Fd and Fv fragments as well as domain antibodies (dAb) consisting of either one VH domain or one VL domain. The VH and the VL domains may be linked together via a synthetic linker to form various types of single chain antibody designs in which the VH/VL domains pair intramolecularly, or intermolecularly in those cases when the VH and VL domains are expressed by separate chains, to form a monovalent antigen binding site, such as single chain Fv (scFv) or diabody; described for example in Int Pat. Publ. No. WO1998/44001. Int. Pat. Publ. No. WO1988/01649; Int. Pat. Publ. No. WO1994/13804; Int. Pat. Publ. No. WO1992/01047.

“Monoclonal antibody” refers to an antibody population with single amino acid composition in each heavy and each light chain, except for possible well known alterations such as removal of C-terminal lysine from the antibody heavy chain. Monoclonal antibodies typically bind one antigenic epitope, except that bispecific monoclonal antibodies bind two distinct antigenic epitopes. Monoclonal antibodies may have heterogeneous glycosylation within the antibody population. Monoclonal antibody may be monospecific or multispecific, or monovalent, bivalent or multivalent. A bispecific antibody is included in the term monoclonal antibody.

“Isolated” refers to a homogenous population of molecules (such as synthetic polynucleotides or a protein such as an antibody) which have been substantially separated and/or purified away from other components of the system the molecules are produced in, such as a recombinant cell, as well as a protein that has been subjected to at least one purification or isolation step. “Isolated antibody specifically binding HLA-DR” refers to an antibody that is substantially free of other cellular material and/or chemicals and encompasses antibodies that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

“Humanized antibody” refers to an antibody in which the antigen binding sites are derived from non-human species and the variable region frameworks are derived from immunoglobulin sequences of human origin. Humanized antibody may include substitutions in the framework so that the framework may not be an exact copy of expressed human immunoglobulin or human immunoglobulin germline gene sequences.

“Human antibody” refers to an antibody having heavy and light chain variable domains in which both the framework and the antigen binding sites are derived from sequences of human origin. If the antibody contains a constant domain or a portion of the constant domain, the constant domain is also derived from sequences of human origin.

Human antibody comprises heavy or light chain variable domains that are “derived from” sequences of human origin if the variable domains of the antibody are obtained from a system that uses human germline immunoglobulin or rearranged immunoglobulin genes. Such exemplary systems are human immunoglobulin gene libraries displayed on phage or on mammalian cells, and transgenic non-human animals such as mice or rats carrying human immunoglobulin loci as described herein. “Human antibody” may contain amino acid differences when compared to the human germline immunoglobulin or rearranged immunoglobulin genes due to for example naturally occurring somatic mutations or intentional introduction of substitutions into the framework or antigen binding site, or both. Typically. “human antibody” is at least about 80%, 81%, 82%, 83%, 84%, 85%, 86%, 87%, 88%, 89%, 90%, 91%, 92%, 93%, 94%, 95%, 96%, 97%, 98%, 99% or 100% identical in amino acid sequence to an amino acid sequence encoded by human germline immunoglobulin or rearranged immunoglobulin genes. In some cases. “human antibody” may contain consensus framework sequences derived from human framework sequence analyses, for example as described in Knappik et al., (2000) J Mol Biol 296:57-86, or synthetic HCDR3 incorporated into human immunoglobulin gene libraries displayed on phage, for example as described in Shi et al., (2010) J Mol Biol 397:385-96, and in Int. Patent Publ. No. WO2009/085462.

Human antibodies derived from human immunoglobulin sequences may be generated using systems such as phage display incorporating synthetic CDRs and/or synthetic frameworks, or may be subjected to in vitro mutagenesis to improve antibody properties, resulting in antibodies that are not expressed by the human antibody germline repertoire in vivo.

Antibodies in which antigen binding sites are derived from a non-human species are not included in the definition of “human antibody”.

“Recombinant” refers to antibodies and other proteins that are prepared, expressed, created or isolated by recombinant means. “Recombinant antibody” includes all antibodies that are prepared, expressed, created or isolated by recombinant means, such as antibodies isolated from an animal (e.g., a mouse) that is transgenic or transchromosomal for human immunoglobulin genes or a hybridoma prepared therefrom (described further below), antibodies isolated from a host cell transformed to express the antibody, antibodies isolated from a recombinant, combinatorial antibody library, and antibodies prepared, expressed, created or isolated by any other means that involve splicing of human immunoglobulin gene sequences to other DNA sequences, or antibodies that are generated in vitro using Fab arm exchange such as bispecific antibodies.

“Epitope” refers to a portion of an antigen to which an antibody specifically binds. Epitopes typically consist of chemically active (such as polar, non-polar or hydrophobic) surface groupings of moieties such as amino acids or polysaccharide side chains and may have specific three-dimensional structural characteristics, as well as specific charge characteristics. An epitope may be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule.

“Paratope” refers to a portion of an antibody to which an antigen specifically binds. A paratope may be linear in nature or may be discontinuous, formed by a spatial relationship between non-contiguous amino acids of an antibody rather than a linear series of amino acids. A “light chain paratope” and a “heavy chain paratope” or “light chain paratope amino acid residues” and “heavy chain paratope amino acid residues” refer to antibody light chain and heavy chain residues in contact with an antigen, respectively, or in general, “antibody paratope residues” refer to those antibody amino acids that are in contact with antigen.

“Bispecific” refers to an antibody that specifically binds two distinct antigens or two distinct epitopes within the same antigen. The bispecific antibody may have cross-reactivity to other related antigens or can bind an epitope that is shared between two or more distinct antigens.

“Multispecific” refers to an antibody that specifically binds at least two distinct antigen or at least two distinct epitopes within the same antigen. Multispecific antibody may bind for example two, three, four or five distinct antigens or distinct epitopes within the same antigen.

“Polynucleotide” refers to a synthetic molecule comprising a chain of nucleotides covalently linked by a sugar-phosphate backbone or other equivalent covalent chemistry. cDNA is a typical example of a synthetic polynucleotide.

“Polypeptide” or “protein” refers to a molecule that comprises at least two amino acid residues linked by a peptide bond to form a polypeptide.

“Peptide” refers to a short polypeptide up to 30 amino acids long.

“Variant” refers to a polypeptide or a polynucleotide that differs from a reference polypeptide or a reference polynucleotide by one or more modifications, for example one or more substitutions, insertions or deletions.

“Vector” refers to a polynucleotide capable of being duplicated within a biological system or that can be moved between such systems. Vector polynucleotides typically contain elements, such as origins of replication, polyadenylation signal or selection markers that function to facilitate the duplication or maintenance of these polynucleotides in a biological system, such as a cell, virus, animal, plant, and reconstituted biological systems utilizing biological components capable of duplicating a vector. The vector polynucleotide may be DNA or RNA molecules or a hybrid of these, single stranded or double stranded.

“Expression vector” refers to a vector that can be utilized in a biological system or in a reconstituted biological system to direct the translation of a polypeptide encoded by a polynucleotide sequence present in the expression vector.

“About” means within an acceptable error range for the particular value as determined by one of ordinary skill in the art, which will depend in part on how the value is measured or determined. i.e., the limitations of the measurement system. Unless explicitly stated otherwise within the Examples or elsewhere in the Specification in the context of a particular assay, result or embodiment “about” means within one standard deviation per the practice in the art, or a range of up to 5%, whichever is larger.

“Sample” refers to a collection of similar fluids, cells, or tissues isolated from a subject, as well as fluids, cells, or tissues present within a subject. Exemplary samples are biological fluids such as blood, serum and serosal fluids, plasma, lymph, urine, saliva, cystic fluid, tear drops, feces, sputum, mucosal secretions of the secretory tissues and organs, vaginal secretions, ascites fluids, fluids of the pleural, pericardial, peritoneal, abdominal and other body cavities, fluids collected by bronchial lavage, synovial fluid, liquid solutions contacted with a subject or biological source, for example, cell and organ culture medium including cell or organ conditioned medium, lavage fluids and the like, tissue biopsies, fine needle aspirations, surgically resected tissue, organ cultures or cell cultures.

“In combination with” means that two or more therapeutics are administered to a subject together in a mixture, concurrently as single agents or sequentially as single agents in any order.

“Antagonist” refers to a molecule that, when bound to a cellular protein, suppresses at least one reaction or activity that is induced by a natural ligand of the protein. A molecule is an antagonist when the at least one reaction or activity is suppressed by at least about 30%, 40%, 45%, 50%, 55%, 60%, 65%, 70%, 75%, 80%, 85%, 90%, 95%, or 100% more than the at least one reaction or activity suppressed in the absence of the antagonist (e.g., negative control), or when the suppression is statistically significant when compared to the suppression in the absence of the antagonist. An exemplary antagonist is an antibody specifically binding HLA-DR that inhibits activation of T cells, for example proliferation of CD4⁺ T cells.

“Subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. “Patient” and “subject” are used interchangeably herein.

“Human leukocyte antigen HLA-DR” or “HLA-DR” refers to a major histocompatibility complex (MHC) class II cell surface receptor. HLA-DR is a heterodimer of α and β chains with each subunit spanning the membrane once. HLA-DR α chain is encoded by HLA-DRA1 and HLA-DR β chain is encoded by HLA-DRB1 or one of its paralogues HLA-DRB3, HLA-DRB4, or HLA-DRB5. HLA-DRB1, as is well known, is hyperpolymorphic. Nomenclature. cDNA and amino acid sequences of various HLA-DR α and HLA-DR β chains are well known. For example, the international ImMunoGeneTics information System® (IMGT®) database provides the amino acid sequences of the proteins encoded by HLA-DRA1 and HLA-DRB as well as their amino acid alignments. HLA Nomenclature provides HLA gene and protein sequences and statistics for HLA allele numbers that can be found at Http:_/_hla_alleles_org and cited in Robinson et al., Nucleic Acids Research (2015) 43:D423-431 and March et al., Tissue Antigens (2010) 75:291-455.

“HLA-DR4” or “DR4” refers to particular HLA antigens within serological group 4. HLA-DR4α chain is encoded by HLA-DRA1*01 and HLA-DR4 β chain is encoded by HLA-DRB1*04. HLA-DRB1*04 is polymorphic and encodes various variants including HLA-DRB1*04:01. HLA-DRB1*04:02, HLA-DRB1*04:03, HLA-DRB1*04:04, HLA-DRB1*04:05, etc, well known to those in the field.

“HLA-DR1” or “DR1” refers to particular HLA antigens within serological group 1. HLA-DR1α chain is encoded by HLA-DRA1*01 and HLA-DR1 β chain is encoded by the HLA-DRB1*01 gene. HLA-DRB1*01 is polymorphic and encodes various variants including HLA-DRB1*01:01. HLA-DRB1*01:02. HLA-DRB1*01:03. HLA-DRB1*01:04, HLA-DRB1*01:05, etc, well known to those in the field.

“HLA-DR3” or “DR3” refers to particular HLA antigens within serological group 3. HLA-DR3α chain is encoded by HLA-DRA1*01 and HLA-DR3 β chain is encoded by the HLA-DRB1*03 gene. HLA-DRB1*03 is polymorphic and encodes various variants including HLA-DRB1*03:01. HLA-DRB1*03:02, HLA-DRB1*03:03, HLA-DRB1*03:04, HLA-DRB1*03:05, etc, well known to those in the field.

“HLA-DR10” or “DR10” refers to particular HLA antigens within serological group 10. HLA-DR10α chain is encoded by HLA-DRA1*01 and HLA-DR10β chain is encoded by the HLA-DRB1*10 gene. HLA-DRB1*10 is polymorphic and encodes various variants including HLA-DRB1*10:01, HLA-DRB1*10:02, HLA-DRB1*10:03, HLA-DRB1*10:04. HLA-DRB1*10:05. etc. well known to those in the field.

“HLA-DR15” or “DR15” refers to particular HLA antigens within serological group 15. HLA-DR15α chain is encoded by HLA-DRA1*01 and HLA-DR15 β chain is encoded by the HLA-DRB1*15 gene. HLA-DRB1*15 is polymorphic and encodes various HLA-DRB1 proteins including HLA-DRB1*15:01, HLA-DRB1*15:02. HLA-DRB1*15:03. HLA-DRB1*15:04. HLA-DRB1*15:05, etc, well known to those in the field.

“Shared epitope” refers to a common structural motif shared by certain HLA-DRB1 alleles in the third hypervariable region of their β chains. This common motif extends five amino acids on the side of the peptide binding pocket (residues 70-74) and has the amino acid sequence of QKRAA (SEQ ID NO: 66), QRRAA (SEQ ID NO: 67) or RRRAA (SEQ ID NO: 68). The shared epitope is present in HLA-DRB1 alleles HLA-DRB1*01:01, *01:02, *04:01, *04:04, *04:05, *04:08, and *10:01.

“Apoptosis”, as used herein refers to the process of programmed cell death (PCD) that may occur in a cell.

“Death of B cells” refers to B cell death by an accidental manner (necrosis), which is a form of cell death that results from acute tissue injury and provokes an inflammatory response, cell death by apoptosis, or by any other means.

“In complex” or “complexed” refers to the complex of HLA-DR α chain. HLA-DR β chain and one peptide residing in the well-known peptide binding groove in the HLA-DR molecule. In vivo, the peptide/HLA-DR interaction is non-covalent. In vitro, the peptide may be covalently coupled for example to the N-terminus of the β chain. Therefore, “in complex” encompasses HLA-DR complexes with both non-covalently and covalently bound peptides.

“T cell activation” refers to one or more cellular responses of a T cell, for example a CD4⁺ T cell, such as proliferation, differentiation, cytokine secretion, cytotoxic effector molecule release, cytotoxic activity and expression of activation markers.

“HLA-DRB1-associated autoimmune disease” refers to an autoimmune disease in which genetic association has been or will be identified with certain HLA-DRB1 allele, alleles or haplotypes.

“HLA-DR-mediated disease” refers to a disease that is mediated at least part by HLA-DR binding to T cell receptor (TCR).

The numbering of amino acid residues in the antibody constant region throughout the specification is according to the EU index as described in Kabat et al., Sequences of Proteins of Immunological Interest, 5th Ed. Public Health Service. National Institutes of Health. Bethesda, Md. (1991), unless otherwise explicitly stated.

Conventional one and three-letter amino acid codes are used herein as shown in Table 1.

TABLE 1

| | Three-letter | One-letter |
|---|---|---|
| Amino acid | code | code |
| Alanine | Ala | A |
| Arginine | Arg | R |
| Asparagine | Asn | N |
| Aspartate | Asp | D |
| Cysteine | Cys | C |
| Glutamate | Gln | E |
| Glutamine | Glu | Q |
| Glycine | Gly | G |
| Histidine | His | H |
| Isoleucine | Ile | I |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

### Compositions of Matter

The present invention provides antibodies specifically binding HLA-DR which inhibit CD4⁺ T cell activation. The antibodies optionally are non-depleting and demonstrate no binding HLA-DP or HLA-DQ and therefore may provide an improved safety profile by interfering only with the presentation of self-peptides associated with autoimmune diseases while having no effect on presentation of other peptides on HLA-DP or HLA-DQ needed to generate immune responses during infection. The present invention provides polypeptides and polynucleotides encoding the antibodies of the invention or complementary nucleic acids thereof, vectors, host cells, and methods of making and using them.

The invention provides an isolated antibody or an antigen-binding fragment thereof specifically binding HLA-DR, wherein the antibody or the antigen-binding fragment thereof competes for binding to HLA-DR with an antibody comprising
a) a heavy chain variable domain (VH) of SEQ ID NO: 58 and a light chain variable domain (VL) of SEQ ID NO: 61;
b) the VH of SEQ ID NO: 56 and the VL of SEQ ID NO: 60;
c) the VH of SEQ ID NO: 57 and the VL of SEQ ID NO: 61;
d) the VH of SEQ ID NO: 137 and the VL of SEQ ID NO: 61;
e) the VH of SEQ ID NO: 138 and the VL of SEQ ID NO: 61;
f) the VH of SEQ ID NO: 139 and the VL of SEQ ID NO: 61;
g) the VH of SEQ ID NO: 140 and the VL of SEQ ID NO: 142; or
h) the VH of SEQ ID NO: 141 and the VL of SEQ ID NO: 61.

Antibodies comprising the VH and the VL of SEQ ID NOs: 58 and 61 (mAb DR4B127) or 56 and 60 (mAb DR4B117), respectively, were identified to inhibit CD4⁺ T cell activation by a unique mechanism. DR4B127 and DR4B117 bound HLA-DR on the CD4 binding site instead of interfering with interaction of HLA-DR with cognate T cell receptor (TCR). By not wishing to be bound by any particular theory, DR4B127 and DR4B117 may induce conformational and/or spatial changes in the HLA-DR molecule hence preventing the interaction between HLA-DR and cognate T cell receptor or between HLA-DR and the T cell co-receptor CD4. DR4B127 and DR4B117 thereby may acutely disrupt T cell signaling, but may also induce long-term suppression of HLA-DR-restricted T cells. Prolonged lack of memory T cell contact with HLA-DR due to the presence of the antibody could lead to loss from the T cell pool. Antibodies that bind HLA-DR and interfere with the association of CD4 may allow continued unproductive HLA-DR/TCR engagement without the association of CD4, abrogating costimulation and resulting in anergy. Therefore antibodies that prevent T cell activation by blocking HLA-DR at non-TCR site (e.g. DR4B127 and DR4B117 and antibodies that cross-compete for binding to HLA-DR with DR4B127 and DR4B117) may be beneficial in not only treatment but also in prevention of autoimmune diseases. Exemplary antibodies that cross-compete for binding to HLA-DR are antibodies DR4B30, DR4B117, DR4B127, DR4B78, DR4B38, DR4B70. DR4B22 and DR4B33. As was demonstrated herein, control antibodies DR4B4, DR4B5 and DR4B6 blocked HLA-DR binding to TCR.

Competition for binding to HLA-DR with antibodies or antigen-binding fragments thereof of the invention comprising certain VH and VL sequences may be assayed in vitro using known methods. For example, binding of MSD Sulfo-Tag™ NHS-ester-labeled antibody to soluble recombinant HLA-DR in the presence of an unlabeled antibody maybe assessed by ELISA, or Biacore analyses or flow cytometry may be used to demonstrate competition with the antibodies of the current invention. In an exemplary assay, 5 μl of 10 μg/ml of soluble HLA-DR molecule DR4G89 or DR4G99 (described herein) are absorbed on Meso Scale Discovery (MSD) HighBind plates (Gaithersburg, Md.) for 2 hours then washed 3× with 150 μl 0.1M HEPES. Plate is blocked with 5% BSA buffer overnight at 4° C. The next day, plates are washed 3× with 0.1 M HEPES buffer, pH 7.4, followed by the addition of the mixture of Ruthenium (Ru)-labeled reference HLA-DR mAb which is pre-incubated at room temperature for 30 minutes with 1 mM of the test HLA-DR mAbs. After incubation with gentle shaking at room temperature 2 hours, plates are washed 3× with 0.1M HEPES buffer (pH 7.4). MSD Read Buffer T is diluted with distilled water (4-fold) and dispensed into each well then analyzed with a SECTOR Imager 6000 (Meso Scale Discovery, Gaithersburg, Md.). The test antibodies compete for binding to HLA-DR with the reference antibody when the test antibody inhibits binding of the reference antibody to HLA-DR by 80% or more, for example 85% or more, 90% or more, or 95% or more.

In some embodiments, the antibody or the antigen-binding fragment thereof of the invention is an antagonist of HLA-DR.

In some embodiments, the antibody or the antigen-binding fragment thereof of the invention inhibits T cell activation.

T cell activation may be T cell proliferation, differentiation, cytokine secretion, cytotoxic effector molecule release, cytotoxic activity or expression of activation markers. T cell may be a CD4⁺ T cell. Exemplary antibodies that inhibit T cell activation are antibodies DR4B30, DR4B117, DR4B127, DR4B78, DR4B38, DR4B70. DR4B22, DR4B98 and DR4B33 described herein.

T cell activation may be determined using a mixed lymphocyte reaction (MLR) in which dendritic cells or other antigen-presenting cells are co-cultured with CD4⁺ T cells, and the proliferation of the cell is determined by 3H-thymidine incorporation and by using methods described herein. The antibody “inhibits T cell activation” when at least one characteristics of T cell activation (e.g. proliferation, differentiation, cytokine secretion cytotoxic effector molecule release, cytotoxic activity or expression of activation markers) is inhibited by 30%, 40%, 45%, 50%, 55%, 60%, 650%, 70%, 75%, 80%, 85%, 90%, 95%, or 100% when compared to the isotype control, or is inhibited in a statistically significant manner when compared to inhibition in the presence of an isotype control. “Isotype control” is well known. Alternatively, activation of CD4⁺ T cells may be assessed by measuring, increased interferon-γ (IFN-γ) or TNF-α secretion in the MLR assay.

In some embodiments, the antibody or the antigen-binding fragment thereof of the invention inhibits CD4⁺ T cell proliferation at antibody concentration of 1 μg/ml by at least 30% in a co-culture of human CD4⁺ T cells and dendritic cells isolated from transgenic animals expressing human HLA-DR4.

Exemplary transgenic animals expressing human HLA-DR4 are mice strain 4149. Taconic Biosciences. These mice express human HLA-DRA1*01 and HLA-DRB1*04:01 engineered to membrane proximal domains of mouse I-E (H2-E).

In some embodiments, the antibody or the antigen-binding fragment thereof of the invention does not inhibit HLA-DR binding to a cognate T cell receptor (TCR).

In some embodiments, the antibody or the antigen-binding fragment thereof of the invention does not inhibit binding of HLA-DR4 comprising HLA-DR α chain of SEQ ID NO: 13 and HLA-DR β chain of SEQ ID NO: 14 in complex with the hemagglutinin peptide of SEQ ID NO: 7 to the cognate TCR.

In some embodiments, the antibody or the antigen-binding fragment thereof of the invention inhibits binding of HLA-DR to CD4.

“Inhibit binding” refers to a measurable reduction in binding of HLA-DR to CD4 or the cognate TCR in the presence of the antibody when compared to the isotype control. Inhibition may for example 30%, 40%, 45%, 50%, 55%, 60%, 65%, 70%, 75%, 80%, 85%, 90%, 95%, or 100% reduction in binding when compared to the isotype control, or inhibition in a statistically significant manner when compared to inhibition in the presence of an isotype control. Thus, the antibody does not inhibit HLA-DR binding to the cognate TCR when the inhibition is less than 29% or statistically insignificant when compared to the isotype control.

Inhibition of binding of HLA-DR to TCR may be conducted using standard ELISA experiments. In an exemplary assay, 50 μl of HLA-DR antigen DR4G134 (described herein) at 5 μg/ml is coated on MDS plates, the plates are shaken for 10 minutes at room temperature and incubated overnight at 4° C. The plates are blocked in assay buffer (1×DPBS, 1% BSA, 0.05% tween 20) and a mixture or test antibodies at concentration range of 10⁻² to 10² mg/ml, 5 μg/ml of recombinant TCR (DRG79, described herein), 10 μg/ml anti-histidine antibody and 2 μg/ml SulfoTag-SA are added onto wells. The plates are incubated for 1 hour, washed, and read in MSD after addition of 150 μl of MSD read buffer.

In some embodiments, the antibody or the antigen-binding fragment thereof has one, two, three, four or five of the following properties:
a) binds HLA-DR4 comprising HLA-DR α chain of SEQ ID NO: 13 and HLA-DR β chain of SEQ ID NO: 14 in complex with the hemagglutinin peptide of SEQ ID NO: 7 with an equilibrium dissociation constant (K_{D}) of 5×10⁻⁸ M or less, wherein K_{D} is measured using ProteOn XPR36 system at 25° C. in a buffer containing DPBS, 0.01% (w/v) polysorbate 20 (PS-20) and 100 μg/ml BSA;
b) binds HLA-DR1 comprising HLA-DR α chain of SEQ ID NO: 13 and the HLA-DR β chain of SEQ ID NO: 15 in complex with the hemagglutinin peptide of SEQ ID NO: 7 with an equilibrium dissociation constant (K_{D}) of 5×10⁻⁸ M or less, wherein K_{D} is measured using ProteOn XPR36 system at 25° C. in a buffer containing DPBS, 0.01% (w/v) PS-20 and 100 μg/ml BSA;
c) lacks an ability to induce apoptosis of B cells, wherein apoptosis is determined by measuring frequency of CD3⁻ CD20⁺ annexinV⁺live/dead⁻ B cells in a sample of human peripheral blood cells (PBMC) using flow cytometry;
d) lacks an ability to induce death of B cells, wherein death of B cells is determined by measuring frequency of CD3⁻ CD20⁺ annexinV⁺live/dead⁺ B cells in the sample of human PBMC using flow cytometry; or
e) inhibits binding of HLA-DR to CD4.

Exemplary antibodies that lack the ability to induce apoptosis of B cells are antibodies DR4B117. DR4B30, DR4B127, DR4B98 and DR4B33 described herein. These antibodies may have an improved safety profile when compared to the antibodies that induce death of B cells, such as the control antibodies DR4B4, DR4B5 and DR4B6.

B cell apoptosis may be measured using flow cytometry and identifying apoptotic B cells as CD3⁻ CD20⁺ annexinV⁺live/dead⁻ B cells in a sample, for example in a sample of human peripheral blood mononuclear cells (PBMCs). The antibodies of the invention “lack the ability to induce apoptosis of B cells” when there is statistically insignificant increase in B cell apoptosis in a sample treated with the test antibody when compared to a sample treated with isotype control. “Live/dead” refers to a fluorescent dye which discriminates between live and dead cells regardless of the mechanism of cell death, such as eF660 from BioLegend.

Exemplary antibodies that lack the ability to induce death of B cells are antibodies DR4B117, DR4B30. DR4B127. DR4B98 and DR4B33 described herein. These antibodies may have an improved safety profile when compared to the antibodies that induce death of B cells, such as the control antibodies DR4B4, DR4B5 and DR4B6.

B cell death may be measured using flow cytometry and identifying dead B cells as CD3⁻ CD20⁺ annexinV⁺live/dead⁺ B cells in a sample, for example in a sample of human peripheral blood cells (PBMCs). The antibodies of the invention “lack the ability to induce death of B cells” when there is statistically insignificant increase in B cell death in a sample treated with the test antibody when compared to a sample treated with isotype control.

Inhibition of binding of HLA-DR to CD4 may be measured using ELISA using known protocols and HLA-DR antigens described herein.

In some embodiments, HLA-DR is HLA-DR4, HLA-DR1, HLA-DR3, HLA-DR10 or HLA-DR15.

In some embodiments. HLA-DR4 comprises HLA-DRA*01:02 of SEQ ID NO: 13 and HLA-DRB1*04:01 of SEQ ID NO: 14.

In some embodiments. HLA-DR1 comprises HLA-DRA1*01:02 of SEQ ID NO: 13 and HLA-DRB1*01:01 of SEQ ID NO: 15.

In some embodiments, HLA-DR4 comprises HLA-DRA1*01:02 of SEQ ID NO: 13 and HLA-DRB1*04:02 of SEQ ID NO: 106.

In some embodiments, HLA-DR3 comprises HLA-DRA1*01:02 of SEQ ID NO: 13 and HLA-DRB1*03:01 of SEQ ID NO: 105.

In some embodiments, HLA-DR10 comprises HLA-DRA1*01:02 of SEQ ID NO: 13 and HLA-DRB1*10:01 of SEQ ID NO: 107.

In some embodiments, HLA-DR15 comprises HLA-DRA1*01:02 of SEQ ID NO: 13 and HLA-DRB1*15:01 of SEQ ID NO: 108.

The nomenclature and amino acid sequences of various HLA-DR α and β chains are well known. The antibodies of the invention, given that HLA-DRB1 is hyperpolymorphism, may specifically bind multiple HLA-DR molecules.

The invention also provides for an isolated antibody or an antigen-binding fragment thereof specifically binding HLA-DR4 wherein the antibody or the antigen-binding fragment thereof binds HLA-DR4 with an equilibrium dissociation constant (K_{D}) of less than about 5×10⁻⁸ M or less.

The affinity of an antibody or the antigen-binding fragment thereof to HLA-DR4 or to other HLA-DR molecules may be determined experimentally using any suitable method. Such methods may utilize ProteOn XPR36, Biacore 3000 or KinExA instrumentation, ELISA or competitive binding assays known to those skilled in the art. The measured affinity of a particular antibody/HLA-DR interaction may vary if measured under different conditions (e.g., osmolarity, pH). Thus, measurements of affinity and other binding parameters (e.g., K_{D}, Kₒₙ, K_{off}) are typically made with standardized conditions and a standardized buffer, such as the buffer described herein. The internal error for affinity measurements for example using Biacore 3000 or ProteOn (measured as standard deviation. SD) may typically be within 5-33% for measurements within the typical limits of detection. Therefore the term “about” in the context of K_{D} reflects the typical standard deviation in the assay. For example, the typical SD for a K_{D} of 1×10⁻⁹ M is up to ±0.33×10⁻⁹ M.

The HLA-DR molecules used in the experiments described herein may be expressed as soluble Fc-fusion proteins. A peptide that is presented on HLA-DR may be covalently coupled to the N-terminus of the HLA-DR β chain to facilitate expression. Tags such as hexahistidine (SEQ ID NO: 3) or StrepII tag (SEQ ID NO: 6) may be covalently linked to the a and/or p chain or to the Fc to facilitate purification of the expressed protein. Linkers may be inserted between the presented peptide, a and/or J chain, the Fc portion and/or the various tags. Suitable linkers may be a glycine/serine linker (SEQ ID NO: 1 or 4), tobacco etch virus Nia protease cleavage site (SEQ ID NO: 2), or human rhinovirus 3C protease cleavage site (SEQ ID NO: 5). Suitable peptides that may be presented on HLA-DR may be a hemagglutinin peptide HA_304-318 (SEQ ID NO: 7), collagen II peptides CII_1236-1249 or CII_257-273 (SEQ ID NO: 8 and SEQ ID NO: 9, respectively) vimentin peptide (SEQ ID NO: 71), aggrecan peptide (SEQ ID NO: 72), CLIP peptide (SEQ ID NO: 104) or collagen II peptide CII_259-273 (SEQ ID NO: 122). Exemplary HLA-DR molecules that may be expressed may have following configurations:

α chain: extracellular domain or the particular α chain, linker of SEQ ID NO: 1, linker of SEQ ID NO: 2, linker of SEQ ID NO: 1, Fc domain, tag of SEQ ID NO: 3
β chain: peptide of SEQ ID NO: 7, linker of SEQ ID NO: 4, linker of SEQ ID NO: 5, extracellular domain of the particular β chain, linker of SEQ ID NO: 4, linker of SEQ ID NO: 2, linker of SEQ ID NO: 4, Fc domain, tag of SEQ ID NO: 6. The α and β chains are co-expressed, and the resulting heterodimers may be purified for example using the His6 and StrepII tags using standard methods. HLA-DP and HLA-DQ molecules may be similarly expressed.

The invention also provides for an isolated antibody or an antigen-binding fragment thereof specifically binding HLA-DR, wherein HLA-DR contains a shared epitope consisting of an amino acid sequence QKRAA (SEQ ID NO: 66), QRRAA (SEQ ID NO: 67), or RRRAA (SEQ ID NO: 68).

The Shared Epitope on HLA-DR alleles HLA-DRB1*01:01, *01:02, *04:01, *0404, *04:05, *04:08, and *10:01 is a motif of five amino acid residues QKRAA (SEQ ID NO: 66). QRRAA (SEQ ID NO: 67) or RRRAA (SEQ ID NO: 68) at residue positions 70-74 in the HLA-DR β chain.

HLA-DR alleles with the shared epitope are associated with autoimmune diseases such as RA, and they have been shown to present citrullinated peptides recognized as non-self by T cells with high affinity. For example, in RA, autoantibodies recognizing citrullinated proteins (ACPA) are present in the serum before the onset of disease (up to 14 years prior to disease) and show a marked increase ˜2 years prior to RA diagnosis (Rantapaa-Dahlqvist et al., Arthritis Rheum 2003; 48(10):2741-9; Nielen et al., Arthritis Rheum 2004; 50(2):380-6; Van de Stadt et al., Arthritis Rheum 2011; 63(11):3226-33). HLA-DRB1 has been found to be associated with the risk to progress from a healthy ACPA+ individual to an ACPA+ individual with RA (Hensvold et al., Ann Rheum Dis 2015; 74(2):375-80). Therefore the detection of ACPA prior to the onset of RA presents a window of opportunity in which treatment with antibodies specifically binding HLA-DR could abrogate T cell activation to prevent further increases in serum autoantibody levels and dampen the increasing inflammation that leads to RA diagnosis. Antibodies inhibiting T cell activation by either blocking the interaction between HLA-DR molecule containing the shared epitope and a cognate T cell receptor or by inducing conformational (and/or spatial changes) in the HLA-DR molecule, thus preventing the interaction between HLA-DR and cognate T cell receptor, may be beneficial in not only treatment but also in prevention of autoimmune diseases.

Exemplary antibodies that bind HLA-DR containing the shared epitope are antibodies DR4B117, DR4B30, DR4B127, DR4B98, DR4B78, DR4B38. DR4B70, DR4B22 and DR4B33 described herein.

In some embodiments, HLA-DR is in complex with a peptide.

In some embodiments, the peptide comprises an amino acid sequence of SEQ ID NOs: 7, 8 or 9, 71, 72, 104 or 122.

In some embodiments, the peptide consists of an amino acid sequence of SEQ ID NOs: 7, 8 or 9, 71, 72, 104 or 122.

The invention also provides for an isolated antibody or an antigen-binding fragment thereof specifically binding HLA-DR comprising a heavy chain complementarity determining region 1, 2 and 3 (a HCDR1, a HCDR2 and a HCDR3) of SEQ ID NOs: 73, 74 and 75, respectively.

SEQ ID NOs: 73, 74 and 75 represent the HDR1, the HCDR2 and the HCDR3 genus amino acid sequences of the antibodies of the antigen-binding fragments thereof specifically binding HLA-DR, respectively. Antibodies within the genus inhibit T cell activation and lack the ability to induce death of B cells. Exemplary such antibodies are antibodies DR4B127 and DR4B98 as described herein.

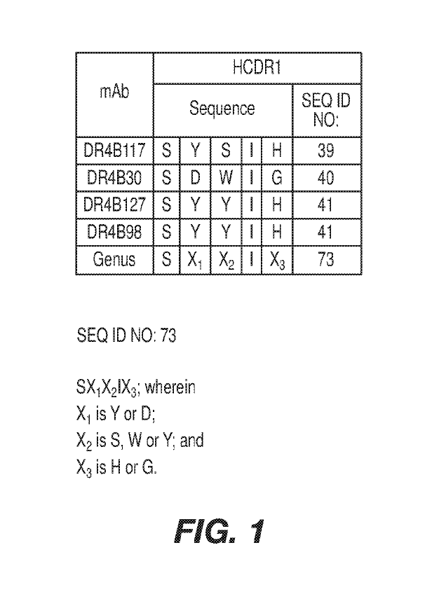

FIG. 1 shows the alignment of HCDR1 amino acid sequences and the HCDR1 genus sequence.

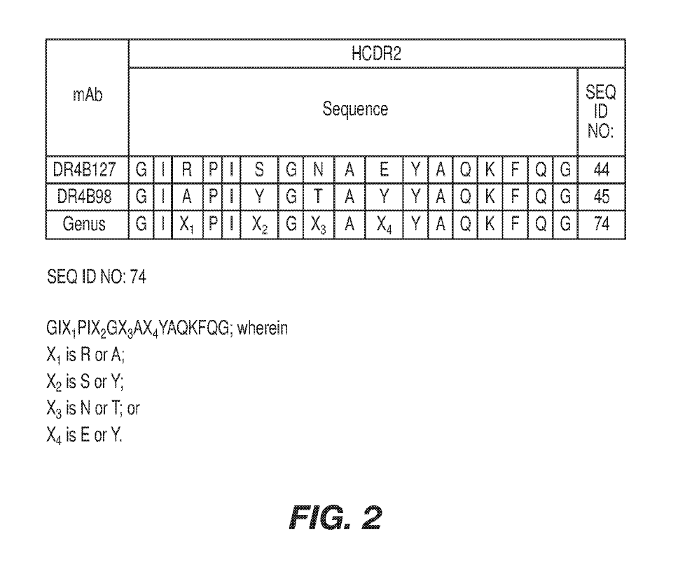

FIG. 2 shows the alignment of HCDR2 amino acid sequences and the HCDR2 genus sequence.

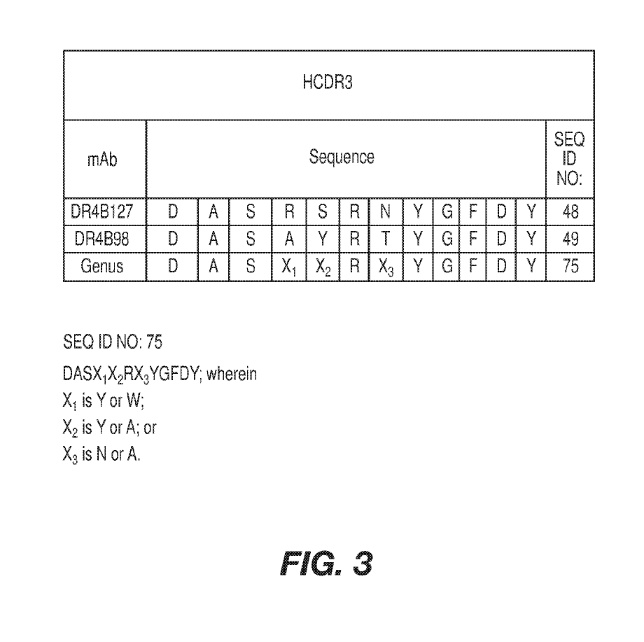

FIG. 3 shows the alignment of HCDR3 amino acid sequences and the HCDR3 genus sequence.

### SEQ ID NO: 73

SX₁X₂IX₃; wherein

### X₁ is Y or D;

### X₂ is S, W or Y; and

### X₃ is H or G.

### SEQ ID NO: 74

GIX₁PIX₂GX₃AX₄YAQKFQG; wherein

### X₁ is R or A:

### X₂ is S or Y;

### X₃ is N or T; or

### X₄ is E or Y.

### SEQ ID NO: 75

DASX₁X₂RX₃YGFDY; wherein

### X₁ is Y or W;

### X₂ is Y or A; or

### X₃ is N or A.

The invention also provides for an isolated antibody or an antigen-binding fragment thereof specifically binding HLA-DR comprising a light chain complementarity determining region 1, 2 and 3 (a LCDR1, a LCDR2 and a LCDR3) of SEQ ID NOs: 76, 77 and 78, respectively.

SEQ ID NOs: 76, 77 and 78 represent the LCDR1, the LCDR2 and the LCDR3 genus amino acid sequences of the antibodies or the antigen-binding fragments thereof specifically binding HLA-DR, respectively. Antibodies within the genus inhibit T cell activation and lack the ability to induce apoptosis and/or death of B cells. Exemplary such antibodies are antibodies DR4B117, DR4B30, DR4B127 and DR4B98 described herein.

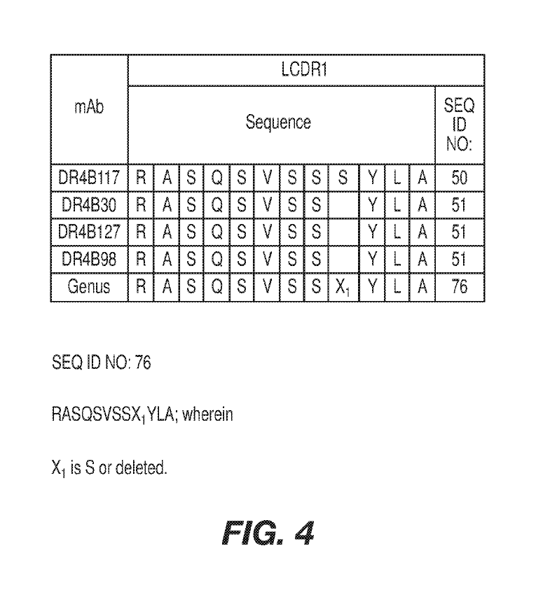

FIG. 4 shows the alignment of LCDR1 amino acid sequences and the LCDR1 genus sequence.

FIG. 5 shows the alignment of LCDR2 amino acid sequences and the LCDR2 genus sequence.

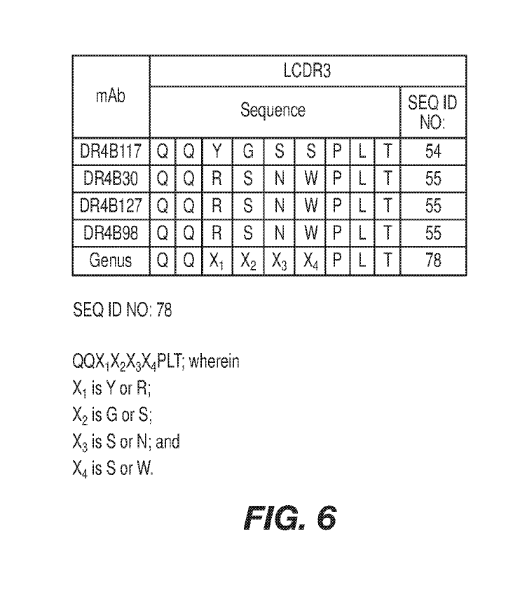

FIG. 6 shows the alignment of LCDR3 amino acid sequences and the LCDR3 genus sequence.

### SEQ ID NO: 76

RASQSVSSX₁YLA; wherein
X₁ is S or deleted.

### SEQ ID NO: 77

X₁ASX₂RAT; wherein

### X₁ is G or D; and

### X₂ is S or N.

### SEQ ID NO: 78

QQX₁X₂X₃X₄PLT; wherein

### X₁ is Y or R:

### X₂ is G or S:

### X₃ is S or N; and

### X₄ is S or W.

The invention also provides for an isolated antibody or an antigen-binding fragment thereof specifically binding HLA-DR comprising a heavy chain complementarity determining region 1, 2 and 3 (a HCDR1, a HCDR2 and a HCDR3) of SEQ ID NOs: 73, 74 and 75, respectively and a light chain complementarity determining region 1, 2 and 3 (a LCDR1, a LCDR2 and a LCDR3) of SEQ ID NOs: 76, 77 and 78, respectively.

The invention also provides for an isolated antibody or an antigen-binding fragment thereof specifically binding HLA-DR comprising the HCDR1, the HCDR2 and the HCDR3 contained in a heavy chain variable region (VH) of SEQ ID NOs: 56, 57, 58, 59, 137, 138, 139, 140 or 141, wherein the HCDR1, the HCDR2 and the HCDR3 are defined by Kabat. IMGT or Chothia.

The invention also provides for an isolated antibody or an antigen-binding fragment thereof specifically binding HLA-DR comprising the LCDR1, the LCDR2 and the LCDR3 contained in a light chain variable region (VL) of SEQ ID NOs: 60, 61 or 142, wherein the LCDR1, the LCDR2 and the LCDR3 are defined by Kabat. IMGT or Chothia.

The invention also provides for an isolated antibody or an antigen-binding fragment thereof specifically binding HLA-DR comprising the HCDR1 of SEQ ID NOs: 39, 40, 41, 123, 124 or 125.

The invention also provides for an isolated antibody or an antigen-binding fragment thereof specifically binding HLA-DR comprising the HCDR2 of SEQ ID NOs: 42, 43, 44, 45, 126, 127 or 128.

The invention also provides for an isolated antibody or an antigen-binding fragment thereof specifically binding HLA-DR comprising the HCDR3 of SEQ ID NOs: 46, 47, 48, 49, 129, 139, 131, 132 or 133.

The invention also provides for an isolated antibody or an antigen-binding fragment thereof specifically binding HLA-DR comprising the LCDR1 of SEQ ID NOs: 50, 51 or 134.

The invention also provides for an isolated antibody or an antigen-binding fragment thereof specifically binding HLA-DR comprising the LCDR2 of SEQ ID NOs: 52, 53 or 135.

The invention also provides for an isolated antibody or an antigen-binding fragment thereof specifically binding HLA-DR comprising the LCDR3 of SEQ ID NOs: 54, 55 or 136.

In some embodiments, the antibody or the antigen-binding fragment thereof specifically binding HLA-DR comprises the HCDR1, the HCDR2, the HCDR3, the LCDR1, the LCDR2 and the LCDR3 of
SEQ ID NOs: 39, 42, 46, 50, 52 and 54, respectively;
SEQ ID NOs: 40, 43, 47, 51, 53 and 55, respectively;
SEQ ID NOs: 41, 44, 48, 51, 53 and 55, respectively;
SEQ ID NOs: 41, 45, 49, 51, 53 and 55, respectively;
SEQ ID NOs: 123, 126, 129, 51, 53 and 55, respectively;
SEQ ID NOs: 123, 126, 130, 51, 53 and 55, respectively;
SEQ ID NOs: 123, 126, 131, 51, 53 and 55, respectively;
SEQ ID NOs: 124, 127, 132, 134, 135 and 136, respectively; or
SEQ ID NOs: 125, 128, 133, 51, 53 and 55, respectively.

In some embodiments, the antibody comprises the VH and the VL of
SEQ ID NOs: 56 and 60, respectively;
SEQ ID NOs: 57 and 61, respectively;
SEQ ID NOs: 58 and 61, respectively;
SEQ ID NOs: 59 and 61, respectively;
SEQ ID NOs: 137 and 61, respectively;
SEQ ID NOs: 138 and 61, respectively;
SEQ ID NOs: 139 and 61, respectively;
SEQ ID NOs: 140 and 142, respectively; or
SEQ ID NOs: 141 and 61, respectively.

In some embodiments, the VH and the VL are encoded by polynucleotides comprising the polynucleotide sequence of
SEQ ID NOs: 79 and 80, respectively;
SEQ ID NOs: 81 and 82, respectively;
SEQ ID NOs: 83 and 82, respectively;
SEQ ID NOs: 121 and 82, respectively;
SEQ ID NOs: 143 and 82, respectively;
SEQ ID NOs: 144 and 82, respectively;
SEQ ID NOs: 145 and 82, respectively;
SEQ ID NOs: 146 and 148, respectively; or
SEQ ID NOs: 147 and 82, respectively;

In some embodiments, the antibody comprises the HC and the LC of
SEQ ID NOs: 84 and 88, respectively;
SEQ ID NOs: 85 and 89, respectively;
SEQ ID NOs: 86 and 89, respectively;
SEQ ID NOs: 87 and 89, respectively;
SEQ ID NOs: 96 and 88, respectively;
SEQ ID NOs: 97 and 89, respectively;
SEQ ID NOs: 98 and 89, respectively;
SEQ ID NOs: 99 and 89, respectively;
SEQ ID NOs: 149 and 89, respectively;
SEQ ID NOs: 150 and 89, respectively;
SEQ ID NOs: 151 and 89, respectively;
SEQ ID NOs: 152 and 154, respectively; or
SEQ ID NOs: 153 and 89, respectively.

The invention also provides for an isolated antibody or an antigen-binding fragment thereof specifically binding HLA-DR comprising the HCDR1, the HCDR2, the HCDR3, the LCDR1, the LCDR2 and the LCDR3 of SEQ ID NOs: 39, 42.46, 50, 52 and 54, respectively.

In some embodiments, the antibody heavy chain framework is derived from IGHV1-69 (SEQ ID NO: 62) and the antibody light chain framework is derived IGKV3-20 (SEQ ID NO: 64).

In some embodiments, the antibody or the antigen-binding fragment thereof binds HLA-DRA1*01:02 of SEQ ID NO: 13 at amino acid residues E3, F108, D110 and R140 and HLA-DRB1*04:01 of SEQ ID NO: 14 at amino acid residues V143 and Q149.

In some embodiments, the antibody or the antigen-binding fragment thereof binds HLA-DRA1*01:02 of SEQ ID NO: 13 at amino acid residues K2, E3, V6, E88, V89, T90, F108, D110, K111, R140, L144, R146 and K176 and HLA-DRB1*04:01 of SEQ ID NO: 14 at amino acid residues L114, K139, V142, V143, S144, T145, L147, I148, Q149 and E162.

Antibodies or antigen-binding fragments thereof binding HLA-DR at these amino acid residues bind HLA-DR at CD4 binding site and do not block HLA-DR binding to cognate TCR.

In some embodiments, the antibody or the antigen-binding fragment thereof comprises a heavy chain variable domain (VH) of SEQ ID NO: 56 and a light chain variable domain (VL) of SEQ ID NO: 60.

In some embodiments, the antibody VH is encoded by a polynucleotide of SEQ ID NO: 79 and the VL is encoded by a polynucleotide of SEQ ID NO: 80.

In some embodiments, the antibody is an IgG1 isotype.

In some embodiments, the antibody is an IgG2 isotype.

In some embodiments, the antibody is an IgG3 isotype.

In some embodiments, the antibody is an IgG4 isotype.

In some embodiments, the antibody comprises at least one substitution in an Fc region that modulates binding of the antibody to an FcγR or FcRn.

In some embodiments, the antibody has at least one substitution in the Fc region that results in reduced binding of the antibody to FcγRI, FcγRIIa, FcγRIIb, FcγRIIIa or FcγRIIIb.

In some embodiments, the antibody is an IgG2 isotype comprising V234A, G237A, P238S, H268A, V309L, A330S and P331S substitutions when compared to the wild-type IgG2.

In some embodiments, the antibody is an IgG1 isotype comprising L234A, L235A, G237A, P238S, H268A, A330S and P331S substitutions when compared to the wild-type IgG1.

In some embodiments, the antibody is an IgG1 isotype comprising L234A and L235A substitutions when compared to the wild-type IgG1.

In some embodiments, the antibody is an IgG4 isotype comprising S228P, F234A and L235A substitutions when compared to the wild-type IgG4.

In some embodiments, the antibody comprises the HC of SEQ ID NO: 84 and the LC of SEQ ID NO: 88.

In some embodiments, the antibody comprises the HC of SEQ ID NO: 96 and the LC of SEQ ID NO: 88.

The invention also provides for an isolated antibody or an antigen-binding fragment thereof specifically binding HLA-DR comprising
a) the HCDR1, the HCDR2, the HCDR3 of
   i) SEQ ID NOs: 40, 43 and 47, respectively;
   ii) SEQ ID NOs: 41, 44 and 48, respectively; or
   iii) SEQ ID NOs: 41, 45 and 49, respectively; and
b) the LCDR1, the LCDR2 and the LCDR3 of SEQ ID NOs: 51, 53 and 55, respectively.

The invention also provides for an isolated antibody or an antigen-binding fragment thereof specifically binding HLA-DR comprising the HCDR1, the HCDR2, the HCDR3, the LCDR1, the LCDR2 and the LCDR3 of SEQ ID NOs: 40, 43, 47, 51, 53 and 55, respectively.

In some embodiments, the antibody heavy chain framework is derived from IGHV5-51 (SEQ ID NO: 63) and the antibody light chain framework is derived from IGKV3-11 (SEQ ID NO: 65).

In some embodiments, the antibody or the antigen-binding fragment thereof comprises a heavy chain variable domain (VH) of SEQ ID NO: 57 and a light chain variable domain (VL) of SEQ ID NO: 61.

In some embodiments, the VH is encoded by a polynucleotide of SEQ ID NO: 81 and the VL is encoded by a polynucleotide of SEQ ID NO: 82.

In some embodiments, the antibody is an IgG1 isotype.

In some embodiments, the antibody is an IgG2 isotype.

In some embodiments, the antibody is an IgG3 isotype.

In some embodiments, the antibody is an IgG4 isotype.

In some embodiments, the antibody comprises at least one substitution in an Fc region that modulates binding of the antibody to an FcγR or FcRn.

In some embodiments, the antibody has at least one substitution in the Fc region that results in reduced binding of the antibody to FcγRI, FcγRIIa, FcγRIIb, FcγRIIIa or FcγRIIIb.

In some embodiments, the antibody is an IgG2 isotype comprising V234A, G237A, P238S, H268A, V309L, A330S and P331S substitutions when compared to the wild-type IgG2.

In some embodiments, the antibody is an IgG1 isotype comprising L234A, L235A, G237A, P238S, H268A, A330S and P331S substitutions when compared to the wild-type IgG1.

In some embodiments, the antibody is an IgG1 isotype comprising L234A and L235A substitutions when compared to the wild-type IgG1.

In some embodiments, the antibody is an IgG4 isotype comprising S228P, F234A and L235A substitutions when compared to the wild-type IgG4.

In some embodiments, the antibody comprises the HC of SEQ ID NO: 85 and the LC of SEQ ID NO: 89.

In some embodiments, the antibody comprises the HC of SEQ ID NO: 97 and the LC of SEQ ID NO: 89.

The invention also provides for an isolated antibody or an antigen-binding fragment thereof specifically binding HLA-DR comprising the HCDR1, the HCDR2, the HCDR3, the LCDR1, the LCDR2 and the LCDR3 of SEQ ID NOs: 41, 44, 48, 51, 53 and 55, respectively.

In some embodiments, the antibody heavy chain framework is derived from IGHV1-69 (SEQ ID NO: 62) and the antibody light chain framework is derived from IGKV3-11 (SEQ ID NO: 65).

In some embodiments, the antibody or the antigen-binding fragment thereof binds HLA-DRA1*01:02 of SEQ ID NO: 13 at amino acid residue K2 and HLA-DRB1*04:01 of SEQ ID NO: 14 at residues D41, S126, R130, V142 and Q149.

In some embodiments, the antibody or the antigen-binding fragment thereof binds HLA-DRA1*01:02 of SEQ ID NO: 13 at amino acid residues I1, K2, E3, D27, R140, E141, D142 and H143 and HLA-DRB1*04:01 of SEQ ID NO: 14 at amino acid residues H16, F17, R23, R25, R29, R39, D41, D43, V44, V50, G125, S126, E128, V129, R130, V142, G146, L147, Q149 and V159.

Antibodies or antigen-binding fragments thereof binding HLA-DR at these amino acid residues bind HLA-DR at CD4 binding site and do not block HLA-DR binding to cognate TCR.

In some embodiments, the antibody or the antigen-binding fragment thereof comprises a heavy chain variable domain (VH) of SEQ ID NO: 58 and a light chain variable domain (VL) of SEQ ID NO: 61.

In some embodiments, the VH is encoded by a polynucleotide of SEQ ID NO: 83 and the VL is encoded by a polynucleotide of SEQ ID NO: 82.

In some embodiments, the antibody is an IgG1 isotype.

In some embodiments, the antibody is an IgG2 isotype.

In some embodiments, the antibody is an IgG3 isotype.

In some embodiments, the antibody is an IgG4 isotype.

In some embodiments, the antibody comprises at least one substitution in an Fc region that modulates binding of the antibody to an FcγR or FcRn.

In some embodiments, the antibody has at least one substitution in the Fc region that results in reduced binding of the antibody to FcγRI, FcγRIIa, FcγRIIb, FcγRIIIa or FcγRIIIb.

In some embodiments, the antibody is an IgG2 isotype comprising V234A, G237A, P238S, H268A, V309L, A330S and P331S substitutions when compared to the wild-type IgG2.

In some embodiments, the antibody is an IgG1 isotype comprising L234A, L235A, G237A, P238S, H268A, A330S and P331S substitutions when compared to the wild-type IgG1.

In some embodiments, the antibody is an IgG1 isotype comprising L234A and L235A substitutions when compared to the wild-type IgG1.

In some embodiments, the antibody is an IgG4 isotype comprising S228P, F234A and L235A substitutions when compared to the wild-type IgG4.

In some embodiments, the antibody comprises the heavy chain (HC) of SEQ ID NO: 86 and a light chain (LC) of SEQ ID NO: 89.

In some embodiments, the antibody comprises the heavy chain (HC) of SEQ ID NO: 98 and a light chain (LC) of SEQ ID NO: 89.

The invention also provides for an isolated antibody or an antigen-binding fragment thereof specifically binding HLA-DR comprising the HCDR1, the HCDR2, the HCDR3, the LCDR1, the LCDR2 and the LCDR3 of SEQ ID NOs: 41, 45, 49, 51, 53 and 55, respectively.

In some embodiments, the antibody heavy chain framework is derived from IGHV1-69 (SEQ ID NO: 62) and the antibody light chain framework is derived from IGKV3-11 (SEQ ID NO: 65).

In some embodiments, the antibody or the antigen-binding fragment thereof comprises a heavy chain variable domain (VH) of SEQ ID NO: 59 and a light chain variable domain (VL) of SEQ ID NO: 61.

In some embodiments, the VH is encoded by a polynucleotide of SEQ ID NO: 121 and the VL is encoded by a polynucleotide of SEQ ID NO: 82.

In some embodiments, the antibody is an IgG1 isotype.

In some embodiments, the antibody is an IgG2 isotype.

In some embodiments, the antibody is an IgG3 isotype.

In some embodiments, the antibody is an IgG4 isotype.

In some embodiments, the antibody comprises at least one substitution in an Fc region that modulates binding of the antibody to an FcγR or FcRn.

In some embodiments, the antibody has at least one substitution in the Fc region that results in reduced binding of the antibody to FcγRI, FcγRIIa, FcγRIIb, FcγRIIIa or FcγRIIIb.

In some embodiments, the antibody is an IgG2 isotype comprising V234A, G237A, P238S, H268A, V309L, A330S and P331S substitutions when compared to the wild-type IgG2.

In some embodiments, the antibody is an IgG1 isotype comprising L234A, L235A, G237A, P238S, H268A, A330S and P331S substitutions when compared to the wild-type IgG1.

In some embodiments, the antibody is an IgG1 isotype comprising L234A and L235A substitutions when compared to the wild-type IgG1.

In some embodiments, the antibody is an IgG4 isotype comprising S228P, F234A and L235A substitutions when compared to the wild-type IgG4.

In some embodiments, the antibody comprises the heavy chain (HC) of SEQ ID NO: 87 and a light chain (LC) of SEQ ID NO: 89.

In some embodiments, the antibody comprises the heavy chain (HC) of SEQ ID NO: 99 and a light chain (LC) of SEQ ID NO: 89.

Table 2 shows the SEQ ID NOs: for Kabat CDR amino acid sequences of select HLA-DR antibodies.

TABLE 2

| mAb | HCDR1 | HCDR2 | HCDR3 | LCDR1 | LCDR2 | LCDR3 |
|---|---|---|---|---|---|---|
| DR4B117 | 39 | 42 | 46 | 50 | 52 | 54 |
| DR4B30 | 40 | 43 | 47 | 51 | 53 | 55 |
| DR4B127 | 41 | 44 | 48 | 51 | 53 | 55 |
| DR4B98 | 41 | 45 | 49 | 51 | 53 | 55 |
| DR4B78 | 123 | 126 | 129 | 51 | 53 | 55 |
| DR4B70 | 123 | 126 | 130 | 51 | 53 | 55 |
| DR4B38 | 123 | 126 | 131 | 51 | 53 | 55 |
| DR4B33 | 124 | 127 | 132 | 134 | 135 | 136 |
| DR4B22 | 125 | 128 | 133 | 51 | 53 | 55 |

The invention also provides for an isolated antibody or an antigen-binding fragment thereof specifically binding HLA-DR, wherein the antibody comprises a heavy chain framework derived from IGHV1-69 (SEQ ID NO: 62), IGHV5-51 (SEQ ID NO: 63) or IGHV3_3-23 (SEQ ID NO: 161).

The invention also provides for an isolated antibody or an antigen-binding fragment thereof specifically binding HLA-DR, wherein the antibody comprises a light chain framework derived from IGKV3-20 (SEQ ID NO: 64), IGKV3-11 (SEQ ID NO: 65) or IGKV1-39 (SEQ ID NO: 162).

The invention also provides for an isolated antibody or an antigen-binding fragment thereof specifically binding HLA-DR, wherein the heavy chain framework is derived from IGHV1-69 (SEQ ID NO: 62) and the light chain framework is derived from IGKV3-20 (SEQ ID NO: 64).

The invention also provides for an isolated antibody or an antigen-binding fragment thereof specifically binding HLA-DR wherein the heavy chain framework is derived from IGHV5-51 (SEQ ID NO: 63) and the light chain framework is derived from IGKV3-11 (SEQ ID NO: 65).

The invention also provides for an isolated antibody or an antigen-binding fragment thereof specifically binding HLA-DR wherein the heavy chain framework is derived from IGHV1-69 (SEQ ID NO: 62) and the light chain framework is derived from IGKV3-11 (SEQ ID NO: 65).

The invention also provides for an isolated antibody or an antigen-binding fragment thereof specifically binding HLA-DR wherein the heavy chain framework is derived from IGHV3-23 (SEQ ID NO: 161) and the light chain framework is derived from IGKV3-11 (SEQ ID NO: 65).

The invention also provides for an isolated antibody or an antigen-binding fragment thereof specifically binding HLA-DR wherein the heavy chain framework is derived from IGHV5-51 (SEQ ID NO: 63) and the light chain framework is derived from IGKV1-39 (SEQ ID NO: 162).

The antibodies of the invention comprising heavy or light chain variable regions “derived from” a particular framework or germline sequence refer to antibodies obtained from a system that uses human germline immunoglobulin genes, such as from transgenic mice or from phage display libraries as discussed herein. An antibody that is “derived from” a particular framework or germline sequence may contain amino acid differences when compared to the sequence it was derived from, due to, for example, naturally-occurring somatic mutations or intentional substitutions. Exemplary antibodies specifically biding HLA-DR having certain VH and VL framework sequences are shown in Table 17.

The invention also provides for an isolated antibody or an antigen-binding fragment thereof specifically binding HLA-DR comprising the VH of SEQ ID NOs: 56, 57, 58, 59, 137, 138, 139, 140 or 141.

The invention also provides for an isolated antibody or an antigen-binding fragment thereof specifically binding HLA-DR comprising the VL of SEQ ID NOs: 60, 61 or 142.

The invention also provides for an isolated antibody or an antigen-binding fragment thereof specifically binding HLA-DR comprising the VH of SEQ ID NO: 56 and the VL of SEQ ID NO: 60.

The invention also provides for an isolated antibody or an antigen-binding fragment thereof specifically binding HLA-DR comprising the VH of SEQ ID NO: 57 and the VL of SEQ ID NO: 61.

The invention also provides for an isolated antibody or an antigen-binding fragment thereof specifically binding HLA-DR comprising the VH of SEQ ID NO: 58 and the VL of SEQ ID NO: 61.

The invention also provides for an isolated antibody specifically binding HLA-DR comprising the VH of SEQ ID NO: 59 and the VL of SEQ ID NO: 61.

The invention also provides for an isolated antibody or an antigen-binding fragment thereof specifically binding HLA-DR comprising the VH of SEQ ID NOs: 57, 58 or 59, and the VL of SEQ ID NO: 61.

The VH and the VL amino acid sequences of exemplary antibodies or antigen-binding fragments thereof specifically binding HLA-DR are shown in Table 14. Table 15 and Table 16.

Although the embodiments illustrated in the Examples comprise pairs of variable domains, one from a heavy chain and one from a light chain, a skilled artisan will recognize that alternative embodiments may comprise single heavy or light chain variable domains. The single variable domain may be used to screen for variable domains capable of forming a two-domain specific antigen-binding fragment capable of, for example, binding to HLA-DR. The screening may be accomplished by phage display screening methods using for example hierarchical dual combinatorial approach disclosed in Int. Patent Publ. No. WO1992/01047. In this approach, an individual colony containing either a VH or a VL chain clone is used to infect a complete library of clones encoding the other chain (VL or VH), and the resulting two-chain specific antigen-binding domain is selected in accordance with phage display techniques using known methods and those described herein. Therefore, the individual VH and VL polypeptide chains are useful in identifying additional antibodies specifically binding to HLA-DR using the methods disclosed in Int. Patent Publ. No. WO1992/01047.

### Homologous Antibodies

Variants of the antibodies or antigen-binding fragments thereof specifically binding HLA-DR of the invention comprising VH or VL amino acid sequences shown in Table 14, Table 15 and Table 16 are within the scope of the invention. For example, variants may comprise one, two, three, four, five, six, seven, eight, nine, ten, eleven, twelve, thirteen, fourteen or fifteen amino acid substitutions in the VH and/or the VL as long as the homologous antibodies retain or have improved functional properties when compared to the parental antibodies. In some embodiments, the sequence identity may be about 90%, 91%, 92%, 93%, 94%, 95%, 96%, 97%, 98% or 99% to a VH or the VL amino acid sequence of the invention.

The homologous antibodies or antigen-binding fragments thereof specifically binding HLA-DR are antagonists and have one, two, three, four or five of the following properties:
a) bind HLA-DR4 comprising HLA-DR α chain of SEQ ID NO: 13 and HLA-DR β chain of SEQ ID NO: 14 in complex with the hemagglutinin peptide of SEQ ID NO: 7 with an equilibrium dissociation constant (K_{D}) of 5×10⁻⁸ M or less, wherein K_{D} is measured using ProteOn XPR36 system at 25° C. in a buffer containing DPBS, 0.01% (w/v) polysorbate 20 (PS-20) and 100 μg/ml BSA;
b) bind HLA-DR1 comprising HLA-DR α chain of SEQ ID NO: 13 and the HLA-DR β chain of SEQ ID NO: 15 in complex with the hemagglutinin peptide of SEQ ID NO: 7 with an equilibrium dissociation constant (K_{D}) of 5×10⁻⁸ M or less, wherein K_{D} is measured using ProteOn XPR36 system at 25° C. in a buffer containing DPBS, 0.01% (w/v) PS-20 and 100 μg/ml BSA;
c) lack an ability to induce apoptosis of B cells, wherein apoptosis is determined by measuring frequency of CD3⁻ CD20⁺ annexinV⁺live/dead⁻ B cells in a sample of human peripheral blood cells (PBMC) using flow cytometry;
d) lack an ability to induce death of B cells death of B cells is determined by measuring frequency of CD3⁻ CD20⁺ annexinV⁺live/dead⁺ B cells in the sample of human PBMC using flow cytometry; or
e) inhibit binding of HLA-DR to CD4.

The invention also provides for an isolated antibody or an antigen-binding fragment thereof specifically binding HLA-DR comprising the VH of SEQ ID NO: 56 and the VL of SEQ ID NO: 60, wherein the VH, the VL or both the VH and the VL optionally comprise one, two, three, four, five, six, seven, eight, nine, ten, eleven, twelve, thirteen, fourteen or fifteen amino acid substitutions. Optionally, any substitutions are not within the CDRs.

The invention also provides for an isolated antibody or an antigen-binding fragment thereof specifically binding HLA-DR comprising the VH of SEQ ID NO: 57 and the VL of SEQ ID NO: 61, wherein the VH, the VL or both the VH and the VL optionally comprise one, two, three, four, five, six, seven, eight, nine, ten, eleven, twelve, thirteen, fourteen or fifteen amino acid substitutions. Optionally, any substitutions are not within the CDRs.

The invention also provides for an isolated antibody or an antigen-binding fragment thereof specifically binding HLA-DR comprising the VH of SEQ ID NO: 58 and the VL of SEQ ID NO: 61, wherein the VH, the VL or both the VH and the VL optionally comprise one, two, three, four, five, six, seven, eight, nine, ten, eleven, twelve, thirteen, fourteen or fifteen amino acid substitutions. Optionally, any substitutions are not within the CDRs.

The invention also provides for an isolated antibody or an antigen-binding fragment thereof specifically binding HLA-DR comprising the VH of SEQ ID NO: 59 and the VL of SEQ ID NO: 61, wherein the VH, the VL or both the VH and the VL optionally comprise one, two, three, four, five, six, seven, eight, nine, ten, eleven, twelve, thirteen, fourteen or fifteen amino acid substitutions. Optionally, any substitutions are not within the CDRs.

The invention also provides for an isolated antibody or an antigen-binding fragment thereof specifically binding HLA-DR comprising the VII of SEQ ID NO: 137 and the VL of SEQ ID NO: 61, wherein the VH, the VL or both the VH and the VL optionally comprise one, two, three, four, five, six, seven, eight, nine, ten, eleven, twelve, thirteen, fourteen or fifteen amino acid substitutions. Optionally, any substitutions are not within the CDRs.

The invention also provides for an isolated antibody or an antigen-binding fragment thereof specifically binding HLA-DR comprising the VII of SEQ ID NO: 138 and the VL of SEQ ID NO: 61, wherein the VII, the VL or both the VH and the VL optionally comprise one, two, three, four, five, six, seven, eight, nine, ten, eleven, twelve, thirteen, fourteen or fifteen amino acid substitutions. Optionally, any substitutions are not within the CDRs.

The invention also provides for an isolated antibody or an antigen-binding fragment thereof specifically binding HLA-DR comprising the VH of SEQ ID NO: 139 and the VL of SEQ ID NO: 61, wherein the VH, the VL or both the VH and the VL optionally comprise one, two, three, four, five, six, seven, eight, nine, ten, eleven, twelve, thirteen, fourteen or fifteen amino acid substitutions. Optionally, any substitutions are not within the CDRs.

The invention also provides for an isolated antibody or an antigen-binding fragment thereof specifically binding HLA-DR comprising the VH of SEQ ID NO: 140 and the VL of SEQ ID NO: 142, wherein the VH, the VL or both the VH and the VL optionally comprise one, two, three, four, five, six, seven, eight, nine, ten, eleven, twelve, thirteen, fourteen or fifteen amino acid substitutions. Optionally, any substitutions are not within the CDRs.

The invention also provides for an isolated antibody or an antigen-binding fragment thereof specifically binding HLA-DR comprising the VII of SEQ ID NO: 141 and the VL of SEQ ID NO: 61, wherein the VH, the VL or both the VH and the VL optionally comprise one, two, three, four, five, six, seven, eight, nine, ten, eleven, twelve, thirteen, fourteen or fifteen amino acid substitutions. Optionally, any substitutions are not within the CDRs.

The invention also provides for an isolated antibody or an antigen-binding fragment thereof specifically binding HLA-DR comprising the VH having the amino acid sequence at least 90%, 91%, 92%, 93%, 94%, 95%, 96%, 97%, 98% or 99% identical to the VH of SEQ ID NOs: 56, 57, 58, 59, 137, 138, 139, 140 or 141. Optionally, any variation from the sequences of the SEQ ID NOs is not within the CDRs.

The invention also provides for an isolated antibody or an antigen-binding fragment thereof specifically binding HLA-DR comprising the VL having the amino acid sequence at least 90%, 91%, 92%, 93%, 94%, 95%, 96%, 97%, 98% or 99% identical to the VL of SEQ ID NOs: 60, 61 or 142. Optionally, any variation from the sequences of the SEQ ID NOs is not within the CDRs.

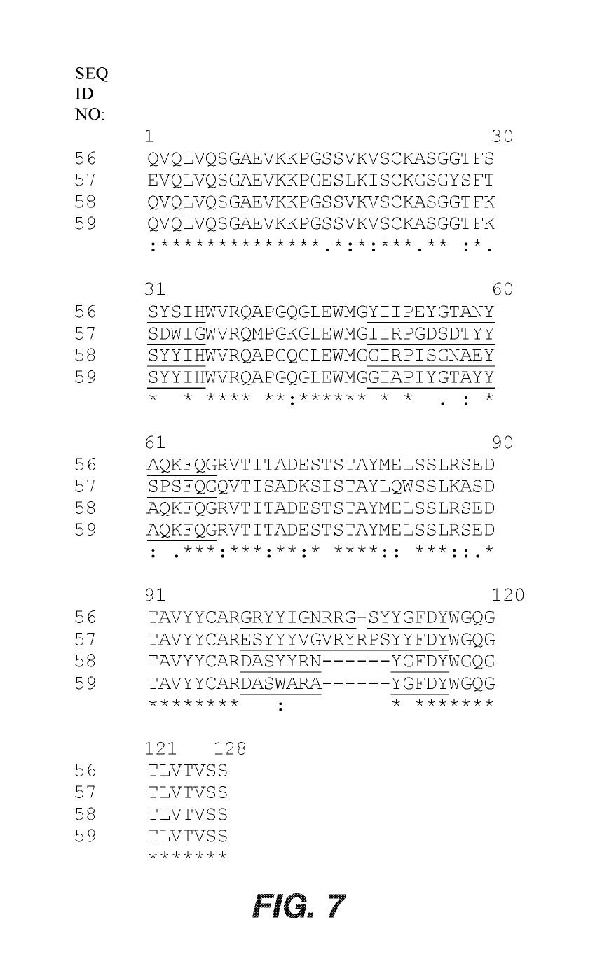

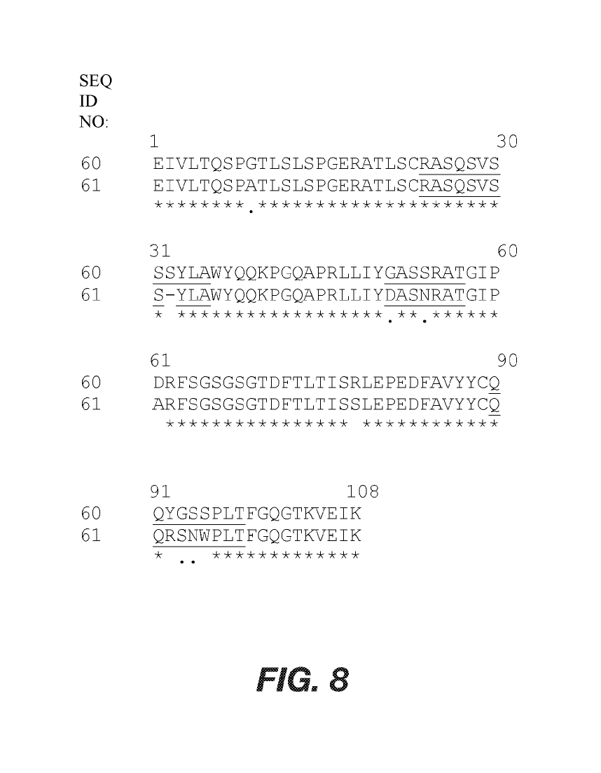

The alignment of the amino acid sequences of the VH domains of select antibodies specifically binding HLA-DR are shown in FIG. 7, and the alignment of the amino acid sequences of the VL domains of select antibodies are shown in FIG. 8. The VII and the VL chains are identified by their SEQ ID NO: at the beginning of each row. Possible sites of substitutions in the VH and/or the VL are the residue positions that differ between the antibodies. For example substitutions may be made at residue positions 1, 16, 18, 20, 24, 27, 28, 30, 40, 43, 67, 71, 74, 76, 81, 82, 83, 87, 88, 89 in the VH of SEQ ID NOs: 56, 57, 58 or 59. Exemplary substitutions that may be made are conservative amino acid substitutions, or substitutions with amino acid residues present in the corresponding residue position in each antibody specifically binding HLA-DR. For example, in the VH of SEQ ID NO: 58, amino acid residue positions 1, 16, 18, 20, 24, 27, 28, 30, 40, 43, 67, 71, 74, 76, 81, 82, 83, 87, 88, 89 may be substituted with corresponding residues present in the VH of SEQ ID NOs: 56, 57 or 59, or with amino acids resulting in conservative modifications as described herein. Similarly, in the VL of SEQ ID NO: 61, amino acid residue positions 9, 61, 78 may be substituted with corresponding residues present in the VL of SEQ ID NO: 60 or substitutions with amino acid residues resulting in conservative modifications as described herein.

The percent identity between the two sequences is a function of the number of identical positions shared by the sequences (i.e., % identity=number of identical positions/total number of positions×100), taking into account the number of gaps, and the length of each gap, which need to be introduced for optimal alignment of the two sequences.

The percent identity between two amino acid sequences may be determined using the algorithm of E. Meyers and W. Miller (Comput Appl Biosci 4:11-17 (1988)) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch (J Mol Biol 48:444-453 (1970)) algorithm which has been incorporated into the GAP program in the GCG software package (available at http://_www_gcg_com), using either a Blossum 62 matrix or a PAM250 matrix, and a gap weight of 16, 14, 12, 10, 8, 6, or 4 and a length weight of 1, 2, 3, 4, 5, or 6.

Antibodies with Conservative Modifications

The invention also provides for an isolated antibody or an antigen-binding fragment thereof specifically binding HLA-DR comprising the VH comprising the HCDR1, the HCDR2 and the HCDR3 sequences and the VL comprising the LCDR1, the LCDR2 and the LCDR3 sequences, wherein one or more of the CDR sequences comprise specified amino acid sequences based on the antibodies described herein (e.g., antibodies shown in Table 2, or Table 14 or conservative modifications thereof, and wherein the antibodies retain the desired functional properties of the parental antibodies specifically binding HLA-DR.

The antibodies or the antigen-binding fragments thereof specifically binding HLA-DR having conservative modifications are antagonists and have one, two, three, four or five of the following properties:
a) bind HLA-DR4 comprising HLA-DR α chain of SEQ ID NO: 13 and HLA-DR β chain of SEQ ID NO: 14 in complex with the hemagglutinin peptide of SEQ ID NO: 7 with an equilibrium dissociation constant (K_{D}) of 5×10⁻⁸ M or less, wherein K_{D} is measured using ProteOn XPR36 system at 25° C. in a buffer containing DPBS, 0.01% (w/v) polysorbate 20 (PS-20) and 100 μg/ml BSA;
b) bind HLA-DR1 comprising HLA-DR α chain of SEQ ID NO: 13 and the HLA-DR β chain of SEQ ID NO: 15 in complex with the hemagglutinin peptide of SEQ ID NO: 7 with an equilibrium dissociation constant (K_{D}) of 5×10⁻⁸ M or less, wherein K_{D} is measured using ProteOn XPR36 system at 25° C. in a buffer containing DPBS, 0.01% (w/v) PS-20 and 100 μg/ml BSA;
c) lack an ability to induce apoptosis of B cells, wherein apoptosis is determined by measuring frequency of CD3⁻ CD20⁺ annexinV⁺live/dead⁻ B cells in a sample of human peripheral blood cells (PBMC) using flow cytometry;
d) lack an ability to induce death of B cells death of B cells is determined by measuring frequency of CD3⁻ CD20⁺ annexinV⁺live/dead⁺ B cells in the sample of human PBMC using flow cytometry; or
e) inhibit binding of HLA-DR to CD4.

The invention also provides for an isolated antibody or an antigen-binding fragment thereof specifically binding HLA-DR comprising the HCDR1, the HCDR2, the HCDR3 of SEQ ID NOs: 39, 42 and 46, respectively, and the LCDR, the LCDR2 and the LCDR3 of SEQ ID NOs: 50, 52 and 54, respectively, and conservative modifications thereof.

The invention also provides for an isolated antibody or an antigen-binding fragment thereof specifically binding HLA-DR comprising the HCDR1, the HCDR2, the HCDR3, the LCDR1, the LCDR2 and the LCDR3 of SEQ ID NOs: 40, 43, 47, 51, 53 and 55, respectively, and conservative modifications thereof.

The invention also provides for an isolated antibody or an antigen-binding fragment thereof specifically binding HLA-DR comprising the HCDR1, the HCDR2, the HCDR3, the LCDR1, the LCDR2 and the LCDR3 of SEQ ID NOs: 41, 44, 48, 51, 53 and 55, respectively, and conservative modifications thereof.

The invention also provides for an isolated antibody or an antigen-binding fragment thereof specifically binding HLA-DR comprising the HCDR1, the HCDR2, the HCDR3, the LCDR1, the LCDR2 and the LCDR3 of SEQ ID NOs: 41, 45, 49, 51, 53 and 55, respectively, and conservative modifications thereof.

The invention also provides for an isolated antibody or an antigen-binding fragment thereof specifically binding HLA-DR comprising the HCDR1, the HCDR2, the HCDR3, the LCDR1, the LCDR2 and the LCDR3 of SEQ ID NOs: 123, 126, 129, 51, 53 and 55, respectively, and conservative modifications thereof.

The invention also provides for an isolated antibody or an antigen-binding fragment thereof specifically binding HLA-DR comprising the HCDR1, the HCDR2, the HCDR3, the LCDR1, the LCDR2 and the LCDR3 of SEQ ID NOs: 123, 126, 130, 51, 53 and 55, respectively, and conservative modifications thereof.

The invention also provides for an isolated antibody or an antigen-binding fragment thereof specifically binding HLA-DR comprising the HCDR1, the HCDR2, the HCDR3, the LCDR1, the LCDR2 and the LCDR3 of SEQ ID NOs: 123, 126, 131, 51, 53 and 55, respectively, and conservative modifications thereof.

The invention also provides for an isolated antibody or an antigen-binding fragment thereof specifically binding HLA-DR comprising the HCDR1, the HCDR2, the HCDR3, the LCDR1, the LCDR2 and the LCDR3 of SEQ ID NOs: 124, 127, 132, 134, 135 and 136, respectively, and conservative modifications thereof.

The invention also provides for an isolated antibody or an antigen-binding fragment thereof specifically binding HLA-DR comprising the HCDR1, the HCDR2, the HCDR3, the LCDR1, the LCDR2 and the LCDR3 of SEQ ID NOs: 125, 128, 133, 51, 53 and 55, respectively, and conservative modifications thereof.

The invention also provides for an isolated antibody or an antigen-binding fragment thereof specifically binding HLA-DR comprising the VH of SEQ ID NO: 56 and the VL of SEQ ID NO: 60, and conservative modifications thereof.

The invention also provides for an isolated antibody or an antigen-binding fragment thereof specifically binding HLA-DR comprising the VH of SEQ ID NO: 57 and the VL of SEQ ID NO: 61, and conservative modifications thereof.

The invention also provides for an isolated antibody or an antigen-binding fragment thereof specifically binding HLA-DR comprising the VH of SEQ ID NO: 58 and the VL of SEQ ID NO: 61, and conservative modifications thereof.

The invention also provides for an isolated antibody or an antigen-binding fragment thereof specifically binding HLA-DR comprising the VH of SEQ ID NO: 59 and the VL of SEQ ID NO: 61, and conservative modifications thereof.

The invention also provides for an isolated antibody or an antigen-binding fragment thereof specifically binding HLA-DR comprising the VH of SEQ ID NOs: 137 and the VL of SEQ ID NO: 61, and conservative modifications thereof.

The invention also provides for an isolated antibody or an antigen-binding fragment thereof specifically binding HLA-DR comprising the VH of SEQ ID NOs: 138 and the VL of SEQ ID NO: 61, and conservative modifications thereof.

The invention also provides for an isolated antibody or an antigen-binding fragment thereof specifically binding HLA-DR comprising the VH of SEQ ID NOs: 139 and the VL of SEQ ID NO: 61, and conservative modifications thereof.

The invention also provides for an isolated antibody or an antigen-binding fragment thereof specifically binding HLA-DR comprising the VI of SEQ ID NOs: 140 and the VL of SEQ ID NO: 142, and conservative modifications thereof.

The invention also provides for an isolated antibody or an antigen-binding fragment thereof specifically binding HLA-DR comprising the VH of SEQ ID NOs: 141 and the VL of SEQ ID NO: 61, and conservative modifications thereof.

“Conservative modifications” refer to amino acid modifications that do not significantly affect or alter the binding characteristics of the antibody containing the amino acid modifications. Conservative modifications include amino acid substitutions, additions and deletions. Conservative amino acid substitutions are those in which the amino acid is replaced with an amino acid residue having a similar side chain. The families of amino acid residues having similar side chains are well defined and include amino acids with acidic side chains (e.g., aspartic acid, glutamic acid), basic side chains (e.g., lysine, arginine, histidine), nonpolar side chains (e.g., alanine, valine, leucine, isoleucine, proline, phenylalanine, methionine), uncharged polar side chains (e.g., glycine, asparagine, glutamine, cysteine, serine, threonine, tyrosine, tryptophan), aromatic side chains (e.g., phenylalanine, tryptophan, histidine, tyrosine), aliphatic side chains (e.g., glycine, alanine, valine, leucine, isoleucine, serine, threonine), amide (e.g., asparagine, glutamine), beta-branched side chains (e.g., threonine, valine, isoleucine) and sulfur-containing side chains (cysteine, methionine). Furthermore, any native residue in the polypeptide may also be substituted with alanine, as has been previously described for alanine scanning mutagenesis (MacLennan et al., (1988) Acta Physiol Scand Suppl 643:55-67; Sasaki et al., (1988) Adv Biophys 35:1-24). Amino acid substitutions to the antibodies of the invention may be made by known methods for example by PCR mutagenesis (U.S. Pat. No. 4,683,195). Alternatively, libraries of variants may be generated for example using random (NNK) or non-random codons, for example DVK codons, which encode 11 amino acids (Ala, Cys, Asp, Glu, Gly, Lys, Asn, Arg, Ser, Tyr, Trp). The resulting antibody variants may be tested for their characteristics using assays described herein.

### Engineered and Modified Antibodies

The antibodies or the antigen-binding fragments thereof of the invention may be further engineered to generate modified antibodies with similar or altered properties when compared to the parental antibodies. The VH, the VL, the VH and the VL, the constant regions, the heavy chain framework, the light chain framework, or any or all of the six CDRs may be engineered in the antibodies of the invention.

The antibodies of the invention may be engineered by CDR grafting. One or more CDR sequences of the antibodies of the invention may be grafted to a different framework sequence. CDR grafting may be done using known methods and methods described herein.

The framework sequences that may be used may be obtained from public DNA databases or published references that include germline antibody gene sequences. For example, germline DNA and the encoded protein sequences for human heavy and light chain variable domain genes may be found at IMGT®, the international ImMunoGeneTics information System® http://_www-imgt_org. Framework sequences that may be used to replace the existing framework sequences of the antibodies of the invention may be those that show the highest percent (%) identity to the parental variable domains over the entire length of the VH or the VL, or over the length of the FR1, FR2, FR3 and FR4. In addition, suitable frameworks may further be selected based on the VH and the VL CDR1 and CDR2 lengths or identical LCDR1. LCDR2, LCDR3, HCDR1 and HCDR2 canonical structure. Suitable frameworks may be selected using known methods, such as human framework adaptation described in U.S. Pat. No. 8,748,356 or superhumanization described in U.S. Pat. No. 7,709,226.

The framework sequences of the parental and engineered antibodies may further be modified, for example by backmutations to restore and/or improve binding of the generated antibodies to the antigen as described for example in U.S. Pat. No. 6,180,370. The framework sequences of the parental or engineered antibodies may further be modified by mutating one or more residues within the framework region (or alternatively within one or more CDR regions) to remove T-cell epitopes to thereby reduce the potential immunogenicity of the antibody. This approach is also referred to as “deimmunization” and described in further detail in U.S. Patent Publ. No. US20070014796.

The CDR residues of the antibodies of the invention may be mutated to improve affinity of the antibodies to HLA-DR.

The CDR residues of the antibodies of the invention may be mutated to minimize risk of post-translational modifications. Amino acid residues of putative motifs for deamination (NS), acid-catalyzed hydrolysis (DP), isomerization (DS), or oxidation (W) may be substituted with any of the naturally occurring amino acids to mutagenize the motifs, and the resulting antibodies may be tested for their functionality and stability using methods described herein.

Antibodies of the invention may be modified to improve stability, selectivity, cross-reactivity, affinity, immunogenicity or other desirable biological or biophysical property are within the scope of the invention. Stability of an antibody is influenced by a number of factors, including (1) core packing of individual domains that affects their intrinsic stability, (2) protein/protein interface interactions that have impact upon the HC and LC pairing, (3) burial of polar and charged residues, (4) H-bonding network for polar and charged residues; and (5) surface charge and polar residue distribution among other intra- and inter-molecular forces (Worn et al., (2001) J Mol Biol 305:989-1010). Potential structure destabilizing residues may be identified based upon the crystal structure of the antibody or by molecular modeling in certain cases, and the effect of the residues on antibody stability may be tested by generating and evaluating variants harboring mutations in the identified residues. One of the ways to increase antibody stability is to raise the thermal transition midpoint (Tₘ) as measured by differential scanning calorimetry (DSC). In general, the protein Tₘ is correlated with its stability and inversely correlated with its susceptibility to unfolding and denaturation in solution and the degradation processes that depend on the tendency of the protein to unfold (Remmele et al., (2000) Biopharm 13:36-46). A number of studies have found correlation between the ranking of the physical stability of formulations measured as thermal stability by DSC and physical stability measured by other methods (Gupta et al., (2003) AAPS Pharm Sci 5E8; Zhang et al., (2004) J Pharm Sci 93:3076-89; Maa et al., (1996) Int J Pharm 140:155-68; Bedu-Addo et al., (2004) Pharm Res 21:1353-61; Remmele et al., (1997) Pharm Res 15:200-8). Formulation studies suggest that a Fab Tₘ has implication for long-term physical stability of a corresponding mAb.

C-terminal lysine (CTL) may be removed from injected antibodies by endogenous circulating carboxypeptidases in the blood stream (Cai et al., (2011) Biotechnol Bioeng 108:404-412). During manufacturing, CTL removal may be controlled to less than the maximum level by control of concentration of extracellular Zn²⁺, EDTA or EDTA-Fe³⁺ as described in U.S. Patent Publ. No. US20140273092. CTL content in antibodies may be measured using known methods.

In some embodiments, the antibodies specifically binding HLA-DR have a C-terminal lysine content of about 10% to about 90%, about 20% to about 80%, about 40% to about 70%, about 55% to about 70%, or about 60%.

Fc substitutions may be made to the antibodies of the invention to modulate antibody effector functions and/or pharmacokinetic properties. In traditional immune function, the interaction of antibody-antigen complexes with cells of the immune system results in a wide array of responses, ranging from effector functions such as antibody-dependent cytotoxicity, mast cell degranulation, and phagocytosis to immunomodulatory signals such as regulating lymphocyte proliferation and antibody secretion. All of these interactions are initiated through the binding of the Fc domain of antibodies or immune complexes to specialized cell surface receptors on cells. The diversity of cellular responses triggered by antibodies and immune complexes results from the heterogeneity of the Fc receptors: FcγRI (CD64), FcγRIIa (CD32A), and FcγRI (CD16) are activating Fcγ receptors (i e, immune system enhancing) whereas FcγRIIb (CD32B) is an inhibitory Fcγ receptor (i.e., immune system dampening). Binding to the FcRn receptor modulates antibody half-life.

In some embodiments, the antibodies specifically binding HLA-DR of the invention comprise at least one substitution in an Fc region.

In some embodiments, the antibodies specifically binding HLA-DR of the invention comprise one, two, three, four, five, six, seven, eight, nine, ten, eleven, twelve, thirteen fourteen or fifteen substitutions in the Fc region.

Fc positions that may be substituted to modulate antibody half-life are those described for example in Dall'Acqua et al., (2006) J Biol Chem 281:23514-240, Zalevsky et al., (2010) Nat Biotechnol 28:157-159, Hinton et al., (2004) J Biol Chem 279(8):6213-6216, Hinton et al., (2006) J Immunol 176:346-356, Shields et al. (2001) J Biol Chem 276:6591-6607, Petkova et al., (2006). Int Inmunol 18:1759-1769, Datta-Mannan et al., (2007) Drug Aletab Dispos, 35:86-94, 2007. Vaccaro et al., (2005) Nat Biotechnol 23:1283-1288, Yeung et al., (2010) Cancer Res, 70:3269-3277 and Kim et al., (1999) Eur J Immunol 29: 2819, and include positions 250, 252, 253, 254, 256, 257, 307, 376, 380, 428, 434 and 435. Exemplary substitutions that may be made singularly or in combination are substitutions T250Q, M252Y, I253A, S254T, T256E, P257I, T307A, D376V, E380A, M428L, H433K, N434S, N434A, N434H, N434F, H435A and H435R. Exemplary singular or combination substitutions that may be made to increase the half-life of the antibody are substitutions M428L/N434S, M252Y/S254T/T256E, T250Q/M428L, N434A and T307A/E380A/N434A. Exemplary singular or combination substitutions that may be made to reduce the half-life of the antibody are substitutions H435A, P257I/N434H, D376V/N434H, M252Y/S254T/T256E/H433K/N434F, T308P/N434A and H435R.

In some embodiments, the antibodies specifically binding HLA-DR of the invention comprise at least one substitution in the antibody Fc at amino acid position 250, 252, 253, 254, 256, 257, 307, 376, 380, 428, 434 or 435.

In some embodiments, the antibodies specifically binding HLA-DR of the invention comprise at least one substitution in the antibody Fc selected from the group consisting of T250Q, M252Y, I253A, S254T, T256E, P257I, T307A, D376V, E380A, M428L, H433K, N434S, N434A, N434H, N434F, H435A and H435R.

In some embodiments, the antibodies specifically binding HLA-DR of the invention comprise at least one substitution in the antibody Fc selected from the group consisting of M428L/N434S, M252Y/S254T/T256E, T250Q/M428L, N434A, T307A/E380A/N434A, H435A, P257I/N434H, D376V/N434H, M252Y/S254T/T256E/H433K/N434F, T308P/N434A and H435R.

In some embodiments, the antibodies specifically binding HLA-DR of the invention comprise at least one substitution in the Fc region that reduces binding of the antibody to an activating Fcγ receptor (FcγR) and/or reduces Fc effector functions such as C1q binding, complement dependent cytotoxicity (CDC), antibody-dependent cell-mediated cytotoxicity (ADCC) or phagocytosis (ADCP).

Fc positions that may be substituted to reduce binding of the antibody to the activating FcγR and subsequently to reduce effector function are those described for example in Shields et al., (2001) J Biol Chem 276:6591-6604, Intl. Patent Publ. No. WO02011/066501, U.S. Pat. Nos. 6,737,056 and 5,624,821, Xu et al., (2000) Cell Immunol, 200:16-26, Alegre et al., (1994) Transplantation 57:1537-1543, Bolt et al., (1993) Eur J Immunol 23:403-411. Cole et al., (1999) Transplantation, 68:563-571, Rother et al., (2007) Nat Biotechnol 25:1256-1264, Ghevaert et al., (2008) J Clin Invest 118:2929-2938, An et al., (2009) mAbs, 1:572-579) and include positions 214, 233, 234, 235, 236, 237, 238, 265, 267, 268, 270, 295, 297, 309, 327, 328, 329, 330, 331 and 365. Exemplary substitutions that may be made singularly or in combination are substitutions K214T, E233P, L234V, L234A, deletion of G236, V234A, F234A, L235A, G237A, P238A, P238S, D265A, S267E, H268A, H268Q, Q268A, N297A, A327Q, P329A, D270A, Q295A, V309L, A327S, L328F, A330S and P331S in IgG1, IgG2, IgG3 or IgG4. Exemplary combination substitutions that result in antibodies with reduced ADCC are substitutions L234A/L235A on IgG1, V234A/G237A/P238S/H268A/V309L/A330S/P331S on IgG2, F234A/L235A on IgG4, S228P/F234A/L235A on IgG4, N297A on all Ig isotypes, V234A/G237A on IgG2, K214T/E233P/L234V/L235A/G236-deleted/A327G/P331A/D365E/L358M on IgG1, H268Q/V309L/A330S/P331S on IgG2, S267E/L328F on IgG1, L234F/L235E/D265A on IgG1, L234A/L235A/G237A/P238S/H268A/A330S/P331S on IgG1, S228P/F234A/L235A/G237A/P238S on IgG4, and S228P/F234A/L235A/G236-deleted/G237A/P238S on IgG4. Hybrid IgG2/4 Fc domains may also be used, such as Fc with residues 117-260 from IgG2 and residues 261-447 from IgG4.

Well-known S228P substitution may be made in IgG4 antibodies to enhance IgG4 stability.

In some embodiments, the antibodies specifically binding HLA-DR of the invention comprise a substitution in at least one residue position 214, 233, 234, 235, 236, 237, 238, 265, 267, 268, 270, 295, 297, 309, 327, 328, 329, 330, 331 or 365, wherein residue numbering is according to the EU Index.

In some embodiments, the antibodies specifically binding HLA-DR of the invention comprise at least one substitution selected from the group consisting of K214T, E233P, L234V, L234A, deletion of G236, V234A, F234A, L235A, G237A, P238A, P238S, D265A, S267E, H268A, H268Q, Q268A, N297A, A327Q, P329A, D270A, Q295A, V309L, A327S, L328F, A330S and P331S, wherein residue numbering is according to the EU Index.

In some embodiments, the antibodies specifically binding HLA-DR of the invention comprise a substitution in at least one residue position 228, 234, 235, 237, 238, 268, 330 or 331, wherein residue numbering is according to the EU Index.

In some embodiments, the antibodies specifically binding HLA-DR of the invention comprise a S228P substitution, wherein residue numbering is according to the EU Index.

In some embodiments, the antibodies specifically binding HLA-DR of the invention comprise a V234A substitution, wherein residue numbering is according to the EU Index.

In some embodiments, the antibodies specifically binding HLA-DR of the invention comprise a F234A substitution, wherein residue numbering is according to the EU Index.

In some embodiments, the antibodies specifically binding HLA-DR of the invention comprise a G237A substitution, wherein residue numbering is according to the EU Index.

In some embodiments, the antibodies specifically binding HLA-DR of the invention comprise a P238S substitution, wherein residue numbering is according to the EU Index.

In some embodiments, the antibodies specifically binding HLA-DR of the invention comprise a H268A substitution, wherein residue numbering is according to the EU Index.

In some embodiments, the antibodies specifically binding HLA-DR of the invention comprise a Q268A substitution, wherein residue numbering is according to the EU Index.

In some embodiments, the antibodies specifically binding HLA-DR of the invention comprise an A330S substitution, wherein residue numbering is according to the EU Index.

In some embodiments, the antibodies specifically binding HLA-DR of the invention comprise a P331S substitution, wherein residue numbering is according to the EU Index.

In some embodiments, the antibodies specifically binding HLA-DR of the invention comprise L234A, L235A, G237A, P238S, H268A, A330S and P331S substitutions, wherein residue numbering is according to the EU Index.

In some embodiments, the antibodies specifically binding HLA-DR of the invention comprise V234A, G237A, P238S, H268A, V309L, A330S and P331S substitutions, wherein residue numbering is according to the EU Index.

In some embodiments, the antibodies specifically binding HLA-DR of the invention comprise F234A, L235A, G237A, P238S and Q268A substitutions, wherein residue numbering is according to the EU Index.

In some embodiments, the antibodies specifically binding HLA-DR of the invention comprise L234A, L235A or L234A and L235A substitutions, wherein residue numbering is according to the EU Index.

In some embodiments, the antibodies specifically binding HLA-DR of the invention comprise F234A, L235A or F234A and L235A substitutions, wherein residue numbering is according to the EU Index.

In some embodiments, the antibodies specifically binding HLA-DR of the invention comprise S228P, F234A and L235A substitutions, wherein residue numbering is according to the EU Index.

In some embodiments, the antibodies specifically binding HLA-DR of the invention comprise a S228P substitution, wherein residue numbering is according to the EU Index.

Methods of Generating Homologous Antibodies, Antibodies with Conservative Modifications, and Engineered and Modified Antibodies

The antibodies of the invention that have altered amino acid sequences when compared to the parental antibodies may be generated using standard cloning and expression technologies. For example, site-directed mutagenesis or PCR-mediated mutagenesis may be performed to introduce the mutation(s) and the effect on antibody binding or other property of interest may be evaluated using well known methods and the methods described herein in the Examples.

### Antibody Isotypes and Allotypes

The antibodies of the invention may be an IgG1, IgG2. IgG3 or IgG4 isotype.

In some embodiments, the antibodies specifically binding HLA-DR of the invention are an IgG1 isotype.

In some embodiments, the antibodies specifically binding HLA-DR of the invention are an IgG2 isotype.

In some embodiments, the antibodies specifically binding HLA-DR of the invention are an IgG3 isotype.

In some embodiments, the antibodies specifically binding HLA-DR of the invention are of IgG4 isotype.

Immunogenicity of therapeutic antibodies is associated with increased risk of infusion reactions and decreased duration of therapeutic response (Baert et al., (2003) N Engl J Med 348:602-08). The extent to which therapeutic antibodies induce an immune response in the host may be determined in part by the allotype of the antibody (Stickler et al., (2011) Genes and Immunity 12:213-21). Antibody allotype is related to amino acid sequence variations at specific locations in the constant region sequences of the antibody. Table 3 shows select IgG1. IgG2 and IgG4 allotypes.

In some embodiments, the antibodies specifically binding HLA-DR of the invention are an G2m(n) allotype.

In some embodiments, the antibodies specifically binding HLA-DR of the invention are an G2m(n−) allotype.

In some embodiments, the antibodies specifically binding HLA-DR of the invention are an G2m(n)/(n−) allotype.

In some embodiments, the antibodies specifically binding HLA-DR of the invention are an nG4m(a) allotype.

In some embodiments, the antibodies specifically binding HLA-DR of the invention are an G1m(17) allotype.

In some embodiments, the antibodies specifically binding HLA-DR of the invention are an G1m(17,1) allotype.

TABLE 3

| | Amino acid residue at position of diversity (residue | | | | | | | |
|---|---|---|---|---|---|---|---|---|
| | numbering: EU Index) | | | | | | | |
| | IgG2 | | IgG4 | | IgG1 | | | |
| Allotype | 189 | 282 | 309 | 422 | 214 | 356 | 358 | 431 |
| G2m(n) | T | M | | | | | | |
| G2m(n−) | P | V | | | | | | |
| G2m(n)/(n−) | T | V | | | | | | |
| nG4m(a) | | | L | R | | | | |
| G1m(17) | | | | | K | E | M | A |
| G1m(17,1) | | | | | K | D | L | A |

### Anti-Idiotypic Antibodies

The invention also provides an anti-idiotypic antibody specifically binding to the antibodies specifically binding HLA-DR of the invention.

The invention also provides an anti-idiotypic antibody specifically binding the antibody comprising the VH of SEQ ID NO: 56 and the VL of SEQ ID NO: 60.

The invention also provides an anti-idiotypic antibody specifically binding the antibody comprising the VH of SEQ ID NO: 57 and the VL of SEQ ID NO: 61.

The invention also provides an anti-idiotypic antibody specifically binding the antibody comprising the VH of SEQ ID NO: 58 and the VL of SEQ ID NO: 61.

The invention also provides an anti-idiotypic antibody specifically binding the antibody comprising the VH of SEQ ID NO: 59 and the VL of SEQ ID NO: 61.

The invention also provides an anti-idiotypic antibody specifically binding the antibody comprising the VI of SEQ ID NOs: 137 and the VL of SEQ ID NO: 61.

The invention also provides an anti-idiotypic antibody specifically binding the antibody comprising the VI of SEQ ID NOs: 138 and the VL of SEQ ID NO: 61.

The invention also provides an anti-idiotypic antibody specifically binding the antibody comprising the VI of SEQ ID NOs: 139 and the VL of SEQ ID NO: 61.

The invention also provides an anti-idiotypic antibody specifically binding the antibody comprising the VH of SEQ ID NOs: 140 and the VL of SEQ ID NO: 142.

The invention also provides an anti-idiotypic antibody specifically binding the antibody comprising the VII of SEQ ID NOs: 141 and the VL of SEQ ID NO: 61.

An anti-idiotypic (Id) antibody is an antibody which recognizes the antigenic determinants (e.g. the paratope or CDRs) of the antibody. The Id antibody may be antigen-blocking or non-blocking. The antigen-blocking Id may be used to detect the free antibody in a sample (e.g. anti-HLA-DR antibody of the invention described herein). The non-blocking Id may be used to detect the total antibody (free, partially bond to antigen, or fully bound to antigen) in a sample. An Id antibody may be prepared by immunizing an animal with the antibody to which an anti-Id is being prepared.

An anti-Id antibody may also be used as an immunogen to induce an immune response in yet another animal, producing a so-called anti-anti-Id antibody. An anti-anti-Id may be epitopically identical to the original mAb, which induced the anti-Id. Thus, by using antibodies to the idiotypic determinants of a mAb, it is possible to identify other clones expressing antibodies of identical specificity. Anti-Id antibodies may be varied (thereby producing anti-Id antibody variants) and/or derivatized by any suitable technique, such as those described elsewhere herein with respect to the antibodies specifically binding HLA-DR antibodies.

### Conjugates of the Antibodies Specifically Binding HLA-DR of the Invention

The invention also provides an isolated antibody or an antigen-binding fragment thereof specifically binding HLA-DR conjugated to a heterologous molecule(s).

In some embodiments, the heterologous molecule is a detectable label or a cytotoxic agent.

The invention also provides an isolated antibody or antigen-binding fragment thereof specifically binding HLA-DR conjugated to a detectable label.

The invention also provides an isolated antibody or antigen-binding fragment thereof specifically binding HLA-DR conjugated to a cytotoxic agent.

Antibodies or antigen-binding fragments thereof that bind HLA-DR may be used to direct therapeutics to HLA-DR-expressing cells. Tumor cells that overexpress HLA-DR may be targeted with an antibody specifically binding HLA-DR conjugated to a cytotoxic agent that kills the cell upon internalization of the HLA-DR antibody. Alternatively, HLA-DR expressing malignant cells could be targeted with an HLA-DR antibody coupled to a therapeutic intended to modify cell function once internalized (e.g., a transcription factor inhibitor). Blood cancer cells as well as tissue cancer cells have been reported to express HLA-DR (Cabrera et al., Scand J Immunol 1995; 41: 398-406; Altomonte et al., Oncogene 2003; 22: 6564-6569), therefore using an antibody to target these cells may provide therapeutic benefit.

The antibodies of the invention are internalized by the cells however they optionally do not induce apoptosis and/or death of B cells. These antibodies may be conjugated to a cytotoxic agent and used to treat HLA-DR positive tumors such as hematological malignancies.

In some embodiments, the detectable label is also a cytotoxic agent.

The isolated antibody or the antigen-binding fragment thereof specifically binding HLA-DR of the invention conjugated to a detectable label may be used to evaluate expression of HLA-DR on a variety of samples.

Detectable label includes compositions that when conjugated to the isolated antibody or the antigen-binding fragment thereof specifically binding HLA-DR of the invention renders the latter detectable, via spectroscopic, photochemical, biochemical, immunochemical, or chemical means.

Exemplary detectable labels include radioactive isotopes, magnetic beads, metallic beads, colloidal particles, fluorescent dyes, electron-dense reagents, enzymes (for example, as commonly used in an ELISA), biotin, digoxigenin, haptens, luminescent molecules, chemiluminescent molecules, fluorochromes, fluorophores, fluorescent quenching agents, colored molecules, radioactive isotopes, scintillates, avidin, streptavidin, protein A, protein G, antibodies or fragments thereof, polyhistidine, Ni²⁺, Flag tags, myc tags, heavy metals, enzymes, alkaline phosphatase, peroxidase, luciferase, electron donors/acceptors, acridinium esters, and colorimetric substrates.

A detectable label may emit a signal spontaneously, such as when the detectable label is a radioactive isotope. In other cases the detectable label emits a signal as a result of being stimulated by an external field.

Exemplary radioactive isotopes may be γ-emitting, Auger-emitting, β-emitting, an alpha-emitting or positron-emitting radioactive isotope. Exemplary radioactive isotopes include ³H, ¹¹C, ¹³C, ¹⁵N, ¹⁸F, ¹⁹F, ⁵⁵Co, ⁵⁷Co, ⁶⁰Co, ⁶¹Cu, ⁶²Cu, ⁶⁴Cu, ⁶⁷Cu, ⁶⁸Ga, ⁷²As, ⁷⁵Br, ⁸⁶Y, ⁸⁹Zr, ⁹⁰Sr, ^{94m}Tc, ^{99m}Tc, ¹¹⁵In, ¹²³I, ¹²⁴I, ¹²⁵I, ¹³¹I, ²¹¹At, ²¹²Bi, ²¹³Bi, ²²³Ra, ²²⁶Ra, ²²⁵Ac and ²²⁷Ac.

Exemplary metal atoms are metals with an atomic number greater than 20, such as calcium atoms, scandium atoms, titanium atoms, vanadium atoms, chromium atoms, manganese atoms, iron atoms, cobalt atoms, nickel atoms, copper atoms, zinc atoms, gallium atoms, germanium atoms, arsenic atoms, selenium atoms, bromine atoms, krypton atoms, rubidium atoms, strontium atoms, yttrium atoms, zirconium atoms, niobium atoms, molybdenum atoms, technetium atoms, ruthenium atoms, rhodium atoms, palladium atoms, silver atoms, cadmium atoms, indium atoms, tin atoms, antimony atoms, tellurium atoms, iodine atoms, xenon atoms, cesium atoms, barium atoms, lanthanum atoms, hafnium atoms, tantalum atoms, tungsten atoms, rhenium atoms, osmium atoms, iridium atoms, platinum atoms, gold atoms, mercury atoms, thallium atoms, lead atoms, bismuth atoms, francium atoms, radium atoms, actinium atoms, cerium atoms, praseodymium atoms, neodymium atoms, promethium atoms, samarium atoms, europium atoms, gadolinium atoms, terbium atoms, dysprosium atoms, holmium atoms, erbium atoms, thulium atoms, ytterbium atoms, lutetium atoms, thorium atoms, protactinium atoms, uranium atoms, neptunium atoms, plutonium atoms, americium atoms, curium atoms, berkcelium atoms, californium atoms, einsteinium atoms, fermium atoms, mendelevium atoms, nobelium atoms, or lawrencium atoms.

In some embodiments, the metal atoms may be alkaline earth metals with an atomic number greater than twenty.

In some embodiments, the metal atoms may be lanthanides.

In some embodiments, the metal atoms may be actinides.

In some embodiments, the metal atoms may be transition metals.

In some embodiments, the metal atoms may be poor metals.

In some embodiments, the metal atoms may be gold atoms, bismuth atoms, tantalum atoms, and gadolinium atoms.

In some embodiments, the metal atoms may be metals with an atomic number of 53 (i.e. iodine) to 83 (i.e. bismuth).

In some embodiments, the metal atoms may be atoms suitable for magnetic resonance imaging.

The metal atoms may be metal ions in the form of +1, +2, or +3 oxidation states, such as Ba²⁺, Bi³⁺, Cs⁺, Ca²⁺, Cr²⁺, Cr³⁺, Cr⁶⁺, Co²⁺, Co³⁺, Cu⁺, Cu²⁺, Cu³⁺, Ga³⁺, Gd³⁺, Au⁺, Au³⁺, Fe²⁺, Fe³⁺, F³⁺, Pb²⁺, Mn²⁺, Mn³⁺, Mn⁴⁺, Mn⁷⁺, Hg²⁺, Ni²⁺, Ni²⁺, Ag⁺, Sr²⁺, Sn²⁺, Sn⁴⁺, and Zn²⁺. The metal atoms may comprise a metal oxide, such as iron oxide, manganese oxide, or gadolinium oxide.

Suitable dyes include any commercially available dyes such as, for example, 5(6)-carboxyfluorescein. IRDye 680RD maleimide or IRDye 800CW, ruthenium polypyridyl dyes, and the like.

Suitable fluorophores are fluorescein isothiocyante (FITC), fluorescein thiosemicarbazide, rhodamine, Texas Red, CyDyes (e.g., Cy3, Cy5, Cy5.5), Alexa Fluors (e.g., Alexa488, Alexa555, Alexa594; Alexa647), near infrared (NIR) (700-900 nm) fluorescent dyes, and carbocyanine and aminostyryl dyes.

The isolated antibodies or the antigen-binding fragments thereof specifically binding HLA-DR of the invention conjugated to a detectable label may be used as an imaging agent to evaluate tumor distribution, diagnosis for the presence of HLA-DR expressing cells.

In some embodiments, the isolated antibodies or the antigen-binding fragments thereof specifically binding HLA-DR of the invention are conjugated to a cytotoxic agent.

In some embodiments, the cytotoxic agent is a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate).

The isolated antibodies or the antigen binding fragments thereof specifically binding HLA-DR of the invention conjugated to a cytotoxic agent may be used in the targeted delivery of the cytotoxic agent to for example AML, ALL or MM cells and intracellular accumulation therein, wherein systemic administration of these unconjugated cytotoxic agents may result in unacceptable levels of toxicity to normal cells.

In some embodiments, the cytotoxic agent is daunomycin, doxorubicin, methotrexate, vindesine, bacterial toxins such as diphtheria toxin, ricin, geldanamycin, maytansinoids or calicheamicin. The cytotoxic agent may elicit their cytotoxic and cytostatic effects by mechanisms including tubulin binding, DNA binding, or topoisomerase inhibition.

In some embodiments, the cytotoxic agent is an enzymatically active toxin such as diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, nutogellin, restrictocin, phenonycin, enomycin, and the tricothecenes.

In some embodiments, the cytotoxic agent is a radionuclide, such as ²¹²Bi, ¹³¹I, ¹³¹In, ⁹⁰Y, and ¹⁸⁶Re.

In some embodiments, the cytotoxic agent is dolastatins or dolostatin peptidic analogs and derivatives, auristatin or monomethyl auristatin phenylalanine. Exemplary molecules are disclosed in U.S. Pat. Nos. 5,635,483 and 5,780,588. Dolastatins and auristatins have been shown to interfere with microtubule dynamics, GTP hydrolysis, and nuclear and cellular division (Woyke et al (2001) Antimicrob Agents and Chemother. 45(12):3580-3584) and have anticancer and antifungal activity. The dolastatin or auristatin drug moiety may be attached to the FN3 domain of the invention through the N (amino) terminus or the C (carboxyl) terminus of the peptidic drug moiety (WO02/088172), or via any cysteine engineered into the FN3 domain.

The isolated antibodies or the antigen-binding fragments thereof specifically binding HLA-DR of the invention of the invention may be conjugated to a detectable label using known methods.

In some embodiments, the detectable label is complexed with a chelating agent.

In some embodiments, the detectable label is conjugated to the antibodies or the antigen-binding fragments thereof specifically binding HLA-DR of the invention via a linker.

The detectable label or the cytotoxic moiety may be linked directly, or indirectly, to the antibodies or antigen-binding fragments thereof specifically binding HLA-DR of the invention using known methods. Suitable linkers are known in the art and include, for example, prosthetic groups, non-phenolic linkers (derivatives of N-succimidyl-benzoates; dodecaborate), chelating moieties of both macrocyclics and acyclic chelators, such as derivatives of 1,4,7,10-tetraazacyclododecane-1,4,7,10,tetraacetic acid (DOTA), derivatives of diethylenetriaminepentaacetic avid (DTPA), derivatives of S-2-(4-Isothiocyanatobenzyl)-1,4,7-triazacyclononane-1,4,7-triacetic acid (NOTA) and derivatives of 1,4,8,11-tetraazacyclodocedan-1,4,8,11-tetraacetic acid (TETA), N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene) and other chelating moieties. Suitable peptide linkers are well known.

In some embodiments, the antibodies or the antigen-binding fragments thereof specifically binding HLA-DR are removed from the blood via renal clearance.

### Generation of Antibodies of the Invention

Antibodies or antigen-binding fragments thereof of the invention specifically binding HLA-DR may be generated using various technologies. For example, the hybridoma method of Kohler and Milstein, Nature 256:495, 1975 may be used to generate monoclonal antibodies. In the hybridoma method, a mouse or other host animal, such as a hamster, rat or monkey, is immunized with human or cyno HLA-DR antigens expressed as Fc fusion proteins in complex with a peptide as described herein, followed by fusion of spleen cells from immunized animals with myeloma cells using standard methods to form hybridoma cells (Goding, Monoclonal Antibodies: Principles and Practice, pp. 59-103 (Academic Press, 1986)). Colonies arising from single immortalized hybridoma cells may be screened for production of antibodies with desired properties, such as specificity of binding, cross-reactivity or lack thereof, and affinity for the antigen.

Exemplary humanization techniques including selection of human acceptor frameworks include CDR grafting (U.S. Pat. No. 5,225,539), SDR grafting (U.S. Pat. No. 6,818,749). Resurfacing (Padlan, (1991) Mol Immunol 28:489-499), Specificity Determining Residues Resurfacing (U.S. Patent Publ. No. 2010/0261620), human framework adaptation (U.S. Pat. No. 8,748,356) or superhumanization (U.S. Pat. No. 7,709,226). In these methods, CDRs of parental antibodies are transferred onto human frameworks that may be selected based on their overall homology to the parental frameworks, based on similarity in CDR length, or canonical structure identity, or a combination thereof.

Humanized antibodies may be further optimized to improve their selectivity or affinity to a desired antigen by incorporating altered framework support residues to preserve binding affinity (backmutations) by techniques such as those described in Int. Patent Publ. Nos. WO1090/007861 and WO1992/22653, or by introducing variation at any of the CDRs for example to improve affinity of the antibody.

Transgenic animals, such as mice or rat carrying human immunoglobulin (Ig) loci in their genome may be used to generate human antibodies against a HLA-DR protein, and are described in for example U.S. Pat. No. 6,150,584, Int. Patent Publ. No. WO99/45962. Int. Patent Publ. Nos. WO2002/066630, WO2002/43478, WO2002/043478 and WO1990/04036, Lonberg et al (1994) Nature 368:856-9; Green et al (1994) Nature Genet. 7:13-21; Green & Jakobovits (1998) Exp. Med 188:483-95; Lonberg and Huszar (1995) Int Rev Immunol 13:65-93; Bruggemann et al., (1991) Eur J Immunol 21:1323-1326; Fishwild et al., (1996) Nat Biotechnol 14:845-851; Mendez et al., (1997) Nat Genet 15:146-156; Green (1999) J Immunol Methods 231:11-23; Yang et al., (1999) Cancer Res 59:1236-1243; Brüggemann and Taussig (1997) Curr Opin Biotechnol 8:455-458. The endogenous immunoglobulin loci in such animal may be disrupted or deleted, and at least one complete or partial human immunoglobulin locus may be inserted into the genome of the animal using homologous or non-homologous recombination, using transchromosomes, or using minigenes. Companies such as Regeneron (http://_www_regeneron_com), Harbour Antibodies (http://_www_harbourantibodies_com), Open Monoclonal Technology, Inc. (OMT) (http://_www_omtinc_net), KyMab (http://_www_kymab_com), Trianni (http://_www.trianni_com) and Ablexis (http://_www_ablexis_com) may be engaged to provide human antibodies directed against a selected antigen using technologies as described above.

Human antibodies may be selected from a phage display library, where the phage is engineered to express human immunoglobulins or portions thereof such as Fabs, single chain antibodies (scFv), or unpaired or paired antibody variable regions (Knappik et al., (2000) J Mol Biol 296:57-86; Krebs et al., (2001) J Immunol Meth 254:67-84; Vaughan et al., (1996) Nature Biotechnology 14:309-314; Sheets et al., (1998) PITAS (USA) 95:6157-6162; Hoogenboom and Winter (1991) J Mol Biol 227:381; Marks et al., (1991) J Mol Biol 222:581). The antibodies of the invention may be isolated for example from phage display library expressing antibody heavy and light chain variable regions as fusion proteins with bacteriophage pIX coat protein as described in Shi et al., (2010) J Mol Biol 397:385-96, and Int. Patent Publ. No. WO09/085462). The libraries may be screened for phage binding to human and/or cyno HLA-DR and the obtained positive clones may be further characterized, the Fabs isolated from the clone lysates, and expressed as full length IgGs. Such phage display methods for isolating human antibodies are described in for example: U.S. Pat. Nos. 5,223,409, 5,403,484, 5,571,698, 5,427,908, 5,580,717, 5,969,108, 6,172,197, 5,885,793; 6,521,404; 6,544,731; 6,555,313; 6,582,915 and 6,593,081.

Antibodies that compete for binding to HLA-DR with reference antibodies may be generated by isolating antibodies specifically binding human HLA-DR using phage display libraries, and screening the generated antibodies for their ability to compete for binding to HLA-DR with the reference antibodies.

Preparation of immunogenic antigens and monoclonal antibody production may be performed using any suitable technique, such as recombinant protein production. The immunogenic antigens may be administered to an animal in the form of purified protein, or protein mixtures including whole cells or cell or tissue extracts, or the antigen may be formed de novo in the animal's body from nucleic acids encoding said antigen or a portion thereof.

In some embodiments, the antibody or the antigen-binding fragment thereof specifically binding HLA-DR of the invention is a bispecific antibody.

In some embodiments, the antibody or the antigen-binding fragment thereof of the invention is a multispecific antibody.

The monospecific antibodies specifically binding HLA-DR of the invention may be engineered into bispecific antibodies which are also encompassed within the scope of the invention. The VL and/or the VH regions of the antibodies of the invention may be engineered using published methods into single chain bispecific antibodies as structures such as TandAb® designs (Int Pat. Publ. No. WO1999/57150; U.S. Pat. Publ. No. 201110206672) or into bispecific scFVs as structures such as those disclosed in U.S. Pat. No. 5,869,620; Int. Pat. Publ. No. WO1995/15388, Int. Pat Publ. No. WO1997/14719 or Int. Pat Publ. No. WO2011/036460.

The VL and/or the VH regions of the antibodies of the invention may be engineered into bispecific full length antibodies, where each antibody arm binds a distinct antigen or epitope. Such bispecific antibodies may be made by modulating the CH3 interactions between the two antibodies heavy chains to form bispecific antibodies using technologies such as those described in U.S. Pat. No. 7,695,936; Int. Pat. Publ. No. WO2004/111233; U.S. Pat. Publ. No. 2010/0015133; U.S. Pat Publ. No. 2007/0287170; Int. Pat Publ. No. WO2008/119353; U.S. Pat. Publ. No. 2009/0182127; U.S. Pat. Publ. No. 2010/0286374; U.S. Pat. Publ. No. 2011/0123532; Int. Pat Publ. No. WO02011/131746; Int. Pat. Publ. No. WO2011/143545; or U.S. Pat. Publ. No. 2012/0149876.

For example, bispecific antibodies may be generated in vitro in a cell-free environment by introducing asymmetrical mutations in the CH3 regions of two monospecific homodimeric antibodies and forming the bispecific heterodimeric antibody from the two parent monospecific homodimeric antibodies in reducing conditions to allow disulfide bond isomerization according to methods described in Intl. Pat. Publ. No. WO2011/131746. In the methods, two monospecific bivalent antibodies are engineered to have certain substitutions at the CH3 domain that promote heterodimer stability; the antibodies are incubated together under reducing conditions sufficient to allow the cysteines in the hinge region to undergo disulfide bond isomerization thereby generating the bispecific antibody by Fab arm exchange. The incubation conditions may optimally be restored to non-reducing. Exemplary reducing agents that may be used are 2-mercaptoethylamine (2-MEA), dithiothreitol (DTI), dithioerythritol (DTE), glutathione, tris(2-carboxyethyl)phosphine (TCEP), L-cysteine and beta-mercaptoethanol, preferably a reducing agent selected from the group consisting of: 2-mercaptoethylamine, dithiothreitol and tris(2-caboxyethyl)phosphine. For example, incubation for at least 90 min at a temperature of at least 20° C. in the presence of at least 25 mM 2-MEA or in the presence of at least 0.5 mM dithiothreitol at a pH of from 5-8, for example at pH of 7.0 or at pH of 7.4 may be used.

Exemplary CH3 mutations that may be used in a first heavy chain and in a second heavy chain of the bispecific antibody are K409R and F405L.

Additional multispecific structures into which the VL and/or the VH regions of the antibodies of the invention may be incorporated are for example Dual Variable Domain Immunoglobulins (DVD) (Int. Pat. Publ. No. WO2009/134776), or structures that include various dimerization domains to connect the two antibody arms with different specificity, such as leucine zipper or collagen dimerization domains (Int. Pat. Publ. No. WO2012/022811, U.S. Pat. No. 5,932,448, U.S. Pat. No. 6,833,441). DVDs are full length antibodies comprising the heavy chain having a structure VH1-linker-VH2-CH and the light chain having the structure VL1-linker-VL2-CL; linker being optional.

### Polynucleotides, Vectors and Host Cells

The invention also provides for an antibody or an antigen-binding fragment thereof that specifically binds HLA-DR having certain VH and VL sequences, wherein the antibody VH is encoded by a first polynucleotide and the antibody VL is encoded by a second polynucleotide. The polynucleotide may be a complementary deoxynucleic acid (cDNA), and may be codon optimized for expression in suitable host. Codon optimization is a well-known technology.

The invention also provides for an isolated polynucleotide encoding the VH of the antibody of the invention, the VL of the antibody of the invention, the heavy chain of the antibody of the invention or the light chain of the antibody of the invention.

The invention also provides for an isolated polynucleotide encoding the VI of SEQ ID NOs: 56, 57, 58, 59, 137, 138, 139, 140 or 141.

The invention also provides for an isolated polynucleotide encoding the VL of SEQ ID NOs: 60, 61 or 142.

The invention also provides for an isolated polynucleotide encoding the VH of SEQ ID NOs: 56, 57, 58, 59, 137, 138, 139, 140 or 141 and the VL of SEQ ID NOs: 60, 61 or 142.

The invention also provides for an isolated polynucleotide encoding the heavy chain of SEQ ID NOs: 84, 85, 86, 87, 96, 97, 98, 99, 149, 150, 151, 152 or 153.

The invention also provides for an isolated polynucleotide encoding the light chain of SEQ ID NOs: 88, 89 or 154.

The invention also provides for an isolated polynucleotide encoding the heavy chain of SEQ ID NOs: 84, 85, 86, 87, 96, 97, 98, 99, 149, 150, 151, 152 or 153 and a light chain of SEQ ID NOs: 88, 89 or 154.

The invention also provides for an isolated polynucleotide comprising the polynucleotide sequence of SEQ ID NOs: 79, 80, 81, 82, 83, 90, 91, 92, 93, 94, 95, 100, 101, 102, 103, 121, 143, 144, 145, 146, 147, 148, 155, 156, 157, 158, 159 or 160.

The polynucleotide sequences encoding the VH and/or the VL or an antigen-binding fragment thereof of the antibodies of the invention, or the heavy chain and the light chain of the antibodies of the invention may be operably linked to one or more regulatory elements, such as a promoter or enhancer, that allow expression of the nucleotide sequence in the intended host cell. The polynucleotide may be a cDNA.

The invention also provides for a vector comprising the polynucleotide of the invention. Such vectors may be plasmid vectors, viral vectors, vectors for baculovirus expression, transposon based vectors or any other vector suitable for introduction of the synthetic polynucleotide of the invention into a given organism or genetic background by any means. For example, polynucleotides encoding light and/or heavy chain variable regions of the antibodies of the invention, optionally linked to constant regions, are inserted into expression vectors. The light and/or heavy chains may be cloned in the same or different expression vectors. The DNA segments encoding immunoglobulin chains may be operably linked to control sequences in the expression vector(s) that ensure the expression of immunoglobulin polypeptides. Such control sequences include signal sequences, promoters (e.g. naturally associated or heterologous promoters), enhancer elements, and transcription termination sequences, and are chosen to be compatible with the host cell chosen to express the antibody. Once the vector has been incorporated into the appropriate host, the host is maintained under conditions suitable for high level expression of the proteins encoded by the incorporated poly nucleotides.

In some embodiments, the vector comprises the polynucleotide of SEQ ID NO: 79 and the polynucleotide of SEQ ID NO: 80.

In some embodiments, the vector comprises the polynucleotide of SEQ ID NO: 81 and the polynucleotide of SEQ ID NO: 82.

In some embodiments, the vector comprises the polynucleotide of SEQ ID NO: 83 and the polynucleotide of SEQ ID NO: 82.

In some embodiments, the vector comprises the polynucleotide of SEQ ID NO: 121 and the polynucleotide of SEQ ID NO: 82.

In some embodiments, the vector comprises the polynucleotide of SEQ ID NO: 143 and the polynucleotide of SEQ ID NO: 82.

In some embodiments, the vector comprises the polynucleotide of SEQ ID NO: 144 and the polynucleotide of SEQ ID NO: 82.

In some embodiments, the vector comprises the polynucleotide of SEQ ID NO: 145 and the polynucleotide of SEQ ID NO: 82.

In some embodiments, the vector comprises the polynucleotide of SEQ ID NO: 146 and the polynucleotide of SEQ ID NO: 148.

In some embodiments, the vector comprises the polynucleotide of SEQ ID NO: 147 and the polynucleotide of SEQ ID NO: 82.

Suitable expression vectors are typically replicable in the host organisms either as episomes or as an integral part of the host chromosomal DNA. Commonly, expression vectors contain selection markers such as ampicillin-resistance, hygromycin-resistance, tetracycline resistance, kanamycin resistance or neomycin resistance to permit detection of those cells transformed with the desired DNA sequences.

Suitable promoter and enhancer elements are known in the art. For expression in a eukarotic cell, exemplary promoters include light and/or heavy chain immunoglobulin gene promoter and enhancer elements; cytomegalovirus immediate early promoter, herpes simplex virus thymidine kinase promoter, early and late SV40 promoters; promoter present in long terminal repeats from a retrovirus; mouse metallothionein-I promoter, and various known tissue specific promoters. Selection of the appropriate vector and promoter is well within the level of ordinary skill in the art.

Exemplary vectors that may be used are Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala. Sweden). Eukaryotic: pWLneo, pSV2cat, pOG44, PXR1, pSG (Stratagene) pSVK3, pBPV, pMSG and pSVL (Pharmacia), pEE6.4 (Lonza) and pEE12.4 (Lonza).

The invention also provides for a host cell comprising one or more vectors of the invention. “Host cell” refers to a cell into which a vector has been introduced. It is understood that the term host cell is intended to refer not only to the particular subject cell but to the progeny of such a cell, and also to a stable cell line generated from the particular subject cell. Because certain modifications may occur in succeeding generations due to either mutation or environmental influences, such progeny may not be identical to the parent cell, but are still included within the scope of the term “host cell” as used herein. Such host cells may be eukaryotic cells, prokaryotic cells, plant cells or archeal cells. Escherichia coli, bacilli, such as Bacillus subtilis, and other enterobacteriaceae, such as Salmonella, Serratia, and various Pseudomonas species are examples of prokaryotic host cells. Other microbes, such as yeast, are also useful for expression. Saccharomyces (for example, S. cerevisiae) and Pichia are examples of suitable yeast host cells. Exemplary eukaryotic host cells may be of mammalian, insect, avian or other animal origins. Mammalian eukaryotic host cells include immortalized cell lines such as hybridomas or myeloma cell lines such as SP2/0 (American Type Culture Collection (ATCC), Manassas, Va., CRL-1581), NS0 (European Collection of Cell Cultures (ECACC), Salisbury. Wiltshire, UK, ECACC No. 85110503). FO (ATCC CRL-1646) and Ag653 (ATCC CRL-1580) murine cell lines. An exemplary human myeloma cell line is U266 (ATTC CRL-TIB-196). Other useful cell lines include those derived from Chinese Hamster Ovary (CHO) cells such as CHOK1SV (Lonza Biologics, Walkersville, Md.), Potelligent® CHOK2SV (Lonza), CHO-K1 (ATCC CRL-61) or DG44.

The invention also provides for a method of producing the antibody or the antigen-binding fragment thereof of the invention comprising culturing the host cell of the invention in conditions that the antibody is expressed, and recovering the antibody produced by the host cell. Methods of making antibodies and purifying them are well known in the art Once synthesized (either chemically or recombinantly), the whole antibodies, their dimers, individual light and/or heavy chains, or other antibody fragments such as VH and/or VL, may be purified according to standard procedures, including ammonium sulfate precipitation, affinity columns, column chromatography, high performance liquid chromatography (HPLC) purification, gel electrophoresis, and the like (see generally Scopes. Protein Purification (Springer-Verlag. N.Y., (1982)). A subject antibody may be substantially pure, for example, at least about 80% to 85% pure, at least about 85% to 90% pure, at least about 90% to 95% pure, or at least about 98% to 99%, or more, pure, for example, free from contaminants such as cell debris, macromolecules. etc. other than the subject antibody.

The invention also provides for a method of producing the antibody of the antigen-binding fragment thereof specifically binding HLA-DR of the invention, comprising:
incorporating the first polynucleotide encoding the VH of the antibody and the second polynucleotide encoding the VL of the antibody into an expression vector,
transforming a host cell with the expression vector;
culturing the host cell in culture medium under conditions wherein the VL and the VH are expressed and form the antibody; and
recovering the antibody from the host cell or culture medium.

The polynucleotides encoding certain VH or VL sequences of the invention may be incorporated into vectors using standard molecular biology methods. Host cell transformation, culture, antibody expression and purification are done using well known methods.

### Pharmaceutical Compositions/Administration

The invention provides for pharmaceutical compositions comprising the antibodies or the antigen-binding fragments thereof of the invention and a pharmaceutically acceptable carrier. For therapeutic use, the antibodies of the invention may be prepared as pharmaceutical compositions containing an effective amount of the antibodies as an active ingredient in a pharmaceutically acceptable carrier. “Carrier” refers to a diluent, adjuvant, excipient, or vehicle with which the antibody of the invention is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine may be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the antibodies or the antigen-binding fragments thereof of the invention in such pharmaceutical formulation may vary, from less than about 0.5%, usually to at least about 1% to as much as 15 or 20% by weight and may be selected primarily based on required dose, fluid volumes, viscosities, etc., according to the particular mode of administration selected. Suitable vehicles and formulations, inclusive of other human proteins, e.g., human serum albumin, are described, for example, in e.g. Remington: The Science and Practice of Pharmacy, 21^{st} Edition, Troy, D. B. ed., Lipincott Williams and Wilkins, Philadelphia, Pa. 2006, Part 5, Pharmaceutical Manufacturing pp 691-1092, See especially pp. 958-989.

The mode of administration for therapeutic use of the antibodies or the antigen-binding fragments thereof of the invention may be any suitable route that delivers the antibody to the host, such as parenteral administration. e.g., intradermal, intramuscular, intraperitoneal, intravenous or subcutaneous, pulmonary, transmucosal (oral, intranasal, intravaginal, rectal), using a formulation in a tablet, capsule, solution, powder, gel, particle; and contained in a syringe, an implanted device, osmotic pump, cartridge, micropump; or other means appreciated by the skilled artisan, as well known in the art. Site specific administration may be achieved by for example intratumoral, intrarticular, intrabronchial, intraabdominal, intracapsular, intracartilaginous, intracavitary, intracelial, intracerebellar, intracerebroventricular, intracolic, intracervical, intragastric, intrahepatic, intracardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravascular, intravesical, intralesional, vaginal, rectal, buccal, sublingual, intranasal, or transdermal delivery.

The antibodies or the antigen-binding fragments thereof of the invention may be administered to a subject by any suitable route, for example parentally by intravenous (i.v.) infusion or bolus injection, intramuscularly or subcutaneously or intraperitoneally, i.v. infusion may be given over for example 15, 30, 60, 90, 120, 180, or 240 minutes, or from 1, 2, 3, 4, 5, 6, 7, 8, 9, 10, 11 or 12 hours.

The dose given to a subject is sufficient to alleviate, at least partially arrest, or prevent the disease being treated (“therapeutically effective amount”) and may be sometimes 0.005 mg to about 100 mg/kg, e.g. about 0.05 mg to about 30 mg/kg or about 5 mg to about 25 mg/kg, or about 4 mg/kg, about 8 mg/kg, about 16 mg/kg or about 24 mg/kg, or for example about 1, 2, 3, 4, 5, 6, 7, 8, 9 or 10 mg/kg, but may even higher, for example about 15, 16, 17, 18, 19, 20, 21, 22, 23, 24, 25, 30, 40, 50, 60, 70, 80, 90 or 100 mg/kg.

A fixed unit dose may also be given, for example, 50, 100, 200, 500 or 1000 mg, or the dose may be based on the patient's surface area, e.g., 500, 400, 300, 250, 200, or 100 mg/m². Usually between 1 and 8 doses, (e.g., 1, 2, 3, 4, 5, 6, 7 or 8) may be administered to treat the patient, but 9, 10, 11, 12, 13, 14, 15, 16, 17, 18, 19, 20 or more doses may be given.

The administration of the antibodies or the antigen-binding fragments thereof of the invention may be repeated after one day, two days, three days, four days, five days, six days, one week, two weeks, three weeks, one month, five weeks, six weeks, seven weeks, two months, three months, four months, five months, six months or longer. Repeated courses of treatment are also possible, as is chronic administration. The repeated administration may be at the same dose or at a different dose. For example, the antibodies or the antigen-binding fragments thereof of the invention described herein may be administered at 8 mg/kg or at 16 mg/kg at weekly interval for 8 weeks, followed by administration at 8 mg/kg or at 16 mg/kg every two weeks for an additional 16 weeks, followed by administration at 8 mg/kg or at 16 mg/kg every four weeks by intravenous infusion.

For example, the antibodies or the antigen-binding fragments thereof of the invention may be provided as a daily dosage in an amount of about 0.1-100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20 after initiation of treatment, or any combination thereof, using single or divided doses of every 24, 12, 8, 6, 4, or 2 hours, or any combination thereof.

The antibodies or the antigen-binding fragments thereof of the invention may also be administered prophylactically in order to reduce the risk of developing an autoimmune disease and/or delay the onset of the symptoms.

The antibodies or the antigen-binding fragments thereof of the invention may be lyophilized for storage and reconstituted in a suitable carrier prior to use. This technique has been shown to be effective with conventional protein preparations and well known lyophilization and reconstitution techniques can be employed.

### Methods and Uses

The antibodies or the antigen-binding fragments thereof of the invention have in vitro and in vivo diagnostic, as well as therapeutic and prophylactic utilities. For example, the antibodies of the invention may be administered to cells in culture, in vitro or ex vivo, or to a subject to treat, prevent, and/or diagnose a variety of disorders, such as HLA-DR-mediated diseases such as an autoimmune diseases, or HLA-DR expressing tumors.

The invention also provides a method of treating or preventing HLA-DR-mediated disease, comprising administering to a subject in need thereof a therapeutically effective amount of the antibody or the antigen-binding fragment thereof specifically binding HLA-DR of the invention for a time sufficient to treat HLA-DR-mediated disease.

The invention also provides a method of preventing HLA-DR-mediated disease, comprising administering to a subject in need thereof a therapeutically effective amount of the antibody or the antigen-binding fragment thereof specifically binding HLA-DR of the invention for a time sufficient to treat HLA-DR-mediated disease.

In some embodiments, HLA-DR-mediated disease is an autoimmune disease.

In some embodiments, the autoimmune disease is arthritis.

In some embodiments, arthritis is juvenile arthritis, rheumatoid arthritis, psoriatic arthritis. Reiter's syndrome, ankylosing spondylitis, or gouty arthritis.

In some embodiments, the autoimmune disease is systemic juvenile idiopathy arthritis.

In some embodiments, the autoimmune disease is Grave's disease.

In some embodiments, the autoimmune disease is Hashimoto's thyroiditis.

In some embodiments, the autoimmune disease is myasthenia gravis.

In some embodiments, the autoimmune disease is multiple sclerosis.

In some embodiments, the autoimmune disease is lupus.

In some embodiments, lupus is systemic lupus erythematosus (SLE) or cutaneous lupus erythematosus (CLE).

In some embodiments, the subject has lupus nephritis.

In some embodiments, the autoimmune disease is type 1 diabetes.

In some embodiments, the autoimmune disease is inflammatory bowel disease.

In some embodiments, inflammatory bowel disease is Crohn's disease.

In some embodiments, inflammatory bowel disease is ulcerative colitis.

The invention also provides a method of treating a HLA-DRB1-associated autoimmune disease, comprising administering to a subject in need thereof a therapeutically effective amount of the antibody or the antigen-binding fragment thereof specifically binding HLA-DR of the invention for a time sufficient to treat the autoimmune disease.

The invention also provides a method of preventing an HLA-DRB1-associated autoimmune disease, comprising administering to a subject in need thereof a therapeutically effective amount of the antibody or the antigen-binding fragment thereof specifically binding HLA-DR of the invention for a time sufficient to prevent the autoimmune disease.

In some embodiments, the autoimmune disease is rheumatoid arthritis, systemic juvenile idiopathic arthritis, Grave's disease, Hashimoto's thyroiditis, myasthenia gravis, multiple sclerosis, lupus or type 1 diabetes.

The invention also provides a method of suppressing an immune response towards a self-antigen, comprising administering to a subject in need thereof the antibody or the antigen-binding fragment thereof specifically binding HLA-DR of the invention for a time sufficient to suppress the immune response towards a self-antigen.

The invention also provides a method of treating an HLA-DRB1-associated autoimmune disease, comprising administering to a subject in need thereof a therapeutically effective amount of the antibody or the antigen-binding fragment thereof specifically binding HLA-DR comprising the HCDR1, the HCDR2, the HCDR3, the LCDR1, the LCDR2 and the LCDR3 of SEQ ID NOs: 39, 42, 46, 50, 52 and 54, respectively for a time sufficient to treat the HLA-DRB1-associated autoimmune disease.

In some embodiments, the antibody comprises the VH of SEQ ID NO: 56 and the VL of SEQ ID NO: 60.

The invention also provides a method of treating an HLA-DRB1-associated autoimmune disease, comprising administering to a subject in need thereof a therapeutically effective amount of the antibody or the antigen-binding fragment thereof specifically binding HLA-DR comprising the HCDR1, the HCDR2, the HCDR3, the LCDR1, the LCDR2 and the LCDR3 of SEQ ID NOs: 40, 43, 47, 51, 53 and 55, respectively for a time sufficient to treat the HLA-DRB1-associated autoimmune disease.

In some embodiments, the antibody comprises the VH of SEQ ID NO: 57 and the VL of SEQ ID NO: 61.

The invention also provides a method of treating an HLA-DRB1-associated autoimmune disease, comprising administering to a subject in need thereof a therapeutically effective amount of the antibody or the antigen-binding fragment thereof specifically binding HLA-DR comprising the HCDR1, the HCDR2, the HCDR3, the LCDR1, the LCDR2 and the LCDR3 of SEQ ID NOs: 41, 44, 48, 51, 53 and 55, respectively for a time sufficient to treat the HLA-DRB1-associated autoimmune disease.

In some embodiments, the antibody comprises the VH of SEQ ID NO: 58 and the VL of SEQ ID NO: 61.

The invention also provides a method of treating an HLA-DRB1-associated autoimmune disease, comprising administering to a subject in need thereof a therapeutically effective amount of the antibody or the antigen-binding fragment thereof specifically binding HLA-DR comprising the HCDR1, the HCDR2, the HCDR3, the LCDR1, the LCDR2 and the LCDR3 of SEQ ID NOs: 41, 45, 49, 51, 53 and 55, respectively for a time sufficient to treat the HLA-DRB1-associated autoimmune disease.

In some embodiments, the antibody comprises the VH of SEQ ID NO: 59 and the VL of SEQ ID NO: 61.

The invention also provides a method of treating HLA-DR expressing tumor, comprising administering to a subject in need thereof a therapeutically effective amount of the antibody or the antigen-binding fragment thereof of the invention conjugated to a cytotoxic agent for a time sufficient to treat HLA-DR expressing tumor.

In some embodiments. HLA-expressing tumor is a hematological malignancy.

In some embodiments hematological malignancy is B cell non-Hodgkin's lymphoma, B cell lymphoma. B cell acute lymphoid leukemia, Burkitt's lymphoma. Hodgkin's lymphoma, hairy cell leukemia, acute myeloid leukemia, T cell lymphoma, T cell non-Hodgkin's lymphoma, chronic myeloid leukemia, chronic lymphoid leukemia, multiple myeloid leukemia or acute monoblastic leukemia (AMoL).

In some embodiments, HLA-expressing tumor is a glioma.

In some embodiments, HLA-expressing tumor is an ovarian cancer.

In some embodiments, HLA-expressing tumor is a colorectal cancer.

In some embodiments, HLA-expressing tumor is an osteosarcoma.

In some embodiments, HLA-expressing tumor is a cervical cancer.

In some embodiments, HLA-expressing tumor is a stomach cancer.

In some embodiment, a subject has tumor in colon, larynx, skeletal muscle, breast or lung.

HLA-DR expression has been identified in these cancers (see e.g. Diao et al., Int J Clin Exp Pathol 2015; 8(5): 5483-90; Rangel et al., Cancer Biol ther 2004; 3(10): 1021-7; Cabrera et al., Scand J Immunol 1995; 41: 398-406; Matsushita et al., Cancer Sci 2005; 97(1): 57-63; Trieb et al., Pathol res Practices 1998; 194: 679-684)

“Therapeutically effective amount” of the antibody or the antigen-binding fragment thereof specifically binding HLA-DR of the invention effective in the treatment of HLA-mediated disease, an autoimmune disease and/or cancer may be determined by standard research techniques. For example, in vitro assays may be employed to help identify optimal dosage ranges. Optionally, the dosage of the antibodies or the antigen-binding fragments thereof specifically binding HLA-DR of the invention that may be effective in the treatment of autoimmune diseases such as arthritis or rheumatoid arthritis, or cancer, may be determined by administering the antibodies specifically binding HLA-DR to relevant animal models known in the art. Selection of a particular effective dose may be determined (e.g., via clinical trials) by those skilled in the art based upon the consideration of several factors. Such factors include the disease to be treated or prevented, the symptoms involved, the patient's body mass, the patient's immune status and other factors known by the skilled artisan. The precise dose to be employed in the formulation will also depend on the route of administration, and the severity of disease, and should be decided according to the judgment of the practitioner and each patient's circumstances. Effective doses can be extrapolated from dose-response curves derived from in vitro or animal model test systems. The antibodies of the invention may be tested for their efficacy and effective dosage using any of the models described herein.

### Combination Therapies

The antibodies or antigen-binding fragments thereof specifically binding HLA-DR in the methods of the invention may be administered in combination with a second therapeutic agent simultaneously, sequentially or separately.

The antibodies or antigen-binding fragments thereof specifically binding HLA-DR of the invention may be administered in combination with any known therapy for autoimmune diseases, including any agent or combination of agents that are known to be useful, or which have been used or are currently in use, for treatment of autoimmune diseases. Such therapies and therapeutic agents include surgery or surgical procedures (e.g. splenectomy, lymphadenectomy, thyroidectomy, plasmapheresis, leukophoresis, cell, tissue, or organ transplantation, intestinal procedures, organ perfusion, and the like), radiation therapy, therapy such as steroid therapy and non-steroidal therapy, hormone therapy, cytokine therapy, therapy with dermatological agents (for example, topical agents used to treat skin conditions such as allergies, contact dermatitis, and psoriasis), immunosuppressive therapy, and other anti-inflammatory monoclonal antibody therapy.

The second therapeutic agent may be a corticosteroid, an antimalarial drug, an immunosuppressant, a cytotoxic drug, or a B-cell modulator.

In some embodiments, the second therapeutic agent is prednisone, prednisolone, methylprednisolone, deflazcort, hydroxychloroquine, azathioprine, methotrexate, cyclophosphamide, mycophenolate mofetil (MMF), mycophenolate sodium, cyclosporine, leflunomide, tacrolimus, rituximab (Rituxan®), or belimumab (Benlysta®).

In some embodiments, the second therapeutic agent is corticosteroids, nonsteroidal anti-inflammatory drugs (NSAIDs), salicylates, sulfasalazine, cytotoxic drugs, immunosuppressive drugs, mizoribine, chlorambucil, cyclosporine, tacrolimus (FK506; ProGrafrM), mycophenolate mofetil, sirolimus (rapamycin), deoxyspergualin, leflunomide and its malononitriloamide analogs, clobetasol, halobetasol, hydrocortisone, triamcinolone, betamethasone, fluocinole, fluocinonide, medications containing mesalamine (known as 5-ASA agents), celecoxib, diclofenac, etodolac, fenprofen, flurbiprofen, ibuprofen, ketoprofen, meclofamate, meloxicam, nabumetone, naproxen, oxaprozin, piroxicam, rofecoxib, salicylates, sulindac, tolmetin; phosphodiesterase-4 inhibitors, anti-TNFα antibodies infliximab (REMICADE®), golimumab (SIMPONI®) and adalimumab (HUMIRA®), thalidomide or its analogs such as lenalidomide.

Treatment effectiveness or RA may be assessed using effectiveness as measured by clinical responses defined by the American College of Rheumatology criteria, the European League of Rheumatism criteria, or any other criteria. See for example, Felson et al. (1995) Arthritis Rheum. 38: 727-35 and van Gestel et al. (19%) Arthritis Rheum. 39: 34-40.

The antibodies or antigen-binding fragments thereof specifically binding HLA-DR of the invention or the antibodies or antigen-binding fragments thereof specifically binding HLA-DR conjugated to a cytotoxic agent may be administered in combination with any known cancer therapies, such as therapies used to treat hematological malignancies.

### Diagnostic Uses and Kits

### Kits

The invention also provides a kit comprising the antibody or the antigen-binding fragment thereof specifically binding HLA-DR of the invention.

The kit may be used for therapeutic uses and as diagnostic kits.

The kit may be used to detect the presence of HLA-DR in a sample.

In some embodiments, the kit comprises the antibody or the antigen-binding fragment thereof of the invention and reagents for detecting the antibody. The kit can include one or more other elements including: instructions for use; other reagents, e.g., a label, a therapeutic agent, or an agent useful for chelating, or otherwise coupling, an antibody to a label or therapeutic agent, or a radioprotective composition; devices or other materials for preparing the antibody for administration; pharmaceutically acceptable carriers; and devices or other materials for administration to a subject.

In some embodiments, the kit comprises the antibody or the antigen-binding fragment thereof of the invention in a container and instructions for use of the kit.

In some embodiments, the antibody in the kit is labeled.

In some embodiments, the kit comprises the antibody or the antigen-binding fragment thereof specifically binding HLA-DR comprising the VH of SEQ ID NO: 56 and the VL of SEQ ID NO: 60.

In some embodiments, the kit comprises the antibody or the antigen-binding fragment thereof specifically binding HLA-DR comprising the VH of SEQ ID NO: 57 and the VL of SEQ ID NO: 61.

In some embodiments, the kit comprises the antibody or the antigen-binding fragment thereof specifically binding HLA-DR comprising the VI of SEQ ID NO: 58 and the VL of SEQ ID NO: 61.

In some embodiments, the kit comprises the antibody or the antigen-binding fragment thereof specifically binding HLA-DR comprising the VI of SEQ ID NO: 59 and the VL of SEQ ID NO: 61.

In some embodiments, the kit comprises the antibody or the antigen-binding fragment thereof specifically binding HLA-DR comprising the VH of SEQ ID NO: 137 and the VL of SEQ ID NO: 61.

In some embodiments, the kit comprises the antibody or the antigen-binding fragment thereof specifically binding HLA-DR comprising the VII of SEQ ID NO: 138 and the VL of SEQ ID NO: 61.

In some embodiments, the kit comprises the antibody or the antigen-binding fragment thereof specifically binding HLA-DR comprising the VH of SEQ ID NO: 139 and the VL of SEQ ID NO: 61.

In some embodiments, the kit comprises the antibody or the antigen-binding fragment thereof specifically binding HLA-DR comprising the VH of SEQ ID NO: 140 and the VL of SEQ ID NO: 142.

In some embodiments, the kit comprises the antibody or the antigen-binding fragment thereof specifically binding HLA-DR comprising the VH of SEQ ID NO: 141 and the VL of SEQ ID NO: 61.

### Methods of Detecting HLA-DR

The invention also provides a method of detecting HLA-DR in a sample, comprising obtaining the sample, contacting the sample with the antibody or the antigen-binding fragment thereof specifically binding HLA-DR of the invention, and detecting the antibody bound to HLA-DR in the sample.

In some embodiments, the sample may be derived from urine, blood, serum, plasma, saliva, ascites, circulating cells, circulating tumor cells, cells that are not tissue associated (i.e., free cells), tissues (e.g., surgically resected tumor tissue, biopsies, including fine needle aspiration), histological preparations, and the like.

The antibodies or the antigen-binding fragments thereof of the invention bound to HLA-DR may be detected using known methods. Exemplary methods include direct labeling of the antibodies using fluorescent or chemiluminescent labels, or radiolabels, or attaching to the antibodies of the invention a moiety which is readily detectable, such as biotin, enzymes or epitope tags. Exemplary labels and moieties are ruthenium, ¹¹¹In-DOTA. ¹¹¹In-diethylenetriaminepentaacetic acid (DTPA), horseradish peroxidase, alkaline phosphatase and beta-galactosidase, poly-histidine (HIS tag), acridine dyes, cyanine dyes, fluorone dyes, oxazin dyes, phenanthridine dyes, rhodamine dyes and Alexafluor dyes.

The antibodies of the invention may be used in a variety of assays to detect HLA-DR in the sample. Exemplary assays are western blot analysis, radioimmunoassay, surface plasmon resonance, immunoprecipitation, equilibrium dialysis, immunodiffusion, electrochemiluminescence (ECL) immunoassay, immunohistochemistry, fluorescence-activated cell sorting (FACS) or ELISA assay.

### Further Embodiments of the Invention

Set out below are certain further embodiments of the invention according to the disclosures elsewhere herein. Features from embodiments of the invention set out above described as relating to the invention disclosed herein also relate to each and every one of these further numbered embodiments.
1) An isolated antibody specifically binding HLA-DR comprising an HLA-DRα chain and an HLA-DRβ chain.
2) The antibody of claim 1, wherein HLA-DR is HLA-DR4.
3) The antibody of claim 2, wherein the HLA-DRα chain comprises an amino acid sequence of SEQ ID NO: 13 and the HLA-DRβ chain comprises an amino acid sequence of SEQ ID NO: 14.
4) The antibody of claim 1, wherein HLA-DR is HLA-DR1.
5) The antibody of claim 4, wherein the HLA-DRα chain comprises an amino acid sequence of SEQ ID NO: 13 and the HLA-DRβ chain comprises an amino acid sequence of SEQ ID NO: 15.
6) The antibody of any one of claims 1-5, wherein the antibody lacks an ability to induce apoptosis of B cells.
7) The antibody of claim 6, wherein apoptosis is determined by measuring frequency of CD3⁻ CD20⁺ annexinV⁺live/dead⁻ B cells in a sample of human peripheral blood cells (PBMC) using flow cytometry.
8) The antibody of any one of claims 1-7, wherein the antibody lacks the ability to induce death of B cells.
9) The antibody of claim 7, wherein the death of B cells is determined by measuring frequency of CD3⁻ CD20⁺ annexinV⁺live/dead⁺ B cells in the sample of human PBMC using flow cytometry.
10) The antibody of claim 1, wherein HLA-DR contains a shared epitope.
11) The antibody of claim 10, wherein the shared epitope comprises an amino acid sequence QKRAA (SEQ ID NO: 66). QRRAA (SEQ ID NO: 67), or RRRAA (SEQ ID NO: 68).
12) The antibody of claim 10 or 11, wherein the shared epitope consists of an amino acid sequence QKRAA (SEQ ID NO: 66). QRRAA (SEQ ID NO: 67), or RRRAA (SEQ ID NO: 68).
13) The antibody of any one of claims 1-12, wherein HLA-DR is in complex with a peptide.
14) The antibody of claim 13, wherein the peptide is a peptide fragment of collagen II (SEQ ID NO: 69), hemagglutinin (SEQ ID NO: 70). NY-ESO1 (SEQ ID NO: 71) or insulin (SEQ ID NO: 72).
15) The antibody of claim 13 or 14, wherein the peptide comprises an amino acid sequence of SEQ ID NOs: 7, 8, 9, 10 or 11.
16) The antibody of any one of claims 13-15, wherein the peptide consists of an amino acid sequence of SEQ ID NOs: 7, 8, 9, 10 or 11.
17) The antibody of any one of claims 1-16, wherein the antibody inhibits T cell activation.
18) The antibody of any one of claims 1-17, wherein the antibody inhibits CD4⁺ T cell proliferation at a concentration of 1 μg/ml by at least 30% in a co-culture of human CD4⁺ T cells and dendritic cells isolated from transgenic animals expressing human HLA-DR4 using assay described in Example 4.
19) The antibody of any one of claims 1-18, comprising a heavy chain complementarity determining region 1, 2 and 3 (a HCDR1, a HCDR2 and a HCDR3) of SEQ ID NOs: 73, 74 and 75, respectively.
20) The antibody of any one of claims 1-18, comprising a light chain complementarity determining region 1, 2 and 3 (a LCDR1, a LCDR2 and a LCDR3) of SEQ ID NOs: 76, 77 and 78, respectively.
21) The antibody of any one of claims 1-20, comprising the HCDR1, the HCDR2 and the HCDR3 contained in a heavy chain variable region (VH) of SEQ ID NOs: 56, 57, 58 or 59, wherein the HCDR1, the HCDR2 and the HCDR3 are defined by Kabat, IMGT or Chothia.
22) The antibody of any one of claims 1-21, comprising the LCDR1, the LCDR2 and the LCDR3 contained in a light chain variable region (VL) of SEQ ID NOs: 60 or 61, wherein the LCDR1, the LCDR2 and the LCDR3 are defined by Kabat, IMGT or Chothia.
23) The antibody of any one of claims 1-22, comprising the HCDR1 of SEQ ID NOs: 39, 40 or 41.
24) The antibody of any one of claims 1-23, comprising the HCDR2 of SEQ ID NOs: 42, 43, 44 or 45.
25) The antibody of any one of claims 1-24, comprising the HCDR3 of SEQ ID NOs: 46, 47, 48 or 49.
26) The antibody of any one of claims 1-25, comprising the LCDR1 of SEQ ID NOs: 50 or 51.
27) The antibody of any one of claims 1-26, comprising the LCDR2 of SEQ ID NOs: 52 or 53.
28) The antibody of any one of claims 1-27, comprising the LCDR3 of SEQ ID NOs: 54 or 55.
29) The antibody of any one of claims 1-28, comprising the HCDR1, the HCDR2, the HCDR3 of SEQ ID NOs: 39, 42 and 46, respectively, and the LCDR1, the LCDR2 and the LCDR3 of SEQ ID NOs: 50, 52 and 54, respectively.
30) The antibody of any of one claims 1-28, comprising
   a) the HCDR1, the HCDR2, the HCDR3 of
      i) SEQ ID NOs: 40, 43 and 47, respectively;
      ii) SEQ ID NOs: 41.44 and 48, respectively; or
      iii) SEQ ID NOs: 41.45 and 49, respectively; and
   b) the LCDR1, the LCDR2 and the LCDR3 of SEQ ID NOs: 51, 53 and 55, respectively.
31) The antibody of claim 30, comprising the HCDR1, the HCDR2, the HCDR3, the LCDR1, the LCDR2 and the LCDR3 of SEQ ID NOs: 40, 43, 47, 51, 53 and 55, respectively.
32) The antibody of claim 30, comprising the HCDR1, the HCDR2, the HCDR3, the LCDR1, the LCDR2 and the LCDR3 of SEQ ID NOs: 41, 44, 48, 51, 53 and 55, respectively.
33) The antibody of claim 30, comprising the HCDR1, the HCDR2, the HCDR3, the LCDR1, the LCDR2 and the LCDR3 of SEQ ID NOs: 41, 45, 49, 51, 53 and 55, respectively.
34) The antibody of any one of claims 1-33, wherein the antibody comprises a heavy chain framework derived from IGHV1-69 (SEQ ID NO: 62) or IGHV5-51 (SEQ ID NO: 63).
35) The antibody of any one of claims 1-34, wherein the antibody comprises a light chain framework derived from IGKV3-20 (SEQ ID NO: 64) or IGKV3-11 (SEQ ID NO: 65).
36) The antibody of claim 34 or 35, wherein the heavy chain framework is derived from IGHV1-69 (SEQ ID NO: 62) and the light chain framework is derived from IGKV3-20 (SEQ ID NO: 64).
37) The antibody of claim 34 or 35, wherein the heavy chain framework is derived from IGHV5-51 (SEQ ID NO: 63) and the light chain framework is derived from IGKV3-11 (SEQ ID NO: 65).
38) The antibody of claim 34 or 35, wherein the heavy chain framework is derived from IGHV1-69 (SEQ ID NO: 62) and the light chain framework is derived from IGKV3-11 (SEQ ID NO: 65).
39) The antibody of any one of claims 1-38, comprising the VH that is at least 84%, 85%, 86%, 87%, 88%, 89%, 90%, 91%, 92%, 93%, 94%, 95%, %, 97%, 98%, 99% or 100% identical to the amino acid sequence of SEQ ID NOs: 56, 57, 58 or 59.
40) The antibody of any one of claims 1-38, comprising the VL that is at least 90%, 91%, 92%, 93%, 94%, 95%, 96%, 97%, 98%, 99% or 100% identical to the amino acid sequence of SEQ ID NOs: 60 or 61.
41) The antibody of any one of claims 1-40, comprising the VH of SEQ ID NOs: 56, 57, 58 or 59, the VH optionally having 1, 2, 3, 4, 5, 6, 7, 8, 9, 10, I1, 12, 13, 14, 15, 16, 17 or 18 amino acid substitutions.
42) The antibody of any one of claims 1-41, comprising the VL of SEQ ID NOs: 60 or 61, the VL optionally having 1, 2, 3, 4, 5, 6, 7 or 8 amino acid substitutions.
43) The antibody of any one of claims 1-42, comprising the VH of SEQ ID NO: 56 and the VL of SEQ ID NO: 60.
44) The antibody of any one of claims 1-42, comprising the VH of SEQ ID NO: 57 and the VL of SEQ ID NO: 61.
45) The antibody of any one of claims 1-42, comprising the VH of SEQ ID NO: 58 and the VL of SEQ ID NO: 61.
46) The antibody of any one of claims 1-42, comprising the VH of SEQ ID NO: 59 and the VL of SEQ ID NO: 61.
47) The antibody of any one of claims 1-42, comprising the VH of SEQ ID NOs: 57, 58 or 59, and the VL of SEQ ID NO: 61.
48) The antibody of any one of claims 1-47, wherein the antibody is human or humanized.
49) The antibody of any one of claims 1-48, wherein the antibody is of IgG1, IgG2. IgG3 or IgG4 isotype.
50) The antibody of any one of claims 1-49, comprising one, two, three, four, five, six, seven, eight, nine or ten substitutions in the antibody Fc.
51) The antibody of claim 50, wherein the one, two, three, four, five, six, seven, eight, nine or ten substitutions result in reduced binding of the antibody to an activating Fcγ receptor (FcγR).
52) The antibody of claim 51, wherein the activating FcγR is FcγRI, FcγRIIa, FcγRIIIa, or FcγRIIIb.
53) The antibody of claim 52, comprising
   a) L234A, L235A, G237A, P238S, H268A, A330S and P331S substitutions;
   b) V234A, G237A, P238S, H268A, V309L, A330S and P331S substitutions;
   c) F234A, L235A, G237A, P238S and Q268A substitutions;
   d) L234A, L235A or L234A and L235A substitutions;
   e) F234A, L235A or F234A and L235A substitutions; or
   f) V234A substitution, wherein residue numbering is according to the EU Index.
54) The antibody of claim 53, comprising S228P substitution, wherein residue numbering is according to the EU Index.
55) A pharmaceutical composition comprising the antibody of any one of claims 1-54 and a pharmaceutically accepted carrier.
56) A polynucleotide encoding the antibody VH, the antibody VL, or the antibody VH and the antibody VL of any of the claims 19-54.
57) A vector comprising the polynucleotide of claim 56.
58) A host cell comprising the vector of claim 57.
59) A method of producing the antibody of any of the claims 19-54, comprising culturing the host cell of claim 58 in conditions that the antibody is expressed, and recovering the antibody produced by the host cell.
60) A method of treating a subject having an HLA-DRB1-associated autoimmune disease, comprising administering to a subject in need thereof a therapeutically effective amount of the antibody of any of the claims 1-54 for a time sufficient to treat the HLA-DRB1-associated autoimmune disease.
61) The method of claim 60, wherein the HLA-DRB1-associated autoimmune disease is Rheumatoid Arthritis, Systemic juvenile idiopathic arthritis. Grave's Disease, Hashimoto's Thyroiditis, Myasthenia Gravis, Multiple Sclerosis, Systemic Lupus Erythematosus or Type 1 Diabetes.
62) A method of suppressing an immune response towards a self-antigen, comprising administering to a subject in need thereof the antibody of any of the claims 1-54 for a time sufficient to suppress the immune response towards a self-antigen.
63) The method of claim 62, wherein the self-antigen is present in a patient with an autoimmune disease.
64) The method of claim 63, wherein the autoimmune disease is Rheumatoid Arthritis. Systemic juvenile idiopathic arthritis. Grave's Disease, Hashimoto's Thyroiditis, Myasthenia Gravis, Multiple Sclerosis, Systemic Lupus Erythematosus or Type 1 Diabetes.
65) An anti-idiotypic antibody binding to the antibody of any one of claims 43-47.
66) A kit comprising the antibody of any one of claims 43-47.
67) The kit of claim 66, further comprising reagents for detecting the antibody and instructions of use.

While having described the invention in general terms, the embodiments of the invention will be further disclosed in the following examples that should not be construed as limiting the scope of the claims.

### Example 1. Generation of Antigens and Control Antibodies

HLA-DR, HLA-DQ and HLA-DP heterodimeric antigens were expressed as Fc fusion proteins with covalently linked hemagglutinin, collagen, insulin or NY-ESO peptides coupled to the N-terminus of the HLA β chain via cleavable linker. The α and the β chains were expressed in format as follows:

α chain: ECD-G₄S-TEV-G₄S-Fc-His₆
β chain: peptide-3xGS-HRV3C-ECD-G₄S-TEV-G₄S-
Fc-StrepII
ECD: extracellular domain of the expressed HLA
chain
G₄S:

(SEQ ID NO: 1)

GGGGS
TEV:

(SEQ ID NO: 2)

EDLYFQ;
tobacco etch virus Nia protease cleavage site
His₆:

(SEQ ID NO: 3)

HHHHHH
3xGS:

(SEQ ID NO: 4)

GSGSGS
HRV3C:

(SEQ ID NO: 5)

LEVLFQGP;
human rhinovirus 3C protease cleavage site
StrepII:

(SEQ ID NO: 6)

WSHPQFEK;
StrepII tag
Hemagglutinin peptide HA_304-318:

(SEQ ID NO: 7)

ACPKYVKQNTLKLAT
Collagen II peptide CII_1236-1249:

(SEQ ID NO: 8)

LQYMRADQAAGGLR
Collagen II peptide CII_257-273:

(SEQ ID NO: 9)

EPGIAGFKGEQGPKGEP
Insulin peptide INS_1-15:

(SEQ ID NO: 10)

FVNQLCGSHLVEAL
NY-ESO peptide NY-ESO_157-169:

(SEQ ID NO: 11)

SLLMWITQCFLPV
PLP peptide PLP_178-186:

(SEQ ID NO: 37)

NTWTTCQSI
Fc: modified IgG4

(SEQ ID NO: 12)

CPPCPAPEAAGGPSVFLFPPKPKDTLMISRTPEVTCVVVDVSQEDPEVQF
NWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPS
DIAVEWESNGQPENNYKTTPPVLDSDGSFFLYSRLTVDKSRWQEGNWSCS
VMHEALHNHYTQKSLSLSL
HLA-DRA1*01:02

(SEQ ID NO: 13)

IKEEHVIIQAEFYLNPDQSGEFMFDFDGDEIFHVDMAKKETVWRLEEFGR
FASFEAQGALANIAVDKANLEIMTKRSNYTPITNVPPEVTVLTNSPVELR
EPNVLICFIDKFTPPVVNVTWLRNGKPVTTGVSETVFLPREDHLFRKFHY
LPFLPSTEDVYDCRVEHWGLDEPLLKHWEFDAPSPLPETTE
HLA-DRB1*04:01

(SEQ ID NO: 14)

GDTRPRFLEQVKHECHFFNGTERVRFLDRYFYHQEEYVRFDSDVGEYRAV
TELGRPDAEYWNSQKDLLEQKRAAVDTYCRHNYGVGESFTVQRRVYPEVT
VYPAKTQPLQHHNLLVCSVNGFYPGSIEVRWFRNGQEEKTGVVSTGLIQN
GDWTFQTLVMLETVPRSGEVYTCQVEHPSLTSPLTVEWRARSESAQSK
HLA-DRB1*01:01

(SEQ ID NO: 15)

GDTRPRFLWQLKFECHFFNGTERVRLLERCIYNQEESVRFDSDVGEYRAV
TELGRPDAEYWNSQKDLLEQRRAAVDTYCRHNYGVGESFTVQRRVEPKVT
VYPSKTQPLQHHNLLVCSVSGFYPGSIEVRWFRNGQEEKAGVVSTGLIQN
GDWTFQTLVMLETVPRSGEVYTCQVEHPSVTSPLTVEWRARSESAQSK
HLA-DQA1

(SEQID NO: 16)

EDIVADHVASCGVNLYQFYGPSGQYTHEFDGDEQFYVDLERKETAWRWPE
FSKFGGFDPQGALRNMAVAKHNLNIMIKRYNSTAATNEVPEVTVFSKSPV
TLGQPNTLICLVDNIFPPVVNITWLSNGQSVTEGVSETSFLSKSDHSFEK
ISYLTFLPSADEIYDCKVEHWGLDQPLLKHWEPEIPAPMSELTE
HLA_DQB1*06:02

(SEQ ID NO: 17)

RDSPEDFVFQFKGMCYFTNGTERVRLVTRYIYNREEYARFDSDVGVYRAV
TPQGRPDAEYWNSQKEVLEGTRAELDTVCRHNYEVAFRGILQRRVEPTVT
TSPSRTEALNHHNLLVCSVTDFWGQIKVRWFRNDQEETAGVVSTPLIRNG
DWTFQILVMVLEMTPQRGDVYTCHVEHPSLQSPITVEWRAQSESAQSK
HLA-DPA1

(SEQ ID NO: 18)

AGAIKADHVSTYAAFVQTHRPTGEFMFEFDEDEMFYVDLDKKETVWHLEE
FGQAFSFEAQGGLANIAILNNNLNTLIQRSNHTQATNDPPEVTVFPKEPV
ELGQPNTLICHIDKFFPPVLNVTWLCNGELVTEGVAESLFLPRTDYSFHK
FHYLTFVPSAEDFYDCRVEHWGLDQPLLKHWEAQEPIQMPETTE
HLA-DPB1*04:01

(SEQ ID NO: 19)

RATPENYLFQGRQECYAFNGTQRFLERYIYNREEFARFDSDVGEFRAVTE
LGRPAAEYWNSQKDILEEKRAVPDRMCRHNYELGGPMTLQRRVQPRVNVS
PSKKGPLQHHNLLVCHVTDFYPGSIQVRWTLNGQEETAGVVSTNLIRNGD
WTFQILVMLEMTPQQGDVYTCQVEHTSLDSPVTVEWKAQSDSARSK

Table 4 shows the format of the expressed HLA fusion proteins. Table 5 shows the amino acid sequences of both the α and β chains. For expression and purification. HLA α and β ECD-Fc fusions were co-transfected in HEK 293 Expi cells, the soluble HLA-ECD Fc fusion proteins were purified via ProteinA/SEC. All the HLA-DR antigens were conjugated to biotin using EZ-Link™ Sulfo-NHS-LC-Biotin and Labeling Kit (Thermo, cat no 21327), the success of the biotinylation was analyzed by HABA-avidin assay (Thermo, cat no 46610) and Octet.

TABLE 4

| Protein | |
|---|---|
| name | Protein Description |
| DR4G89 | Human HLA-DRA1*01:02/DRB1*04:01 ECD with hemagglutinin peptide |
| | HA_304-318 (HA) in the format of |
| | Alpha chain: ECD_G4S + TEV + G4S + MMB + 6xHisTag; |
| | Beta chain: HA + 3XGS + HRV3C + ECD + G4S + TEV + G4S + MMB + StrepII |
| DR4G90 | Human HLA-DRA1*01:02/DRB1*04:01 ECD with collagen II peptide |
| | CII_1236-1249 (CII_1236) in the format of |
| | Alpha chain: ECD_G4S + TEV + G4S + MMB + 6xHisTag; |
| | Beta chain: CII_1236 + 3XGS + HRV3C + ECD + G4S + TEV + G4S + MMB + StrepII |
| DR4G92 | Human HLA-DRA1*01:02/DRB1*04:01 ECD with collagen II peptide |
| | CII_257-273 (CII_257) in the format of |
| | Alpha chain: ECD_G4S + TEV + G4S + MMB + 6xHisTag; |
| | Beta chain: CII_257 + 3XGS + HRV3C + ECD + G4S + TEV + G4S + MMB + StrepII |
| DR4G93 | Human HLA-DRA1*01:02/DRB1*01:01 ECD with hemagglutinin peptide |
| | HA_304-318 (HA) in the format of |
| | Alpha chain: ECD_G4S + TEV + G4S + MMB + 6xHisTag; |
| | Beta chain: HA + 3XGS + HRV3C + ECD + G4S + TEV + G4S + MMB + StrepII |
| DR4G99 | Human HLA-DRA1*01:02/DRB1*01:01 ECD with collagen peptide |
| | CII_1236-1249 (CII_1236) in the format of |
| | Alpha chain: ECD_G4S + TEV + G4S + MMB + 6xHisTag; |
| | Beta chain: CII_1236 + 3XGS + HRV3C + ECD + G4S + TEV + G4S + MMB + StrepII |
| DR4G102 | Human HLA-DRA1*01:02/DRB1*01:01 ECD with collagen II peptide |
| | CII_257-273 (CII_257) in the format of |
| | Alpha chain: ECD_G4S + TEV + G4S + MMB + 6xHisTag; |
| | Beta chain: CII_257 + 3XGS + HRV3C + ECD + G4S + TEV + G4S + MMB + StrepII |
| DR4G111 | Human HLA-DQA1*01:02/DQB1*06:02 ECD with insulin peptide INS_1-15 |
| | in the format of |
| | Alpha chain: ECD_G4S + TEV + G4S + MMB + 6xHisTag; |
| | Beta chain: INS_1-15 + 3XGS + HRV3C + ECD + G4S + TEV + G4S + MMB + StrepII |
| DR4G112 | Human HLA-DQA1*01:02/DQB1*06:02 ECD with PLP peptide PLP_178-186 |
| | (PLP_178) in the format of |
| | Alpha chain: ECD_G4S + TEV + G4S + MMB + 6xHisTag; |
| | Beta chain: PLP_178 + 3XGS + HRV3C + ECD + G4S + TEV + G4S + MMB + StrepII, |
| DR4G113 | Human HRA-DPA1*03/DPB1*04:01 ECD with NY-ESO peptide NY-ESO_157-169 |
| | (NYESO-1) in the format of |
| | Alpha chain: ECD_G4S + TEV + G4S + MMB + 6xHisTag; |
| | Beta chain: NYESO-1 + 3XGS + HRV3C + ECD + G4S + TEV + G4S + MMB + StrepII |

TABLE 5

| Protein | Alpha Chain amino | Beta Chain amino acid |
|---|---|---|
| name | acid sequence | sequence |
| DR4G89 | IKEEHVIIQAEFYLNPDQS | ACPKYVKQNTLKLATGSGSG |
| (alpha | GEFMFDFDGDEIFHVDM | SLEVLFQGPGDTRPRFLEQVK |
| chain: | AKKETVWKLEEFGRFAS | HECHFFNGTERVRFLDRYFY |
| SEQ ID | FEAQGALANIAVDKANL | HQEEYVRFDSDVGEYRAVTE |
| NO: 20; | EIMTKRSNYTPITNVPPE | LGRPDAEYWNSQKDLLEQKR |
| beta | VTVLTNSPVELREPNVLI | AAVDTYCRHNYGVGESFTVQ |
| chain: | CFIDKFTPPVVNVTWLR | RRVYPEVTVYPAKTQPLQHH |
| SEQ ID | NGKPVTTGVSETVFLPRE | NLLVCSVNGFYPGSIEVRWFR |
| NO: 21) | DHLFRKFHYLPFLPSTED | NGQEEKTGVVSTGLIQNGDW |
| | VYDCRVEHWGLDEPLLK | TFQTLVMLETVPRSGEVYTC |
| | HWEFDAPSPLPETTEGG | QVEHPSLTSPLTVEWRARSES |
| | GGSEDLYFQSGGGGSCP | AQSKGGGGSEDLYFQSGGGG |
| | PCPAPEAAGGPSVFLFPP | SCPPCPAPEAAGGPSVFLFPP |
| | KPKDTLMISRTPEVTCVV | KPKDTLMISRTPEVTCVVVD |
| | VDVSQEDPEVQFNWYV | VSQEDPEVQFNWYVDGVEV |
| | DGVEVHNAKTKPREEQF | HNAKTKPREEQFNSTYRVVS |
| | NSTYRVVSVLTVLHQDW | VLTVLHQDWLNGKEYKCKV |
| | LNGKEYKCKVSNKGLPS | SNKGLPSSIEKTISKAKGQPRE |
| | SIEKTISKAKGQPREPQV | PQVYTLPPSQEEMTKNQVSL |
| | YTLPPSQEEMTKNQVSL | TCLVKGFYPSDIAVEWESNG |
| | TCLVKGFYPSDTAVEWES | QPENNYKTTPPVLDSDGSFFL |
| | NGQPENNYKTTPPVLDS | YSRLTVDKSRWQEGNVFSCS |
| | DGSFFLYSRLTVDKSRW | VMHEALHNHYTQKSLSLSLG |
| | QEGNVFSCSVMHEALHN | KWSHPQFEK |
| | HYTQKSLSLSLGKHHHH | |
| | HH | |
| DR4G90 | IKEEHVIIQAEFYLNPDQS | LQYMRADQAAGGLRGSGSG |
| (alpha | GEFMFDFDGDEIFHVDM | SLEVLFQGPGDTRPRFLEQVK |
| chain: | AKKETVWRLEEFGRFAS | HECHFFNGTERVRFLDRYFY |
| SEQ ID | FEAQGALANIAVDKANL | HQEEYVRFDSDVGEYRAVTE |
| NO: 20; | EIMTKRSNYTPITNVPPE | LGRPDAEYWNSQKDLLEQKR |
| beta | VTVLTNSPVELREPNVLI | AAVDTYCRHNYGVGESFTVQ |
| chain: | CFIDKFTPPVVNVTWLR | RRVYPEVTVYPAKTQPLQHH |
| SEQ ID | NGKPVTTGVSETVFLPRE | NLLVCSVNGFYPGSIEVRWFR |
| NO: 22) | DHLFRKFHYLPFLPSTED | NGQEEKTGVVSTGLIQNGDW |
| | VYDCRVEHWGLDEPLLK | TFQTLVMLETVPRSGEVYTC |
| | HWEFDAPSPLPETTEGG | QVEHPSLTSPLTVEWRARSES |
| | GGSEDLYFQSGGGGSCP | AQSKGGGGSEDLYFQSGGGG |
| | PCPAPEAAGGPSVFLFPP | SCPPCPAPEAAGGPSVFLFPP |
| | KPKDTLMISRTPEVTCVV | KPKDTLMISRTPEVTCVVVD |
| | VDVSQEDPEVQFNWYV | VSQEDPEVQFNWYVDGVEV |
| | DGVEVHNAKTKPREEQF | HNAKTKPREEQFNSTYRVVS |
| | NSTYRVVSVLTVLHQDW | VLTVLHQDWLNGKEYKCKV |
| | LNGKEYKCKVSNKGLPS | SNKGLPSSIEKTISKAKGQPRE |
| | SIEKTISKAKGQPREPQV | PQVYTLPPSQEEMTKNQVSL |
| | YTLPPSQEEMTKNQVSL | TCLVKGFYPSDIAVEWESNG |
| | TCLVKGFYPSDIAVEWES | QPENNYKTTPPVLDSDGSFFL |
| | NGQPENNYKTTPPVLDS | YSRLTVDKSRWQEGNVFSCS |
| | DGSFFLYSRLTVDKSRW | VMHEALHNHYTQKSLSLSLG |
| | QEGNVFSCSVMHEALHN | KWSHPQFEK |
| | HYTQKSLSLSLGKHHHH | |
| | HH | |
| DR4G92 | IKEEHVIIQAEFYLNPDQS | EPGIAGFKGEQGPKGEPGSGS |
| (alpha | GEFMFDFDGDEIFHVDM | GSLEVLFQGPGDTRPRFLEQV |
| chain: | AKKETVWRLEEFGRFAS | KHECHFFNGTERVRFLDRYF |
| SEQ ID | FEAQGALANIAVDKANL | YHQEEYVRFDSDVGEYRAVT |
| NO: 20; | EIMTKRSNYTPITNVPPE | ELGRPDAEYWNSQKDLLEQK |
| beta | VTVLTNSPVELREPNVLI | RAAVDTYCRHNYGVGESFTV |
| chain: | CFIDKFTPPVVNVTWLR | QRRVYPEVTVYPAKTQPLQH |
| SEQ ID | NGKPVTTGVSETVFLPRE | HNLLVCSVNGFYPGSIEVRW |
| NO: 23) | DHLFRKFHYLPFLPSTED | FRNGQEEKTGVVSTGLIQNG |
| | VYDCRVEHWGLDEPLLK | DWTFQTLVMLETVPRSGEVY |
| | HWEFDAPSPLPETTEGG | TCQVEHPSLTSPLTVEWRARS |
| | GGSEDLYFQSGGGGSCP | ESAQSKGGGGSEDLYFQSGG |
| | PCPAPEAAGGPSVFLFPP | GGSCPPCPAPEAAGGPSVFLF |
| | KPKDTLMISRTPEVTCVV | PPKPKDTLMISRTPEVTCVVV |
| | VDVSQEDPEVQFNWYV | DVSQEDPEVQFNWYVDGVE |
| | DGVEVHNAKTKPREEQF | VHNAKTKPREEQFNSTYRVV |
| | NSTYRVVSVLTVLHQDW | SVLTVLHQDWLNGKEYKCK |
| | LNGKEYKCKVSNKGLPS | VSNKGLPSSIEKTISKAKGQP |
| | SIEKTISKAKGQPREPQV | REPQVYTLPPSQEEMTKNQV |
| | YTLPPSQEEMTKNQVSL | SLTCLVKGFYPSDIAVEWESN |
| | TCLVKGFYPSDIAVEWES | GQPENNYKTTPPVLDSDGSFF |
| | NGQPENNYKTTPPVLDS | LYSRLTVDKSRWQEGNVFSC |
| | DGSFFLYSRLTVDKSRW | SVMHEALHNHYTQKSLSLSL |
| | QEGNVFSCSVMHEALHN | GKWSHPQFEK |
| | HYTQKSLSLSLGKHHHH | |
| | HH | |
| DR4G93 | IKEEHVIIQAEFYLNPDQS | ACPKYVKQNTLKLATGSGSG |
| (alpha | GEFMFDFDGDEIFHVDM | SLEVLFQGPGDTRPRFLWQL |
| chain: | AKKETVWRLEEFGRFAS | KFECHFFNGTERVRLLERCIY |
| SEQ ID | FEAQGALANIAVDKANL | NQEESVRFDSDVGEYRAVTE |
| NO: 20; | EIMTKRSNYTPITNVPPE | LGRPDAEYWNSQKDLLEQRR |
| beta | VTVLTNSPVELREPNVLI | AAVDTYCRHNYGVGESFTVQ |
| chain: | CFIDKFTPPVVNVTWLR | RRVEPKVTVYPSKTQPLQHH |
| SEQ ID | NGKPVTTGVSETVFLPRE | NLLVCSVSGFYPGSIEVRWFR |
| NO: 24) | DHLFRKFHYLPFLPSTED | NGQEEKAGVVSTGLIQNGDW |
| | VYDCRVEHWGLDEPLLK | TFQTLVMLETVPRSGEVYTC |
| | HWEFDAPSPLPETTEGG | QVEHPSVTSPLTVEWRARSES |
| | GGSEDLYFQSGGGGSCP | AQSKGGGGSEDLYFQSGGGG |
| | PCPAPEAAGGPSVFLFPP | SCPPCPAPEAAGGPSVFLFPP |
| | KPKDTLMISRTPEVTCVV | KPKDTLMISRTPEVTCVVVD |
| | VDVSQEDPEVQFNWYV | VSQEDPEVQFNWYVDGVEV |
| | DGVEVHNAKTKPREEQF | HNAKTKPREEQFNSTYRVVS |
| | NSTYRVVSVLTVLHQDW | VLTVLHQDWLNGKEYKCKV |
| | LNGKEYKCKVSNKGLPS | SNKGLPSSIEKTISKAKGQPRE |
| | SIEKTISKAKGQPREPQV | PQVYTLPPSQEEMTKNQVSL |
| | YTLPPSQEEMTKNQVSL | TCLVKGFYPSDIAVEWESNG |
| | TCLVKGFYPSDIAVEWES | QPENNYKTTPPVLDSDGSFFL |
| | NGQPENNYKTTPPVLDS | YSRLTVDKSRWQEGNVFSCS |
| | DGSFFLYSRLTVDKSRW | VMHEALHNHYTQKSLSLSLG |
| | QEGNVFSCSVMHEALHN | KWSHPQFEK |
| | HYTQKSLSLSLGKHHHH | |
| | HH | |
| DR4G99 | IKEEHVIIQAEFYLNPDQS | LQYMRADQAAGGLRGSGSG |
| (alpha | GEFMFDFDGDEIFHVDM | SLEVLFQGPGDTRPRFLWQL |
| chain: | AKKETVWRLEEFGRFAS | KFECHFFNGTERVRLLERCIY |
| SEQ ID | FEAQGALANIAVDKANL | NQEESVRFDSDVGEYRAVTE |
| NO: 20; | EIMTKRSNYTPTINVPPE | LGRPDAEYWNSQKDLLEQRR |
| beta | VTVLTNSPVELREPNVLI | AAVDTYCRHNYGVGESFTVQ |
| chain: | CFIDKFTPPVVNVTWLR | RRVEPKVTVYPSKTQPLQHH |
| SEQ ID | NGKPVTTGVSETVFLPRE | NLLVCSVSGFYPGSIEVRWFR |
| NO: 25) | DHLFRKFHYLPFLPSTED | NGQEEKAGVVSTGLIQNGDW |
| | VYDCRVEHWGLDEPLLK | TFQTLVMLETVPRSGEVYTC |
| | HWEFDAPSPLPETTEGG | QVEHPSVTSPLTVEWRARSES |
| | GGSEDLYFQSGGGGSCP | AQSKGGGGSEDLYFQSGGGG |
| | PCPAPEAAGGPSVFLFPP | SCPPCPAPEAAGGPSVFLFPP |
| | KPKDTLMISRTPEVTCVV | KPKDTLMISRTPEVTCVVVD |
| | VDVSQEDPEVQFNWYV | VSQEDPEVQFNWYVDGVEV |
| | DGVEVHNAKTKPREEQF | HNAKTKPREEQFNSTYRVVS |
| | NSTYRVVSVLTVLHQDW | VLTVLHQDWLNGKEYKCKV |
| | LNGKEYKCKVSNKGLPS | SNKGLPSSIEKTISKAKGQPRE |
| | SIEKTISKAKGQPREPQV | PQVYTLPPSQEEMTKNQVSL |
| | YTLPPSQEEMTKNQVSL | TCLVKGFYPSDIAVEWESNG |
| | TCLVKGFYPSDIAVEWES | QPENNYKTTPPVLDSDGSFFL |
| | NGQPENNYKTTPPVLDS | YSRLTVDKSRWQEGNVFSCS |
| | DGSFFLYSRLTVDKSRW | VMHEALHNHYTQKSLSLSLG |
| | QEGNVFSCSVMHEALHN | KWSHPQFEK |
| | HYTQKSLSLSLGKHHHH | |
| | HH | |
| DR4G102 | IKEEHVIIQAEFYLNPDQS | EPGIAGFKGEQGPKGEPGSGS |
| (alpha | GEFMFDFDGDEIFHVDM | GSLEVLFQGPGDTRPRFLWQ |
| chain: | AKKETVWRLEEFGRFAS | LKFECHFFNGTERVRLLERCI |
| SEQ ID | FEAQGALANIAVDKANL | YNQEESVRFDSDVGEYRAVT |
| NO: 20; | EIMTKRSNYTPITNVPPE | ELGRPDAEYWNSQKDLLEQR |
| beta | VTVLTNSPVELREPNVLI | RAAVDTYCRHNYGVGESFTV |
| chain: | CFIDKFTPPVVNVTWLR | QRRVEPKVTVYPSKTQPLQH |
| SEQ ID | NGKPVTTGVSETVFLPRE | HNLLVCSVSGFYPGSIEVRWF |
| NO: 26) | DHLFRKFHYLPFLPSTED | RNGQEEKAGVVSTGLIQNGD |
| | VYDCRVEHWGLDEPLLK | WTFQTLVMLETVPRSGEVYT |
| | HWEFDAPSPLPETTEGG | CQVEHPSVTSPLTVEWRARS |
| | GGSEDLYFQSGGGGSCP | ESAQSKGGGGSEDLYFQSGG |
| | PCPAPEAAGGPSVFLFPP | GGSCPPCPAPEAAGGPSVFLF |
| | KPKDTLMISRTPEVTCVV | PPKPKDTLMISRTPEVTCVVV |
| | VDVSQEDPEVQFNWYV | DVSQEDPEVQFNWYVDGVE |
| | DGVEVHNAKTKPREEQF | VHNAKTKPREEQFNSTYRVV |
| | NSTYRVVSVLTVLHQDW | SVLTVLHQDWLNGKEYKCK |
| | LNGKEYKCKVSNKGLPS | VSNKGLPSSIEKTISKAKGQP |
| | SIEKTISKAKGQPREPQV | REPQVYTLPPSQEEMTKNQV |
| | YTLPPSQEEMTKNQVSL | SLTCLVKGFYPSDIAVEWESN |
| | TCLVKGFYPSDIAVEWES | GQPENNYKTTPPVLDSDGSFF |
| | NGQPENNYKTTPPVLDS | LYSRLTVDKSRWQEGNVFSC |
| | DGSFFLYSRLTVDKSRW | SVMHEALHNHYTQKSLSLSL |
| | QEGNVFSCSVMHEALHN | GKWSHPQFEK |
| | HYTQKSLSLSLGKHHHH | |
| | HH | |
| DR4G111 | EDIVADHVASCGVNLYQ | FVNQHLCGSHLVEALGSGSG |
| (alpha | FYGPSGQYTHEFDGDEQ | SLEVLFQGPRDSPEDFVFQFK |
| chain: | FYVDLERKETAWRWPEF | GMCYFTNGTERVRLVTRYIY |
| SEQ ID | SKFGGFDPQGALRNMAV | NREEYARFDSDVGVYRAVTP |
| NO: 27; | AKHNLNIMIKRYNSTAA | QGRPDAEYWNSQKEVLEGTR |
| beta | TNEVPEVTVFSKSPVTLG | AELDTVCRHNYEVAFRGILQ |
| chain: | QPNTLICLVDNIFPPVVNI | RRVEPTVTISPSRTEALNHHN |
| SEQ ID | TWLSNGQSVTEGVSETS | LLVCSVTDFYPGQIKVRWFR |
| NO: 28) | FLSKSDHSFFKISYLTFLP | NDQEETAGVVSTPLIRNGDW |
| | SADEIYDCKVEHWGLDQ | TFQILVMLEMTPQRGDVYTC |
| | PLLKHWEPEIPAPMSELT | HVEHPSLQSPITVEWRAQSES |
| | EGGGGSEDLYFQSGGGG | AQSKGGGGSEDLYFQSGGGG |
| | SCPPCPAPEAAGGPSVFL | SCPPCPAPEAAGGPSVFLFPP |
| | FPPKPKDTLMISRTPEVT | KPKDTLMISRTPEVTCVVVD |
| | CVVVDVSQEDPEVQFN | VSQEDPEVQFNWYVDGVEV |
| | WYVDGVEVHNAKTKPR | HNAKTKPREEQFNSTYRVVS |
| | EEQFNSTYRVVSVLTVL | VLTVLHQDWLNGKEYKCKV |
| | HQDWLNGKEYKCKVSN | SNKGLPSSIEKTISKAKGQPRE |
| | KGLPSSIEKTISKAKGQP | PQVYTLPPSQEEMTKNQVSL |
| | REPQVYTLPPSQEEMTK | TCLVKGFYPSDIAVEWESNG |
| | NQVSLTCLVKGFYPSDIA | QPENNYKTTPPVLDSDGSFFL |
| | VEWESNGQPENNYKTTP | YSRLTVDKSRWQEGNVFSCS |
| | PVLDSDGSFFLYSRLTVD | VMHEALHNHYTQKSLSLSLG |
| | KSRWQEGNVFSCSVMHE | KWSHPQFEK |
| | ALHNHYTQKSLSLSLGG | |
| | SHHHHHH | |
| DR4G112 | EDIVADHVASCGVNLYQ | NTWTTCQSIGSGSGSLEVLFQ |
| (alpha | FYGPSGQYTHEFDGDEQ | GPRDSPEDFVFQFKGMCYFT |
| chain: | FYVDLERKETAWRWPEF | NGTERVRLVTRYIYNREEYA |
| SEQ ID | SKFGGFDPQGALRNMAV | RFDSDVGVYRAVTPQGRPDA |
| NO: 27; | AKHNLNIMIKRYNSTAA | EYWNSQKEVLEGTRAELDTV |
| beta | TNEVPEVTVFSKSPVTLG | CRHNYEVAFRGILQRRVEPT |
| chain: | QPNTLICLVDNIFPPVVNI | VTISPSRTEALNHHNLLVCSV |
| SEQ ID | TWLSNGQSVTEGVSETS | TDFYPGQIKVRWFRNDQEET |
| NO: 38) | FLSKSDHSFFKISYLTFLP | AGVVSTPLIRNGDWTFQILV |
| | SADEIYDCKVEHWGLDQ | MLEMTPQRGDVYTCHVEHPS |
| | PLLKHWEPEIPAPMSELT | LQSPITVEWRAQSESAQSKGG |
| | EGGGGSEDLYFQSGGGG | GGSEDLYFQSGGGGSCPPCPA |
| | SCPPCPAPEAAGGPSVFL | PEAAGGPSVFLFPPKPKDTLM |
| | FPPKPKDTLMISRTPEVT | ISRTPEVTCVVVDVSQEDPEV |
| | CVVVDVSQEDPEVQFN | QFNWYVDGVEVHNAKTKPR |
| | WYVDGVEVHNAKTKPR | EEQFNSTYRVVSVLTVLHQD |
| | EEQFNSTYRVVSVLTVL | WLNGKEYKCKVSNKGLPSSI |
| | HQDWLNGKEYKCKVSN | EKTISKAKGQPREPQVYTLPP |
| | KGLPSSIEKTISKAKGQP | SQEEMTKNQVSLTCLVKGFY |
| | REPQVYTLPPSQEEMTK | PSDIAVEWESNGQPENNYKT |
| | NQVSLTCLVKGFYPSDIA | TPPVLDSDGSFFLYSRLTVDK |
| | VEWESNGQPENNYKTTP | SRWQEGNVFSCSVMHEALH |
| | PVLDSDGSFFLYSRLTVD | NHYTQKSLSLSLGKWSHPQF |
| | KSRWQEGNVFSCSVMHE | EK |
| | ALHNHYTQKSLSLSLGG | |
| | SHHHHHH | |
| DR4G113 | AGAIKADHVSTYAAFVQ | SLLMWITQCFLPVGSGSGSLE |
| (alpha | THRPTGEFMFEFDEDEM | VLFQGPRATPENYLFQGRQE |
| chain: | FYVDLDKKETVWHLEEF | CYAFNGTQRFLERYIYNREEF |
| SEQ ID | GQAFSFEAQGGLANIAIL | ARFDSDVGEFRAVTELGRPA |
| NO: 29; | NNNLNTLIQRSNHTQAT | AEYWNSQKDILEEKRAVPDR |
| beta | NDPPEVTVFPKEPVELGQ | MCRHNYELGGPMTLQRRVQ |
| chain: | PNTLICHIDKFFPPVLNVT | PRVNVSPSKKGPLQHHNLLV |
| SEQ ID | WLCNGELVTEGVAESLF | CHVTDFYPGSIQVRWFLNGQ |
| NO: 30) | LPRTDYSFHKFHYLTFVP | EETAGVVSTNLIRNGDWTFQI |
| | SAEDFYDCRVEHWGLD | LVMLEMTPQQGDVYTCQVE |
| | QPLLKHWEAQEPIQMPE | HTSLDSPVTVEWKAQSDSAR |
| | TTEGGGGSEDLYFQSGG | SKGGGGSEDLYFQSGGGGSC |
| | GGSCPPCPAPEAAGGPSV | PPCPAPEAAGGPSVFLFPPKP |
| | FLFPPKPKDTLMISRTPE | KDTLMISRTPEVTCVVVDVS |
| | VTCVVVDVSQEDPEVQF | QEDPEVQFNWYVDGVEVHN |
| | NWYVDGVEVHNAKTKP | AKTKPREEQFNSTYRVVSVL |
| | REEQFNSTYRVVSVLTV | TVLHQDWLNGKEYKCKVSN |
| | LHQDWLNGKEYKCKVS | KGLPSSIEKTISKAKGQPREPQ |
| | NKGLPSSIEKTISKAKGQ | VYTLPPSQEEMTKNQVSLTC |
| | PREPQVYTLPPSQEEMTK | LVKGFYPSDIAVEWESNGQP |
| | NQVSLTCLVKGFYPSDIA | ENNYKTTPPVLDSDGSFFLYS |
| | VEWESNGQPENNYKTTP | RLTVDKSRWQEGNVFSCSVM |
| | PVLDSDGSFFLYSRLTVD | HEALHNHYTQKSLSLSLGKW |
| | KSRWQEGNVFSCSVMHE | SHPQFEK |
| | ALHNHYTQKSLSLSLGG | |
| | SHHHHHH | |

Antibodies Lym-1, apolizumab (1D10) and L243 were used as control antibodies after re-engineering the constant domains as IgG2sigma isotypes. The engineered IgG2sigma mAbs were renamed DR4B4 (Lym-1). DR4B5 (apolizumab) and DR4B6 (L243). IgG2sigma is an effector silent Fc and has substitutions V234A, G237A, P238S, H268A, V309L, A330S and P331S when compared to the wild type IgG2. IgG2sigma is described in U.S. Pat. No. 8,961,967.

Lym-1 VH

(SEQ ID NO: 31)

QVQLKESGPGLVAPSQSLSITCTISGFSLTSYGVHWVRQPPGKGLEWLV
VIWSDGSTTYNSALKSRLSISKDNSKSQVFLKMNSLQTDDTAIYYCASH
YGSTLAFASWGHGTLVTVSA
Lym-1 VL

(SEQ ID NO: 32)

DIQMTQSPASLSASVGETVTIICRASVNIYSYLAWYQQKQGKSPQLLVY
NAKILAEGVPSRFSGSGSGTQFSLKINSLQPEDFGSYYCQHHYGPFTFG
SGTKLEIK
Apolizumab VH

(SEQ ID NO: 33)

QVQLQESGPGLVKPSETLSLTCTVSGFSLTNYGVHWVRQSPGKGLEWIG
VKWSGGSTEYNAAFISRLTISKDTSKNQVSLKLNSLTAADTAVYYCARN
DRYAMDYWGQGTLVTVSS
Apolizumab VL

(SEQ ID NO: 34)

DIQMTQSPSSLSASVGDRVTITCRASENIYSYLAWYQQKPGKAPKLLVS
NAKTLAEGVPSRFSGSGSGKQFTLTISSLQPEDFATYYCQHHYGNSYPF
GQGTKLEIK
L243 VH

(SEQ ID NO: 35)

QIQLVQSGPELKKPGETVKISCKASGFTFTNYGMNWVKQAPGKGLKWMG
WINTYTREPTYADDFKGRFAFSLETSASTAYLQINNLKNEDTAKYFCAR
DITAVVPTGFDYWGQGTTLTVSS
L243 VL

(SEQ ID NO: 36)

DIQMTQSPASLSVSVGETVTITCRASENIYSNLAWYRQKQGKSPQLLVF
AASNLADGVPSRFSGSGSGTQYSLKINSLQSEDFGDYYCQHFWTTPWAF
GGGTNLEIK

### Example 2. Isolation of Antibodies which Bind to HLA-DR from Phage Display Libraries

HLA-DR binding Fabs were selected from two sets of de novo pIX phage display libraries as described in Shi et al., J Mol Biol 397:385-%, 2010; Int. Pat. Publ. No. WO2009/085462). Briefly, two sets of libraries, referred to as V3.0 and V5.0, were generated by diversifying human scaffolds where germline VH genes IGHV1-69*01, IGHV3-23*01, and IGHV5-51*01 were recombined with the human IGHJ-4 minigene via the H3 loop (IGHJ-6 minigene was also used in V5.0), and human germline VLkappa genes O12 (IGKV1-39*01). L6 (IGKV3-11*01). A27 (IGKV3-20*01), and B3 (IGKV4-1*01) were recombined with the IGKJ-1 minigene to assemble complete VH and VL domains. The positions in the heavy and light chain variable regions around H1, H2, L1, L2 and L3 loops corresponding to positions identified to be frequently in contact with protein and peptide antigens were chosen for diversification. Sequence diversity at selected positions was limited to residues occurring at each position in the IGHV or IGLV germline gene families of the respective IGHV or IGLV genes. Diversity at the H3 loop was generated by utilizing short to mid-sized synthetic loops of lengths 7-14 amino acids for V3.0 libraries, and lengths 6-19 amino acids for V5.0 libraries. The amino acid distribution at H3 was designed to mimic the observed variation of amino acids in human antibodies. The scaffolds utilized to generate libraries were named according to their human VH and VL germline gene origin. For both V3.0 and V5.0 sets, each of the three heavy chain libraries were combined with the four germline light chains or germline light chain libraries to generate 12 unique VH:VL combinations for each set of libraries which are used for selection experiments against recombinant cell line expressing HLA-DR or extracellular domain of HLA-DR fused to Fc fragment and displaying a specific peptide.

In the “cell-based” selections, subtractive strategy was employed, which was based on an initial depletion step against unwanted epitopes or native cells (parental cell line, U937) followed by a selection step for the target epitope or transfected cells (recombinant cell line). The recombinant cell line expressing HLA-DR15 (Uniprot: P01911) was produced by stable transfection in U937 cells. This subtractive strategy avoided the selection of phage that bound to the overabundance of other cell surface receptors that were not of interest. In the phage selections using purified recombinant antigens, biotinylated HLA-DR4 with HA peptide HA_304-318 (DR4G89) or HLA-DR4 with collagen II peptide CII_257-273 (DR4G92) were used as bait to capture and immobilize the phage binders. After several selection rounds, a polyclonal phage ELISA using purified antigens was performed to detect the specific enrichment of individual panning experiments. The phage collected from those panning experiments which demonstrated enrichment for binders to HLA-DR were further screened with a monoclonal Fab ELISA in which Fab proteins expressed from individual Fab clones were used as binders to several different biotinylated HLA-DR antigens (DR4G89, DR4G90, DR4G92. DR4G102) as well as biotinylated HLA-DP (DR4G113) and HLA-DQ (DR4G111 and DR4G112). The Fab clones with binding signal to HLA-DR five times higher than the negative control Fabs and to HLA-DP or HLA-DQ less than five times higher than the negative control Fabs were selected for further analyses. The selected Fabs were cloned as IgG2sigma/Kappa and characterized further using MSD assay.

### Example 3. The Anti-HLA-DR Antibodies Bind Soluble HLA-DR Antigens Irrespective of the Peptide Presented

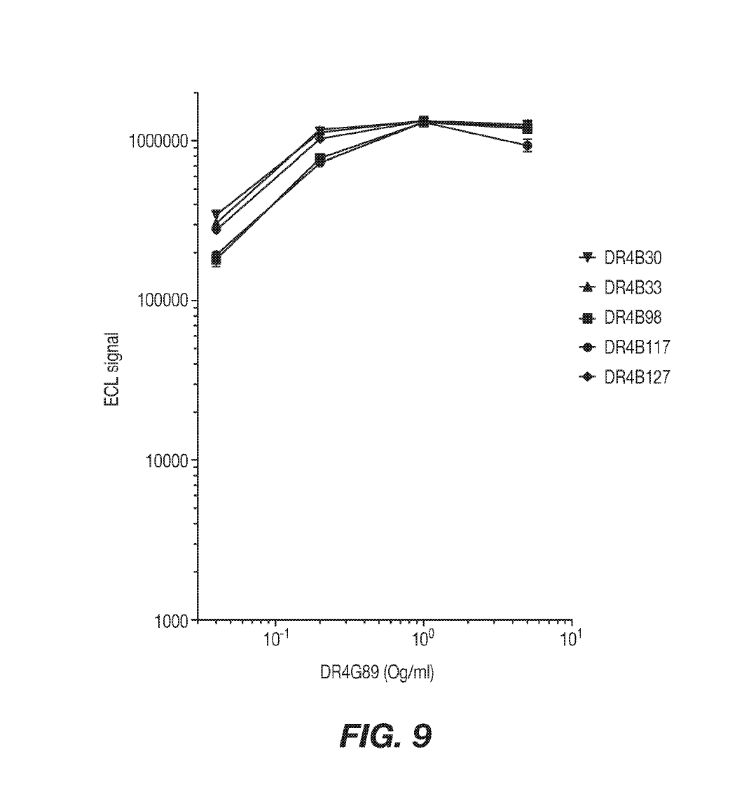

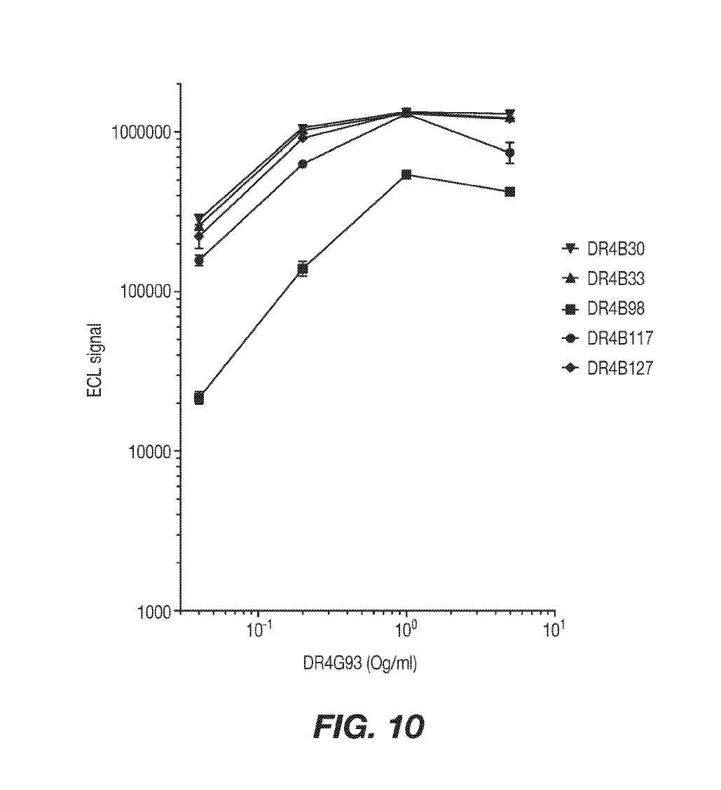

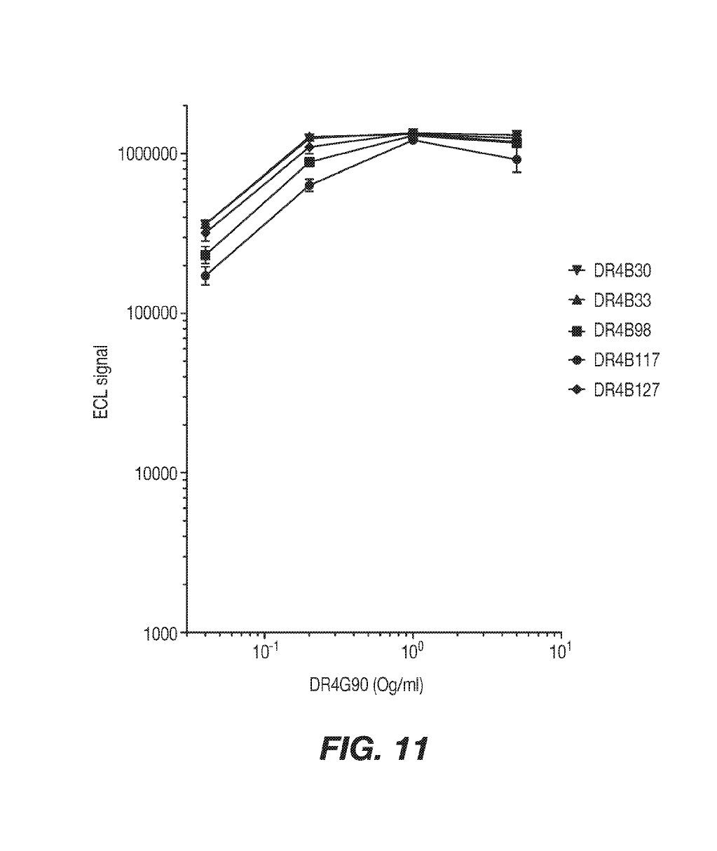

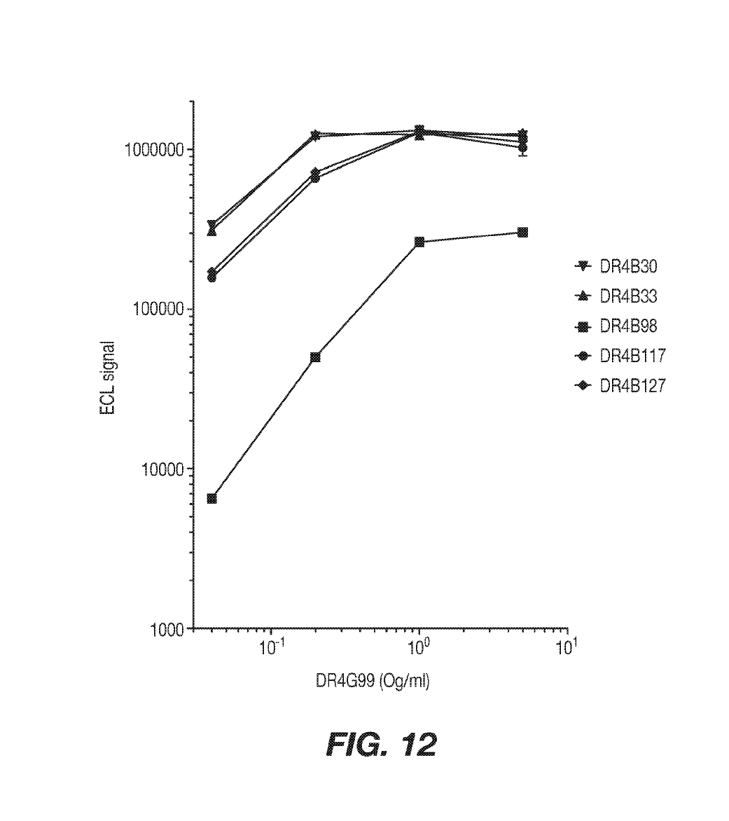

Select generated antibodies were characterized for their binding to soluble HLA-DR, HLA-DQ or HLA-DP antigens with various peptides attached to the N-terminus of the beta chains. In addition, control antibodies DR4B4. DR4B5 and DR4B6 were also tested. The soluble antigens of DR, DP and DQ were coated on MSD standard plates (Meso Scale Discovery, Cat. No. L15XA-3) at 5 μg/ml at 4° C. overnight. The following day, the plates were washed for three times with PBST at an automatic plate washer (Bio Tek), blocked with StartingBlock™ (Thermo Scientific, Cat No. 37543) for 30 minutes and incubated with the antibodies for 1 hour. Binding to DR antigens was tested with four antibody concentrations ranging from 0.04-5 μg/ml. Binding to DP and DQ antigens was tested with one concentration at 5 μg/ml. After three washes, SulfoTag anti-human/NHP Kappa secondary antibody (Meso Scale Discovery, Cat. No. D20TF-6) was added and incubated for 1 hour. After another three washes, the plates were read under MSD reader (Meso Scale Discovery), and electrochemiluminescence (ECL) was measured. Because MSD assay has high reproducibility, duplicates were run for each data point Results of binding of the antibodies to DR, DP or DQ antigens (expressed as the ECL signal) are shown in Table 6 at an antibody concentration of 5 μg/ml for binding to DP and DQ, and at 0.2 μg/ml for binding to DR alleles and are reported as average of the two replicates. The dose response curve for antibody binding to DR4G89 (HLA-DR4 with HA_304-318 peptide) is shown in FIG. 9. The dose response curve for antibody binding to DR4G93 (HLA-DR1 with HA_304-318 peptide) is shown in FIG. 10. The dose response curve for antibody binding to DR4G90 (HLA-DR4 with CII_1236-1249 peptide) is shown in FIG. 11. The dose response curve for antibody binding to DR4G99 (HLA-DR1 with CII_1236-1249 peptide) is shown in FIG. 12.

The generated antibodies bound to HLA-DR4 and HLA-DR1 irrespective of the peptide presented on HLA-DR except that DR4B98 demonstrated reduced binding to HLA-DR with the CII_1236-1249 peptide. The antibodies demonstrated minimal binding to HLA-DQ and HLA-DP.

TABLE 6

| | HLA | | | | | |
|---|---|---|---|---|---|---|
| | DQB6:02 | DP4:01 | DRB1*04:01 | | DRB1*01:01 | |
| | Antigen name | | | | | |
| | DR4G111 | DR4G113 | DR4G89 | DR4G90 | DR4G93 | DR4G99 |
| | Attached peptide | | | | | |
| | | NY- | HA_304- | CII_1236- | HA_304- | CII_1236- |
| | INS_1-15 | ESO_157-169 | 318 | 1249 | 318 | 1249 |
| DR4B30 | 12905 | 1969 | 1128453 | 1248862 | 1066233 | 1199978 |
| DR4B98 | 941 | 817 | 774933 | 889767 | 140066 | 49986 |
| DR4B117 | 927 | 27683 | 733860 | 636052 | 631598 | 658790 |
| DR4B127 | 941 | 702 | 1029857 | 1097830 | 918166 | 718806 |
| DR4B4 | 128 | 138 | 1276361 | 1086146 | 887698 | 15503 |
| DR4B5 | 195 | 120 | 1260358 | 1306176 | 1295750 | 1183017 |
| DR4B6 | 282 | 298 | 1337274 | 1353100 | 1322424 | 1242429 |

### Example 4. Characterization of Anti-HLA-DR Antibodies

The generated antibodies were tested for their ability to inhibit antigen-specific T cell activation, for their binding to dendritic cells isolated from HLA-DR4 transgenic animals and to peripheral blood mononuclear cells (PBMC), and their effect on B cell viability.

### Methods

### Inhibition of Antigen Specific T Cells: HLA-DR4 Transgenic Mouse Dendritic Cell Mixed Lymphocyte Reaction (MLR) Assay (“HLA-DR4 DC MLR”)

A MLR assay was used to assess the ability of the generated antibodies to inhibit T cell activation measuring inhibition of cell proliferation in co-cultures of human CD4⁺ T cells and dendritic cells isolated from transgenic animals expressing human HLA-DR4.

The dendritic cells were derived from Abb Knockout/Transgenic HLA-DR4 mouse bone marrow (strain 4149, Taconic Biosciences). These mice express human HLA-DRA and HLA-DRB1*04:01 engineered to membrane proximal domains of mouse I-E (H2-E). Bone marrow was prepared from the mice and frozen at −80° C. The bone marrow was thawed and the cells were resuspended in 10 ml of dendritic cell (DC) media (RPMI-1640/Glutamax containing 1% Penicillin/Streptomycin, 1% sodium pyruvate, 1% Minimum Essential Media (MEM) non-essential amino acids (NEAA) solution, 10% heat-inactivated fetal bovine serum (all purchased from Thermo Fisher Scientific), and 50 μM 2-Mercaptoethanol (Sigma-Aldrich). The cells were centrifuged at 1200 rpm for 10 minutes, then resuspended in 10 ml DC media, counted and spun again at 1200 rpm for 8 minutes. The cells were diluted to 0.3×10⁶ cells/ml in DC media supplemented with 20 ng/ml recombinant mouse GM-CSF (Peprotech). Six ml of the diluted cells were transferred to each well of a 6-well plate; the plates were then incubated at 37° C./5% CO₂ for 96 hours. Three nil of the media was removed from each well and replaced with 3 ml of fresh DC media+20 ng/ml GM-CSF. The plates were incubated for an additional 48 h at 37° C./5% CO₂. Three ml of the media was removed from each well and replaced with 3 ml of fresh DC media+20 ng/ml GM-CSF+2 μg/ml LPS (for a final concentration of 1 μg/ml LPS) (Enzo Life Sciences). The plates were then incubated at 37° C./5% CO₂ for 18 hours.

The human CD4⁺ T cells used in the MLR assay were isolated from frozen human PBMCs (Hemacare). The cells were thawed, transferred to a 50 ml conical, washed with 40 ml of complete media (RPMI-1640/Glutamax containing 1% Penicillin/Streptomycin, 10% heat-inactivated fetal bovine serum (all purchased from Thermo Fisher Scientific) and 50 μM 2-Mercaptoethanol (Sigma-Aldrich). The cells were centrifuged at 1000 rpm for 8 minutes, the supernatant was aspirated, and the cells were resuspended in 40 ml EasySep buffer (PBS+2% heat-inactivated fetal bovine serum+1 mM EDTA). The cells were centrifuged at 1200 rpm for 8 minutes and resuspended in EasySep buffer at a concentration of 5×10⁷ cells/ml and transferred to a 15 ml polystyrene round-bottom tube. Human CD4⁺ T cells were isolated using the EasySep Human CD4⁺ T Cell Isolation Kit according to manufacturer's instructions (Stemcell Technologies). The isolated cells were resuspended in complete media at a concentration of 1×10⁶ cell/ml.

The LPS-matured mouse bone marrow-derived dendritic cells were harvested from the plates and combined into a 50 m conical tube. The plate wells were washed with 2 ml of PBS, and then 2 ml PBS+3 mM EDTA (Thermo Fisher Scientific) was added to each well for 10 minutes at 37° C./5% CO₂ to harvest the remaining dendritic cells. The cells were collected from the plate and transferred into the 50 ml conical. The cells were washed three times with 40 ml complete media (centrifugation at 1200 rpm for 8 minutes). The DCs were resuspended in complete media to a concentration of 2.5×10⁵ cells/ml. Fifty μl of cells were added to each well of a 96-well round bottom plate. The anti-HLA-DR antibody was added at single dose of 10 μg/ml or serially diluted in complete media at 4× the final concentration, and 50 μl of the antibody dilution was added to each well. Control wells received 50 dl of media. The T cells were added to each well (100 μl/well of 1×10⁶ T cells/ml), resulting in a total volume of 200 μl/well. The plates were incubated at 37° C./5% CO₂ for 5 days. After incubation, 25 μl of complete media containing 1.0 mCi/well 3H-thymidine (Perkin Elmer) was added to all wells and incubate for 6 hours at 37° C./5% CO₂. The cells were harvested onto Unifilter-96, GF/C plates (Perkin Elmer), which were allowed to dry overnight at RT. Fifty μl of Microscint-20 (Perkin Elmer) was added to each well and counted using the TopCount instrument (Perkin Elmer).

Antibody Binding to Dendritic Cells from HLA-DR4 Transgenic Mice

The binding of anti-HLA-DR antibodies to dendritic cells from HLA-DR4 transgenic mice was assessed. HLA-DR4 DCs were derived as described above. DCs (5×10⁵ cells/well) were plated in Complete media (RPMI-1640/Glutamax containing 1% Penicillin/Streptomycin+10% fetal bovine serum—all purchased from Thermo Fisher Scientific) into a 96 well round bottom plate. Cells were resuspended in 50 μl Complete media containing TruStain FcX (BioLegend) and incubated at room temperature for 10 minutes. Anti-HLA-DR mAbs and the isotype control mAb were diluted in Complete media to 2× the final concentration to be tested (final concentration 10 μg/ml). Fifty μl of the diluted mAbs were added to the wells. The plates were incubated at 37° C. for 30 minutes. The cells were washed twice with 200 μl azide- and serum/protein-free PBS and centrifuged at 1400 rpm for 5 minutes at 4° C. Cells were resuspended in 100 μl PBS containing Fixable Viability Dye eFluor 450 (eBioscience) diluted 1:4000 or PBS alone. The plates were incubated for 30 minutes at 2-8° C., protected from light. The cells were washed with 150 μl of FACS buffer and centrifuged at 1400 rpm for 5 minutes at 4° C. Fifty μl FACS buffer containing hamster anti-mouse CD11c-PE-Cy7 (BD Biosciences; 1:20, 5 μl/test) and AF647 AffiniPure F(ab′)₂ Fragment Goat anti-human IgG, Fcγ Fragment Specific (Jackson Immunoresearch; 1:2000 dilution) was added to each well, and the plates were incubated for 30 minutes in the dark and on ice. Cells were washed with 150 μl FACS buffer per well and centrifuged at 1400 rpm for 5 minutes at 4° C. The cells were resuspended cells in 200 μl 4% paraformaldehyde solution (Affymetrix) and incubated on ice for 15 minutes. The cells were centrifuged at 1800 rpm for 5 minutes and resuspended in 200 μl of FACS buffer. The events were collected on an LSR II flow cytometer (BD Biosciences). Mean fluorescence intensities (MFIs, GeoMean) were determined for were determined for Live/Dead⁻ CD11c⁺ dendritic cells using Flowjo software. The level of binding for anti-HLA-DR mAbs was compared to the isotype control.

### Human B Cell Viability Assay

Blood was collected from Johnson & Johnson employee donors using Clinical Protocol: NOCOMPOUNDNAP1001 “Generation of reagents from human whole blood for the development and control of laboratory assays and procedures”. The blood was collected into BD Vacutainers containing sodium heparin. The blood was diluted 1:1 to 1:3 in PBS. Fifteen ml of Ficoll-Paque (GE Healthcare) was added to a 50 ml conical, and 30 ml of diluted blood was gently layered over the Ficoll by pipetting slowly down the side of the tilted tube. The conical was centrifuged for 30 minutes at 400 g without the brake at RT. The PBMC layer was collected into a 50 ml conical tube, which was then filled with PBS and centrifuged at 1200 rpm for 10 minutes. The cells were washed an additional time with PBS. The cells were resuspended in complete media (RPMI-1640/Glutamax containing 1% Penicillin/Streptomycin, 1% sodium pyruvate, 1% NEAA, 1% HEPES, and 10% heat-inactivated fetal bovine serum, all purchased from Thermo Fisher Scientific) and counted. The cells were plated in 96-well round bottom plates at a concentration of 800.000 cells per well. The anti-HLA-DR antibodies were added to the wells at concentrations of 0.2 μg/ml and 2 μg/ml. The plates were incubated for 20 h at 37° C./5% CO₂. The cells were then resuspended in 100 μl FACS buffer (2% heat-inactivated fetal bovine serum in PBS. ThermoFisher Scientific) with 100 μg/ml human IgG (Sigma-Aldrich) for 15 minutes at RT. The cells were pelleted by centrifugation and resuspended in 50 μl antibody cocktail for 20 minutes on ice. The antibody cocktail contained the following: Brilliant stain buffer (BD Biosciences), anti-CD3-PE Cy7 clone OKT3 (BioLegend), anti-CD20-APC Cy7 clone 2H7 (BioLegend), anti-CD16-BV605 clone 3G8 (BioLegend), and anti-CD14-BV785 clone M5E2 (BioLegend). The cells were then washed 2× in PBS, resuspended in 100 μl Live/Dead stain-eF660 (L/D) (eBioscience; 1 μl per ml of PBS), and incubated for 20 minutes on ice. The cells were washed once in PBS and then once in Annexin V binding buffer (BioLegend). The cells were resuspended in 100 μl Annexin V binding buffer+5 μl Annexin V-Pacific Blue (BioLegend) for 20 minutes at RT. The cells were washed once in Annexin V binding buffer and resuspended in 100 μl CytoFix (BD Biosciences) for 10 minutes on ice. The cells were washed once in FACS buffer and then resuspended in 200 μl FACS buffer. The events were collected on an LSR II flow cytometer (BD Biosciences); Ultracomp beads (eBisocience) were used to set up single-color compensations. The frequencies of Live/Dead (L/D) and Annexin V+/−B cells were determined using Flowjo software and graphed in GraphPad Prism 6. The frequency of dead B cells was calculated as the percentage of CD3⁻ CD20⁺ cells that were also eF660⁺ and Annexin V⁺. The frequency of apoptotic B cells was calculated as the percentage of CD3⁻ CD20⁺ cells that were also eF660⁻ and Annexin V⁺. Statistical significance was determined using a t test.

### Antibody Binding to Human PBMCs

The binding of anti-HLA-DR antibodies to human PBMCs was assessed. Human PBMC were isolated as described above. PBMC from each donor were plated in 96 well round-bottom plates at 500,000 cells per well. Cells were resuspended in 100 μl FACS buffer (2% heat-inactivated fetal bovine serum in PBS) with 100 μg/ml human IgG (Sigma-Aldrich) for 15 minutes at RT. One μg of anti-HLA-DR mAb in 25 μl Brilliant stain buffer (BD Biosciences) was added to wells, then 25 μl of antibody cocktail was added to wells. The antibody cocktail contained Brilliant stain buffer (BD Biosciences) and each of the following at 2 μl/test: anti-CD3-PE Cy7 clone OKT3 (BioLegend), anti-CD20-APC Cy7 clone 2H7 (BioLegend), anti-CD16-BV605 clone 3G8 (BioLegend), and anti-CD14-BV785 clone M5E2 (BioLegend). Cells were incubated for 20 min on ice, then washed twice in FACS buffer. The cells were then resuspended in 50 μl of a 1:200 dilution of AF488-labeled Affinipure F(ab)′2 fragment goat anti-human IgG, Fcγ fragment specific (Jackson Immunoresearch), for 20 minutes on ice. The cells were washed twice in PBS, and resuspended in 100 μl of Live/Dead stain (eBioscience, 0.5 μl per nil PBS) for 30 minutes on ice. The cells were washed twice in FACS buffer, resuspended in 100 μl Cytofix (BD) for 10 minutes on ice, washed once in FACS buffer, and then resuspended in 200 μl FACS buffer. The events were collected on an LSR II flow cytometer (BD Biosciences); Cells from Donor 1 were used to set up single-color compensations. Mean fluorescence intensities (MFIs. GeoMean) were determined were determined using Flowjo software and graphed in GraphPad Prism 6. The level of binding for anti-HLA-DR mAbs was compared to the isotype control.

### Results

All tested antibodies inhibited T cell activation in the MLR assay at a single dose concentration of 10 μg/ml. The antibodies inhibited cell proliferation in the MLR assay with an ICs values ranging from 0.11-5.36 μg/ml. The control antibody DR4B6 inhibited the MLR in a dose-dependent manner whereas the control antibodies DR4B4 and DR4B5 did not reach 100% inhibition at the highest 10 μg/ml concentration tested and therefore the IC, value could not be calculated for these antibodies.

All tested antibodies bound to human PBMCs and also to DCs from human HLA-DR4 transgenic animals. One control antibody, DR4B5, demonstrated low binding to the HLA-DR4 transgenic DCs.

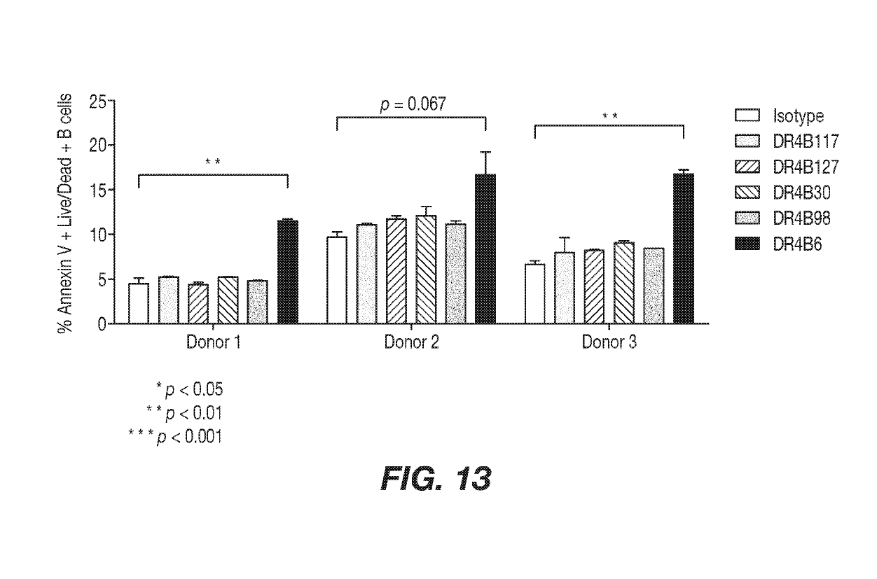

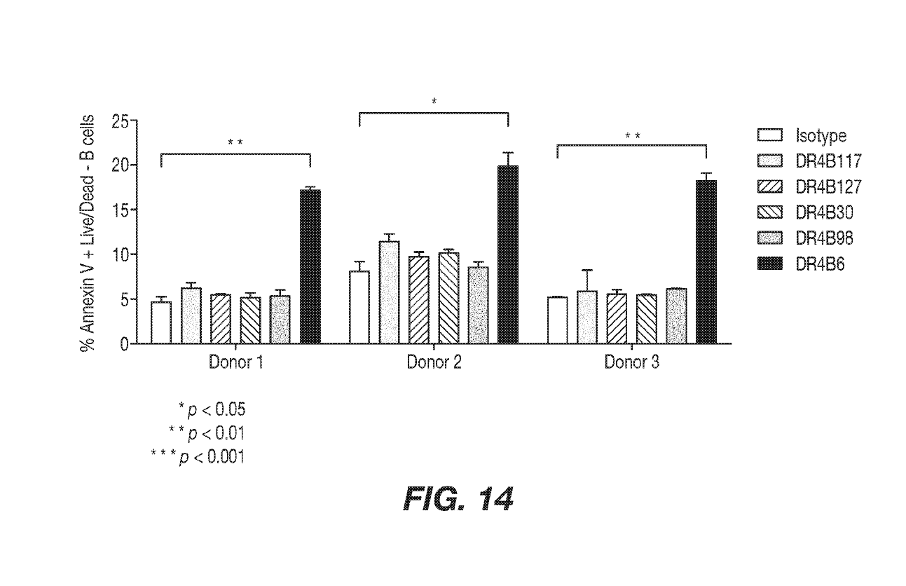

The generated antibodies differed from the test antibodies in their inability to induce death or apoptosis of B cells. FIG. 13 shows that the generated anti-HLA-DR antibodies had no effect on the frequency of dead B cells in three separate donors when compared to the isotype control, whereas the control antibody DR4B6 demonstrated a statistically significant increase in the frequency of dead B cells. Similarly, FIG. 14 shows that the generated anti-HLA-DR antibodies did not induce apoptosis in B cells from three separate donors, whereas the control antibody DR4B6 did.

Table 7 shows the characteristics of select anti-HLA-DR antibodies.

DR4B4 (Lym-l) and DR4B5 (apolizumab) have been shown to induce B cell apoptosis and death (Zhang et al., Cancer Biother Radiopharm 22:342-56, 2007; Mone et al., Blood 103: 1846-54, 2004).

TABLE 7

| | HLA-DR4 DC | | HLA-DR4 DC | | | | |
|---|---|---|---|---|---|---|---|
| | MLR | | MLR | | | | |
| | Average percent | | Average percent | | | | |
| | (%) inhibition at | | (%) inhibition at | | HLA- | Human | Human B |
| | 10 μg/ml mAb* | | 1 μg/ml mAb* | | DR4 DC | PBMC | cell |
| mAb | Donor 1 | Donor 2 | Donor 1 | Donor 2 | Binding | Binding | viability |
| DR4B117 | 42.1% | 37.6% | 25.2% | 28.8% | High | High | No effect |
| DR4B30‡ | 30.2% | 48.6% | 8.3% | 2.8% | High | High | No effect |
| DR4B127 | 74.4% | 92.6% | 77.8% | 71.8% | High | High | No effect |
| DR4B98 | 72.6% | 82.4% | 40.5% | 50.6% | High | High | No effect |
| DR4B6 | | | 89.3% | 90.1% | High | High | Induced |
| | | | | | | | cell death |
| | Donor 3 | Donor 4 | Donor 3 | Donor 4 | | | |
| DR4B4 | 46.4% | 26.9% | 26.3% | 22.4% | | | |
| DR4B5 | 49.1% | 73.4% | 25.7% | 67.7% | Low | | |
| DR4B6 | NT | NT | 91.4% | 100% | High | High | Induced |
| | | | | | | | cell death |

*Average percent inhibition measured in triplicate wells
‡DR4B30 demonstrated 39.8% and 69.9% inhibition at 30 μg/ml in Donors 1 & 2, respectively

### Example 5. Structural Characterization of Anti-HLA-DR Antibodies

The cDNA sequences and amino acid translations of the antibodies were obtained using standard techniques. After polypeptide sequence determination, some antibody cDNAs encoding the variable regions or full length antibodies were codon optimized using standard methods for scale-up expression.

Table 8 shows the HCDR1 amino acid sequences of select anti-HLA-DR antibodies.
Table 9 shows the HCDR2 amino acid sequences of select anti-HLA-DR antibodies.
Table 10 shows the HCDR3 amino acid sequences of select anti-HLA-DR antibodies.
Table 11 shows the LCDR1 amino acid sequences of select anti-HLA-DR antibodies.
Table 12 shows the LCDR2 amino acid sequences of select anti-HLA-DR antibodies.
Table 13 shows the LCDR3 amino acid sequences of select anti-HLA-DR antibodies.
Table 14 shows the protein SEQ ID NOs: for the VH, the VL, the HC and the LC pairs of select anti-HLA-DR antibodies.
Table 15 shows the polynucleotide SEQ ID NOs: encoding the VH, the VL, the HC and the LC of select anti-HLA-DR antibodies.
Table 16 shows the amino acid sequences of the VH, the VL, the HC and the LC of select anti-HLA-DR antibodies and polynucleotide sequences encoding them.
Table 17 shows the frameworks of select anti-HLA-DR antibodies.

TABLE 8

HCDR1

| | | | | | | SEQ ID |
|---|---|---|---|---|---|---|
| mAb | Sequence | | | | | NO: |
| DR4B117 | S | Y | S | I | H | 39 |
| DR4B30 | S | D | W | I | G | 40 |
| DR4B127 | S | Y | Y | I | H | 41 |
| DR4B98 | S | Y | Y | I | H | 41 |
| DR4B78 | S | Y | A | M | S | 123 |
| DR4B70 | S | Y | A | M | S | 123 |
| DR4B38 | S | Y | A | M | S | 123 |
| DR4B33 | S | A | Y | I | N | 124 |
| DR4B22 | S | Y | A | M | N | 125 |

TABLE 9

| | HCDR2 | | | | | | | | | | | | | | | | | |
|---|---|---|---|---|---|---|---|---|---|---|---|---|---|---|---|---|---|---|
| | | | | | | | | | | | | | | | | | | SEQ |
| | | | | | | | | | | | | | | | | | | ID |
| mAb | Sequence | | | | | | | | | | | | | | | | | NO: |
| DR4B117 | Y | I | I | P | E | Y | G | T | A | N | Y | A | Q | K | F | Q | G | 42 |
| DR4B30 | I | I | R | P | G | D | S | D | T | Y | Y | S | P | S | F | Q | G | 43 |
| DR4B127 | G | I | R | P | I | S | G | N | A | E | Y | A | Q | K | F | Q | G | 44 |
| DR4B98 | G | I | A | P | I | Y | G | T | A | Y | Y | A | Q | K | F | Q | G | 45 |
| DR4B78 | A | I | S | G | S | G | G | S | T | Y | Y | A | D | S | V | K | G | 126 |
| DR4B70 | A | I | S | G | S | G | G | S | T | Y | Y | A | D | S | V | K | G | 126 |
| DR4B38 | A | I | S | G | S | G | G | S | T | Y | Y | A | D | S | V | K | G | 126 |
| DR4B33 | I | I | R | P | G | D | S | R | T | R | Y | S | P | S | F | Q | G | 127 |
| DR4B22 | A | I | S | G | S | G | G | Y | T | N | Y | A | D | S | V | K | G | 128 |

TABLE 10

| | | | | | | | | | | | | | | | | | | | | SEQ |
|---|---|---|---|---|---|---|---|---|---|---|---|---|---|---|---|---|---|---|---|---|
| | | | | | | | | | | | | | | | | | | | | ID |
| mAb | HCDR3 sequence | | | | | | | | | | | | | | | | | | | NO: |
| DR4B117 | G | R | Y | Y | I | G | N | R | R | G | S | Y | Y | G | F | D | Y | | | 46 |
| DR4B30 | E | S | Y | Y | Y | V | G | V | R | Y | R | P | S | Y | Y | F | D | Y | | 47 |
| DR4B127 | D | A | S | Y | Y | R | N | Y | G | F | D | Y | | | | | | | | 48 |
| DR4B98 | D | A | S | W | A | R | A | Y | G | F | D | Y | | | | | | | | 49 |
| DR4B78 | D | G | G | Y | Y | R | Y | V | R | T | I | S | G | D | Y | A | F | D | Y | 129 |
| DR4B70 | D | S | S | Y | Y | R | Y | I | G | R | Y | L | G | D | Y | A | F | D | Y | 130 |
| DR4B38 | D | S | G | Y | Y | R | L | A | A | I | G | R | S | D | Y | A | F | D | Y | 131 |
| DR4B33 | D | G | Y | Y | F | V | G | S | I | I | Y | Y | G | M | D | V | | | | 132 |
| DR4B22 | D | G | G | Y | Y | R | Y | V | Y | R | Y | P | G | D | Y | A | F | G | Y | 133 |

TABLE 11

| | LCDR1 | | | | | | | | | | | | |
|---|---|---|---|---|---|---|---|---|---|---|---|---|---|
| | | | | | | | | | | | | | SEQ |
| | | | | | | | | | | | | | ID |
| mAb | Sequence | | | | | | | | | | | | NO: |
| DR4B117 | R | A | S | Q | S | V | S | S | S | Y | L | A | 50 |
| DR4B30 | R | A | S | Q | S | V | S | S | Y | L | A | | 51 |
| DR4B127 | R | A | S | Q | S | V | S | S | Y | L | A | | 51 |
| DR4B98 | R | A | S | Q | S | V | S | S | Y | L | A | | 51 |
| DR4B78 | R | A | S | Q | S | V | S | S | Y | L | A | | 51 |
| DR4B70 | R | A | S | Q | S | V | S | S | Y | L | A | | 51 |
| DR4B38 | R | A | S | Q | S | V | S | S | Y | L | A | | 51 |
| DR4B33 | R | A | S | Q | S | I | S | S | Y | L | N | | 134 |
| DR4B22 | R | A | S | Q | S | V | S | S | Y | L | A | | 51 |

TABLE 12

| | LCDR2 | | | | | | | |
|---|---|---|---|---|---|---|---|---|
| | | | | | | | | SEQ |
| | | | | | | | | ID |
| mAb | Sequence | | | | | | | NO: |
| DR4B117 | G | A | S | S | R | A | T | 52 |
| DR4B30 | D | A | S | N | R | A | T | 53 |
| DR4B127 | D | A | S | N | R | A | T | 53 |
| DR4B98 | D | A | S | N | R | A | T | 53 |
| DR4B78 | D | A | S | N | R | A | T | 53 |
| DR4B70 | D | A | S | N | R | A | T | 53 |
| DR4B38 | D | A | S | N | R | A | T | 53 |
| DR4B33 | A | A | S | S | L | Q | S | 135 |
| DR4B22 | D | A | S | N | R | A | T | 53 |

TABLE 13

| | LCDR3 | | | | | | | | | |
|---|---|---|---|---|---|---|---|---|---|---|
| | | | | | | | | | | SEQ ID |
| mAb | Sequence | | | | | | | | | NO: |
| DR4B117 | Q | Q | Y | G | S | S | P | L | T | 54 |
| DR4B30 | Q | Q | R | S | N | W | P | L | T | 55 |
| DR4B127 | Q | Q | R | S | N | W | P | L | T | 55 |
| DR4B98 | Q | Q | R | S | N | W | P | L | T | 55 |
| DR4B78 | Q | Q | R | S | N | W | P | L | T | 55 |
| DR4B70 | Q | Q | R | S | N | W | P | L | T | 55 |
| DR4B38 | Q | Q | R | S | N | W | P | L | T | 55 |
| DR4B33 | Q | Q | S | Y | S | T | P | L | T | 136 |
| DR4B22 | Q | Q | R | S | N | W | P | L | T | 55 |

TABLE 14

| | | VH | | VL | HC | LC |
|---|---|---|---|---|---|---|
| | | protein | | protein | protein | protein |
| | VH | SEQ ID | VL | SEQ ID | SEQ ID | SEQ ID |
| mAb | name | NO: | name | NO: | NO: | NO: |
| DR4B117 | DR4H4 | 56 | PH9L1 | 60 | 84 | 88 |
| DR4B30 | DR4H39 | 57 | PH9L3 | 61 | 85 | 89 |
| DR4B127 | DR4H7 | 58 | PH9L3 | 61 | 86 | 89 |
| DR4B98 | DR4H50 | 59 | PH9L3 | 61 | 87 | 89 |
| DR4B78 | DR4H62 | 137 | PH9L3 | 61 | 149 | 89 |
| DR4B70 | DR4H29 | 138 | PH9L3 | 61 | 150 | 89 |
| DR4B38 | DR4H56 | 139 | PH9L3 | 61 | 151 | 89 |
| DR4B33 | DR4H58 | 140 | PH9L4 | 142 | 152 | 154 |
| DR4B22 | DR4H16 | 141 | PH9L3 | 61 | 153 | 89 |

TABLE 15

| | SEQ ID NOs: for polynucleotides | | | | | |
|---|---|---|---|---|---|---|
| | | VH | | VL | HC | LC |
| | | SEQ | | SEQ | SEQ | SEQ ID |
| mAb | VH name | ID NO: | VL name | ID NO: | ID NO: | NO: |
| DR4B117 | DR4H4 | 79 | PH9L1 | 80 | 90 | 94 |
| DR4B30 | DR4H39 | 81 | PH9L3 | 82 | 91 | 95 |
| DR4B127 | DR4H7 | 83 | PH9L3 | 82 | 92 | 95 |
| DR4B98 | DR4H50 | 121 | PH9L3 | 82 | 93 | 95 |
| DR4B78 | DR4H62 | 143 | PH9L3 | 82 | 155 | 95 |
| DR4B70 | DR4H29 | 144 | PH9L3 | 82 | 156 | 95 |
| DR4B38 | DR4H56 | 145 | PH9L3 | 82 | 157 | 95 |
| DR4B33 | DR4H58 | 146 | PH9L4 | 148 | 158 | 160 |
| DR4B22 | DR4H16 | 147 | PH9L3 | 82 | 159 | 95 |

TABLE 16

| Sequence name | Sequence | SEQ ID NO: |
|---|---|---|
| DR4B117 VH | QVQLVQSGAEVKKPGSSVKVSCKASGGTFSSYSIH | 56 |
| (DR4H4) | WVRQAPGQGLEWMGYIIPEYGTANYAQKFQGRVTI | |
| amino acid | TADESTSTAYMELSSLRSEDTAVYYCARGRYYIGN | |
| | RRGSYYGFDYWGQGTLVTVSS | |
| DR4B30 VH | EVQLVQSGAEVKKPGESLKISCKGSGYSFTSDWIG | 57 |
| (DR4H39) | WVRQMPGKGLEWMGIIRPGDSDTYYSPSFQGQVTI | |
| amino acid | SADKSISTAYLQWSSLKASDTAVYYCARESYYYVG | |
| | VRYRPSYYFDYWGQGTLVTVSS | |
| DR4B127 VH | QVQLVQSGAEVKKPGSSVKVSCKASGGTFKSYYIH | 58 |
| (DR4H7) | WVRQAPGQGLEWMGGIRPISGNAEYAQKFQGRVTI | |
| amino acid | TADESTSTAYMELSSLRSEDTAVYYCARDASYYRN | |
| | YGFDYWGQGTLVTVSS | |
| DR4B98 VH | QVQLVQSGAEVKKPGSSVKVSCKASGGTFKSYYIH | 59 |
| (DR4H50) | WVRQAPGQGLEWMGGIAPIYGTAYYAQKFQGRVT | |
| amino acid | ITADESTSTAYMELSSLRSEDTAVYYCARDASWAR | |
| | AYGFDYWGQGTLVTVSS | |
| DR4B117 VL | EIVLTQSPGTLSLSPGERATLSCRASQSVSSSYLAWY | 60 |
| (PH9L1) amino | QQKPGQAPRLLIYGASSRATGIPDRFSGSGSGTDFTL | |
| acid | TISRLEPEDFAVYYCQQYGSSPLTFGQGTKVEIK | |
| DR4B30, | EIVLTQSPATLSLSPGERATLSCRASQSVSSYLAWY | 61 |
| DR4B127 and | QQKPGQAPRLLIYDASNRATGIPARFSGSGSGTDFT | |
| DR4B98 VL | LTISSLEPEDFAVYYCQQRSNWPLTFGQGTKVEIK | |
| (PH9L3) amino | | |
| acid | | |
| DR4B117 HC | QVQLVQSGAEVKKPGSSVKVSCKASGGTFSSYSIH | 84 |
| amino acid | WVRQAPGQGLEWMGYIIPEYGTANYAQKFQGRVTI | |
| | TADESTSTAYMELSSLRSEDTAVYYCARGRYYIGN | |
| | RRGSYYGFDYWGQGTLVTVSSASTKGPSVFPLAPC | |
| | SRSTSESTAALGCLVKDYFPEPVTVSWNSGALTSGV | |
| | HTFPAVLQSSGLYSLSSVVTVPSSNFGTQTYTCNVD | |
| | HKPSNTKVDKTVERKCCVECPPCPAPPAAASSVFLF | |
| | PPKPKDTLMISRTPEVTCVVVDVSAEDPEVQFNWY | |
| | VDGVEVHNAKTKPREEQFNSTFRVVSVLTVLHQD | |
| | WLNGKEYKCKVSNKGLPSSIEKTISKTKGQPREPQV | |
| | YTLPPSREEMTKNQVSLTCLVKGFYPSDIAVEWESN | |
| | GQPENNYKTTPPMLDSDGSFFLYSKLTVDKSRWQQ | |
| | GNVFSCSVMHEALHNHYTQKSLSLSPGK | |
| DR4B30 HC | EVQLVQSGAEVKKPGESLKISCKGSGYSFTSDWIG | 85 |
| amino acid | WVRQMPGKGLEWMGIIRPGDSDTYYSPSFQGQVTI | |
| | SADKSISTAYLQWSSLKASDTAVYYCARESYYYVG | |
| | VRYRPSYYFDYWGQGTLVTVSSASTKGPSVFPLAP | |
| | CSRSTSESTAALGCLVKDYFPEPVTVSWNSGALTSG | |
| | VHTFPAVLQSSGLYSLSSVVTVPSSNFGTQTYTCNV | |
| | DHKPSNTKVDKTVERKCCVECPPCPAPPAAASSVFL | |
| | FPPKPKDTLMISRTPEVTCVVVDVSAEDPEVQFNW | |
| | YVDGVEVHNAKTKPREEQFNSTFRVVSVLTVLHQD | |
| | WLNGKEYKCKVSNKGLPSSIEKTISKTKGQPREPQV | |
| | YTLPPSREEMTKNQVSLTCLVKGFYPSDIAVEWESN | |
| | GQPENNYKTTPPMLDSDGSFFLYSKLTVDKSRWQQ | |
| | GNVFSCSVMHEALHNHYTQKSLSLSPGK | |
| DR4B127 HC | QVQLVQSGAEVKKPGSSVKVSCKASGGTFKSYYIH | 86 |
| amino acid | WVRQAPGQGLEWMGGIRPISGNAEYAQKFQGRVTI | |
| | TADESTSTAYMELSSLRSEDTAVYYCARDASYYRN | |
| | YGFDYWGQGTLVTVSSASTKGPSVFPLAPCSRSTSE | |
| | STAALGCLVKDYFPEPVTVSWNSGALTSGVHTFPA | |
| | VLQSSGLYSLSSVVTVPSSNFGTQTYTCNVDHKPSN | |
| | TKVDKTVERKCCVECPPCPAPPAAASSVFLFPPKPK | |
| | DTLMISRTPEVTCVVVDVSAEDPEVQFNWYVDGVE | |
| | VHNAKTKPREEQFNSTFRVVSVLTVLHQDWLNGKE | |
| | YKCKVSNKGLPSSIEKTISKTKGQPREPQVYTLPPSR | |
| | EEMTKNQVSLTCLVKGFYPSDIAVEWESNGQPENN | |
| | YKTTPPMLDSDGSFFLYSKLTVDKSRWQQGNVFSC | |
| | SVMHEALHNHYTQKSLSLSPGK | |
| DR4B98 HC | QVQLVQSGAEVKKPGSSVKVSCKASGGTFKSYYIH | 87 |
| amino acid | WVRQAPGQGLEWMGGIAPIYGTAYYAQKFQGRVT | |
| | ITADESTSTAYMELSSLRSEDTAVYYCARDASWAR | |
| | AYGFDYWGQGTLVTVSSASTKGPSVFPLAPCSRSTS | |
| | ESTAALGCLVKDYFPEPVTVSWNSGALTSGVHTFP | |
| | AVLQSSGLYSLSSVVTVPSSNFGTQTYTCNVDHKPS | |
| | NTKVDKTVERKCCVECPPCPAPPAAASSVFLFPPKP | |
| | KDTLMISRTPEVTCVVVDVSAEDPEVQFNWYVDGV | |
| | EVHNAKTKPREEQFNSTFRVVSVLTVLHQDWLNGK | |
| | EYKCKVSNKGLPSSIEKTISKTKGQPREPQVYTLPPS | |
| | REEMTKNQVSLTCLVKGFYPSDIAVEWESNGQPEN | |
| | NYKTTPPMLDSDGSFFLYSKLTVDKSRWQQGNVFS | |
| | CSVMHEALHNHYTQKSLSLSPGK | |
| DR4B117LC | EIVLTQSPGTLSLSPGERATLSCRASQSVSSSYLAWY | 88 |
| amino acid | QQKPGQAPRLLIYGASSRATGIPDRFSGSGSGTDFTL | |
| | TISRLEPEDFAVYYCQQYGSSPLTFGQGTKVEIKRT | |
| | VAAPSVFIFPPSDEQLKSGTASVVCLLNNFYPREAK | |
| | VQWKVDNALQSGNSQESVTEQDSKDSTYSLSSTLT | |
| | LSKADYEKHKVYACEVTHQGLSSPVTKSFNRGEC | |
| DR4B30, | EIVLTQSPATLSLSPGERATLSCRASQSVSSYLAWY | 89 |
| DR4B127 and | QQKPGQAPRLLIYDASNRATGIPARFSGSGSGTDFT | |
| DR4B98 LC | LTISSLEPEDFAVYYCQQRSNWPLTFGQGTKVEIKR | |
| amino acid | TVAAPSVFIFPPSDEQLKSGTASVVCLLNNFYPREA | |
| | KVQWKVDNALQSGNSQESVTEQDSKDSTYSLSSTL | |
| | TLSKADYEKHKVYACEVTHQGLSSPVTKSFNRGEC | |
| DR4B117 VH | CAGGTGCAGCTGGTGCAGAGCGGCGCGGAAGTG | 79 |
| (DR4H4) | AAAAAACCGGGCAGCAGCGTGAAAGTGAGCTGC | |
| polynucleotide | AAAGCGAGCGGCGGCACCTTTAGCAGCTATTCCA | |
| | TTCACTGGGTGCGCCAGGCGCCGGGCCAGGGCCT | |
| | GGAATGGATGGGCTACATTATTCCGGAGTACGGG | |
| | ACTGCCAATTACGCGCAGAAATTTCAGGGCCGCG | |
| | TGACCATTACCGCTGATGAAAGCACCAGCACCGC | |
| | GTATATGGAACTGAGCAGCCTGCGCAGCGAAGAT | |
| | ACCGCGGTGTATTATTGCGCGCGCGGCCGATACT | |
| | ATATCGGCAACCGTCGTGGCAGTTATTACGGTTTT | |
| | GACTATTGGGGCCAGGGCACCCTGGTGACCGTCT | |
| | CGAGT | |
| DR4B30 VH | GAAGTGCAGCTGGTGCAGAGCGGCGCGGAAGTG | 81 |
| (DR4H39) | AAAAAACCGGGCGAAAGCCTGAAAATTAGCTGC | |
| polynucleotide | AAAGGCAGCGGCTATAGCTTTACCAGCGACTGGA | |
| | TTGGTTGGGTGCGCCAGATGCCGGGCAAAGGCTT | |
| | GGAATGGATGGGTATCATTCGCCCGGGCGATAGC | |
| | GATACGTATTACAGCCCGAGCTTTCAGGGCCAGG | |
| | TGACCATTAGCGCGGATAAAAGCATTAGCACCGC | |
| | GTATCTGCAGTGGAGCAGCCTGAAAGCGAGCGAT | |
| | ACCGCGGTGTATTATTGCGCGCGTGAATCCTATT | |
| | ATTACGTTGGCGTGCGTTACCGTCCAAGCTATTAT | |
| | TTCGATTACTGGGGCCAGGGCACCCTGGTGACCG | |
| | TCTCGAGT | |
| DR4B1.27 VH | CAGGTGCAGCTGGTGCAGAGCGGCGCGGAAGTG | 83 |
| (DR4H7) | AAAAAACCGGGCAGCAGCGTGAAAGTGAGCTGC | |
| polynucleotide | AAAGCGAGCGGCGGCACCTTTAAATCCTACTACA | |
| | TTCACTGGGTGCGCCAGGCGCCGGGCCAGGGCCT | |
| | GGAATGGATGGGTGGTATTCGTCCGATCAGCGGG | |
| | AATGCTGAGTACGCGCAGAAATTTCAGGGCCGCG | |
| | TGACCATTACCGCTGATGAAAGCACCAGCACCGC | |
| | GTATATGGAACTGAGCAGCCTGCGCAGCGAAGAT | |
| | ACCGCGGTGTATTATTGCGCGCGCGATGCAAGCT | |
| | ATTATCGTAATTACGGTTTTGACTACTGGGGCCA | |
| | GGGCACCCTGGTGACCGTCTCGAGT | |
| DR4B98 VH | CAGGTGCAGCTGGTGCAGAGCGGCGCGGAAGTG | 121 |
| (DR4H50) | AAAAAACCGGGCAGCAGCGTGAAAGTGAGCTGC | |
| polynucleotide | AAAGCGAGCGGCGGCACCTTTAAGTCCTATTATA | |
| | TTCATTGGGTGCGCCAGGCGCCGGGCCAGGGCCT | |
| | GGAATGGATGGGCGGTATTGCACCAATTTACGGC | |
| | ACCGCTTACTACGCGCAGAAATTTCAGGGCCGCG | |
| | TGACCATTACCGCTGATGAAAGCACCAGCACCGC | |
| | GTATATGGAACTGAGCAGCCTGCGCAGCGAAGAT | |
| | ACCGCGGTGTATTATTGCGCGCGTGATGCAAGTT | |
| | GGGCACGTGCATACGGTTTTGATTATTGGGGCCA | |
| | GGGCACCCTGGTGACCGTCTCGAGT | |
| DR4B117 VL | GAGATCGTGCTGACCCAGAGCCCCGGCACCCTGA | 80 |
| (PH9L1) | GCCTGAGCCCCGGCGAGCGGGCCACCCTGAGCTG | |
| polynucleotide | CCGGGCCAGCCAGAGCGTGAGCAGCAGCTACCTG | |
| | GCCTGGTACCAGCAGAAGCCCGGCCAGGCCCCCC | |
| | GGCTGCTGATCTACGGCGCCAGCAGCCGGGCCAC | |
| | CGGCATCCCCGACCGGTTCAGCGGCAGCGGCAGC | |
| | GGCACCGACTTCACCCTGACCATCAGCCGGCTGG | |
| | AGCCCGAGGACTTCGCCGTGTACTACTGCCAGCA | |
| | GTACGGCAGCAGCCCCCTGACCTTCGGCCAGGGC | |
| | ACCAAGGTGGAGATCAAG | |
| DR4B30, | GAGATCGTGCTGACCCAGAGCCCCGCCACCCTGA | 82 |
| DR4B127 and | GCCTGAGCCCCGGCGAGCGGGCCACCCTGAGCTG | |
| DR4B98 VL | CCGGGCCAGCCAGAGCGTGAGCAGCTACCTGGCC | |
| (PH9L3) | TGGTACCAGCAGAAGCCCGGCCAGGCCCCCCGGC | |
| polynucleotide | TGCTGATCTACGACGCCAGCAACCGGGCCACCGG | |
| | CATCCCCGCCCGGTTCAGCGGCAGCGGCAGCGGC | |
| | ACCGACTTCACCCTGACCATCAGCAGCCTGGAGC | |
| | CCGAGGACTTCGCCGTGTACTACTGCCAGCAGCG | |
| | GAGCAACTGGCCCCTGACCTTCGGCCAGGGCACC | |
| | AAGGTGGAGATCAAG | |
| DR4B117 HC | CAGGTGCAGCTGGTGCAGAGCGGCGCGGAAGTG | 90 |
| polynucleotide | AAAAAACCGGGCAGCAGCGTGAAAGTGAGCTGC | |
| | AAAGCGAGCGGCGGCACCTTTAGCAGCTATTCCA | |
| | TTCACTGGGTGCGCCAGGCGCCGGGCCAGGGCCT | |
| | GGAATGGATGGGCTACATTATTCCGGAGTACGGG | |
| | ACTGCCAATTACGCGCAGAAATTTCAGGGCCGCG | |
| | TGACCATTACCGCTGATGAAAGCACCAGCACCGC | |
| | GTATATGGAACTGAGCAGCCTGCGCAGCGAAGAT | |
| | ACCGCGGTGTATTATTGCGCGCGCGGCCGATACT | |
| | ATATCGGCAACCGTCGTGGCAGTTATTACGGTTTT | |
| | GACTATTGGGGCCAGGGCACCCTGGTGACCGTCT | |
| | CGAGTGCCTCCACCAAGGGCCCATCGGTCTTCCC | |
| | CCTGGCGCCCTGCTCCAGGAGCACCTCCGAGAGC | |
| | ACAGCCGCCCTGGGCTGCCTGGTCAAGGACTACT | |
| | TCCCCGAACCGGTGACGGTGTCGTGGAACTCAGG | |
| | CGCTCTGACCAGCGGCGTGCACACCTTCCCAGCT | |
| | GTCCTACAGTCCTCAGGACTCTACTCCCTCAGCA | |
| | GCGTGGTGACCGTGCCCTCCAGCAACTTCGGCAC | |
| | CCAGACCTACACCTGCAACGTAGATCACAAGCCC | |
| | AGCAACACCAAGGTGGACAAGACAGTTGAGCGC | |
| | AAATGTTGTGTCGAGTGCCCACCGTGCCCAGCAC | |
| | CACCTGCCGCAGCCAGCTCAGTCTTCCTCTTCCCC | |
| | CCAAAACCCAAGGACACCCTCATGATCTCCCGGA | |
| | CCCCTGAGGTCACGTGCGTGGTGGTGGACGTGAG | |
| | CGCCGAAGACCCCGAGGTCCAGTTCAACTGGTAC | |
| | GTGGACGGCGTGGAGGTGCATAATGCCAAGACA | |
| | AAGCCACGGGAGGAGCAGTTCAACAGCACGTTCC | |
| | GTGTGGTCAGCGTCCTCACCGTTCTGCACCAGGA | |
| | CTGGCTGAACGGCAAGGAGTACAAGTGCAAGGT | |
| | CTCCAACAAAGGCCTCCCATCCTCCATCGAGAAA | |
| | ACCATCTCCAAAACCAAAGGGCAGCCCCGAGAA | |
| | CCACAGGTGTACACCCTGCCCCCATCCCGGGAGG | |
| | AGATGACCAAGAACCAGGTCAGCCTGACCTGCCT | |
| | GGTCAAAGGCTTCTACCCCAGCGACATCGCCGTG | |
| | GAGTGGGAGAGCAATGGGCAGCCGGAGAACAAC | |
| | TACAAGACCACACCTCCCATGCTGGACTCCGACG | |
| | GCTCCTTCTTCCTCTACAGCAAGCTCACCGTGGAC | |
| | AAGAGCAGGTGGCAGCAGGGGAACGTCTTCTCAT | |
| | GCTCCGTGATGCATGAGGCTCTGCACAACCACTA | |
| | CACGCAGAAGAGCCTCTCCCTGTCTCCGGGTAAA | |
| DR4B30 HC | GAAGTGCAGCTGGTGCAGAGCGGCGCGGAAGTG | 91 |
| polynucleotide | AAAAAACCGGGCGAAAGCCTGAAAATTAGCTGC | |
| | AAAGGCAGCGGCTATAGCTTTACCAGCGACTGGA | |
| | TTGGTTGGGTGCGCCAGATGCCGGGCAAAGGCTT | |
| | GGAATGGATGGGTATCATTCGCCCGGGCGATAGC | |
| | GATACGTATTACAGCCCGAGCTTTCAGGGCCAGG | |
| | TGACCATTAGCGCGGATAAAAGCATTAGCACCGC | |
| | GTATCTGCAGTGGAGCAGCCTGAAAGCGAGCGAT | |
| | ACCGCGGTGTATTATTGCGCGCGTGAATCCTATT | |
| | ATTACGTTGGCGTGCGTTACCGTCCAAGCTATTAT | |
| | TTCGATTACTGGGGCCAGGGCACCCTGGTGACCG | |
| | TCTCGAGTGCCTCCACCAAGGGCCCATCGGTCTT | |
| | CCCCCTGGCGCCCTGCTCCAGGAGCACCTCCGAG | |
| | AGCACAGCCGCCCTGGGCTGCCTGGTCAAGGACT | |
| | ACTTCCCCGAACCGGTGACGGTGTCGTGGAACTC | |
| | AGGCGCTCTGACCAGCGGCGTGCACACCTTCCCA | |
| | GCTGTCCTACAGTCCTCAGGACTCTACTCCCTCAG | |
| | CAGCGTGGTGACCGTGCCCTCCAGCAACTTCGGC | |
| | ACCCAGACCTACACCTGCAACGTAGATCACAAGC | |
| | CCAGCAACACCAAGGTGGACAAGACAGTTGAGC | |
| | GCAAATGTTGTGTCGAGTGCCCACCGTGCCCAGC | |
| | ACCACCTGCCGCAGCCAGCTCAGTCTTCCTCTTCC | |
| | CCCCAAAACCCAAGGACACCCTCATGATCTCCCG | |
| | GACCCCTGAGGTCACGTGCGTGGTGGTGGACGTG | |
| | AGCGCCGAAGACCCCGAGGTCCAGTTCAACTGGT | |
| | ACGTGGACGGCGTGGAGGTGCATAATGCCAAGA | |
| | CAAAGCCACGGGAGGAGCAGTTCAACAGCACGT | |
| | TCCGTGTGGTCAGCGTCCTCACCGTTCTGCACCAG | |
| | GACTGGCTGAACGGCAAGGAGTACAAGTGCAAG | |
| | GTCTCCAACAAAGGCCTCCCATCCTCCATCGAGA | |
| | AAACCATCTCCAAAACCAAAGGGCAGCCCCGAG | |
| | AACCACAGGTGTACACCCTGCCCCCATCCCGGGA | |
| | GGAGATGACCAAGAACCAGGTCAGCCTGACCTGC | |
| | CTGGTCAAAGGCTTCTACCCCAGCGACATCGCCG | |
| | TGGAGTGGGAGAGCAATGGGCAGCCGGAGAACA | |
| | ACTACAAGACCACACCTCCCATGCTGGACTCCGA | |
| | CGGCTCCTTCTTCCTCTACAGCAAGCTCACCGTGG | |
| | ACAAGAGCAGGTGGCAGCAGGGGAACGTCTTCTC | |
| | ATGCTCCGTGATGCATGAGGCTCTGCACAACCAC | |
| | TACACGCAGAAGAGCCTCTCCCTGTCTCCGGGTA | |
| | AA | |
| DR4B127 HC | CAGGTGCAGCTGGTGCAGAGCGGCGCGGAAGTG | 92 |
| polynucleotide | AAAAAACCGGGCAGCAGCGTGAAAGTGAGCTGC | |
| | AAAGCGAGCGGCGGCACCTTTAAATCCTACTACA | |
| | TTCACTGGGTGCGCCAGGCGCCGGGCCAGGGCCT | |
| | GGAATGGATGGGTGGTATTCGTCCGATCAGCGGG | |
| | AATGCTGAGTACGCGCAGAAATTTCAGGGCCGCG | |
| | TGACCATTACCGCTGATGAAAGCACCAGCACCGC | |
| | GTATATGGAACTGAGCAGCCTGCGCAGCGAAGAT | |
| | ACCGCGGTGTATTATTGCGCGCGCGATGCAAGCT | |
| | ATTATCGTAATTACGGTTTTGACTACTGGGGCCA | |
| | GGGCACCCTGGTGACCGTCTCGAGTGCCTCCACC | |
| | AAGGGCCCATCGGTCTTCCCCCTGGCGCCCTGCT | |
| | CCAGGAGCACCTCCGAGAGCACAGCCGCCCTGGG | |
| | CTGCCTGGTCAAGGACTACTTCCCCGAACCGGTG | |
| | ACGGTGTCGTGGAACTCAGGCGCTCTGACCAGCG | |
| | GCGTGCACACCTTCCCAGCTGTCCTACAGTCCTCA | |
| | GGACTCTACTCCCTCAGCAGCGTGGTGACCGTGC | |
| | CCTCCAGCAACTTCGGCACCCAGACCTACACCTG | |
| | CAACGTAGATCACAAGCCCAGCAACACCAAGGT | |
| | GGACAAGACAGTTGAGCGCAAATGTTGTGTCGAG | |
| | TGCCCACCGTGCCCAGCACCACCTGCCGCAGCCA | |
| | GCTCAGTCTTCCTCTTCCCCCCAAAACCCAAGGA | |
| | CACCCTCATGATCTCCCGGACCCCTGAGGTCACG | |
| | TGCGTGGTGGTGGACGTGAGCGCCGAAGACCCCG | |
| | AGGTCCAGTTCAACTGGTACGTGGACGGCGTGGA | |
| | GGTGCATAATGCCAAGACAAAGCCACGGGAGGA | |
| | GCAGTTCAACAGCACGTTCCGTGTGGTCAGCGTC | |
| | CTCACCGTTCTGCACCAGGACTGGCTGAACGGCA | |
| | AGGAGTACAAGTGCAAGGTCTCCAACAAAGGCCT | |
| | CCCATCCTCCATCGAGAAAACCATCTCCAAAACC | |
| | AAAGGGCAGCCCCGAGAACCACAGGTGTACACC | |
| | CTGCCCCCATCCCGGGAGGAGATGACCAAGAACC | |
| | AGGTCAGCCTGACCTGCCTGGTCAAAGGCTTCTA | |
| | CCCCAGCGACATCGCCGTGGAGTGGGAGAGCAAT | |
| | GGGCAGCCGGAGAACAACTACAAGACCACACCT | |
| | CCCATGCTGGACTCCGACGGCTCCTTCTTCCTCTA | |
| | CAGCAAGCTCACCGTGGACAAGAGCAGGTGGCA | |
| | GCAGGGGAACGTCTTCTCATGCTCCGTGATGCAT | |
| | GAGGCTCTGCACAACCACTACACGCAGAAGAGCC | |
| | TCTCCCTGTCTCCGGGTAAA | |
| DR4B98 HC | CAGGTGCAGCTGGTGCAGAGCGGCGCGGAAGTG | 93 |
| polynucleotide | AAAAAACCGGGCAGCAGCGTGAAAGTGAGCTGC | |
| | AAAGCGAGCGGCGGCACCTTTAAGTCCTATTATA | |
| | TTCATTGGGTGCGCCAGGCGCCGGGCCAGGGCCT | |
| | GGAATGGATGGGCGGTATTGCACCAATTTACGGC | |
| | ACCGCTTACTACGCGCAGAAATTTCAGGGCCGCG | |
| | TGACCATTACCGCTGATGAAAGCACCAGCACCGC | |
| | GTATATGGAACTGAGCAGCCTGCGCAGCGAAGAT | |
| | ACCGCGGTGTATTATTGCGCGCGTGATGCAAGTT | |
| | GGGCACGTGCATACGGTTTTGATTATTGGGGCCA | |
| | GGGCACCCTGGTGACCGTCTCGAGTGCCTCCACC | |
| | AAGGGCCCATCGGTCTTCCCCCTGGCGCCCTGCT | |
| | CCAGGAGCACCTCCGAGAGCACAGCCGCCCTGGG | |
| | CTGCCTGGTCAAGGACTACTTCCCCGAACCGGTG | |
| | ACGGTGTCGTGGAACTCAGGCGCTCTGACCAGCG | |
| | GCGTGCACACCTTCCCAGCTGTCCTACAGTCCTCA | |
| | GGACTCTACTCCCTCAGCAGCGTGGTGACCGTGC | |
| | CCTCCAGCAACTTCGGCACCCAGACCTACACCTG | |
| | CAACGTAGATCACAAGCCCAGCAACACCAAGGT | |
| | GGACAAGACAGTTGAGCGCAAATGTTGTGTCGAG | |
| | TGCCCACCGTGCCCAGCACCACCTGCCGCAGCCA | |
| | GCTCAGTCTTCCTCTTCCCCCCAAAACCCAAGGA | |
| | CACCCTCATGATCTCCCGGACCCCTGAGGTCACG | |
| | TGCGTGGTGGTGGACGTGAGCGCCGAAGACCCCG | |
| | AGGTCCAGTTCAACTGGTACGTGGACGGCGTGGA | |
| | GGTGCATAATGCCAAGACAAAGCCACGGGAGGA | |
| | GCAGTTCAACAGCACGTTCCGTGTGGTCAGCGTC | |
| | CTCACCGTTCTGCACCAGGACTGGCTGAACGGCA | |
| | AGGAGTACAAGTGCAAGGTCTCCAACAAAGGCCT | |
| | CCCATCCTCCATCGAGAAAACCATCTCCAAAACC | |
| | AAAGGGCAGCCCCGAGAACCACAGGTGTACACC | |
| | CTGCCCCCATCCCGGGAGGAGATGACCAAGAACC | |
| | AGGTCAGCCTGACCTGCCTGGTCAAAGGCTTCTA | |
| | CCCCAGCGACATCGCCGTGGAGTGGGAGAGCAAT | |
| | GGGCAGCCGGAGAACAACTACAAGACCACACCT | |
| | CCCATGCTGGACTCCGACGGCTCCTTCTTCCTCTA | |
| | CAGCAAGCTCACCGTGGACAAGAGCAGGTGGCA | |
| | GCAGGGGAACGTCTTCTCATGCTCCGTGATGCAT | |
| | GAGGCTCTGCACAACCACTACACGCAGAAGAGCC | |
| | TCTCCCTGTCTCCGGGTAAA | |
| DR4B117 LC | GAGATCGTGCTGACCCAGAGCCCCGGCACCCTGA | 94 |
| polynucleotide | GCCTGAGCCCCGGCGAGCGGGCCACCCTGAGCTG | |
| | CCGGGCCAGCCAGAGCGTGAGCAGCAGCTACCTG | |
| | GCCTGGTACCAGCAGAAGCCCGGCCAGGCCCCCC | |
| | GGCTGCTGATCTACGGCGCCAGCAGCCGGGCCAC | |
| | CGGCATCCCCGACCGGTTCAGCGGCAGCGGCAGC | |
| | GGCACCGACTTCACCCTGACCATCAGCCGGCTGG | |
| | AGCCCGAGGACTTCGCCGTGTACTACTGCCAGCA | |
| | GTACGGCAGCAGCCCCCTGACCTTCGGCCAGGGC | |
| | ACCAAGGTGGAGATCAAGCGGACCGTGGCCGCC | |
| | CCCAGCGTGTTCATCTTCCCCCCCAGCGACGAGC | |
| | AGCTGAAGAGCGGAACCGCAAGCGTGGTGTGCCT | |
| | GCTGAACAACTTCTACCCCCGGGAGGCCAAGGTG | |
| | CAGTGGAAGGTGGACAACGCCCTGCAGAGCGGC | |
| | AACAGCCAGGAGAGCGTGACCGAGCAGGACAGC | |
| | AAGGACAGCACCTACAGCCTGAGCAGCACCCTGA | |
| | CCCTGAGCAAGGCCGACTACGAGAAGCACAAGG | |
| | TGTACGCTTGCGAGGTGACCCACCAGGGCCTGAG | |
| | CAGCCCCGTGACCAAGAGCTTCAACCGGGGCGAG | |
| | TGC | |
| DR4B30, | GAGATCGTGCTGACCCAGAGCCCCGCCACCCTGA | 95 |
| DR4B127 and | GCCTGAGCCCCGGCGAGCGGGCCACCCTGAGCTG | |
| DR4B98 LC | CCGGGCCAGCCAGAGCGTGAGCAGCTACCTGGCC | |
| polynucleotide | TGGTACCAGCAGAAGCCCGGCCAGGCCCCCCGGC | |
| | TGCTGATCTACGACGCCAGCAACCGGGCCACCGG | |
| | CATCCCCGCCCGGTTCAGCGGCAGCGGCAGCGGC | |
| | ACCGACTTCACCCTGACCATCAGCAGCCTGGAGC | |
| | CCGAGGACTTCGCCGTGTACTACTGCCAGCAGCG | |
| | GAGCAACTGGCCCCTGACCTTCGGCCAGGGCACC | |
| | AAGGTGGAGATCAAGCGGACCGTGGCCGCCCCC | |
| | AGCGTGTTCATCTTCCCCCCCAGCGACGAGCAGC | |
| | TGAAGAGCGGAACCGCAAGCGTGGTGTGCCTGCT | |
| | GAACAACTTCTACCCCCGGGAGGCCAAGGTGCAG | |
| | TGGAAGGTGGACAACGCCCTGCAGAGCGGCAAC | |
| | AGCCAGGAGAGCGTGACCGAGCAGGACAGCAAG | |
| | GACAGCACCTACAGCCTGAGCAGCACCCTGACCC | |
| | TGAGCAAGGCCGACTACGAGAAGCACAAGGTGT | |
| | ACGCTTGCGAGGTGACCCACCAGGGCCTGAGCAG | |
| | CCCCGTGACCAAGAGCTTCAACCGGGGCGAGTGC | |
| DR4B78 VH | EVQLLESGGGLVQPGGSLRLSCAASGFTFSSYAMS | 137 |
| (DR4H62) | WVRQAPGKGLEWVSAISGSGGSTYYADSVKGRFTI | |
| amino acid | SRDNSKNTLYLQMNSLRAEDTAVYYCARDGGYYR | |
| | YVRTISGDYAFDYWGQGTLVTVSS | |
| DR4B70 VH | EVQLLESGGGLVQPGGSLRLSCAASGFTFSSYAMS | 138 |
| (DR4H29) | WVRQAPGKGLEWVSAISGSGGSTYYADSVKGRFTI | |
| amino acid | SRDNSKNTLYLQMNSLRAEDTAVYYCARDSSYYR | |
| | YIGRYLGDYAFDYWGQGTLVTVSS | |
| DR4B38 VH | EVQLLESGGGLVQPGGSLRLSCAASGFTFSSYAMS | 139 |
| (DR4H56) | WVRQAPGKGLEWVSAISGSGGSTYYADSVKGRFTI | |
| amino acid | SRDNSKNTLYLQMNSLRAEDTAVYYCARDSGYYR | |
| | LAAIGRSDYAFDYWGQGTLVTVSS | |
| DR4B33 VH | EVQLVQSGAEVKKPGESLKISCKGSGYSFDSAYINW | 140 |
| (DR4H58) | VRQMPGKGLEWMGIIRPGDSRTRYSPSFQGQVTISA | |
| amino acid | DKSISTAYLQWSSLKASDTAVYYCARDGYYFVGSII | |
| | YYGMDVWGQGTLVTVSS | |
| DR4B22 VH | EVQLLESGGGLVQPGGSLRLSCAASGFTFSSYAMN | 141 |
| (DR4H16) | WVRQAPGKGLEWVSAISGSGGYTNYADSVKGRFTI | |
| amino acid | SRDNSKNTLYLQMNSLRAEDTAVYYCARDGGYYR | |
| | YVYRYPGDYAFGYWGQGTLVTVSS | |
| DR4B33 VL | DIQMTQSPSSLSASVGDRVTITCRASQSISSYLNWYQ | 142 |
| (PH9L4) amino | QKPGKAPKLLIYAASSLQSGVPSRFSGSGSGTDFTLT | |
| acid | ISSLQPEDFATYYCQQSYSTPLTFGQGTKVEIK | |
| DR4B78 VH | GAAGTGCAGCTGCTGGAAAGCGGCGGCGGCCTG | 143 |
| (DR4H62) | GTGCAGCCGGGCGGCAGCCTGCGCCTGAGCTGCG | |
| DNA | CGGCGAGCGGCTTTACCTTTAGCAGCTATGCGAT | |
| | GAGCTGGGTGCGCCAGGCGCCGGGCAAAGGCCT | |
| | GGAATGGGTGAGCGCGATCAGCGGCTCCGGTGGC | |
| | TCCACATATTATGCGGATAGCGTGAAAGGCCGCT | |
| | TTACCATTTCACGAGATAACAGCAAAAACACCCT | |
| | GTATCTGCAGATGAACAGCCTGCGCGCGGAAGAT | |
| | ACCGCGGTGTATTATTGCGCGCGCGATGGCGGTT | |
| | ATTATCGTTATGTGCGTACAATCAGCGGCGATTA | |
| | TGCATTCGACTATTGGGGCCAGGGCACCCTGGTG | |
| | ACCGTCTCGAGT | |
| DR4B70 VH | GAAGTGCAGCTGCTGGAAAGCGGCGGCGGCCTG | 144 |
| (DR4H29) | GTGCAGCCGGGCGGCAGCCTGCGCCTGAGCTGCG | |
| DNA | CGGCGAGCGGCTTTACCTTTAGCAGCTATGCGAT | |
| | GAGCTGGGTGCGCCAGGCGCCGGGCAAAGGCCT | |
| | GGAATGGGTGAGCGCGATCAGCGGCTCCGGTGGC | |
| | TCCACATATTATGCGGATAGCGTGAAAGGCCGCT | |
| | TTACCATTTCACGAGATAACAGCAAAAACACCCT | |
| | GTATCTGCAGATGAACAGCCTGCGCGCGGAAGAT | |
| | ACCGCGGTGTATTATTGCGCGCGCGACTCCAGCT | |
| | ATTATCGTTACATTGGCCGTTATCTGGGCGACTAC | |
| | GCATTCGACTACTGGGGCCAGGGCACCCTGGTGA | |
| | CCGTCTCGAGT | |
| DR4B38 VH | GAAGTGCAGCTGCTGGAAAGCGGCGGCGGCCTG | 145 |
| (DR4H56) | GTGCAGCCGGGCGGCAGCCTGCGCCTGAGCTGCG | |
| DNA | CGGCGAGCGGCTTTACCTTTAGCAGCTATGCGAT | |
| | GAGCTGGGTGCGCCAGGCGCCGGGCAAAGGCCT | |
| | GGAATGGGTGAGCGCGATCAGCGGCTCCGGTGGC | |
| | TCCACATATTATGCGGATAGCGTGAAAGGCCGCT | |
| | TTACCATTTCACGAGATAACAGCAAAAACACCCT | |
| | GTATCTGCAGATGAACAGCCTGCGCGCGGAAGAT | |
| | ACCGCGGTGTATTATTGCGCGCGTGACTCCGGCT | |
| | ATTATCGTCTGGCAGCAATCGGCCGTTCTGATTAC | |
| | GCATTTGATTACTGGGGCCAGGGCACCCTGGTGA | |
| | CCGTCTCGAGT | |
| DR4B33 VH | GAAGTGCAGCTGGTGCAGAGCGGCGCGGAAGTG | 146 |
| (DR4H58) | AAAAAACCGGGCGAAAGCCTGAAAATTAGCTGC | |
| DNA | AAAGGCAGCGGCTATAGCTTCGATAGCGCATACA | |
| | TTAATTGGGTGCGCCAGATGCCGGGCAAAGGCTT | |
| | GGAATGGATGGGTATTATTCGTCCTGGCGATTCC | |
| | CGCACGCGTTACAGCCCGAGCTTTCAGGGCCAGG | |
| | TGACCATTAGCGCGGATAAAAGCATTAGCACCGC | |
| | GTATCTGCAGTGGAGCAGCCTGAAAGCGAGCGAT | |
| | ACCGCGGTGTATTATTGCGCGCGTGACGGCTATT | |
| | ATTTTGTTGGCAGCATCATCTATTACGGTATGGAC | |
| | GTATGGGGCCAGGGCACCCTGGTGACCGTCTCGA | |
| | GT | |
| DR4B22 VH | GAAGTGCAGCTGCTGGAAAGCGGCGGCGGCCTG | 147 |
| (DR4H16) | GTGCAGCCGGGCGGCAGCCTGCGCCTGAGCTGCG | |
| DNA | CGGCGAGCGGCTTTACCTTTTCCTCCTATGCAATG | |
| | AATTGGGTGCGCCAGGCGCCGGGCAAAGGCCTG | |
| | GAATGGGTGAGCGCTATTAGCGGTTCCGGTGGGT | |
| | ATACAAATTATGCGGATAGCGTGAAAGGCCGCTT | |
| | TACCATTTCACGAGATAACAGCAAAAACACCCTG | |
| | TATCTGCAGATGAACAGCCTGCGCGCGGAAGATA | |
| | CCGCGGTGTATTATTGCGCGCGTGACGGTGGTTA | |
| | CTACCGGTATGTGTACCGTTATCCAGGCGACTAT | |
| | GCATTTGGCTATTGGGGCCAGGGCACCCTGGTGA | |
| | CCGTCTCGAGT | |
| DR4B33 VL | GACATCCAGATGACCCAGAGCCCCAGCAGCCTGA | 148 |
| (PH9L4) DNA | GCGCCAGCGTGGGCGACCGGGTGACCATCACCTG | |
| | CCGGGCCAGCCAGAGCATCAGCAGCTACCTGAAC | |
| | TGGTACCAGCAGAAGCCCGGCAAGGCCCCCAAG | |
| | CTGCTGATCTACGCCGCCAGCAGCCTGCAGAGCG | |
| | GCGTGCCCAGCCGGTTCAGCGGCAGCGGCAGCGG | |
| | CACCGACTTCACCCTGACCATCAGCAGCCTGCAG | |
| | CCCGAGGACTTCGCCACCTACTACTGCCAGCAGA | |
| | GCTACAGCACCCCCCTGACCTTCGGCCAGGGCAC | |
| | CAAGGTGGAGATCAAG | |
| DR4B78 HC | EVQLLESGGGLVQPGGSLRLSCAASGFTFSSYAMS | 149 |
| amino acid | WVRQAPGKGLEWVSAISGSGGSTYYADSVKGRFTI | |
| | SRDNSKNTLYLQMNSLRAEDTAVYYCARDGGYYR | |
| | YVRTISGDYAFDYWGQGTLVTVSSASTKGPSVFPL | |
| | APCSRSTSESTAALGCLVKDYFPEPVTVSWNSGALT | |
| | SGVHTFPAVLQSSGLYSLSSVVTVPSSNFGTQTYTC | |
| | NVDHKPSNTKVDKTVERKCCVECPPCPAPPAAASS | |
| | VFLFPPKPKDTLMISRTPEVTCVVVDVSAEDPEVQF | |
| | NWYVDGVEVHNAKTKPREEQFNSTFRVVSVLTVL | |
| | HQDWLNGKEYKCKVSNKGLPSSIEKTISKTKGQPRE | |
| | PQVYTLPPSREEMTKNQVSLTCLVKGFYPSDIAVEW | |
| | ESNGQPENNYKTTPPMLDSDGSFFLYSKLTVDKSR | |
| | WQQGNVFSCSVMHEALHNHYTQKSLSLSPGK | |
| DR4B70 HC | EVQLLESGGGLVQPGGSLRLSCAASGFTFSSYAMS | 150 |
| amino acid | WVRQAPGKGLEWVSAISGSGGSTYYADSVKGRFTI | |
| | SRDNSKNTLYLQMNSLRAEDTAVYYCARDSSYYR | |
| | YIGRYLGDYAFDYWGQGTLVTVSSASTKGPSVFPL | |
| | APCSRSTSESTAALGCLVKDYFPEPVTVSWNSGALT | |
| | SGVHTFPAVLQSSGLYSLSSVVTVPSSNFGTQTYTC | |
| | NVDHKPSNTKVDKTVERKCCVECPPCPAPPAAASS | |
| | VFLFPPKPKDTLMISRTPEVTCVVVDVSAEDPEVQF | |
| | NWYVDGVEVHNAKTKPREEQFNSTFRVVSVLTVL | |
| | HQDWLNGKEYKCKVSNKGLPSSIEKTISKTKGQPRE | |
| | PQVYTLPPSREEMTKNQVSLTCLVKGFYPSDIAVEW | |
| | ESNGQPENNYKTTPPMLDSDGSFFLYSKLTVDKSR | |
| | WQQGNVFSCSVMHEALHNHYTQKSLSLSPGK | |
| DR4B38 HC | EVQLLESGGGLVQPGGSLRLSCAASGFTFSSYAMS | 151 |
| amino acid | WVRQAPGKGLEWVSAISGSGGSTYYADSVKGRFTI | |
| | SRDNSKNTLYLQMNSLRAEDTAVYYCARDSGYYR | |
| | LAAIGRSDYAFDYWGQGTLVTVSSASTKGPSVFPL | |
| | APCSRSTSESTAALGCLVKDYFPEPVTVSWNSGALT | |
| | SGVHTFPAVLQSSGLYSLSSVVTVPSSNFGTQTYTC | |
| | NVDHKPSNTKVDKTVERKCCVECPPCPAPPAAASS | |
| | VFLFPPKPKDTLMISRTPEVTCVVVDVSAEDPEVQF | |
| | NWYVDGVEVHNAKTKPREEQFNSTFRVVSVLTVL | |
| | HQDWLNGKEYKCKVSNKGLPSSIEKTISKTKGQPRE | |
| | PQVYTLPPSREEMTKNQVSLTCLVKGFYPSDIAVEW | |
| | ESNGQPENNYKTTPPMLDSDGSFFLYSKLTVDKSR | |
| | WQQGNVFSCSVMHEALHNHYTQKSLSLSPGK | |
| DR4B33 HC | EVQLVQSGAEVKKPGESLKISCKGSGYSFDSAYINW | 152 |
| amino acid | VRQMPGKGLEWMGIIRPGDSRTRYSPSFQGQVTISA | |
| | DKSISTAYLQWSSLKASDTAVYYCARDGYYFVGSII | |
| | YYGMDVWGQGTLVTVSSASTKGPSVFPLAPCSRST | |
| | SESTAALGCLVKDYFPEPVTVSWNSGALTSGVHTFP | |
| | AVLQSSGLYSLSSVVTVPSSNFGTQTYTCNVDHKPS | |
| | NTKVDKTVERKCCVECPPCPAPPAAASSVFLFPPKP | |
| | KDTLMISRTPEVTCVVVDVSAEDPEVQFNWYVDGV | |
| | EVHNAKTKPREEQFNSTFRVVSVLTVLHQDWLNGK | |
| | EYKCKVSNKGLPSSIEKTISKTKGQPREPQVYTLPPS | |
| | REEMTKNQVSLTCLVKGFYPSDIAVEWESNGQPEN | |
| | NYKTTPPMLDSDGSFFLYSKLTVDKSRWQQGNVFS | |
| | CSVMHEALHNHYTQKSLSLSPGK | |
| DR4B22 HC | EVQLLESGGGLVQPGGSLRLSCAASGFTFSSYAMN | 153 |
| amino acid | WVRQAPGKGLEWVSAISGSGGYTNYADSVKGRFTI | |
| | SRDNSKNTLYLQMNSLRAEDTAVYYCARDGGYYR | |
| | YVYRYPGDYAFGYWGQGTLVTVSSASTKGPSVFPL | |
| | APCSRSTSESTAALGCLVKDYFPEPVTVSWNSGALT | |
| | SGVHTFPAVLQSSGLYSLSSVVTVPSSNFGTQTYTC | |
| | NVDHKPSNTKVDKTVERKCCVECPPCPAPPAAASS | |
| | VFLFPPKPKDTLMISRTPEVTCVVVDVSAEDPEVQF | |
| | NWYVDGVEVHNAKTKPREEQFNSTFRVVSVLTVL | |
| | HQDWLNGKEYKCKVSNKGLPSSIEKTISKTKGQPRE | |
| | PQVYTLPPSREEMTKNQVSLTCLVKGFYPSDIAVEW | |
| | ESNGQPENNYKTTPPMLDSDGSFFLYSKLTVDKSR | |
| | WQQGNVFSCSVMHEALHNHYTQKSLSLSPGK | |
| DR4B33 LC | DIQMTQSPSSLSASVGDRVTITCRASQSISSYLNWYQ | 154 |
| amino acid | QKPGKAPKLLIYAASSLQSGVPSRFSGSGSGTDFTLT | |
| | ISSLQPEDFATYYCQQSYSTPLTFGQGTKVEIKRTVA | |
| | APSVFIFPPSDEQLKSGTASVVCLLNNFYPREAKVQ | |
| | WKVDNALQSGNSQESVTEQDSKDSTYSLSSTLTLS | |
| | KADYEKHKVYACEVTHQGLSSPVTKSFNRGEC | |
| DR4B78 HC | GAAGTGCAGCTGCTGGAAAGCGGCGGCGGCCTG | 155 |
| DNA | GTGCAGCCGGGCGGCAGCCTGCGCCTGAGCTGCG | |
| | CGGCGAGCGGCTTTACCTTTAGCAGCTATGCGAT | |
| | GAGCTGGGTGCGCCAGGCGCCGGGCAAAGGCCT | |
| | GGAATGGGTGAGCGCGATCAGCGGCTCCGGTGGC | |
| | TCCACATATTATGCGGATAGCGTGAAAGGCCGCT | |
| | TTACCATTTCACGAGATAACAGCAAAAACACCCT | |
| | GTATCTGCAGATGAACAGCCTGCGCGCGGAAGAT | |
| | ACCGCGGTGTATTATTGCGCGCGCGATGGCGGTT | |
| | ATTATCGTTATGTGCGTACAATCAGCGGCGATTA | |
| | TGCATTCGACTATTGGGGCCAGGGCACCCTGGTG | |
| | ACCGTCTCGAGTGCCTCCACCAAGGGCCCATCGG | |
| | TCTTCCCCCTGGCGCCCTGCTCCAGGAGCACCTCC | |
| | GAGAGCACAGCCGCCCTGGGCTGCCTGGTCAAGG | |
| | ACTACTTCCCCGAACCGGTGACGGTGTCGTGGAA | |
| | CTCAGGCGCTCTGACCAGCGGCGTGCACACCTTC | |
| | CCAGCTGTCCTACAGTCCTCAGGACTCTACTCCCT | |
| | CAGCAGCGTGGTGACCGTGCCCTCCAGCAACTTC | |
| | GGCACCCAGACCTACACCTGCAACGTAGATCACA | |
| | AGCCCAGCAACACCAAGGTGGACAAGACAGTTG | |
| | AGCGCAAATGTTGTGTCGAGTGCCCACCGTGCCC | |
| | AGCACCACCTGCCGCAGCCAGCTCAGTCTTCCTC | |
| | TTCCCCCCAAAACCCAAGGACACCCTCATGATCT | |
| | CCCGGACCCCTGAGGTCACGTGCGTGGTGGTGGA | |
| | CGTGAGCGCCGAAGACCCCGAGGTCCAGTTCAAC | |
| | TGGTACGTGGACGGCGTGGAGGTGCATAATGCCA | |
| | AGACAAAGCCACGGGAGGAGCAGTTCAACAGCA | |
| | CGTTCCGTGTGGTCAGCGTCCTCACCGTTCTGCAC | |
| | CAGGACTGGCTGAACGGCAAGGAGTACAAGTGC | |
| | AAGGTCTCCAACAAAGGCCTCCCATCCTCCATCG | |
| | AGAAAACCATCTCCAAAACCAAAGGGCAGCCCC | |
| | GAGAACCACAGGTGTACACCCTGCCCCCATCCCG | |
| | GGAGGAGATGACCAAGAACCAGGTCAGCCTGAC | |
| | CTGCCTGGTCAAAGGCTTCTACCCCAGCGACATC | |
| | GCCGTGGAGTGGGAGAGCAATGGGCAGCCGGAG | |
| | AACAACTACAAGACCACACCTCCCATGCTGGACT | |
| | CCGACGGCTCCTTCTTCCTCTACAGCAAGCTCACC | |
| | GTGGACAAGAGCAGGTGGCAGCAGGGGAACGTC | |
| | TTCTCATGCTCCGTGATGCATGAGGCTCTGCACA | |
| | ACCACTACACGCAGAAGAGCCTCTCCCTGTCTCC | |
| | GGGTAAA | |
| DR4B70 HC | GAAGTGCAGCTGCTGGAAAGCGGCGGCGGCCTG | 156 |
| DNA | GTGCAGCCGGGCGGCAGCCTGCGCCTGAGCTGCG | |
| | CGGCGAGCGGCTTTACCTTTAGCAGCTATGCGAT | |
| | GAGCTGGGTGCGCCAGGCGCCGGGCAAAGGCCT | |
| | GGAATGGGTGAGCGCGATCAGCGGCTCCGGTGGC | |
| | TCCACATATTATGCGGATAGCGTGAAAGGCCGCT | |
| | TTACCATTTCACGAGATAACAGCAAAAACACCCT | |
| | GTATCTGCAGATGAACAGCCTGCGCGCGGAAGAT | |
| | ACCGCGGTGTATTATTGCGCGCGCGACTCCAGCT | |
| | ATTATCGTTACATTGGCCGTTATCTGGGCGACTAC | |
| | GCATTCGACTACTGGGGCCAGGGCACCCTGGTGA | |
| | CCGTCTCGAGTGCCTCCACCAAGGGCCCATCGGT | |
| | CTTCCCCCTGGCGCCCTGCTCCAGGAGCACCTCC | |
| | GAGAGCACAGCCGCCCTGGGCTGCCTGGTCAAGG | |
| | ACTACTTCCCCGAACCGGTGACGGTGTCGTGGAA | |
| | CTCAGGCGCTCTGACCAGCGGCGTGCACACCTTC | |
| | CCAGCTGTCCTACAGTCCTCAGGACTCTACTCCCT | |
| | CAGCAGCGTGGTGACCGTGCCCTCCAGCAACTTC | |
| | GGCACCCAGACCTACACCTGCAACGTAGATCACA | |
| | AGCCCAGCAACACCAAGGTGGACAAGACAGTTG | |
| | AGCGCAAATGTTGTGTCGAGTGCCCACCGTGCCC | |
| | AGCACCACCTGCCGCAGCCAGCTCAGTCTTCCTC | |
| | TTCCCCCCAAAACCCAAGGACACCCTCATGATCT | |
| | CCCGGACCCCTGAGGTCACGTGCGTGGTGGTGGA | |
| | CGTGAGCGCCGAAGACCCCGAGGTCCAGTTCAAC | |
| | TGGTACGTGGACGGCGTGGAGGTGCATAATGCCA | |
| | AGACAAAGCCACGGGAGGAGCAGTTCAACAGCA | |
| | CGTTCCGTGTGGTCAGCGTCCTCACCGTTCTGCAC | |
| | CAGGACTGGCTGAACGGCAAGGAGTACAAGTGC | |
| | AAGGTCTCCAACAAAGGCCTCCCATCCTCCATCG | |
| | AGAAAACCATCTCCAAAACCAAAGGGCAGCCCC | |
| | GAGAACCACAGGTGTACACCCTGCCCCCATCCCG | |
| | GGAGGAGATGACCAAGAACCAGGTCAGCCTGAC | |
| | CTGCCTGGTCAAAGGCTTCTACCCCAGCGACATC | |
| | GCCGTGGAGTGGGAGAGCAATGGGCAGCCGGAG | |
| | AACAACTACAAGACCACACCTCCCATGCTGGACT | |
| | CCGACGGCTCCTTCTTCCTCTACAGCAAGCTCACC | |
| | GTGGACAAGAGCAGGTGGCAGCAGGGGAACGTC | |
| | TTCTCATGCTCCGTGATGCATGAGGCTCTGCACA | |
| | ACCACTACACGCAGAAGAGCCTCTCCCTGTCTCC | |
| | GGGTAAA | |
| DR4B38 HC | GAAGTGCAGCTGCTGGAAAGCGGCGGCGGCCTG | 157 |
| DNA | GTGCAGCCGGGCGGCAGCCTGCGCCTGAGCTGCG | |
| | CGGCGAGCGGCTTTACCTTTAGCAGCTATGCGAT | |
| | GAGCTGGGTGCGCCAGGCGCCGGGCAAAGGCCT | |
| | GGAATGGGTGAGCGCGATCAGCGGCTCCGGTGGC | |
| | TCCACATATTATGCGGATAGCGTGAAAGGCCGCT | |
| | TTACCATTTCACGAGATAACAGCAAAAACACCCT | |
| | GTATCTGCAGATGAACAGCCTGCGCGCGGAAGAT | |
| | ACCGCGGTGTATTATTGCGCGCGTGACTCCGGCT | |
| | ATTATCGTCTGGCAGCAATCGGCCGTTCTGATTAC | |
| | GCATTTGATTACTGGGGCCAGGGCACCCTGGTGA | |
| | CCGTCTCGAGTGCCTCCACCAAGGGCCCATCGGT | |
| | CTTCCCCCTGGCGCCCTGCTCCAGGAGCACCTCC | |
| | GAGAGCACAGCCGCCCTGGGCTGCCTGGTCAAGG | |
| | ACTACTTCCCCGAACCGGTGACGGTGTCGTGGAA | |
| | CTCAGGCGCTCTGACCAGCGGCGTGCACACCTTC | |
| | CCAGCTGTCCTACAGTCCTCAGGACTCTACTCCCT | |
| | CAGCAGCGTGGTGACCGTGCCCTCCAGCAACTTC | |
| | GGCACCCAGACCTACACCTGCAACGTAGATCACA | |
| | AGCCCAGCAACACCAAGGTGGACAAGACAGTTG | |
| | AGCGCAAATGTTGTGTCGAGTGCCCACCGTGCCC | |
| | AGCACCACCTGCCGCAGCCAGCTCAGTCTTCCTC | |
| | TTCCCCCCAAAACCCAAGGACACCCTCATGATCT | |
| | CCCGGACCCCTGAGGTCACGTGCGTGGTGGTGGA | |
| | CGTGAGCGCCGAAGACCCCGAGGTCCAGTTCAAC | |
| | TGGTACGTGGACGGCGTGGAGGTGCATAATGCCA | |
| | AGACAAAGCCACGGGAGGAGCAGTTCAACAGCA | |
| | CGTTCCGTGTGGTCAGCGTCCTCACCGTTCTGCAC | |
| | CAGGACTGGCTGAACGGCAAGGAGTACAAGTGC | |
| | AAGGTCTCCAACAAAGGCCTCCCATCCTCCATCG | |
| | AGAAAACCATCTCCAAAACCAAAGGGCAGCCCC | |
| | GAGAACCACAGGTGTACACCCTGCCCCCATCCCG | |
| | GGAGGAGATGACCAAGAACCAGGTCAGCCTGAC | |
| | CTGCCTGGTCAAAGGCTTCTACCCCAGCGACATC | |
| | GCCGTGGAGTGGGAGAGCAATGGGCAGCCGGAG | |
| | AAGAACTACAAGACCACACCTCCCATGCTGGACT | |
| | CCGACGGCTCCTTCTTCCTCTACAGCAAGCTCACC | |
| | GTGGACAAGAGCAGGTGGCAGCAGGGGAACGTC | |
| | TTCTCATGCTCCGTGATGCATGAGGCTCTGCACA | |
| | ACCACTACACGCAGAAGAGCCTCTCCCTGTCTCC | |
| | GGGTAAA | |
| DR4B33 HC | GAAGTGCAGCTGGTGCAGAGCGGCGCGGAAGTG | 158 |
| DNA | AAAAAACCGGGCGAAAGCCTGAAAATTAGCTGC | |
| | AAAGGCAGCGGCTATAGCTTCGATAGCGCATACA | |
| | TTAATTGGGTGCGCCAGATGCCGGGCAAAGGCTT | |
| | GGAATGGATGGGTATTATTCGTCCTGGCGATTCC | |
| | CGCACGCGTTACAGCCCGAGCTTTCAGGGCCAGG | |
| | TGACCATTAGCGCGGATAAAAGCATTAGCACCGC | |
| | GTATCTGCAGTGGAGCAGCCTGAAAGCGAGCGAT | |
| | ACCGCGGTGTATTATTGCGCGCGTGACGGCTATT | |
| | ATTTTGTTGGCAGCATCATCTATTACGGTATGGAC | |
| | GTATGGGGCCAGGGCACCCTGGTGACCGTCTCGA | |
| | GTGCCTCCACCAAGGGCCCATCGGTCTTCCCCCT | |
| | GGCGCCCTGCTCCAGGAGCACCTCCGAGAGCACA | |
| | GCCGCCCTGGGCTGCCTGGTCAAGGACTACTTCC | |
| | CCGAACCGGTGACGGTGTCGTGGAACTCAGGCGC | |
| | TCTGACCAGCGGCGTGCACACCTTCCCAGCTGTC | |
| | CTACAGTCCTCAGGACTCTACTCCCTCAGCAGCG | |
| | TGGTGACCGTGCCCTCCAGCAACTTCGGCACCCA | |
| | GACCTACACCTGCAACGTAGATCACAAGCCCAGC | |
| | AACACCAAGGTGGACAAGACAGTTGAGCGCAAA | |
| | TGTTGTGTCGAGTGCCCACCGTGCCCAGCACCAC | |
| | CTGCCGCAGCCAGCTCAGTCTTCCTCTTCCCCCCA | |
| | AAACCCAAGGACACCCTCATGATCTCCCGGACCC | |
| | CTGAGGTCACGTGCGTGGTGGTGGACGTGAGCGC | |
| | CGAAGACCCCGAGGTCCAGTTCAACTGGTACGTG | |
| | GACGGCGTGGAGGTGCATAATGCCAAGACAAAG | |
| | CCACGGGAGGAGCAGTTCAACAGCACGTTCCGTG | |
| | TGGTCAGCGTCCTCACCGTTCTGCACCAGGACTG | |
| | GCTGAACGGCAAGGAGTACAAGTGCAAGGTCTCC | |
| | AACAAAGGCCTCCCATCCTCCATCGAGAAAACCA | |
| | TCTCCAAAACCAAAGGGCAGCCCCGAGAACCAC | |
| | AGGTGTACACCCTGCCCCCATCCCGGGAGGAGAT | |
| | GACCAAGAACCAGGTCAGCCTGACCTGCCTGGTC | |
| | AAAGGCTTCTACCCCAGCGACATCGCCGTGGAGT | |
| | GGGAGAGCAATGGGCAGCCGGAGAACAACTACA | |
| | AGACCACACCTCCCATGCTGGACTCCGACGGCTC | |
| | CTTCTTCCTCTACAGCAAGCTCACCGTGGACAAG | |
| | AGCAGGTGGCAGCAGGGGAACGTCTTCTCATGCT | |
| | CCGTGATGCATGAGGCTCTGCACAACCACTACAC | |
| | GCAGAAGAGCCTCTCCCTGTCTCCGGGTAAA | |
| DR4B22 HC | GAAGTGCAGCTGCTGGAAAGCGGCGGCGGCCTG | 159 |
| DNA | GTGCAGCCGGGCGGCAGCCTGCGCCTGAGCTGCG | |
| | CGGCGAGCGGCTTTACCTTTTCCTCCTATGCAATG | |
| | AATTGGGTGCGCCAGGCGCCGGGCAAAGGCCTG | |
| | GAATGGGTGAGCGCTATTAGCGGTTCCGGTGGGT | |
| | ATACAAATTATGCGGATAGCGTGAAAGGCCGCTT | |
| | TACCATTTCACGAGATAACAGCAAAAACACCCTG | |
| | TATCTGCAGATGAACAGCCTGCGCGCGGAAGATA | |
| | CCGCGGTGTATTATTGCGCGCGTGACGGTGGTTA | |
| | CTACCGGTATGTGTACCGTTATCCAGGCGACTAT | |
| | GCATTTGGCTATTGGGGCCAGGGCACCCTGGTGA | |
| | CCGTCTCGAGTGCCTCCACCAAGGGCCCATCGGT | |
| | CTTCCCCCTGGCGCCCTGCTCCAGGAGCACCTCC | |
| | GAGAGCACAGCCGCCCTGGGCTGCCTGGTCAAGG | |
| | ACTACTTCCCCGAACCGGTGACGGTGTCGTGGAA | |
| | CTCAGGCGCTCTGACCAGCGGCGTGCACACCTTC | |
| | CCAGCTGTCCTACAGTCCTCAGGACTCTACTCCCT | |
| | CAGCAGCGTGGTGACCGTGCCCTCCAGCAACTTC | |
| | GGCACCCAGACCTACACCTGCAACGTAGATCACA | |
| | AGCCCAGCAACACCAAGGTGGACAAGACAGTTG | |
| | AGCGCAAATGTTGTGTCGAGTGCCCACCGTGCCC | |
| | AGCACCACCTGCCGCAGCCAGCTCAGTCTTCCTC | |
| | TTCCCCCCAAAACCCAAGGACACCCTCATGATCT | |
| | CCCGGACCCCTGAGGTCACGTGCGTGGTGGTGGA | |
| | CGTGAGCGCCGAAGACCCCGAGGTCCAGTTCAAC | |
| | TGGTACGTGGACGGCGTGGAGGTGCATAATGCCA | |
| | AGACAAAGCCACGGGAGGAGCAGTTCAACAGCA | |
| | CGTTCCGTGTGGTCAGCGTCCTCACCGTTCTGCAC | |
| | CAGGACTGGCTGAACGGCAAGGAGTACAAGTGC | |
| | AAGGTCTCCAACAAAGGCCTCCCATCCTCCATCG | |
| | AGAAAACCATCTCCAAAACCAAAGGGCAGCCCC | |
| | GAGAACCACAGGTGTACACCCTGCCCCCATCCCG | |
| | GGAGGAGATGACCAAGAACCAGGTCAGCCTGAC | |
| | CTGCCTGGTCAAAGGCTTCTACCCCAGCGACATC | |
| | GCCGTGGAGTGGGAGAGCAATGGGCAGCCGGAG | |
| | AACAACTACAAGACCACACCTCCCATGCTGGACT | |
| | CCGACGGCTCCTTCTTCCTCTACAGCAAGCTCACC | |
| | GTGGACAAGAGCAGGTGGCAGCAGGGGAACGTC | |
| | TTCTCATGCTCCGTGATGCATGAGGCTCTGCACA | |
| | ACCACTACACGCAGAAGAGCCTCTCCCTGTCTCC | |
| | GGGTAAA | |
| DR4B33 LC | GACATCCAGATGACCCAGAGCCCCAGCAGCCTGA | 160 |
| DNA | GCGCCAGCGTGGGCGACCGGGTGACCATCACCTG | |
| | CCGGGCCAGCCAGAGCATCAGCAGCTACCTGAAC | |
| | TGGTACCAGCAGAAGCCCGGCAAGGCCCCCAAG | |
| | CTGCTGATCTACGCCGCCAGCAGCCTGCAGAGCG | |
| | GCGTGCCCAGCCGGTTCAGCGGCAGCGGCAGCGG | |
| | CACCGACTTCACCCTGACCATCAGCAGCCTGCAG | |
| | CCCGAGGACTTCGCCACCTACTACTGCCAGCAGA | |
| | GCTACAGCACCCCCCTGACCTTCGGCCAGGGCAC | |
| | CAAGGTGGAGATCAAGCGGACCGTGGCCGCCCCC | |
| | AGCGTGTTCATCTTCCCCCCCAGCGACGAGCAGC | |
| | TGAAGAGCGGAACCGCAAGCGTGGTGTGCCTGCT | |
| | GAACAACTTCTACCCCCGGGAGGCCAAGGTGCAG | |
| | TGGAAGGTGGACAACGCCCTGCAGAGCGGCAAC | |
| | AGCCAGGAGAGCGTGACCGAGCAGGACAGCAAG | |
| | GACAGCACCTACAGCCTGAGCAGCACCCTGACCC | |
| | TGAGCAAGGCCGACTACGAGAAGCACAAGGTGT | |
| | ACGCTTGCGAGGTGACCCACCAGGGCCTGAGCAG | |
| | CCCCGTGACCAAGAGCTTCAACCGGGGCGAGTGC | |

TABLE 17

| | | VH | | VL |
|---|---|---|---|---|
| | VH | framework | | framework |
| mAb | framework | SEQ ID NO: | VL framework | SEQ ID NO: |
| DR4B117 | IGHV1_1-69 | 62 | IGKV3-20 (A27) | 64 |
| DR4B30 | IGHV5_5-51 | 63 | IGKV3-11 (L6) | 65 |
| DR4B127 | IGHV1_1-69 | 62 | IGKV3-11 (L6) | 65 |
| DR4B98 | IGHV1_1-69 | 62 | IGKV3-11 (L6) | 65 |
| DR4B78 | IGHV3_3-23 | 161 | IGKV3-11 (L6) | 65 |
| DR4B70 | IGHV3_3-23 | 161 | IGKV3-11 (L6) | 65 |
| DR4B38 | IGHV3_3-23 | 161 | IGKV3-11 (L6) | 65 |
| DR4B33 | IGHV5_5-51 | 63 | IGKV1-39 (O12) | 162 |
| DR4B22 | IGHV3_3-23 | 161 | IGKV3-11 (L6) | 65 |

IGHVI-69

SEQ ID NO: 62

QVQLVQSGAEVKKPGSSVKVSCKASGGTFS SYAIS WVRQAPGQGLEWM
GGIIPIFGTANYAQKFQGRVTITADESTSTAYMELSSLRSEDTAVYYCAR
IGHV5-51

SEQ ID NO: 63

EVQLVQSGAEVKKPGESLKISCKGSGYSFTSYWIGWVRQMPGKGLEWMGI
IYPGDSDTRYSPSFQGQVTISADKSISTAYLQWSSLKASDTAMYYCAR
IGKV3-20 (A27)

SEQ ID NO: 64

EIVLTQSPGTLSLSPGERATLSCRASQSVSSSYLAWYQQKPGQAPRLLIY
GASSRATGIPDRESGSGSGTDFTLTISRLEPEDFAVYYCQQYGSSP
IGKV3-11 (L6)

SEQ ID NO: 65

EIVLTQSPATLSLSPGERATLSCRASQSVSSYLAWYQQKPGQAPRLLIYD
ASNRATGIPARFSGSGSGTDFTLTISSLEPEDFAVYYCQQRSNWP
IGHV3_3-23

SEQ ID NO: 161

EVQLLESGGGLVQPGGSLRLSCAASGFTFSSYAMSWVRQAPGKGLEWVSA
ISGSGGSTYYADSVKGRFTISRDNSKNTLYLQMNSLRAEDTAVYYCAKWG
QGTLVTVSS
IGKV1-39

SEQ ID NO: 162

DIQMTQSPSSLSASVGDRVTITCRASQSISSYLNWYQQKPGKAPKLLIYA
ASSLQSGVPSRFSGSGSGTDFTLTISSLQPEDFATYYCQQSYSTP

### Example 6. Affinity of Anti-HLA-DR Antibodies to HLA-DR

The interactions of anti-HLA-DR antibodies with HLA-DR1 and HLA-DR4 complexes containing either collagen II or hemagglutinin peptides were studied by Surface Plasmon Resonance (SPR) using a ProteOn XPR36 system at 25° C. A biosensor surface was prepared by direct coupling of anti-HLA-DR antibodies to the surface of a GLC sensor chip using the manufacturer instructions for amine-coupling chemistry. At 15 μg/ml of mAbs diluted in coupling buffer, 10 mM sodium acetate pH5.0, approximately 130-500 RU (response units) of mAbs were immobilized. The kinetic experiments were performed at 25° C. in running buffer (DPBS+0.01% P20+100 μg/ml BSA). To perform kinetic experiments, analytes (HLA-DR1 and HLA-DR4 complexes) were injected in horizontal orientation over coupled anti-HLA-DR mAbs sensor at concentration ranging from 3.7 nM to 300 nM (in a 3-fold serial dilution). The association phase was monitored for 6 minutes at 50 μl/min, then followed by 30 minutes of buffer flow (dissociation phase). The chip surface was regenerated with two 18 second pulses of 100 mM Phosphoric acid (H₃PO₄) at 100 μl/min. The collected data were processed using ProteOn Manager software. First, the data was corrected for background using inter-spots. Then, double reference subtraction of the data was performed by using the buffer injection for analyte injections. The kinetic analysis of the data was performed using a Langmuir 1:1 binding model. The result for each mAb was reported in the format of Ka (On-rate), Kd (Off-rate) and K_{D} (equilibrium dissociation constant).

Table 18 shows the affinity parameters of DR4B117 and DR4B127 for binding to HLA-DR4/hemagglutinin peptide complex (DR4G89). Table 19 shows the affinity parameters of DR4B117 and DR4B127 for binding to HLA-DR4/collagen peptide complex (DR4G90). Table 20 shows the affinity parameters of DR4B117 and DR4B127 for binding to HLA-DR1/hemagglutinin peptide complex (DR4G93). Table 21 shows the affinity parameters of DR4B117 and DR4B127 for binding to HLA-DR1/collagen peptide complex (DR4G99).

TABLE 18

| | Antigen: DR4G89 (HLA-DR4 with HA_304-318) | | |
|---|---|---|---|
| Sample | ka (1/Ms) | kd (1/s) | K_{D} (M) |
| DR4B117 | 1.39E+04 | 7.77E−05 | 5.58E−09 |
| DR4B127 | 1.68E+04 | 11.55E−04 | 9.19E−09 |

TABLE 19

| | Antigen: DR4G90 (HLA-DR4 with CII_1236-1249) | | |
|---|---|---|---|
| Sample | ka (1/Ms) | kd (1/s) | K_{D} (M) |
| DR4B117 | 1.60E+04 | 3.28E−05 | 2.05E−09 |
| DR4B127 | 1.41E+04 | 2.26E−04 | 1.60E−08 |

TABLE 20

| | Antigen: DR4G93 (HLA-DR1 with HA_304-318) | | |
|---|---|---|---|
| Sample | ka (1/Ms) | kd (1/s) | K_{D} (M) |
| DR4B117 | 2.05E+04 | 1.36E−04 | 6.62E−09 |
| DR4B127 | 1.93E+04 | 9.20E−04 | 4.77E−08 |

TABLE 21

| | Antigen: DR4G99 (HLA-DR1 with CII_1236-1249) | | |
|---|---|---|---|
| Sample | ka (1/Ms) | kd (1/s) | K_{D} (M) |
| DR4B117 | 1.98E+04 | 11.79E−05 | 9.04E−10 |
| DR4B127 | 2.38E+03 | 3.47E−04 | 1.46E−07* |

*binding signal was weak; K_{D} is an approximate

### Example 7. Effect of Isotype on Antibody Function

Variable regions of the IgG2sigma/κ antibodies DR4B117. DR4B30, DR4B127. DR4B98 and DR4B6 were cloned as wild-type IgG1 to assess possible differences in functionality.

The IgG1/κ antibodies were named DR4B391 (DR4B117 VH/VL on wild-type IgG1). DR4B396 (DR4B30 VH/VL on wild-type IgG1), DR4B392 (DR4B127 VH/VL on wild-type IgG1), DR4B401 (DR4B98 VH/VL on IgG1) and DR4B397 (DR4B6 VH/VL on IgG1). The heavy chain sequences of the antibodies are shown in Table 22. The light chain sequences were identical to the parental antibodies.

TABLE 22

| | | SEQ ID |
|---|---|---|
| Sequence | Sequence | NO: |
| DR4B391 HC | QVQLVQSGAEVKKPGSSVKVSCKASGGTFSSYSIHWV | 96 |
| protein | RQAPGQGLEWMGYITPEYGTANYAQKFQGRVTITADE | |
| | STSTAYMELSSLRSEDTAVYYCARGRYYIGNRRGSYY | |
| | GFDYWGQGTLVTVSSASTKGPSVFPLAPSSKSTSGGTA | |
| | ALGCLVKDYFPEPVTVSWNSGALTSGVHTFPAVLQSS | |
| | GLYSLSSVVTVPSSSLGTQTYICNVNHKPSNTKVDKKV | |
| | EPKSCDKTHTCPPCPAPEAAGASSVFLFPPKPKDTLMIS | |
| | RTPEVTCVVVDVSAEDPEVKFNWYVDGVEVHNAKTK | |
| | PREEQYNSTYRVVSVLTVLHQDWLNGKEYKCKVSNK | |
| | ALPSSIEKTISKAKGQPREPQVYTLPPSREEMTKNQVSL | |
| | TCLVKGFYPSDIAVEWESNGQPENNYKTTPPVLDSDG | |
| | SFFLYSKLTVDKSRWQQGNVFSCSVMHEALHNHYTQ | |
| | KSLSLSPGK | |
| DR4B396 HC | EVQLVQSGAEVKKPGESLKISCKGSGYSFTSDWIGWV | 97 |
| protein | RQMPGKGLEWMGIIRPGDSDTYYSPSFQGQVTISADKS | |
| | ISTAYLQWSSLKASDTAVYYCARESYYYVGVRYRPSY | |
| | YFDYWGQGTLVTVSSASTKGPSVFPLAPSSKSTSGGTA | |
| | ALGCLVKDYFPEPVTVSWNSGALTSGVHTFPAVLQSS | |
| | GLYSLSSVVTVPSSSLGTQTYICNVNHKPSNTKVDKKV | |
| | EPKSCDKTHTCPPCPAPEAAGASSVFLFPPKPKDTLMIS | |
| | RTPEWCVVVDVSAEDPEVKFNWYYVDGVEVHNAKTK | |
| | PREEQYNSTYRWSVLTVLHQDWLNGKEYKCKVSNK | |
| | ALPSSIEKTISKAKGQPREPQVYTLPPSREEMTKNQVSL | |
| | TCLVKGFYPSDIAVEWESNGQPENNYKTTPPVLDSDG | |
| | SFFLYSKLTVDKSRWQQGNVFSCSVMHEALHNHYTQ | |
| | KSLSLSPGK | |
| DR4B392 HC | QVQLVQSGAEVKKPGSSVKVSCKASGGTFKSYYIHW | 98 |
| protein | VRQAPGQGLEWMGGTRPISGNAEYAQKFQGRVTITAD | |
| | ESTSTAYMELSSLRSEDTAVYYCARDASYYRNYGFDY | |
| | WGQGTLVTVSSASTKGPSVFPLAPSSKSTSGGTAALGC | |
| | LVKDYFPEPVTVSWNSGALTSGVHTFPAVLQSSGLYS | |
| | LSSVVTVPSSSLGTQTYICNVNHKPSNTKVDKKVEPKS | |
| | CDKTHTCPPCPAPEAAGASSVFLFPPKPKDTLMISRTPE | |
| | VTCVVVDVSAEDPEVKFNWYVDGVEVHNAKTKPREE | |
| | QYNSTYRVVSVLTVXHQDWLNGKEYKCKVSNKALPS | |
| | SIEKTISKAKGQPREPQVYTLPPSREEMTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK | |
| DR4B401 HC | QVQLVQSGAEVKKPGSSVKVSCKASGGTFKSYYIHW | 99 |
| protein | VRQAPGQGLEWMGGIAPIYGTAYYAQKFQGRVTITAD | |
| | ESTSTAYMELSSLRSEDTAVYYCARDASWARAYGFD | |
| | YWGQGTLVTVSSASTKGPSVFPLAPSSKSTSGGTAALG | |
| | CLVKDYFPEPVTVSWNSGALTSGVHTFPAVLQSSGLY | |
| | SLSSWTVPSSSLGTQTYICNVNHKPSNTKVDKKVEPK | |
| | SCDKTHTCPPCPAPEAAGASSVFLFPPKPKDTLMISRTP | |
| | EVTCVVVDVSAEDPEVKFNWYVDGVEVHNAKTKPRE | |
| | EQYNSTYRVVSVLTVLHQDWLNGKEYKCKVSNKALP | |
| | SSIEKTISKAKGQPREPQVYTLPPSREEMTKNQVSLTCL | |
| | VKGFYPSDIAVEWESNGQPEWTYKTTPPVLDSDGSFFL | |
| | YSKETVTJKSRWQQGNWSCSVMHEALHNHYTQKSLS | |
| | LSPGK | |
| DR4B391 HC | CAGGTGCAGCTGGTGCAGAGCGGCGCGGAAGTGAA | 100 |
| polynucleotide | AAAACCGGGCAGCAGCGTGAAAGTGAGCTGCAAAG | |
| | CGAGCGGCGGCACCTTTAGCAGCTATTCCATTCACT | |
| | GGGTGCGCCAGGCGCCGGGCCAGGGCCTGGAATGG | |
| | ATGGGCTACATTATTCCGGAGTACGGGACTGCCAAT | |
| | TACGCGCAGAAATTTCAGGGCCGCGTGACCATTACC | |
| | GCTGATGAAAGCACCAGCACCGCGTATATGGAACT | |
| | GAGCAGCCTGCGCAGCGAAGATACCGCGGTGTATT | |
| | ATTGCGCGCGCGGCCGATACTATATCGGCAACCGTC | |
| | GTGGCAGTTATTACGGTTTTGACTATTGGGGCCAGG | |
| | GCACCCTGGTGACCGTCTCGAGTGCCTCCACCAAGG | |
| | GCCCATCGGTCTTCCCCCTGGCACCCTCCTCCAAGA | |
| | GCACCTCTGGGGGCACAGCGGCCCTGGGCTGCCTGG | |
| | TCAAGGACTACTTCCCCGAACCGGTGACGGTGTCGT | |
| | GGAACTCAGGCGCCCTGACCAGCGGCGTGCACACCT | |
| | TCCCGGCTGTCCTACAGTCCTCAGGACTCTACTCCCT | |
| | CAGCAGCGTGGTGACCGTGCCCTCCAGCAGCTTGGG | |
| | CACCCAGACCTACATCTGCAACGTGAATCACAAGCC | |
| | CAGCAACACCAAGGTGGACAAGAAAGTTGAGCCCA | |
| | AATCTTGTGACAAAACTCACACATGCCCACCGTGCC | |
| | CAGCACCTGAAGCAGCAGGGGCATCTTCAGTCTTCC | |
| | TCTTCCCCCCAAAACCCAAGGACACCCTCATGATCT | |
| | CCCGGACCCCTGAGGTCACATGCGTGGTGGTGGACG | |
| | TGAGCGCCGAAGACCCTGAGGTCAAGTTCAACTGGT | |
| | ACGTGGACGGCGTGGAGGTGCATAATGCCAAGACA | |
| | AAGCCGCGGGAGGAGCAGTACAACAGCACGTACCG | |
| | TGTGGTCAGCGTCCTCACCGTCCTGCACCAGGACTG | |
| | GCTGAATGGCAAGGAGTACAAGTGCAAGGTCTCCA | |
| | ACAAAGCCCTCCCATCCTCCATCGAGAAAACCATCT | |
| | CCAAAGCCAAAGGGCAGCCCCGAGAACCACAGGTG | |
| | TACACCCTGCCCCCATCCCGGGAGGAGATGACCAAG | |
| | AACCAGGTCAGCCTGACCTGCCTGGTCAAAGGCTTC | |
| | TATCCCAGCGACATCGCCGTGGAGTGGGAGAGCAA | |
| | TGGGCAGCCGGAGAACAACTACAAGACCACGCCTC | |
| | CCGTGCTGGACTCCGACGGCTCCTTCTTCCTCTACAG | |
| | CAAGCTCACCGTGGACAAGAGCAGGTGGCAGCAGG | |
| | GGAACGTCTTCTCATGCTCCGTGATGCATGAGGCTC | |
| | TGCACAACCACTACACGCAGAAGAGCCTCTCCCTGT | |
| | CTCCGGGTAAA | |
| DR4B396 HC | GAAGTGCAGCTGGTGCAGAGCGGCGCGGAAGTGAA | 101 |
| polynucleotide | AAAACCGGGCGAAAGCCTGAAAATTAGCTGCAAAG | |
| | GCAGCGGCTATAGCTTTACCAGCGACTGGATTGGTT | |
| | GGGTGCGCCAGATGCCGGGCAAAGGCTTGGAATGG | |
| | ATGGGTATCATTCGCCCGGGCGATAGCGATACGTAT | |
| | TACAGCCCGAGCTTTCAGGGCCAGGTGACCATTAGC | |
| | GCGGATAAAAGCATTAGCACCGCGTATCTGCAGTGG | |
| | AGCAGCCTGAAAGCGAGCGATACCGCGGTGTATTAT | |
| | TGCGCGCGTGAATCCTATTATTACGTTGGCGTGCGT | |
| | TACCGTCCAAGCTATTATTTCGATTACTGGGGCCAG | |
| | GGCACCCTGGTGACCGTCTCGAGTGCCTCCACCAAG | |
| | GGCCCATCGGTCTTCCCCCTGGCACCCTCCTCCAAG | |
| | AGCACCTCTGGGGGCACAGCGGCCCTGGGCTGCCTG | |
| | GTCAAGGACTACTTCCCCGAACCGGTGACGGTGTCG | |
| | TGGAACTCAGGCGCCCTGACCAGCGGCGTGCACACC | |
| | TTCCCGGCTGTCCTACAGTCCTCAGGACTCTACTCCC | |
| | TCAGCAGCGTGGTGACCGTGCCCTCCAGCAGCTTGG | |
| | GCACCCAGACCTACATCTGCAACGTGAATCACAAGC | |
| | CCAGCAACACCAAGGTGGACAAGAAAGTTGAGCCC | |
| | AAATCTTGTGACAAAACTCACACATGCCCACCGTGC | |
| | CCAGCACCTGAAGCAGCAGGGGCATCTTCAGTCTTC | |
| | CTCTTCCCCCCAAAACCCAAGGACACCCTCATGATC | |
| | TCCCGGACCCCTGAGGTCACATGCGTGGTGGTGGAC | |
| | GTGAGCGCCGAAGACCCTGAGGTCAAGTTCAACTG | |
| | GTACGTGGACGGCGTGGAGGTGCATAATGCCAAGA | |
| | CAAAGCCGCGGGAGGAGCAGTACAACAGCACGTAC | |
| | CGTGTGGTCAGCGTCCTCACCGTCCTGCACCAGGAC | |
| | TGGCTGAATGGCAAGGAGTACAAGTGCAAGGTCTC | |
| | CAACAAAGCCCTCCCATCCTCCATCGAGAAAACCAT | |
| | CTCCAAAGCCAAAGGGCAGCCCCGAGAACCACAGG | |
| | TGTACACCCTGCCCCCATCCCGGGAGGAGATGACCA | |
| | AGAACCAGGTCAGCCTGACCTGCCTGGTCAAAGGCT | |
| | TCTATCCCAGCGACATCGCCGTGGAGTGGGAGAGCA | |
| | ATGGGCAGCCGGAGAACAACTACAAGACCACGCCT | |
| | CCCGTGCTGGACTCCGACGGCTCCTTCTTCCTCTACA | |
| | GCAAGCTCACCGTGGACAAGAGCAGGTGGCAGCAG | |
| | GGGAACGTCTTCTCATGCTCCGTGATGCATGAGGCT | |
| | CTGCACAACCACTACACGCAGAAGAGCCTCTCCCTG | |
| | TCTCCGGGTAAA | |
| DR4B392 HC | CAGGTGCAGCTGGTGCAGAGCGGCGCGGAAGTGAA | 102 |
| polynucleotide | AAAACCGGGCAGCAGCGTGAAAGTGAGCTGCAAAG | |
| | CGAGCGGCGGCACCTTTAAATCCTACTACATTCACT | |
| | GGGTGCGCCAGGCGCCGGGCCAGGGCCTGGAATGG | |
| | ATGGGTGGTATTCGTCCGATCAGCGGGAATGCTGAG | |
| | TACGCGCAGAAATTTCAGGGCCGCGTGACCATTACC | |
| | GCTGATGAAAGCACCAGCACCGCGTATATGGAACT | |
| | GAGCAGCCTGCGCAGCGAAGATACCGCGGTGTATT | |
| | ATTGCGCGCGCGATGCAAGCTATTATCGTAATTACG | |
| | GTTTTGACTACTGGGGCCAGGGCACCCTGGTGACCG | |
| | TCTCGAGTGCCTCCACCAAGGGCCCATCGGTCTTCC | |
| | CCCTGGCACCCTCCTCCAAGAGCACCTCTGGGGGCA | |
| | CAGCGGCCCTGGGCTGCCTGGTCAAGGACTACTTCC | |
| | CCGAACCGGTGACGGTGTCGTGGAACTCAGGCGCCC | |
| | TGACCAGCGGCGTGCACACCTTCCCGGCTGTCCTAC | |
| | AGTCCTCAGGACTCTACTCCCTCAGCAGCGTGGTGA | |
| | CCGTGCCCTCCAGCAGCTTGGGCACCCAGACCTACA | |
| | TCTGCAACGTGAATCACAAGCCCAGCAACACCAAG | |
| | GTGGACAAGAAAGTTGAGCCCAAATCTTGTGACAA | |
| | AACTCACACATGCCCACCGTGCCCAGCACCTGAAGC | |
| | AGCAGGGGCATCTTCAGTCTTCCTCTTCCCCCCAAA | |
| | ACCCAAGGACACCCTCATGATCTCCCGGACCCCTGA | |
| | GGTCACATGCGTGGTGGTGGACGTGAGCGCCGAAG | |
| | ACCCTGAGGTCAAGTTCAACTGGTACGTGGACGGCG | |
| | TGGAGGTGCATAATGCCAAGACAAAGCCGCGGGAG | |
| | GAGCAGTACAACAGCACGTACCGTGTGGTCAGCGTC | |
| | CTCACCGTCCTGCACCAGGACTGGCTGAATGGCAAG | |
| | GAGTACAAGTGCAAGGTCTCCAACAAAGCCCTCCCA | |
| | TCCTCCATCGAGAAAACCATCTCCAAAGCCAAAGGG | |
| | CAGCCCCGAGAACCACAGGTGTACACCCTGCCCCCA | |
| | TCCCGGGAGGAGATGACCAAGAACCAGGTCAGCCT | |
| | GACCTGCCTGGTCAAAGGCTTCTATCCCAGCGACAT | |
| | CGCCGTGGAGTGGGAGAGCAATGGGCAGCCGGAGA | |
| | ACAACTACAAGACCACGCCTCCCGTGCTGGACTCCG | |
| | ACGGCTCCTTCTTCCTCTACAGCAAGCTCACCGTGG | |
| | ACAAGAGCAGGTGGCAGGAGGGGAACGTCTTCTCA | |
| | TGCTCCGTGATGCATGAGGCTCTGCACAACCACTAC | |
| | ACGCAGAAGAGCCTCTCCCTGTCTCCGGGTAAA | |
| DR4B401 | CAGGTGCAGCTGGTGCAGAGCGGCGCGGAAGTGAA | 103 |
| polynucleotide | AAAACCGGGCAGCAGCGTGAAAGTGAGCTGCAAAG | |
| | CGAGCGGCGGCACCTTTAAGTCCTATTATATTCATT | |
| | GGGTGCGCCAGGCGCCGGGCCAGGGCCTGGAATGG | |
| | ATGGGCGGTATTGCACCAATTTACGGCACCGCTTAC | |
| | TACGCGCAGAAATTTCAGGGCCGCGTGACCATTACC | |
| | GCTGATGAAAGCACCAGCACCGCGTATATGGAACT | |
| | GAGCAGCCTGCGCAGCGAAGATACCGCGGTGTATT | |
| | ATTGCGCGCGTGATGCAAGTTGGGCACGTGCATACG | |
| | GTTTTGATTATTGGGGCCAGGGCACCCTGGTGACCG | |
| | TCTCGAGTGCCTCCACCAAGGGCCCATCGGTCTTCC | |
| | CCCTGGCACCCTCCTCCAAGAGCACCTCTGGGGGCA | |
| | CAGCGGCCCTGGGCTGCCTGGTCAAGGACTACTTCC | |
| | CCGAACCGGTGACGGTGTCGTGGAACTCAGGCGCCC | |
| | TGACCAGCGGCGTGCACACCTTCCCGGCTGTCCTAC | |
| | AGTCCTCAGGACTCTACTCCCTCAGCAGCGTGGTGA | |
| | CCGTGCCCTCCAGCAGCTTGGGCACCCAGACCTACA | |
| | TCTGCAACGTGAATCACAAGCCCAGCAACACCAAG | |
| | GTGGACAAGAAAGTTGAGCCCAAATCTTGTGACAA | |
| | AACTCACACATGCCCACCGTGCCCAGCACCTGAAGC | |
| | AGCAGGGGCATCTTCAGTCTTCCTCTTCCCCCCAAA | |
| | ACCCAAGGACACCCTCATGATCTCCCGGACCCCTGA | |
| | GGTCACATGCGTGGTGGTGGACGTGAGCGCCGAAG | |
| | ACCCTGAGGTCAAGTTCAACTGGTACGTGGACGGCG | |
| | TGGAGGTGCATAATGCCAAGACAAAGCCGCGGGAG | |
| | GAGCAGTACAACAGCACGTACCGTGTGGTCAGCGTC | |
| | CTCACCGTCCTGCACCAGGACTGGCTGAATGGCAAG | |
| | GAGTACAAGTGCAAGGTCTCCAACAAAGCCCTCCCA | |
| | TCCTCCATCGAGAAAACCATCTCCAAAGCCAAAGGG | |
| | CAGCCCCGAGAACCACAGGTGTACACCCTGCCCCCA | |
| | TCCCGGGAGGAGATGACCAAGAACCAGGTCAGCCT | |
| | GACCTGCCTGGTCAAAGGCTTCTATCCCAGCGACAT | |
| | CGCCGTGGAGTGGGAGAGCAATGGGCAGCCGGAGA | |
| | ACAACTACAAGACCACGCCTCCCGTGCTGGACTCCG | |
| | ACGGCTCCTTCTTCCTCTACAGCAAGCTCACCGTGG | |
| | ACAAGAGCAGGTGGCAGCAGGGGAACGTCTTCTCA | |
| | TGCTCCGTGATGCATGAGGCTCTGCACAACCACTAC | |
| | ACGCAGAAGAGCCTCTCCCTGTCTCCGGGTAAA | |

In addition to the assays described in Example 4, the antibodies were tested for their ability to inhibit antigen-specific T cells using a T cell hybridoma that specifically recognizes collagen II peptide (amino acids 259-273; GIAGFKGEQGPKGEP, SEQ ID NO: 122) presented by HLA-DR4 (HLA-DR4/Collagen II peptide-restricted T cell hybridoma assay (“HLA-DR4/ColII-Tcell” assay)).

The results of the HLA-DR4 DC MLR assay across four individual PBMC donors are summarized in Table 23. The results of evaluating binding of the mAbs to dendritic cells from HLA-DR4 transgenic mice (“HLA-DR4 DC Binding” assay) or to human PBMCs, effect of the mAbs on viability of human B cells and inhibition of HLA-DR4/CII peptide-restricted T cell hybridomas are shown in Table 24.

Isotype switch from an effector-silent IgG2sigma to the wild-type IgG1 had an effect on antibody functionality. For example isotype switch in DR4B117 to a wild-type IgG1 resulted in improved inhibitory activity of the mAb whereas isotype switch of DR4B127 to a wild-type IgG1 resulted in reduced inhibitory activity of the mAb. There was no effect on the isotype in binding to PBCMs or viability of B cells

DR4B30 and DR4B127 inhibited IL-2 production by HLA-DR4/CII—peptide-restricted T cell hybridomas at 10 μg/ml and 1 μg/ml antibody concentrations. DR4B117 was not inhibitory in this assay, the antibody enhanced IL-2 production at all doses tested. The control antibody DR4B6 was inhibitory at 10 μg/ml and 1 μg/ml antibody concentrations but enhanced IL-2 production at doses below 0.1 μg/ml (Table 23).

### (HLA-DR4/Collagen 11 Peptide-Restricted T Cell Hybridoma Assay (“HLA-DR4/ColII-Tcell” Assay)).

The Boleth B cell line (homozygous for HLA-DRB1*04:01) was obtained from the International Histocompatibility Working Group. The Boleth cells were washed and resuspended in complete media (DMEM/Glutamax+1% Penicillin/Streptomycin+10% fetal calf serum+50 μM 2-mercaptoethanol) at 1.25×10⁵ cells/ml; 50 μl cells were added to each well of a 96-well round bottom plate. The anti-HLA-DR antibodies were added at 4× the final concentration, 50 μl per well, beginning at a concentration of 10 μg/ml. The plates were incubated for 1 hr at 37° C.

The T cell hybridoma line DR4.CII.36.8 was obtained from Dr. Edward Rosloniec at the University of Tennessee Health Science Center. These cells were washed with complete media, resuspended in complete media at a concentration of 2×10⁶ cells/nil, and added (50 pd/well) to the plate containing the Boleth cells. The CII peptide (GIAGFKGEQGPKGEP. SEQ ID NO: 121) was diluted in complete media to 8 μM (4× the final concentration of 2 μM) and added to the plate at 50 μl/well. The total volume in all wells was brought up to 200 μl using complete media. The plates were incubated for 18-21 hr at 37° C. The supernatants were harvested for analysis using the mIL-2 AlphaLISA kit (Perkin Elmer) according to manufacturer's instructions.

TABLE 23

| | HLA-DR4 DC MLR | | | | | | | |
|---|---|---|---|---|---|---|---|---|
| | Average percent (%) inhibition at indicated mAb concentration | | | | | | | |
| | 10 μg/ml mAb | | | | 1 μg/ml mAb | | | |
| mAb | Donor 5 | Donor 6 | Donor 7 | Donor 8 | Donor 5 | Donor 6 | Donor 7 | Donor 8 |
| DR4B117 | 52.4% | 58.2% | 56.3% | 61.8% | 27.5% | 35.4% | 39.1% | 52.3% |
| DR4B391 | 83.3% | 77.5% | 77.2% | 94.7% | 50.9% | 72.8% | 60.6% | 81.4% |
| DR4B127 | 86.9% | 87.6% | 91.8% | 101.0% | 69.9% | 75.2% | 80.6% | 86.0% |
| DR4B392 | 63.4% | 59.2% | 64.6% | 71.9% | 46.1% | 24.2% | 22.3% | 65.2% |
| DR4B6 | 93.4% | 102.3% | 94.8% | 101.0% | 93.3% | 97.3% | 91.1% | 96.3% |

TABLE 24

| | | HLA-DR4/CII peptide- | | | |
|---|---|---|---|---|---|
| | | restricted T cell hybridoma | | | |
| | | assay | | | |
| | | Average percent (%) | | | |
| | HLA- | inhibition at indicated | | | |
| | DR4 | mAb concentration | | Human | Human |
| | DC | 10 μg/ml | 1 μg/ml | PBMC | B cell |
| mAb | Binding | mAb | mAb | Binding | viability |
| DR4B117 | High | 0% | 0% | High | No effect |
| DR4B391 | High | 70.59% | 61.00% | High | No effect |
| DR4F330 | High | 60.69% | 41.24% | High | No effect |
| DR4B396 | | 67.88% | 61.96% | | |
| DR4B127 | High | 78.08% | 77.23% | High | No effect |
| DR4B392 | High | 51.53% | 49.56% | High | No effect |
| DR4B98 | High | | | High | No effect |
| DR4B401 | | 64.54% | 47.50% | | |
| DR4B6 | High | 98.47% | 90.97% | High | Induced |
| | | | | | cell |
| | | | | | death |
| DR4B397 | | 99.59% | 96.86% | | |

High: Mean fluorescent intensity higher than what was detected using DR4B6

### Example 8. The Anti-HLA-DR Antibodies Retain Binding to a Spectrum of HLA-DR Antigens in Complex with Various Peptides

Select antibodies were further characterized for their binding to soluble HLA-DR, HLA-DQ and HLA-DP antigens in complex with various peptides which were covalently attached to the N-terminus of the beta chains. Binding was assessed using protocol described in Example 3. The heterodimeric antigens were expressed as described in Example 1. Table 25 shows the format of the additional expressed HLA fusion proteins and Table 26 shows the amino acid sequences of the α and β chains. All additional HLA proteins had a common α chain of SEQ ID NO: 20. None of the antibodies bound the tested DP and the DQ antigens which were in complex with CLIP, LCAP or PLP peptides. DR4B117 and DR4B127 demonstrated binding to all tested HLA antigens, including DRB1*04:02. DRB1*15:01 and DRB1*03:01 which do not contain the shared epitope. The control antibodies DR4B4 and DR4B5 demonstrated overall reduced binding to DRB1*04:01. DRB1*01:01 and DRB1*10:01 when compared to DR4B117 and DR4B127 and no binding to DRB1*15:01 and DRB1*04:02. DR4B6 showed binding to all tested HLA-peptide complexes. Table 27, Table 28. Table 29 and Table 30 show the results of the binding of the antibodies to the HLA molecules.

Vimentin L70A mutant 66 to 78 peptide (VitL70A)

SEQ ID NO: 71

SAVRARSSVPGVR
AggrecanPeptideN1 (Aggrecan)

SEQ ID NO: 72

EVVLLVATEGRVRVNSAYQDK
CLIP

SEQ ID NO: 104

KMRMATPLLMQALPM;
HLA-DRB1*03:01_P01912

SEQ ID NO: 105

GDTRPRFLEYSTSECHFFNGTERVRYLDRYFHNQEENVRFDSDVGEFRAV
TELGRPDAEYWNSQKDLLEQKRGRVDNYCRHNYGVVESFTVQRRVHPKVT
VYPSKTQPLQHHNLLVCSVSGFYPGSIEVRWFRNGQEEKTGVVSTGLIHN
GDWTFQTLVMLETVPRSGEVYTCQVEHPSVTSPLTVEWRARSESAQSK
HLA-DRB1*04:02_HLA00687 ECD

SEQ ID NO: 106

GDTRPRFLEQVKHECHFFNGTERVRFLDRYFYHQEEYVRFDSDVGEYRAV
TELGRPDAEYWNSQKDILEDERAAVDTYCRHNYGVVESFTVQRRVYPEVT
VYPAKTQPLQHHNLLVCSVNGFYPGSIEVRWFRNGQEEKTGVVSTGLIQN
GDWTFQTLVMLETVPRSGEVYTCQVEHPSLTSPLTVEWRARSESAQSK
HLA-DRB1*10:01_Q30167_ECD 30-227

SEQ ID NO: 107

GDTRPRFLEEVKFECHFFNGTERVRLLERRVHNQEEYARYDSDVGEYRAV
TELGRPDAEYWNSQKDLLERRRAAVDTYCRHNYGVGESFTVQRRVQPKVT
VYPSKTQPLQHHNLLVCSVNGFYPGSIEVRWFRNGQEEKTGVVSTGLIQN
GDWTFQTLVMLETVPQSGEVYTCQVEHPSVMSPLTVEWRARSESAQSK
HLA-DRB1*15:01_P01911_ECD 30-277

SEQ ID NO: 108

GDTRPRFLWQPKRECHFFNGTERVRFLDRYFYNQEESVRFDSDVGEFRAV
TELGRPDAEYWNSQKDILEQARAAVDTYCRHNYGVVESFTVQRRVQPKVT
VYPSKTQPLQHHNLLVCSVSGFYPGSIEVRWFLNGQEEKAGMVSTGLIQN
GDWTFQTLVMLETVPRSGEVYTCQVEHPSVTSPLTVEWRARSESAQSK

TABLE 25

| Antigen | |
|---|---|
| name | Protein Description |
| DR4G143 | Human HLA-DRA1*01:02/DRB1*04:01 ECD_VimentineL70A in the format of |
| | Alpha chain: ECD_G4S + TEV + G4S + MMB + 6xHisTag; |
| | Beta chain: |
| | VimentinL70A + 3XGS + HRV3C + ECD + G4S + TEV + G4S + MMB + 6xHisTag; |
| DR4G142 | Human HLA-DRA1*01:02/DRB1*03:01 ECD_VimentineL70A in the |
| | format of Alpha chain: ECD_G4S + TEV + G4S + MMB + 6xHisTag; |
| | Beta chain: |
| | VimentinL70A + 3XGS + HRV3C + ECD + G4S + TEV + G4S + MMB + StrepII |
| DR4G119 | Human HLA-DRA1*01:02/DRB1*10:01 ECD with hemagglutinin peptide |
| | HA_304-318 (HA) in the format of |
| | Alpha chain: ECD_G4S + TEV + G4S + MMB + 6xHisTag; |
| | Beta chain: HA + 3XGS + HRV3C + ECD + G4S + TEV + G4S + MMB + StrepII |
| DR4G117 | Human HLA-DRA1*01:02/DRB1*03:01 ECD with Collagen II peptide |
| | CII_1236-1249 (CII_1236) in the format of |
| | Alpha chain: ECD_G4S + TEV + G4S + MMB + 6xHisTag; |
| | Beta chain: |
| | CII_1236 + 3XGS + HRV3C + ECD + G4S + TEV + G4S + MMB + StrepII |
| DR4G110 | Human HLA-DRA1*01:02/DRB1*04:01 ECD with aggrecan peptide N1 |
| | (N1) in the format of |
| | Alpha chain: ECD_G4S + TEV + G4S + MMB + 6xHisTag; |
| | Beta chain: |
| | AggrecanPeptideN1 + 3XGS + HRV3C + ECD + G4S + TEV + G4S + MMB + StrepII |
| DR4G104 | Human HLA-DRA1*01:02/DRB1*04:02 ECD with hemagglutinin peptide |
| | HA_304-318 (HA) in the format of |
| | Alpha chain: ECD_G4S + TEV + G4S + MMB + 6xHisTag; |
| | Beta chain: HA + 3XGS + HRV3C + ECD + G4S + TEV + G4S + MMB + StrepII |
| DR4G103 | Human HLA-DRA1*01::02/DPRB1*15:01 ECD with hemagglutinin peptide |
| | HA_304-318 (HA) in the format of |
| | Alpha chain: ECD_G4S + TEV + G4S + MMB + 6xHisTag; |
| | Beta chain: HA + 3XGS + HRV3 + ECD + G4S + TEV + G4S + MMB + StrepII |
| DR4G101 | Human HLA-DRA1*01:02/DRB1*04:02 ECD with collagen II peptide |
| | CII_257-273 (CII_257) in the format of |
| | Alpha chain: ECD_G4S + TEV + G4S + MMB + 6xHisTag; |
| | Beta chain: CII_257 + 3XGS + HRV3C + ECD + G4S + TEV + G4S + MMB + StrepII |
| DR4G98 | Human HLA-DRA1*01:02/DRB1*04:02 ECD with collagen II peptide |
| | CII_1236-1249 (CII_1236) in the format of |
| | Alpha chain: ECD_G4S + TEV + G4S + MMB + 6xHisTag; |
| | Beta chain: |
| | CII_1236 + 3XGS + HRV3C + ECD + G4S + TEV + G4S + MMB + StrepII |
| DR4G97 | Human HLA-DRA1*01:02/DRB1*15:01 ECD with collagen II peptide |
| | CII_1236-1249 (CII_1236) in the format of |
| | Alpha chain: ECD_G4S + TEV + G4S + MMB + 6xHisTag; |
| | Beta chain: |
| | CII_1236 + 3XGS + HRV3C + ECD + G4S + TEV + G4S + MMB + StrepII |
| DR4G96 | Human HLA-DRA1*01:02/DRB1*01:01 ECD with CLIP peptide in the |
| | format of |
| | Alpha chain: ECD_G4S + TEV + G4S + MMB + 6xHisTag; |
| | Beta chain: CLIP + 3XGS + HRV3C + ECD + G4S + TEV + G4S + MMB + StrepII |
| DR4G86 | Human HLA-DRA1*01:02/DRB1*04:01 ECD with CLIP peptide in the format of |
| | Alpha chain: ECD_G4S + TEV + G4S + MMB + 6xHisTag; |
| | Beta chain: CLIP + 3XGS + HRV3C + ECD + G4S + TEV + G4S + MMB + StrepII |

TABLE 26

| Protein name | Beta chain amino acid sequence |
|---|---|
| DR4G143 | SAVRARSSVPGVRGSGSGSLEVLFQGPGDTRPRFLEQVKHE |
| (alpha chain: | CHFFNGTERVRFLDRYFYHQEEYVRFDSDVGEYRAVTELGR |
| SEQ ID NO: | PDAEYWNSQKDLLEQKRAAVDTYCRHNYGVGESFTVQRR |
| 20; beta | VYPEVTVYPAKTQPLQHHNLLVCSVNGFYPGSIEVRWFRNG |
| chain: SEQ ID NO: | QEEKTGVVSTGLTQNGDWTFQTLVMLETVPRSGEVYTCQV |
| 109) | EHPSLTSPLTVEWRARSESAQSKGGGGSEDLYFQSGGGGSC |
| | PPCPAPEAAGGPSVFLFPPKPKDTLMISRTPEVTCWVDVSQ |
| | EDPEVQFNWYVDGVEVHNAKTKPREEQFNSTYRVVSVLTV |
| | LHQDWLNGKEYKCKVSNKGLPSSIEKTISKAKGQPREPQVY |
| | TLPPSQEEMTKNQVSLTCLVKGFYPSDIAVEWESNGQPENN |
| | YKTTPPVLDSDGSFFLYSRLTVDKSRWQEGNVFSCSVMHEA |
| | LHNHYTQKSLSLSLGKWSHPQFEK |
| DR4G142 | SAVRARSSVPGVRGSGSGSLEVLFQGPGDTRPRFLEYSTSEC |
| (alplia chain: | HFFNGTERVRYLDRYFHNQEENWFDSDVGEFRAWELGRP |
| SEQ ID NO: | DAEYWNSQKDLLEQKRGRVDNYCRHNYGVVESFTVQRRV |
| 20; beta | HPKVTVYPSKTQPLQHHNLLVCSVSGFYPGSIEVRWFRNGQ |
| chain: SEQ | EEKTGVVSTGLIHNGDWTFQTLVMLETVPRSGEVYTCQVE |
| ID NO: 110) | HPSVTSPLTVEWRARSESAQSKGGGGSEDLYFQSGGGGSCP |
| | PCPAPEAAGGPSVFLFPPKPKDTLMISRTPEVTCVVVDVSQE |
| | DPEVQFNWYVDGVEVHNAKTKPREEQFNSTYRVVSVLTVL |
| | HQDWLNGKEYKCKVSNKGLPSSIEKTISKAKGQPREPQVYT |
| | LPPSQEEMTKNQVSLTCLVKGFYPSDTAVEWESNGQPENNY |
| | KTTPPVLDSDGSFFLYSRLTVDKSRWQEGNVFSCSVMHEAL |
| | HNHYTQKSLSLSLGKWSHPQFEK |
| DR4G119 | ACPKYVKQNTLKLATGSGSGSLEVLFQGPGDTRPRFLEEVK |
| (alpha chain: | FECHFFNGTERVRLLERRVHNQEEYARYDSDVGEYRAVTEL |
| SEQ ID NO: | GRPDAEYWNSQKDLLERRRAAVDTYCRHNYGVGESFTVQ |
| 20; beta | RRVQPKVTVYPSKTQPLQHHNLLVCSVNGFYPGSIEVRWFR |
| chain: SEQ | NGQEEKTGVVSTGLIQNGDWTFQTXVMLETVPQSGEVYTC |
| ID NO: 111) | QVEHPSVMSPLTVEWRARSESAQSKGGGGSEDLYFQSGGG |
| | GSCPPCPAPEAAGGPSVFLFPPKPKDTLMISRTPEVTCVVVD |
| | VSQEDPEVQFNWYVDGVEVHNAKTKPREEQFNSTYRVVSV |
| | LTVLHQDWLNGKEYKCKVSNKGLPSSIEKTISKAKGQPREP |
| | QVYTLPPSQEEMTKNQVSLTCLVKGFYPSDIAVEWESNGQP |
| | ENNYKTTPPVLDSDGSFFLYSRLTVDKSRWQEGNVFSCSVM |
| | HEALHNHYTQKSLSLSLGKWSHPQFEK |
| DR4G117 | LQYMRADQAAGGLRGSGSGSLEVLFQGPGDTRPRFLEYSTS |
| (alpha cliain: | ECHFFNGTERWYLDRWHNQEENVRFDSDVGEFRAVTELG |
| SEQ ID NO: | RPDAEYWNSQKDLLEQKRGRVDNYCRHNYGVVESFTVQR |
| 20; beta | RVHPKWVYPSKTQPLQHHNLLVCSVSGFYPGSIEVRWFRN |
| chain: SEQ | GQEEKTGWSTGLIHNGDWTFQTLVMLETVPRSGEVYTCQ |
| ID NO: 112) | VEHPSVTSPLTVEWRARSESAQSKGGGGSEDLYFQSGGGGS |
| | CPPCPAPEAAGGPSWLFPPKPKDTLMISRTPEVTCVVVDVS |
| | QEDPEVQFNWYVDGVEVHNAKTKPREEQFNSTYRVVSVLT |
| | VLHQDWLNGKEYKCKVSNKGLPSSIEKTISKAKGQPREPQV |
| | YTLPPSQEEMTKNQVSLTCLVKGFYPSDIAVEWESNGQPEN |
| | NYKTTPPVLDSDGSFFLYSRLTVDKSRWQEGNVFSCSVMHE |
| | ALHNHYTQKSLSLSLGKWSHPQFEK |
| DR4G110 | EWLLVATEGRVRVNSAYQDKGSGSGSLEVLFQGPGDTRPR |
| (alpha cliain: | FLEQVKHECHFFNGTERVRFLDRYFYHQEEYVRFDSDVGEY |
| SEQ ID NO: | RAVTELGRPDAEYWNSQKDLLEQKRAAVDTYCRHNYGVG |
| 20; beta | ESFTVQRRVYPEVTVYPAKTQPLQHHNLLVCSVNGFYPGSI |
| chain: SEQ | EVRWFRNGQEEKTGVVSTGLIQNGDWTFQTLVMLETVPRS |
| ID NO: 113) | GEVYTCQVEHPSLTSPLTVEWRARSESAQSKGGGGSEDLYF |
| | QSGGGGSCPPCPAPEAAGGPSVFLFPPKPKDTXMISRTPEVT |
| | CVVVDVSQEDPEVQFNWYVDGVEVHNAKTKPREEQFNSTY |
| | RVVSVLTVLHQDWLNGKEYKCKVSNKGLPSSIEKTISKAKG |
| | QPREPQVYTLPPSQEEMTKNQVSLTCLVKGFYPSDIAVEWE |
| | SNGQPENNYKTTPPVLDSDGSFFLYSRLTVDKSRWQEGNVF |
| | SCSVMHEALHNHYTQKSLSLSLGKWSHPQFEK |
| DR4G104 | ACPKYVKQNTLKLATGSGSGSLEVLFQGPGDTRPRFLEQVK |
| (alpha chain: | HECHFFNGTERVRFLDRYFYHQEEYVRFDSDVGEYRAVTEL |
| SEQ ID NO: | GRPDAEYWNSQKDILEDERAAVDTYCRHNYGWESFTVQR |
| 20; beta | RVYPEVTVYPAKTQPLQHHNLLVCSVNGFYPGSIEVRWFRN |
| chain: SEQ | GQEEKTGVVSTGLIQNGDWTFQTLVMLETVPRSGEVYTCQ |
| ID NO: 114) | VEHPSLTSPLTVEWRARSESAQSKGGGGSEDLYFQSGGGGS |
| | CPPCPAPEAAGGPSVFLFPPKPKDTLMISRTPEVTCVVVDVS |
| | QEDPEVQFNWYVDGVEVHNAKTKPREEQFNSTYRVVSVLT |
| | VLHQDWLNGKEYKCKVSNKGLPSSIEKTISKAKGQPREPQV |
| | YTLPPSQEEMTKNQVSLTCLVKGFYPSDIAVEWESNGQPEN |
| | NYKTTPPVLDSDGSFFLYSRLTVDKSRWQEGNVFSCSVMHE |
| | ALHNHYTQKSLSLSLGKWSHPQFEK |
| DR4G103 | ACPKYVKQNTLKLATGSGSGSLEVLFQGPGDTRPRFLWQPK |
| (alpha chain: | RECHFFNGTERVRFLDRYFYNQEESVRFDSDVGEFRAVTEL |
| SEQ ID NO: | GRPDAEYWNSQKDILEQARAAVDTYCRHNYGVVESFTVQR |
| 20; beta | RVQPKVTVYPSKTQPLQHHNLLVCSVSGFYPGSIEVRWFLN |
| chain: SEQ | GQEEKAGMVSTGLIQNGDWTFQTLVMLETVPRSGEVYTCQ |
| ID NO: 115) | VEHPSVTSPLTVEWRARSESAQSKGGGGSEDLYFQSGGGGS |
| | CPPCPAPEAAGGPSVFLFPPKPKDTLMISRTPEVTCVVVDVS |
| | QEDPEVQFNWVDGVEVHNAKTKPREEQFNSTYRVVSVLT |
| | VLHQDWLNGKEYKCKVSNKGLPSSIEKTISKAKGQPREPQV |
| | YTLPPSQEEMTKNQVSLTCLVKGFYPSDIAVEWESNGQPEN |
| | NYKTTPPVLDSDGSFFLYSRLTVDKSRWQEGNVFSCSVMHE |
| | ALHNHYTQKSLSLSLGKWSHPQFEK |
| DR4G101 | EPGIAGFKGEQGPKGEPGSGSGSLEVLFQGPGDTRPRFLEQV |
| (alpha chain: | KHECHFFNGTERVRFLDRYFYHQEEYVRFDSDVGEYRAVT |
| SEQ ID NO: | ELGRPDAEYWNSQKDILEDERAAVDTYCRHNYGVVESFTV |
| 20; beta | QRRVYPEVTVYPAKTQPLQHHNLLVCSVNGFYPGSIEVRWF |
| chain: SEQ | RNGQEEKTGWSTGLIQNGDWTFQTLVMLETVPRSGEVYT |
| ID NO: 116) | CQVEHPSLTSPLTVEWRARSESAQSKGGGGSEDLYFQSGGG |
| | GSCPPCPAPEAAGGPSVFLFPPKPKDTLMISRTPEVTCWVD |
| | VSQEDPEVQFNWYVDGVEVHNAKTKPREEQFNSTYRVVSV |
| | LTVLHQDWLNGKEYKCKVSNKGLPSSIEKTISKAKGQPREP |
| | QVYTLPPSQEEMTKNQVSLTCLVKGFYPSDIAVEWESNGQP |
| | ENNYKTTPPVLDSDGSFFLYSRLTVDKSRWQEGNVFSCSVM |
| | HEALHNHYTQKSLSLSLGKWSHPQFEK |
| DR4G98 | LQYMRADQAAGGLRGSGSGSLEVLFQGPGDTRPRFLEQVK |
| (alpha chain: | HECHFFNGTERVRFLDRYFYHQEEYVRFDSDVGEYRAVTEL |
| SEQ ID NO: | GRPDAEYWNSQKDILEDERAAVDTYCRHNYGVVESFTVQR |
| 20; beta | RVYPEVTVYPAKTQPLQHHNLLVCSVNGFYPGSIEVRWFRN |
| chain: SEQ | GQEEKTGVVSTGLIQNGDWTFQTLVMLETVPRSGEVYTCQ |
| ID NO: 117) | VEHPSLTSPLTVEWRARSESAQSKGGGGSEDLYFQSGGGGS |
| | CPPCPAPEAAGGPSVFLFPPKPKDTLMISRTPEVTCVVVDVS |
| | QEDPEVQFNWYVDGVEVHNAKTKPREEQFNSTYRVVSVLT |
| | VLHQDWLNGKEYKCKVSNKGLPSSIEKTISKAKGQPREPQV |
| | YTLPPSQEEMTKNQVSLTCLVKGFYPSDIAVEWESNGQPEN |
| | NYKTTPPVLDSDGSFFLYSRLTVDKSRWQEGNVFSCSVMHE |
| | ALHNHYTQKSLSLSLGKWSHPQFEK |
| DR4G97 | LQYMRADQAAGGLRGSGSGSLEVLFQGPGDTRPRFLWQPK |
| (alpha chain: | RECHFFNGTERVRFLDRYFYNQEESVRFDSDVGEFRAVTEL |
| SEQ ID NO: | GRPDAEYWNSQKDILEQARAAVDTYCRHNYGVVESFTVQR |
| 20; beta | RVQPKVTVYPSKTQPLQHHNLLVCSVSGFYPGSIEVRWFLN |
| chain: SEQ | GQEEKAGMVSTGLIQNGDWTFQTLVMLETVPRSGEVYTCQ |
| ID NO: 118) | VEHPSVTSPLTVEWRARSESAQSKGGGGSEDLYFQSGGGGS |
| | CPPCPAPEAAGGPSVFLFPPKPKDTLMISRTPEVTCVVVDVS |
| | QEDPEVQFNWYVDGVEVHNAKTKPREEQFNSTYRVVSVLT |
| | VLHQDWLNGKEYKCKVSNKGLPSSIEKTISKAKGQPREPQV |
| | YTLPPSQEEMTKNQVSLTCLVKGFYPSDIAVEWESNGQPEN |
| | NYKTTPPVLDSDGSFFLYSRLTVDKSRWQEGNVFSCSVMHE |
| | ALHNHYTQKSLSLSLGKWSHPQFEK |
| DR4G96 | KMRMATPLLMQALPMGSGSGSLEVLFQGPGDTRPRFLWQL |
| (alpha chain: | KFECHFFNGTERVRLLERCIYNQEESVRFDSDVGEYRAVTEL |
| SEQ ID NO: | GRPDAEYWNSQKDLLEQRRAAVDTYCRHNYGVGESFTVQ |
| 20; beta | RRVEPKVTVYPSKTQPLQHHNLLVCSVSGFYPGSIEVRWFR |
| chain: SEQ | NGQEEKAGVVSTGLIQNGDWTFQTLVMLETVPRSGEVYTC |
| ID NO: 119) | QVEHPSVTSPLTVEWRARSESAQSKGGGGSEDLYFQSGGGG |
| | SCPPCPAPEAAGGPSVFLFPPKPKDTLMISRTPEVTCVVVDV |
| | SQEDPEVQFNWYVDGVEVHNAKTKPREEQFNSTYRVVSVL |
| | TVLHQDWLNGKEYKCKVSNKGLPSSIEKTISKAKGQPREPQ |
| | VYTLPPSQEEMTKNQVSLTCLVKGFYPSDIAVEWESNGQPE |
| | NNYKTTPPPVLDSDGSFFLYSRLTVDKSRWQEGNWSCSVMH |
| | EALHNHYTQKSLSLSLGKWSHPQFEK |
| DR4G86 | KMRMATPLLMQALPMGSGSGSLEVLFQGPGDTRPRFLEQV |
| (alpha chain: | KHECHFFNGTERVRFLDRYFYHQEEYVRFDSDVGEYRAVT |
| SEQ ID NO: | ELGRPDAEYWNSQKDLLEQKRAAVDTYCRHNYGVGESFTV |
| 20; beta | QRRVYPEVTVYPAKTQPLQHHNLLVCSVNGFYPGSIEVRWF |
| chain: SEQ | RNGQEEKTGWSTGLIQNGDWTFQTLVMLETVPRSGEVYT |
| ID NO: 120) | CQVEHPSLTSPLTVEWRARSESAQSKGGGGSEDLYFQSGGG |
| | GSCPPCPAPEAAGGPSVFLFPPKPKDTLMISRTPEVTCVVVD |
| | VSQEDPEVQFNWYVDGVEVHNAKTKPREEQFNSTYRVVSV |
| | LTVLHQDWLNGKEYKCKVSNKGLPSSIEKTISKAKGQPREP |
| | QVYTLPPSQEEMTKNQVSLTCLVKGFYPSDIAVEWESNGQP |
| | ENNYKTTPPVLDSDGSFFLYSRLTVDKSRWQEGNVFSCSVM |
| | HEALHNHYTQKSLSLSLGKWSHPQFEK |

TABLE 27

| Antigen | DR4G86 | DR4G92 | DR4G110 | DR4G143 |
|---|---|---|---|---|
| HLA type | DRB1*04:01 | DRB1*04:01 | DRB1*4:01 | DRB1*04:01 |
| Peptide | CLIP | CII_257-27:3 | Aggrecan | VitL70A |
| DR4B98 | 167000 | 310000 | 283000 | 278000 |
| DR4B117 | 784000 | 911000 | 689000 | 1070000 |
| DR4B127 | 1020000 | 1210000 | 954000 | 1160000 |
| DR4B4 | 129000 | 131000 | 112000 | 96200 |
| DR4B5 | 7030 | 49000 | 8040 | 22600 |
| DR4B6 | 588000 | 608000 | 372000 | 520000 |
| Isotype | 335 | 486 | 1030 | 410 |
| control | | | | |
| no mAb | 554 | 516 | 705 | 423 |
| no antigen | 125 | 129 | 49 | 126 |

TABLE 28

| | Ag Name | | | | | |
|---|---|---|---|---|---|---|
| | DR4G96 | DR4G102 | DR4G119 | DR4G104 | DR4G98 | DR4G101 |
| | HLA | | | | | |
| | DRB1*01: | DRB1*01: | DRB1*10: | DRB1*04: | DRB1*04: | DRB1*04: |
| | 01 | 01 | 01 | 02 | 02 | 02 |
| | Peptide | | | | | |
| | CLIP | CII_257 | HA | HA | CII-1236 | CII-257 |
| DR4B98 | 9450 | 15000 | 94700 | 421000 | 350000 | 354000 |
| DR4B117 | 823000 | 1110000 | 1240000 | 1080000 | 1150000 | 1140000 |
| DR4B127 | 803000 | 1080000 | 1240000 | 1270000 | 1260000 | 1240000 |
| DR4B4 | 26500 | 3450 | 572000 | 451 | 521 | 639 |
| DR4B5 | 20600 | 62500 | 132000 | 825 | 469 | 723 |
| DR4B6 | 627000 | 693000 | 580000 | 415000 | 623000 | 615000 |
| Isotype | 781 | 392 | 517 | 1130 | 613 | 1250 |
| control | | | | | | |
| no mAb | 694 | 808 | 958 | 702 | 690 | 1030 |
| no antigen | 91 | 165 | 197 | 147 | 202 | 2380 |

TABLE 29

| Ag Name | DR4G103 | DR4G97 | DR4G117 | DR4G142 | DR4G99 |
|---|---|---|---|---|---|
| HLA | DRB1*15:01 | DRB1*15:01 | DRB1*03:01 | DRB1*03:01 | DRB1*01:01 |
| Peptide | HA | CII_1236 | CII_1236 | VitL70A | CII_1236 |
| DR4B98 | 34700 | 80800 | 105000 | 60500 | 10000 |
| DR4B117 | 294000 | 605000 | 769000 | 872000 | 1170000 |
| DR4B127 | 334000 | 748000 | 966000 | 748000 | 882000 |
| DR4B4 | 287 | 210 | 737 | 862 | 697 |
| DR4B5 | 329 | 256 | 730 | 1330 | 45900 |
| DR4B6 | 142000 | 222000 | 620000 | 399000 | 672000 |
| Isotype | 487 | 393 | 603 | 2220 | 490 |
| control | | | | | |
| no mAb | 419 | 1353 | 590 | 773 | 1070 |
| no antigen | 55 | 137 | 118 | 116 | 132 |

TABLE 30

| | Ag Name | | | | | |
|---|---|---|---|---|---|---|
| | DR4G107 | DR4G115 | DR4G108 | DR4G112 | DR4G145 | DR4G146 |
| | HLA | | | | | |
| | DPB4:01 | DPB4:01 | DQB6:02 | DQB6:02 | DQB2:01 | DQB2:01 |
| | Peptide | | | | | |
| | CLIP | LCAP | CLIP | PLP | CLIP | CLIP |
| DR4B98 | 328 | 198 | 197 | 460 | 5960 | 2500 |
| DR4B117 | 615 | 1110 | 270 | 421 | 2880 | 2960 |
| DR4B127 | 307 | 239 | 248 | 394 | 2070 | 2830 |
| DR4B4 | 65 | 57 | 53 | 69 | 297 | 209 |
| DR4B5 | 100 | 70 | 76 | 123 | 1360 | 736 |
| DR4B6 | 133 | 67 | 76 | 203 | 473 | 1450 |
| Isotype | 153 | 91 | 99 | 250 | 2780 | 1470 |
| control | | | | | | |
| no Ab | 193 | 179 | 294 | 249 | 519 | 485 |
| no Ag | 299 | 185 | | | | |

### Example 9. Crystal Structure of HLA-DR4 ECD in Complex with DR4B117 Fab

The HLA-DR4 construct used for structural studies was DR4G86. The protein was expressed in transiently transfected HEK 293S-GnTi cells. The clarified and concentrated supernatant was loaded onto two tandem 5-mL HiTrap™ MabSelect Sure columns (GE Healthcare Cat#11-0034-95) and eluted with 0.1 M Na acetate, pH 3.5, and dialyzed into DPBS. pH 7.2. The Fab fragment of mAb DR4B117 was transiently transfected in 200 mL Expi293F™ cells. The clarified supernatant was loaded onto a 5-mL HisTrap™ HP column (GE Healthcare Cat#17-5248-02) and eluted using a stepwise gradient of increasing imidazole concentration. The protein was further purified by size exclusion chromatography (SEC) using a HiLoad Superdex™ 200 column (GE Healthcare Cat#28-9893-36) run in 20 mM Tris, 50 mM NaCl, pH 7.4.

To make the antibody-antigen complex, 24 mg DR4G86 in DPBS. pH 7.2 and 11 mg Fab DR4B117 in 20 mM Tris, 50 mM NaCl, pH 7.4 were gently mixed at 1:1 molar ratio and incubated at room temperature for 1 day. The mixture was then treated by TEV protease in TEV buffer (ThermoFisher Cat#12575-023) using 1 unit of enzyme per 3 μg total protein and incubated at 30° C. overnight. To separate the complex from Fc, the cleaved material was loaded onto a 1-mL Protein A column (GE Healthcare Cat#11-0034-93) which was pre-equilibrated in DPBS, pH 7.2. The flow-through containing mostly the DR4:Fab complex was collected in 1-mL fractions, pooled and loaded onto an SEC column (Superdex™ 200, GE Healthcare, Cat#17-1071-01) in 20 mM Tris, 50 mM NaCl, pH 7.4 to remove other contaminants such as residual Fab and TEV protease. The SEC pool containing DR4B117:Fab complex was concentrated to 1 mg/mL. The samples were analyzed by SDS PAGE, A280, SE-HPLC and SEC MALS. The SEC MALS analysis indicated that the molecular weight of the complex is in agreement with that calculated from the sequence.

For crystallization, the DR4:Fab complex was concentrated using an Amicon Ultra 10 kDa MWCO device to 14 mg/mL in 20 mM Tris pH 7.4, 50 mM NaCl. Crystallization of the complex was carried out by the vapor diffusion method in a sitting drop format at 20° C. using a Mosquito robot (TTP Labtech) and MRC 2-well crystallization plates (Swissci). The screening for crystallization conditions was performed using PEGs screen (Qiagen, Cat. No. 130904), Crystal Screen HT (Hampton Research. Cat. No. HR2-130) and an in-house screen (Obmolova et al. (2014) Acta Crystallogr, F70:1107-1115). Diffraction quality crystals were obtained after optimization from 18% PEG 3350, 1.0 M LiCl, 0.1 M MES buffer, pH 6.5. Crystals were harvested in the mother liquor supplemented with 20% glycerol and flash-cooled in liquid nitrogen. X-ray diffraction data were collected at the Advanced Photon Source (Beamline 17-ID) at the Argonne National Laboratory and were processed with XDS (Kabsch, (2010) Acta Crystallogr, D66:125-132) to a resolution of 1.75 Å. The details of the X-ray data are given in Table 31.

The DR4:Fab structure was determined by molecular replacement using the program Phaser (Read, (2001) Acta Crystallogr. D57:1373-1382). Crystal structures from the Protein Data Bank 3na9 for the Fab and 4mcz for DR4 were used as search models. The structure was refined with Phenix (Adams et al., (2004) J. Synchrotron Radiat. 11:53-55) and model fitting was carried out with COOT (Emsley and Cowtan. (2004) Acta Crystallogr, D60:2126-2132). The refinement statistics are given in Table 31. All graphics was generated with Pymol (www.schrodinger.com). All other calculations were carried out with the CCP4 suite (Collaborative Computational Project, Number 4 (1994) Acta Crystallogr. D53:240-255).

TABLE 31
X-ray data and refinement statistics for DR4:B117 complex.

| Crystal data | | |
|---|---|---|
| Space group | P2 | |
| Unit cell axes (Å) | 69.67, 54.78, 116.03 | |
| Unit cell angles (°) | 90, 95.06, 90 | |
| Molecules per asymmetric unit | 1 complex | |
| X-ray data | | |
| Resolution (Å) | 50-1.75 | (1.81-1.75) |
| Number of measured reflections | 289,739 | (29,609) |
| Number of unique reflections | 87,606 | (8,744) |
| Completeness (%) | 99.2 | (99.7) |
| Redundancy | 3.3 | (3.4) |
| R-merge | 0.046 | (1.148) |
| Mean I/σ(1) | 14.2 | (1.5) |
| B factor from Wilson plot (Å²) | 29.8 | |
| Refinement | | |
| Resolution (Å) | 35-1.75 | |
| R-work (%) | 18.7 | |
| R-free (5% data) (%) | 22.2 | |
| Number of all atoms | 6,925 | |
| Number of water molecules | 466 | |
| Bond lengths RMSD (Å) | 0.016 | |
| Bond angles RMSD (°) | 1.4 | |
| Mean B factor from model (Å²) | 40.4 | |

*Numbers in parentheses refer to the highest-resolution shell.

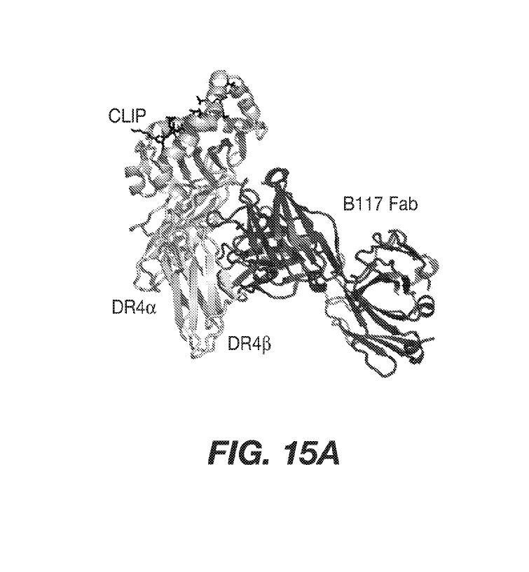

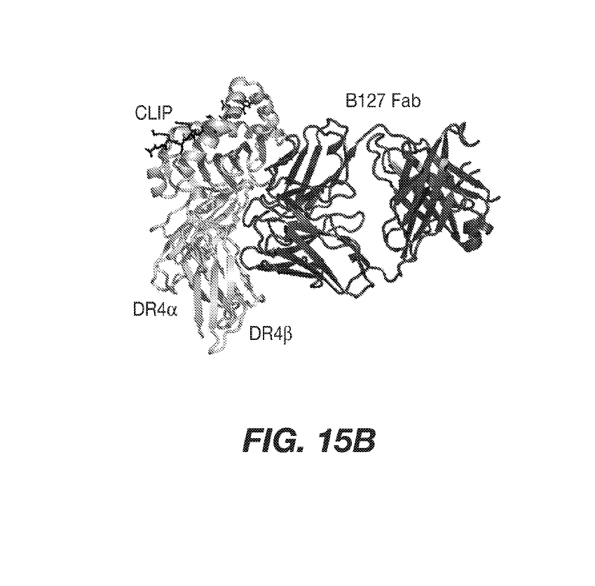

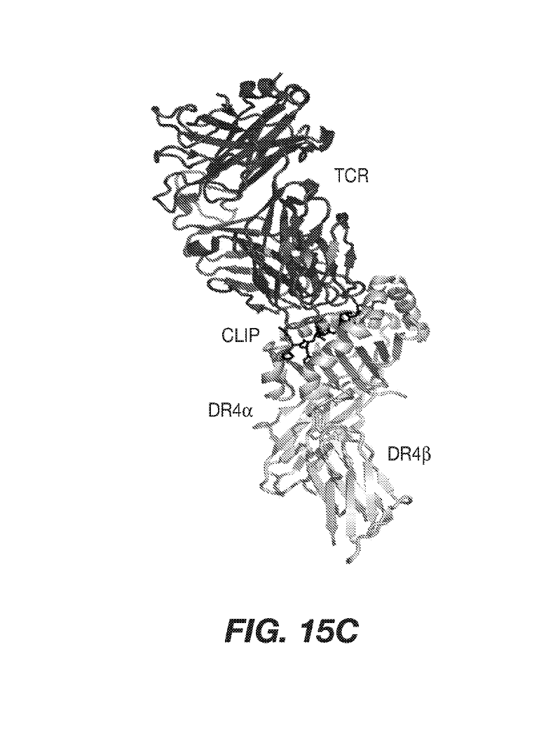

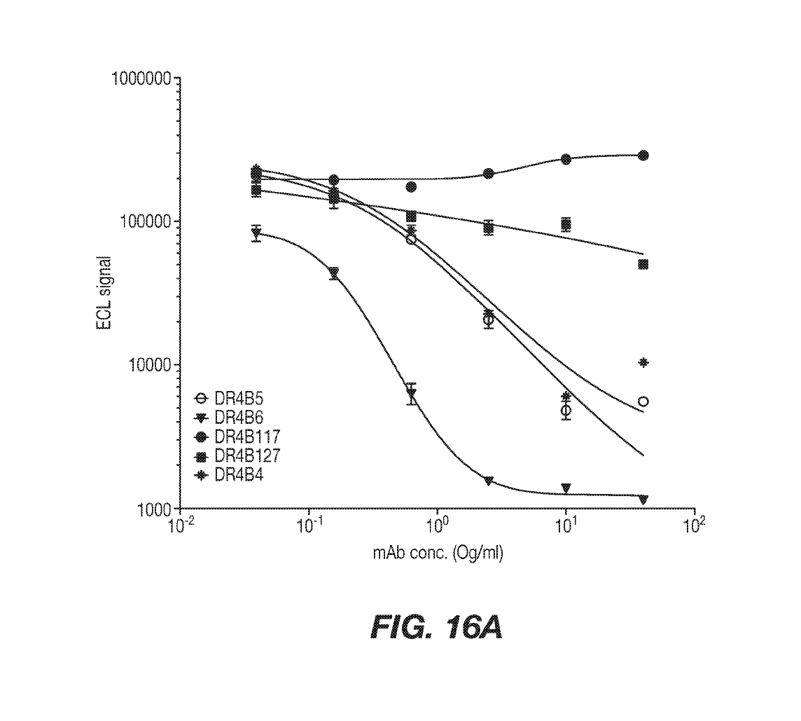

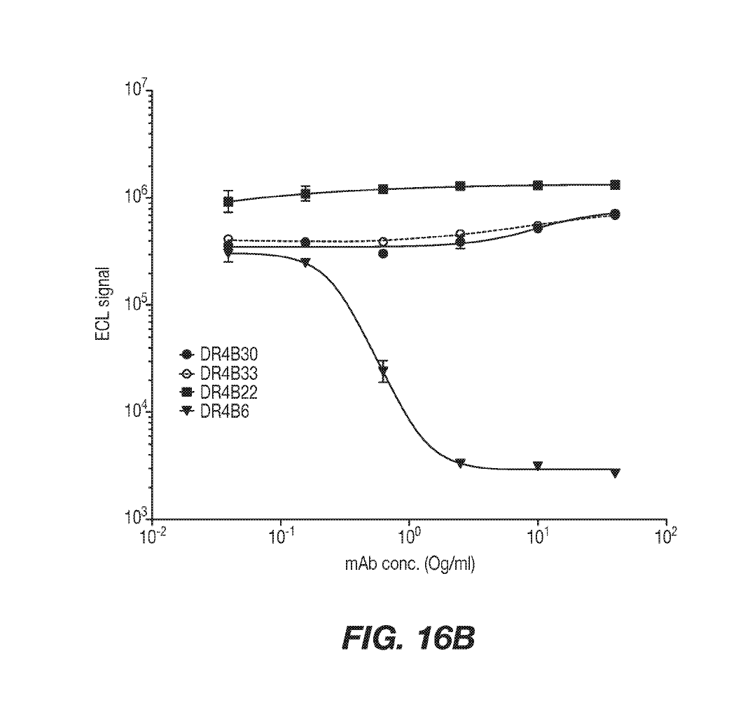

The structure of the complex is shown in FIG. 15A. The structure revealed that the binding epitope of DR4B117 was composed of residues from both the α and β subunits of DR4. The epitope was distal to the CLIP peptide and TCR binding surface. Therefore, DR4B117 did not directly block TCR from binding DR4. The antibody-antigen interface covered over 2500 Å² of solvent accessible surface, out of which 1300 Å² on the antibody and 1200 Å² on DR4 (700 Å² on α and 500 Å² on β). All six CDRs of DR4B117 provided contacts to the antigen. The epitope and paratope residues identified using a 4-A cutoff are given in Table 32. Based on the number of contacts, the most important residues in the epitope were E3, F108, D110, R140 in chain α (residue numbering according to SEQ ID NO: 13) and V143 and Q149 in chain β (residue numbering according to SEQ ID NO: 14)

TABLE 32
Epitope and paratope residues of DR4B117.*

Epitope on DR4α: K2, E3, V6, E88, V89, T90, F108, D110, K111, R140, L144, R146, K176
Epitope on DR4β: L114, K139, V142, V143, S144, T145, L147, I148, Q149, E162
Paratope on VH of B117: F29, S31, P53, E54, Y55, G56, Q62, Y101, Y102, 1103, N105, R106
Paratope on VL of B117: E1, Q27, S31, S32, Y33, Y50, S94, S95, P96
*Residue wintering according to SEQ ID NO: 13 (DR4α), SEQ ID NO: 14 (DR4β), SEQ ID NO: 56 (VH), SEQ ID NO: 60 (VL).

### Example 10. Crystal Structure of HLA-DR4 ECD in Complex with DR4B127 Fab

Protein expression, complex preparation, crystallization, X-ray data collection and structure determination were conducted as described in Example 9 except for the following. The complex was formed by mixing 27 mg DR4W176 in DPBS, pH 7.2 and 13 mg Fab DR4B127 in 20 mM Tris, 50 mM NaCl, pH 7.4 at 1:1 molar ratio. The crystals of the complex were obtained from 18% PEG 3350, 0.1 M Na acetate pH 4.5, 0.2 M Na formate. The structure was determined at 2.75 Å resolution. The X-ray data and refinement statistics are given in Table 33.

TABLE 33
X-ray data and refinement statistics for DR4:B127 complex.

| Crystal data | | |
|---|---|---|
| Space group | C222₁ | |
| Unit cell axes (Å) | 88.97, 132.61, 199.77 | |
| Unit cell angles (°) | 90, 90, 90 | |
| Molecules per asymmetric unit | 1 complex | |
| X-ray data | | |
| Resolution (Å) | 50-2.75 | (2.85-2.75) |
| Number of measured reflections | 206,770 | (21,332) |
| Number of unique reflections | 30,983 | (3,052) |
| Completeness (%) | 99.5 | (99.4) |
| Redundancy | 6.7 | (7.0) |
| R-merge | 0.081 | (1.214) |
| Mean I/σ(I) | 19.3 | (2.4) |
| B factor from Wilson plot (Å²) | 72.8 | |
| Refinement | | |
| Resolution (Å) | 40-2.75 | |
| R-work (%) | 22.5 | |
| R-free (5% data) (%) | 26.1 | |
| Number of all atoms | 6,451 | |
| Number of water molecules | 0 | |
| Bond lengths RMSD (Å) | 0.004 | |
| Bond angles RMSD (°) | 0.8 | |
| Mean B factor from model (Å²) | 72.1 | |

*Numbers in parentheses refer to the highest-resolution shell.

The structure of the complex is shown in FIG. 15B. The structure revealed that the binding epitope of DR4B127 was composed of residues from both the a and B subunits of DR4. The epitope was distal to the CLIP peptide and TCR binding surface. Therefore, DR4B127 did not directly block TCR from binding DR4. The antibody-antigen interface covered over 3200 Å² of solvent accessible surface, out of which 1500 Å² on the antibody and 1700 Å² on DR4. Only four of the six CDRs (CDR-H1, CDR-H3, CDR-L1 and CDR-L2) provided contacts to the antigen. The epitope and paratope residues identified using a 4-A cutoff are given in Table 34. Based on the number of contacts, the most important residues in the epitope were K2 in chain α (residue numbering according to SEQ ID NO: 13) and D41, S126, R130, V142 and Q149 in chain (1 (residue numbering according to SEQ ID NO: 14).

TABLE 34
Epitope and paratope residues of DR4B127.

Epitope on DR4α: I1, K2, E3, D27, R140, E141, D142, H143
Epitope on DR4β: H16, F17, R23, R25, R29, R39, D41, D43, V44,
V50, G125, S126, E128, V129, R130, V142, G146, L147, Q149, V159
Paratope on VH of B127: P53, I54, S55, G56, N57, D99, S101, Y102,
Y103, R104, N105, Y106, G107, D109, Y110
Paratope on VL of B127: S30, S31, Y49, D50, S52, N53, R54, A55, T56,
A60, S63, G64, S65, S67
*Residue numbering according to SEQ ID NO: 13 (DR4α), SEQ ID NO: 14 (DR4β), SEQ ID NO: 58 (VR), SEQ ID NO: 61 (VL).

Comparison of the crystal structures of DR4 in complex with TCR (PDB entry 1j8h; Hennecke and Wiley, (2002) J. Exp. Med. 195:571-581) (FIG. 15C), with Fab DR4B117 (FIG. 15A) and with Fab DR4B127 (FIG. 15B) showed that both antibodies bind DR4 at the site distal from the TCR epitope and therefore did not directly interfere with TCR binding. DR4B117 and DR4B127 bound DR4 close to the cell surface and likely caused the ECD of DR4 to tilt away from the mAb to allow room for the bulky Fc portion between the DR4 molecule and the membrane, resulting in inability of TCR binding to DR4. The Fab fragments of both antibodies, DR4B117 and DR4B127 did not inhibit TCR binding. DR4B117 and DR4B127 block CD4 binding to HLA-DR.

### Example 11. Epitope Binning

Three competition groups were identified in initial matrix cross-competition experiments of 31 HLA-DR antibodies utilizing DRG89 as antigen (HLA-DR1*04:01 in complex with collagen II_1236 peptide) or DR4G99 (HLA-DR1:01 in complex with hemagglutinin peptide) using MDS or IBIS. DR4B4 and DR4B5 bound DR4G89 poorly and could not be used in the assays.

Following competition groups were identified for cross-competition to DRG89:

Group 1: DR4B30, DR4B98. DR4B117, DR4B127, DR4B78. DR4B38, DR4B70, DR4B22 and DR4B33. Group 2: DR4B6.

Following competition groups were identified for cross-competition to DRG99:

Group 1: DR4B30, DR4B117, DR4B127, DR4B78, DR4B38. DR4B70, DR4B22 and DR4B33. Group 2: DR4B6. Group 3: DR4B98. DR4B98, due to its minimal binding to DR4G99 was not mapped to Group 1.

### Samples and Reagents:

### Antigens:

DR4G89 1.264 mg/ml
DR4G99 0.99 mg/ml
Running Buffer for IBIS systems: PBST, degassed.
Cross competition by MSD ELISA:

5 μl of 10 μg/ml of DR4G89 or DR4G99 were absorbed on Meso Scale Discovery (MSD) HighBind plates (Gaithersburg, Md.) for 2 hours then washed 3× with 150 μl 0.1M HEPES. Plate was blocked with 5% BSA buffer overnight at 4° C. The next day, plates were washed 3× with 0.1 M HEPES buffer, pH 7.4, followed by the addition of the mixture of Ruthenium (Ru)-labeled anti-DR4 mAb which was pre-incubated at room temperature for 30 minutes with 1 mM of other anti-DR4 mAbs. After incubation with gentle shaking at room temperature 2 hours, plates were washed 3× with 0.1M HEPES buffer (pH 7.4). MSD Read Buffer T was diluted with distilled water (4-fold) and dispensed into each well then analyzed with a SECTOR Imager 6000 (Meso Scale Discovery. Gaithersburg. Md.).

Epitope binning by IBIS (the Instrument for Biomolecular Interaction Sensing MultipleX 96, IBIS-MX %, Wasatch Microfluidics Inc.):
The protocol was according to the literature (Abdiche, Y. N. et al. (2014), PLoS One 9, fe92451) with some modification as described following:

### Chip Preparation

A CFM 2 (Wasatch Microfluidics) was used to create a microarray of 96 mAbs. It draws forty-eight 70-μl plugs of sample from a 96-well microplate into a fluidic manifold which focuses the solutions into a 4×12 array of 48 micro flow cells on the surface of the SPR substrate (a G-COOH coated prism from Ssens by, NL) and cycles the solutions back and forth at 60 μl/min A 96-well microplate was prepared with 100 μl of each mAb at 30 μg/ml in MES coupling buffer pH 4.5 and loaded into bay 2 of the CFM. A second plate of freshly mixed activating reagents (150 μl 0.4 M EDC and 150 μl 0.1 M sulfo-NHS in a total of 5 ml of MES coupling buffer pH 4.5) was loaded into bay 1. The CFM was then primed with system buffer (PBS+0.01% T20). The set of anti-DR4 mAb plate contained 42 mAbs arrayed in triplicate. Once docked, the activating reagents were cycled over the surface for 7 min and followed immediately by this set of mAbs and cycled for 15 min.

The printed chip was then loaded into the IBIS SPR reader (MX96, IBIS Technologies by), which uses a single flow cell and autosampler configured to address the array with back-and-forth cycled injections of 80 ml per analyte. Once loaded, 1 M ethanolamine was injected across the chip for 15 min to quench the excess reactive esters. The chip was then washed with system buffer and the chip image was used to define the reaction spots (i.e., the 96-ligand array) and the interstitial reference spots (two local reference spots per reaction spot). For classical binning, a co-injection was used, where both antigen (either DR4G89 or DR4G99) and mAb analyte were transported to the flow cell in parallel lines, and injected immediately after one another before continuing with regeneration. For experiments, antigen was injected for 3 min, followed by 20 μg/ml mAb for a further 3 min, and then the surfaces were regenerated. All SPRi experiments were conducted in a 96×96 analyte-on-ligand format.

### Biosensor Data Analysis

Data were processed in SPRint software v. 6.15.2.1 (calibrated, locally referenced, and aligned to zero on the Y-axis prior to the binding step of interest) and analyzed in Wasatch Microfluidics' binning software for heat map generation, sorting and node plotting. Hierarchical clustering was used to group like-behaved mAbs together in the heat map. Heat maps and node plots are alternate ways of visualizing epitope bins and their inter-bin relationships.

### Example 12. HLA-DR Antibodies do not Block HLA-DR Interaction with a Cognate TCR ECD

Crystal structure studies confirmed that DR4B117 and DR1B127 did not block HLA-DR interaction with the cognate TCR. Several additional antibodies were tested for their effect to block recombinant TCR using MDS.

Antibodies DR4B117, DR4B127, DR4B30, DR4B70. DR4B22, DR4B33, DR4B38 and DR4B78 did not inhibit HLA-DR interaction with the cognate TCR whereas DR4B98 partially inhibited the interaction. DR4B4, DR4B5 and DR4B6 inhibited HLA-DR/TCR interaction.

FIG. 16A shows the dose response curve of inhibition of the HLA-DR/TCR interaction for DR4B117, DR4B127. DR4B4, DR4B5 and DR4B6.

FIG. 16B shows the dose response curve of inhibition of the HLA-DR/TCR interaction by DR4B22, DR4B30 and DR4B33.

### Materials

MSD plates (Meso Scale Discovery #L15XA-3)
Dulbecco's Phosphate Buffered Saline (Gibco, #14190-136)
Bovine Albumin Fraction V (Millipore, Cat#820451)
Tween 20 (Sigma, Cat#P1379)
The HisTag Ab-biotin (Genscript, #A00613)
Sulfo-Tag Streptavidin (Meso Scale Discovery, #R32AD-50)
MSD Read Buffer T (MSD. Cat#R92TC-1)
HLA-DR antigen: DR4G134 (DRG89 without hexahistidine tag); Human HLA-DRA1*01:02/DRB1*04:01 ECD with hemagglutinin peptide HA_304-318 (HA) in the format of Alpha chain: ECD_G4S+TEV+G4S+MMB+6×HisTag; Beta chain: HA+3XGS+HRV3C+ECD+G4S+TEV+G4S+MMB+StrepII
DR4G79: TCR receptor ECD; Human HA 1.7 TCR ECD in the format of: Alpha chain ECD+TEV+MMB+HisTag; Beta chain ECD+TEV+huIgG4MMB+Flag+StrepII (DR4G79 HC: SEQ ID NO: 69; LC SEQ ID NO: 70

> DR4G79 HC

SEQ ID NO: 69

QSVTQLGSHVSVSEGALVLLRCNYSSSVPPYLFWYVQYPNQGLQLLLKYT
SAATLVKGINGFEAEFKKSETSFHLTKPSAHMSDAAEYFCAVSESPFGNE
KLTFGTGTRLTIIPNIQNPDPAVYQLRDSKSSDKSVCLFTDFDSQTNVSQ
SKDSDVYITDKTVLDMRSMDFKSNSAVAWSNKSDFACANAFNNSIIPEDT
FFPSPESSCDVKGGGGSEDLYFQSGGGGSCPPCPAPEAAGGPSVFLFPPK
PKDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPP
VLDSDGSFFLYSRLTVDKSRWQEGNVFSCSVMHEALHNHYTQKSLSLSLG
KGGGGSHHHHHH
> DR4G79 LC

SEQ ID NO: 70

GSVKVTQSSRYLVKRTGEKVFLECVQDMDHENMFWYRQDPGLGLRLIYFS
YDVKMKEKGDIPEGYSVSREKKERFSLILESASTNQTSMYLCASSSTGLP
YGYTFGSGTRLTVVEDLNKVFPPEVAVFEPSEAEISHTQKATLVCLATGF
FPDHVELSWWVNGKEVHSGVSTDPQPLKEQPALNDSRYSLSSRLRVSATF
WQNPRNHFRCQVQFYGLSENDEWTQDRAKPVTQIVSAEAWGRADCGFTGG
GGSEDLYFQSGGGGSCPPCPAPEAAGGPSVFLFPPKPKDTLMISRTPEVT
CVVVDVSQEDPEVQFNWYVDGVEVHNAKTKPREEQFNSTYRVVSVLTVLH
QDWLNGKEYKCKVSNKGLPSSIEKTISKAKGQPREPQVYTLPPSQEEMTK
NQVSLTCLVKGFYPSDIAVEWESNGQPENNYKTTPPVLDSDGSFFLYSRL
TVDKSRWQEGNVFSCSVMHEALHNHYTQKSLSLSLGKGGGGSDYKDDDDK
WSHPQFEK

### Protocol

Assay buffer: 1×DPBS+1% BSA+0.05% tween 20
1. Coat MSD plate with 50 μl DR4G134 antigens (at 5 μg/ml), shake for 10 minutes at RT, incubate at 4° C. overnight
2. Empty the plate, block with 150 μl Assay buffer for 1 hour with gentle shaking, in the meantime, premix DRG79, DR4 mAb, anti-His Ab, SulfoTag-SA (final concentration 5 μg/ml, 10 μg/ml and 2 μg/ml, respectively)
3. Empty the plate, add premixture, incubate for 1 hour
4. Wash 3× with 300 μl PBST
5. Add 150 μl MSD read buffer for MSD plates

### 6. Read in MSD

### Example 13. Functional Characterization of DR4B70, DR4B22, DR4B33, DR4B38 and DR4B78

DR4B70, DR4B22, DR4B33, DR4B38 and DR4B78 were characterized using assays described above. All antibodies bound HLA-DR4 or HLA-DR1 and none of the antibodies bound DQ or DP. Table 35 shows the results of binding of the antibodies to various HLA antigens.

TABLE 35

| | HLA α chain | | | | | |
|---|---|---|---|---|---|---|
| | HDQA1*01:02 | DPA1:03 | DRA1*01:02 | | DRA1*01:02 | |
| | HLA β | | | | | |
| | DQB1*06:02 | DPB1*04:01 | DRB1*04:01 | | DRB1*01:01 | |
| | Peptide | | | | | |
| | | NY- | | | | |
| | Insulin peptide | ESO_157- | HA_304- | CII_1236- | HA_304- | CII_1236- |
| | INS_1-15 | 169 | 318 | 1249 | 318 | 1249 |
| | Antigen | | | | | |
| | | | DR4G89, | | DR4G93, | DR4G99, |
| | DR4G111, Ab | DR4G113, | Ab at | DR4G90, | Ab at | Ab at |
| | at 5 μg/ml | Ab at 5 μg/ml | 0.2 μg/ml | Ab at 0.2 μg/ml | 0.2 μg/ml | 0.2 μg/ml |
| DR4B78 | 4250 | 930 | 978810 | 1138295 | 1061118 | 1075472 |
| DR4B38 | 100 | 148 | 1218979 | 1236584 | 1121123 | 1205648 |
| DR4B70 | 3219 | 1266 | 887635 | 1128537 | 971772 | 1084766 |
| DR4B22 | 16300 | 2185 | 1014746 | 1101094 | 896077 | 1027522 |
| DR4B30 | 12905 | 1969 | 1128453 | 1248862 | 1066233 | 1199978 |

Table 36 shows the antibody characteristics in functional assays. All antibodies were antagonistic DR4B78, DR4B38, DR4B70 and DR4B22 induced B cell apoptosis and/or death. DR4B30 did not.

TABLE 36

| | HLA-DR4 DC | | | Human B cell |
|---|---|---|---|---|
| | MLR 10 μg/ml | DR4 Tg DC | PBMC | apoptosis and/or |
| mAb | mAb | Binding* | Binding | death? |
| DR4R78 | YES | High | Medium | Induced cell death |
| DR4B38 | YES | High | High | Induced cell death |
| DR4970 | YES | High | High | Induced cell death |
| DR4B22 | YES | High | Medium | Induced cell death |
| DR4B30 | YES | High | High | No effect |

YES: inhibitory in HLA-DR4 DC MLR assay
*all tested antibodies exhibited bimodal binding
High: Mean fluorescent intensity higher than what was detected using DR4B6
Medium: MFI comparable to what was detected with DR4B6

What is claimed:

1) An isolated antibody or an antigen-binding fragment thereof specifically binding HLA-DR, wherein the antibody or the antigen-binding fragment thereof competes for binding to HLA-DR with an antibody comprising
i) a heavy chain variable domain (VH) of SEQ ID NO: 58 and a light chain variable domain (VL) of SEQ ID NO: 61;
j) the VH of SEQ ID NO: 56 and the VL of SEQ ID NO: 60;
k) the VH of SEQ ID NO: 57 and the VL of SEQ ID NO: 61;
l) the VH of SEQ ID NO: 137 and the VL of SEQ ID NO: 61;
m) the VH of SEQ ID NO: 138 and the VL of SEQ ID NO: 61;
n) the VH of SEQ ID NO: 139 and the VL of SEQ ID NO: 61;
o) the VH of SEQ ID NO: 140 and the VL of SEQ ID NO: 142; or
p) the VH of SEQ ID NO: 141 and the VL of SEQ ID NO: 61.

2) The antibody or the antigen-binding fragment thereof of claim 1, wherein the antibody or the antigen-binding fragment thereof is an antagonist of HLA-DR.

3) The antibody or the antigen-binding fragment thereof of claim 1 or 2, wherein the antibody or the antigen-binding fragment thereof inhibits CD4⁺ T cell proliferation at antibody concentration of 1 μg/ml by at least 30% in a co-culture of human CD4⁺ T cells and dendritic cells isolated from transgenic animals expressing human HLA-DR4.

4) The antibody or the antigen-binding fragment thereof of any one of claims 1-3, wherein the antibody or the antigen-binding fragment thereof does not block interaction of HLA-DR with a cognate T cell receptor.

5) The antibody or the antigen-binding fragment thereof of any one of claims 1-4, wherein the HLA-DR is HLA-DR4 comprising HLA-DR α chain of SEQ ID NO: 13 and HLA-DR β chain of SEQ ID NO: 14 in complex with a hemagglutinin peptide of SEQ ID NO: 7.

6) The antibody or the antigen-binding fragment thereof of any one of claims 1-5, wherein the antibody or the antigen-binding fragment thereof has one, two, three, four or five of the following properties:
a) binds HLA-DR4 comprising HLA-DR α chain of SEQ ID NO: 13 and HLA-DR β chain of SEQ ID NO: 14 in complex with hemagglutinin peptide of SEQ ID NO: 7 with an equilibrium dissociation constant (K_{D}) of 5×10⁻⁸ M or less, wherein K_{D} is measured using ProteOn XPR36 system at 25° C. in a buffer containing DPBS, 0.01% (w/v) polysorbate 20 (PS-20) and 100 μg/ml BSA;
b) binds HLA-DR1 comprising HLA-DR α chain of SEQ ID NO: 13 and HLA-DR β chain of SEQ ID NO: 15 in complex with the hemagglutinin peptide of SEQ ID NO: 7 with an equilibrium dissociation constant (K_{D}) of 5×10⁻⁸ M or less, wherein K_{D} is measured using ProteOn XPR36 system at 25° C. in a buffer containing DPBS, 0.01% (w/v) PS-20 and 100 μg/ml BSA;
c) lacks an ability to induce apoptosis of B cells, wherein apoptosis is determined by measuring frequency of CD3⁻ CD20⁺ annexinV⁺live/dead⁻ B cells in a sample of human peripheral blood cells (PBMC) using flow cytometry;
d) lacks an ability to induce death of B cells, wherein death of B cells is determined by measuring frequency of CD3⁻ CD20⁺ annexinV⁺live/dead⁺ B cells in the sample of human PBMC using flow cytometry; or
e) inhibits binding of HLA-DR to CD4.

7) The antibody or the antigen-binding fragment of any one of claims 1-6, wherein HLA-DR is HLA-DR4, HLA-DR1, HLA-DR3, HLA-DR10 or HLA-DR15.

8) The antibody or the antigen-binding fragment of claim 7, wherein HLA-DR α chain and HLA-DR β comprise amino acid sequences of
a) SEQ ID NOs: 13 and 14, respectively;
b) SEQ ID NOs: 13 and 15, respectively;
c) SEQ ID NOs: 13 and 106, respectively;
d) SEQ ID NOs: 13 and 105, respectively;
e) SEQ ID NOs: 13 and 107, respectively; or
f) SEQ ID NOs: 13 and 108, respectively.

9) The antibody or the antigen-binding fragment thereof of any one of claims 1-8, wherein the antibody or the antigen-binding fragment thereof binds HLA-DR4 with an equilibrium dissociation constant (K_{D}) of less than about 5×10⁻⁸ M.

10) The antibody or the antigen-binding fragment thereof of any one of claims 1-9, wherein HLA-DR contains a shared epitope consisting of amino acid sequences QKRAA (SEQ ID NO: 66), QRRAA (SEQ ID NO: 67) or RRRAA (SEQ ID NO: 68).

11) The antibody or the antigen-binding fragment thereof of any one of claims 1-10, wherein HLA-DR is in complex with a peptide.

12) The antibody or the antigen-binding fragment thereof of claim 11, wherein the peptide comprises an amino acid sequence of SEQ ID NOs: 7, 8, 9, 71, 72, 104 or 122.

13) The antibody or the antigen-binding fragment thereof of claim 12, wherein the peptide consists of the amino acid sequence of SEQ ID NOs: 7, 8, 9, 71, 72, 104 or 122.

14) The antibody or the antigen-binding fragment thereof of any one of claims 1-13, wherein the antibody binds HLA-DRA1*01:02 of SEQ ID NO: 13 at amino acid residues E3, F108, D110 and R140 and HLA-DRB1*04:01 of SEQ ID NO: 14 at amino acid residues V143 and Q149.

15) The antibody or the antigen-binding fragment thereof of claim 14, wherein the antibody binds HLA-DRA1*01:02 of SEQ ID NO: 13 at amino acid residues K2, E3, V6, E88, V89, T90, F108, D110, K111, R140, L144, R146 and K176 and HLA-DRB1*04:01 of SEQ ID NO: 14 at amino acid residues L114, K139, V142, V143, S144, T145, L147, I148, Q149 and E162.

16) The antibody or the antigen-binding fragment thereof of any one of claims 1-13, wherein the antibody binds HLA-DRA1*01:02 of SEQ ID NO: 13 at amino acid residue K2 and HLA-DRB1*04:01 of SEQ ID NO: 14 at amino acid residues D41, S126, R130, V142 and Q149.

17) The antibody or the antigen-binding fragment thereof of claim 16, wherein the antibody binds HLA-DRA1*01:02 of SEQ ID NO: 13 at amino acid residues I1, K2, E3, D27, R140, E141, D142 and H143 and HLA-DRB1*04:01 of SEQ ID NO: 14 at amino acid residues H16, F17, R23, R25, R29, R39, D41, D43, V44, V50, G125, S126, E128, V129, R130, V142, G146, L147, Q149 and V159.

18) The antibody or the antigen-binding fragment thereof of any one of claims 1-17, comprising
a) a heavy chain complementarity determining region 1, 2 and 3 (a HCDR1, a HCDR2 and a HCDR3) of SEQ ID NOs: 73, 74 and 75, respectively, and a light chain complementarity determining region 1, 2 and 3 (a LCDR1, a LCDR2 and a LCDR3) of SEQ ID NOs: 76, 77 and 78, respectively;
b) the HCDR1, the HCDR2, the HCDR3, LCDR1, the LCDR2 and the LCDR3 of SEQ ID NOs: 39, 42, 46, 50, 52 and 54, respectively;
c) the HCDR1, the HCDR2, the HCDR3, the LCDR1, the LCDR2 and the LCDR3 of SEQ ID NOs: 40, 43, 47, 51, 53 and 55, respectively;
d) the HCDR1, the HCDR2, the HCDR3, the LCDR1, the LCDR2 and the LCDR3 of SEQ ID NOs: 41, 44, 48, 51, 53 and 55, respectively;
e) the HCDR1, the HCDR2, the HCDR3, the LCDR1, the LCDR2 and the LCDR3 of SEQ ID NOs: 41, 45, 49, 51, 53 and 55, respectively;
f) the HCDR1, the HCDR2, the HCDR3, the LCDR1, the LCDR2 and the LCDR3 of SEQ ID NOs: 123, 126, 129, 51, 53 and 55, respectively;
g) the HCDR1, the HCDR2, the HCDR3, the LCDR1, the LCDR2 and the LCDR3 of SEQ ID NOs: 123, 126, 130, 51, 53 and 55, respectively;
h) the HCDR1, the HCDR2, the HCDR3, the LCDR1, the LCDR2 and the LCDR3 of SEQ ID NOs: 123, 126, 131, 51, 53 and 55, respectively;
i) the HCDR1, the HCDR2, the HCDR3, the LCDR1, the LCDR2 and the LCDR3 of SEQ ID NOs: 124, 127, 132, 134, 135 and 136, respectively; or
j) the HCDR1, the HCDR2, the HCDR3, the LCDR1, the LCDR2 and the LCDR3 of SEQ ID NOs: 125, 128, 133, 51, 53 and 55, respectively.

19) The antibody or the antigen-binding fragment thereof of any one of claims 1-18, wherein the antibody comprises a heavy chain framework derived from IGHV1-69 (SEQ ID NO: 62), IGHV5-51 (SEQ ID NO: 63) or IGHV3_3-23 (SEQ ID NO: 161).

20) The antibody or the antigen-binding fragment thereof of claim 19, wherein the antibody comprises a light chain framework derived from IGKV3-20 (SEQ ID NO: 64), IGKV3-11 (SEQ ID NO: 65) or IGKV1-39 (SEQ ID NO: 162).

21) The antibody or the antigen-binding fragment thereof of claim 20, wherein the heavy chain framework and the light chain framework are derived from
a) IGHV1-69 (SEQ ID NO: 62) and IGKV3-20 (SEQ ID NO: 64), respectively;
b) IGHV5-51 (SEQ ID NO: 63) and IGKV3-11 (SEQ ID NO: 65), respectively;
c) IGHV1-69 (SEQ ID NO: 62) and IGKV3-11 (SEQ ID NO: 65), respectively;
d) IGHV3_3-23 (SEQ ID NO: 161) and IGKV3-11 (SEQ ID NO: 65), respectively; or
e) IGHV5-51 (SEQ ID NO: 63) and IGKV1-39 (SEQ ID NO: 162).

22) The antibody or the antigen-binding fragment thereof of any one of claims 1-21, comprising the VH that is at least 84%, 85%, 86%, 87%, 88%, 89%, 90%, 91%, 92%, 93%, 94%, 95%, 96%, 97%, 98%, 99% or 100% identical to the amino acid sequence of SEQ ID NOs: 56, 57, 58, 59, 137, 138, 139, 140 or 141.

23) The antibody or the antigen-binding fragment thereof of claim 22, comprising the VL that is at least 90%, 91%, 92%, 93%, 94%, 95%, 96%, 97%, 98%, 99% or 100% identical to the amino acid sequence of SEQ ID NOs: 60, 61 or 142.

24) The antibody or the antigen-binding fragment thereof of any one of claims 1-23, comprising the VH of SEQ ID NOs: 56, 57, 58, 59, 137, 138, 139, 140 or 141 and the VL of SEQ ID NO: 60 or 61 or 142, the VH and the VL optionally having 1, 2, 3, 4, 5, 6, 7, 8, 9, 10, 11, 12, 13, 14, 15, 16, 17 or 18 amino acid substitutions.

25) The antibody or the antigen-binding fragment thereof of claim 24, comprising the VH and the VL of
a) SEQ ID NOs: 56 and 60, respectively;
b) SEQ ID NOs: 57 and 61, respectively;
c) SEQ ID NOs: 58 and 61, respectively;
d) SEQ ID NOs: 59 and 61, respectively;
e) SEQ ID NOs: 137 and 61, respectively;
f) SEQ ID NOs: 138 and 61, respectively;
g) SEQ ID NOs: 139 and 61, respectively;
h) SEQ ID NOs: 140 and 142, respectively; or
i) SEQ ID NOs: 141 and 61, respectively.

26) The antibody or the antigen-binding fragment thereof of claim 25, wherein the VH and the VL are encoded by poly nucleotides comprising
a) SEQ ID NOs: 79 and 80, respectively;
b) SEQ ID NOs: 81 and 82, respectively;
c) SEQ ID NOs: 83 and 82, respectively;
d) SEQ ID NOs: 121 and 82, respectively;
e) SEQ ID NOs: 143 and 82, respectively;
f) SEQ ID NOs: 144 and 82, respectively;
g) SEQ ID NOs: 145 and 82, respectively;
h) SEQ ID NOs: 146 and 148, respectively; or
i) SEQ ID NOs: 147 and 82, respectively.

27) The antibody or the antigen-binding fragment thereof of any one of claims 1-26, wherein the antibody
a) is an IgG1 isotype;
b) is an IgG2 isotype;
c) is an IgG3 isotype;
d) is an IgG4 isotype;
e) comprises at least one substitution in an Fc region that modulates binding of the antibody to FcγR or FcRn;
f) is an IgG2 isotype comprising V234A, G237A, P238S, H268A, V309L, A330S and P331S substitutions when compared to the wild-type IgG2;
g) is an IgG1 isotype comprising L234A, L235A, G237A, P238S, H268A, A330S and P331S substitutions when compared to the wild-type IgG1;
h) is an IgG1 isotype comprising L234A and L235A substitutions when compared to the wild-type IgG1; or
i) is an IgG4 isotype comprising S228P, F234A and L235A substitutions when compared to the wild-type IgG4.

28) The antibody of claim 27, comprising a heavy chain (HC) and a light chain (LC) of
a) SEQ ID NOs: 84 and 88, respectively;
b) SEQ ID NOs: 85 and 89, respectively;
c) SEQ ID NOs: 86 and 89, respectively;
d) SEQ ID NOs: 87 and 89, respectively;
e) SEQ ID NOs: 96 and 88 respectively;
f) SEQ DI NOs: 97 and 89, respectively;
g) SEQ ID NOs: 98 and 89, respectively;
h) SEQ ID NOs: 99 and 89, respectively;
i) SEQ ID NOs: 149 and 89, respectively;
j) SEQ ID NOs: 150 and 89, respectively;
k) SEQ ID NOs: 151 and 89, respectively;
l) SEQ ID NOs: 152 and 154, respectively; or
m) SEQ ID NOs: 153 and 89, respectively.

29) The antibody of claim 28, wherein the HC and the LC are encoded by polynucleotides of
a) SEQ ID NOs: 90 and 94, respectively;
b) SEQ ID NOs: 91 and 95, respectively;
c) SEQ ID NOs: 92 and 95, respectively;
d) SEQ ID NOs: 93 and 95, respectively;
e) SEQ ID NOs: 100 and 94, respectively;
f) SEQ ID NOs: 101 and 95, respectively;
g) SEQ ID NOs: 102 and 95, respectively;
h) SEQ ID NOs: 103 and 95, respectively;
i) SEQ ID NOs: 155 and 95, respectively;
j) SEQ ID NOs: 156 and 95, respectively;
k) SEQ ID NOs: 157 and 95, respectively;
l) SEQ ID NOs: 158 and 160, respectively; or
m) SEQ ID NOs: 159 and 95, respectively.

30) An isolated antibody or an antigen-binding fragment thereof that specifically binds HLA-DR comprising
a) the HCDR1, the HCDR2, the HCDR3, LCDR1, the LCDR2 and the LCDR3 of SEQ ID NOs: 39, 42, 46, 50, 52 and 54, respectively;
b) the VH of SEQ ID NO: 56 and the VL of SEQ ID NO: 60; and/or
c) the HC of SEQ ID NO: 84 or 96 and the LC of SEQ ID NO: 88.

31) An isolated antibody or an antigen-binding fragment thereof that specifically binds HLA-DR comprising
a) the HCDR1, the HCDR2, the HCDR3, the LCDR1, the LCDR2 and the LCDR3 of SEQ ID NOs: 40, 43, 47, 51, 53 and 55, respectively;
b) the VH of SEQ ID NO: 57 and the VL of SEQ ID NO: 61; and/or
c) the HC of SEQ ID NO: 85 or 97 and the LC of SEQ ID NO: 89.

32) An isolated antibody or an antigen-binding fragment thereof that specifically binds HLA-DR comprising
a) the HCDR1, the HCDR2, the HCDR3, the LCDR1, the LCDR2 and the LCDR3 of SEQ ID NOs: 41, 44, 48, 51, 53 and 55, respectively;
b) the VH of SEQ ID NO: 58 and the VL of SEQ ID NO: 61; and/or
c) the HC of SEQ ID NO: 86 or 98 and the LC of SEQ ID NO: 89.

33) An isolated antibody or an antigen-binding fragment thereof that specifically binds HLA-DR comprising
a) the HCDR1, the HCDR2, the HCDR3, the LCDR1, the LCDR2 and the LCDR3 of SEQ ID NOs: 41, 45, 49, 51, 53 and 55, respectively;
b) the VL of SEQ ID NO: 59 and the VL of SEQ ID NO: 61; and/or
c) the HC of SEQ ID NO: 87 or 99 and the LC of SEQ ID NO: 89.

34) The antibody or the antigen-binding fragment thereof of any one of claims 1-33, wherein the antibody is conjugated to a heterologous molecule.

35) The antibody or the antigen-binding fragment thereof of claim 34, wherein the heterologous molecule is a detectable label or a cytotoxic agent.

36) The antibody or the antigen-binding fragment thereof of any one of claims 1-35, wherein the antibody is a multispecific or a bispecific antibody.

37) A pharmaceutical composition comprising the antibody or the antigen-binding fragment of any one of claims 1-36 and a pharmaceutically accepted carrier.

38) A polynucleotide
a) encoding the VH, the VL, the VH and the VL, the HC, the LC or the HC and the LC of SEQ ID NOs: 56, 57, 58, 59, 60, 61, 84, 85, 86, 87, 96, 97, 98, 99, 137, 138, 139, 140, 141, 142, 149, 150, 151, 152, 153 or 154; or
b) comprising the polynucleotide sequence of SEQ ID NOs: 79, 80, 81, 82, 83, 90, 91, 92, 93, 94, 95, 100, 101, 102, 103, 121, 143, 144, 145, 146, 147, 148, 155, 156, 157, 158, 159 or 160.

39) A vector comprising the polynucleotide of claim 38.

40) A host cell comprising the vector of claim 39.

41) A method of producing the antibody or the antigen-binding fragment thereof of claim 25, comprising culturing the host cell of claim 40 in conditions that the antibody is expressed, and recovering the antibody produced by the host cell.

42) A method of treating or preventing HLA-DR-mediated disease, comprising administering to a subject in need thereof a therapeutically effective amount of the antibody or the antigen-binding fragment thereof of any one of claims 1-36 or the pharmaceutical composition of claim 37 for a time sufficient to treat HLA-DR-mediated disease.

43) The method of claim 42, wherein HLA-DR-mediated disease is an autoimmune disease.

44) The method of claim 43, wherein the autoimmune disease is HLA-DRB1-associated autoimmune disease, rheumatoid arthritis, systemic juvenile idiopathic arthritis, Grave's disease, Hashimoto's thyroiditis, myasthenia gravis, multiple sclerosis, systemic lupus erythematosus or Type 1 Diabetes.

45) The method of any one of claims 42-44, wherein the antibody or the antigen-binding fragment thereof is administered in combination with a second therapeutic agent.

46) The method of claim 45, wherein the second therapeutic agent is a corticosteroid or an immunosuppressant.

47) A method of suppressing an immune response towards a self-antigen, comprising administering to a subject in need thereof the antibody or the antigen-binding fragment thereof of any one of claims 1-36 or the pharmaceutical composition of claim 37 for a time sufficient to suppress the immune response towards a self-antigen.

48) The method of claim 47, wherein the self-antigen is present in a patient with an autoimmune disease.

49) The method of claim 48, wherein the autoimmune disease is rheumatoid arthritis, systemic juvenile idiopathic arthritis, Grave's disease, Hashimoto's thyroiditis, myasthenia gravis, multiple sclerosis, systemic lupus erythematosus or Type 1 Diabetes.

50) A method of treating HLA-DR expressing tumor, comprising administering to a subject in need thereof a therapeutically effective amount of the antibody or the antigen-binding fragment thereof of any one of claims 1-36 or the pharmaceutical composition of claim 37 conjugated to a cytotoxic agent for a time sufficient to treat HLA-DR expressing tumor.

51) The method of claim 50, wherein HLA-DR expressing tumor is a hematological malignancy.

52) The method of claim 51, wherein the hematological malignancy is B cell non-Hodgkin's lymphoma, B cell lymphoma, B cell acute lymphoid leukemia, Burkitt's lymphoma, Hodgkin's lymphoma, hairy cell leukemia, acute myeloid leukemia, T cell lymphoma, T cell non-Hodgkin's lymphoma, chronic myeloid leukemia, chronic lymphoid leukemia, multiple myeloid leukemia or acute monoblastic leukemia (AMoL).

53) The method of claim 52, wherein the HLA-DR expressing tumor is a glioma, an ovarian cancer, a colorectal cancer, an osteosarcoma, a cervical cancer, a stomach cancer or a tumor in the colon, larynx, skeletal muscle, breast or lung.

54) An anti-idiotypic antibody binding to the antibody or the antigen-binding fragment thereof of claim 25.

55) A kit comprising the antibody or the antigen-binding fragment of claim 25.

56) The antibody of any one of claims 1-36 or the pharmaceutical composition of claim 37 for use in therapy.

57) The antibody of any one of claims 1-36 or the pharmaceutical composition of claim 37 for use in the treatment of HLA-DR-mediated disease, an autoimmune disease or cancer.


SEQUENCE LISTING (162 sequences)

SEQ ID NO: 1 - protein, 5 aa, Artificial sequence
  Linker
GGGGS

SEQ ID NO: 2 - protein, 6 aa, Artificial sequence
  TEV cleavage site
EDLYFQ

SEQ ID NO: 3 - protein, 6 aa, Artificial sequence
  hexahistidine tag
HHHHHH

SEQ ID NO: 4 - protein, 6 aa, Artificial sequence
  Linker
GSGSGS

SEQ ID NO: 5 - protein, 8 aa, Artificial sequence
  HRV3C cleavage site
LEVLFQGP

SEQ ID NO: 6 - protein, 8 aa, Artificial sequence
  StrepII tag
WSHPQFEK

SEQ ID NO: 7 - protein, 15 aa, Homo sapiens
ACPKYVKQNTLKLAT

SEQ ID NO: 8 - protein, 14 aa, Homo sapiens
LQYMRADQAAGGLR

SEQ ID NO: 9 - protein, 17 aa, Homo sapiens
EPGIAGFKGEQGPKGEP

SEQ ID NO: 10 - protein, 14 aa, Homo sapiens
FVNQLCGSHLVEAL

SEQ ID NO: 11 - protein, 13 aa, Homo sapiens
SLLMWITQCFLPV

SEQ ID NO: 12 - protein, 220 aa, Artificial sequence
  Engineered IgG4 Fc
CPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 13 - protein, 191 aa, Homo sapiens
IKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTE

SEQ ID NO: 14 - protein, 198 aa, Homo sapiens
GDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSK

SEQ ID NO: 15 - protein, 198 aa, Homo sapiens
GDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSK

SEQ ID NO: 16 - protein, 194 aa, Homo sapiens
EDIVADHVASCGVNLYQFYGPSGQYTHEFDGDEQFYVDLERKETAWRWPEFSKFGGFDPQGALRNMAVAKHNLNIMIKRYNSTAATNEVPEVTVFSKSPVTLGQPNTLICLVDNIFPPVVNITWLSNGQSVTEGVSETSFLSKSDHSFFKISYLTFLPSADEIYDCKVEHWGLDQPLLKHWEPEIPAPMSELTE

SEQ ID NO: 17 - protein, 198 aa, Homo sapiens
RDSPEDFVFQFKGMCYFTNGTERVRLVTRYIYNREEYARFDSDVGVYRAVTPQGRPDAEYWNSQKEVLEGTRAELDTVCRHNYEVAFRGILQRRVEPTVTISPSRTEALNHHNLLVCSVTDFYPGQIKVRWFRNDQEETAGVVSTPLIRNGDWTFQILVMLEMTPQRGDVYTCHVEHPSLQSPITVEWRAQSESAQSK

SEQ ID NO: 18 - protein, 194 aa, Homo sapiens
AGAIKADHVSTYAAFVQTHRPTGEFMFEFDEDEMFYVDLDKKETVWHLEEFGQAFSFEAQGGLANIAILNNNLNTLIQRSNHTQATNDPPEVTVFPKEPVELGQPNTLICHIDKFFPPVLNVTWLCNGELVTEGVAESLFLPRTDYSFHKFHYLTFVPSAEDFYDCRVEHWGLDQPLLKHWEAQEPIQMPETTE

SEQ ID NO: 19 - protein, 196 aa, Homo sapiens
RATPENYLFQGRQECYAFNGTQRFLERYIYNREEFARFDSDVGEFRAVTELGRPAAEYWNSQKDILEEKRAVPDRMCRHNYELGGPMTLQRRVQPRVNVSPSKKGPLQHHNLLVCHVTDFYPGSIQVRWFLNGQEETAGVVSTNLIRNGDWTFQILVMLEMTPQQGDVYTCQVEHTSLDSPVTVEWKAQSDSARSK

SEQ ID NO: 20 - protein, 436 aa, Artificial sequence
  DR4G89, DR4G90, DR4G92, DR4G93, DR4G99, DR4G102 alpha chain
IKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTEGGGGSEDLYFQSGGGGSCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKHHHHHH

SEQ ID NO: 21 - protein, 474 aa, Artificial sequence
  DR4G89  beta chain
ACPKYVKQNTLKLATGSGSGSLEVLFQGPGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSKGGGGSEDLYFQSGGGGSCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKWSHPQFEK

SEQ ID NO: 22 - protein, 473 aa, Artificial sequence
  DR4G90 bet achain
LQYMRADQAAGGLRGSGSGSLEVLFQGPGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSKGGGGSEDLYFQSGGGGSCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKWSHPQFEK

SEQ ID NO: 23 - protein, 476 aa, Artificial sequence
  DR4G92 beta chain
EPGIAGFKGEQGPKGEPGSGSGSLEVLFQGPGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSKGGGGSEDLYFQSGGGGSCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKWSHPQFEK

SEQ ID NO: 24 - protein, 474 aa, Artificial sequence
  DR4G93 beta chain
ACPKYVKQNTLKLATGSGSGSLEVLFQGPGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKGGGGSEDLYFQSGGGGSCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKWSHPQFEK

SEQ ID NO: 25 - protein, 473 aa, Artificial sequence
  DR4G99 beta chain
LQYMRADQAAGGLRGSGSGSLEVLFQGPGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKGGGGSEDLYFQSGGGGSCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKWSHPQFEK

SEQ ID NO: 26 - protein, 476 aa, Artificial sequence
  DR4G102 beta chain
EPGIAGFKGEQGPKGEPGSGSGSLEVLFQGPGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKGGGGSEDLYFQSGGGGSCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKWSHPQFEK

SEQ ID NO: 27 - protein, 440 aa, Artificial sequence
  DR4G111, DR4G112 alpha chain
EDIVADHVASCGVNLYQFYGPSGQYTHEFDGDEQFYVDLERKETAWRWPEFSKFGGFDPQGALRNMAVAKHNLNIMIKRYNSTAATNEVPEVTVFSKSPVTLGQPNTLICLVDNIFPPVVNITWLSNGQSVTEGVSETSFLSKSDHSFFKISYLTFLPSADEIYDCKVEHWGLDQPLLKHWEPEIPAPMSELTEGGGGSEDLYFQSGGGGSCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGSHHHHHH

SEQ ID NO: 28 - protein, 474 aa, Artificial sequence
  DR4G111 beta chain
FVNQHLCGSHLVEALGSGSGSLEVLFQGPRDSPEDFVFQFKGMCYFTNGTERVRLVTRYIYNREEYARFDSDVGVYRAVTPQGRPDAEYWNSQKEVLEGTRAELDTVCRHNYEVAFRGILQRRVEPTVTISPSRTEALNHHNLLVCSVTDFYPGQIKVRWFRNDQEETAGVVSTPLIRNGDWTFQILVMLEMTPQRGDVYTCHVEHPSLQSPITVEWRAQSESAQSKGGGGSEDLYFQSGGGGSCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKWSHPQFEK

SEQ ID NO: 29 - protein, 440 aa, Artificial sequence
  DR4G113 alpha chain
AGAIKADHVSTYAAFVQTHRPTGEFMFEFDEDEMFYVDLDKKETVWHLEEFGQAFSFEAQGGLANIAILNNNLNTLIQRSNHTQATNDPPEVTVFPKEPVELGQPNTLICHIDKFFPPVLNVTWLCNGELVTEGVAESLFLPRTDYSFHKFHYLTFVPSAEDFYDCRVEHWGLDQPLLKHWEAQEPIQMPETTEGGGGSEDLYFQSGGGGSCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGSHHHHHH

SEQ ID NO: 30 - protein, 470 aa, Artificial sequence
  DR4G113 beta chain
SLLMWITQCFLPVGSGSGSLEVLFQGPRATPENYLFQGRQECYAFNGTQRFLERYIYNREEFARFDSDVGEFRAVTELGRPAAEYWNSQKDILEEKRAVPDRMCRHNYELGGPMTLQRRVQPRVNVSPSKKGPLQHHNLLVCHVTDFYPGSIQVRWFLNGQEETAGVVSTNLIRNGDWTFQILVMLEMTPQQGDVYTCQVEHTSLDSPVTVEWKAQSDSARSKGGGGSEDLYFQSGGGGSCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKWSHPQFEK

SEQ ID NO: 31 - protein, 118 aa, Mus musculus
QVQLKESGPGLVAPSQSLSITCTISGFSLTSYGVHWVRQPPGKGLEWLVVIWSDGSTTYNSALKSRLSISKDNSKSQVFLKMNSLQTDDTAIYYCASHYGSTLAFASWGHGTLVTVSA

SEQ ID NO: 32 - protein, 106 aa, Mus musculus
DIQMTQSPASLSASVGETVTIICRASVNIYSYLAWYQQKQGKSPQLLVYNAKILAEGVPSRFSGSGSGTQFSLKINSLQPEDFGSYYCQHHYGPFTFGSGTKLEIK

SEQ ID NO: 33 - protein, 116 aa, Artificial sequence
  Apolizumab VH
QVQLQESGPGLVKPSETLSLTCTVSGFSLTNYGVHWVRQSPGKGLEWIGVKWSGGSTEYNAAFISRLTISKDTSKNQVSLKLNSLTAADTAVYYCARNDRYAMDYWGQGTLVTVSS

SEQ ID NO: 34 - protein, 107 aa, Artificial sequence
  Apolizumab VL
DIQMTQSPSSLSASVGDRVTITCRASENIYSYLAWYQQKPGKAPKLLVSNAKTLAEGVPSRFSGSGSGKQFTLTISSLQPEDFATYYCQHHYGNSYPFGQGTKLEIK

SEQ ID NO: 35 - protein, 121 aa, Mus musculus
QIQLVQSGPELKKPGETVKISCKASGFTFTNYGMNWVKQAPGKGLKWMGWINTYTREPTYADDFKGRFAFSLETSASTAYLQINNLKNEDTAKYFCARDITAVVPTGFDYWGQGTTLTVSS

SEQ ID NO: 36 - protein, 107 aa, Mus musculus
DIQMTQSPASLSVSVGETVTITCRASENIYSNLAWYRQKQGKSPQLLVFAASNLADGVPSRFSGSGSGTQYSLKINSLQSEDFGDYYCQHFWTTPWAFGGGTNLEIK

SEQ ID NO: 37 - protein, 9 aa, Homo sapiens
NTWTTCQSI

SEQ ID NO: 38 - protein, 468 aa, Artificial sequence
  DR4G112 beta chain
NTWTTCQSIGSGSGSLEVLFQGPRDSPEDFVFQFKGMCYFTNGTERVRLVTRYIYNREEYARFDSDVGVYRAVTPQGRPDAEYWNSQKEVLEGTRAELDTVCRHNYEVAFRGILQRRVEPTVTISPSRTEALNHHNLLVCSVTDFYPGQIKVRWFRNDQEETAGVVSTPLIRNGDWTFQILVMLEMTPQRGDVYTCHVEHPSLQSPITVEWRAQSESAQSKGGGGSEDLYFQSGGGGSCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKWSHPQFEK

SEQ ID NO: 39 - protein, 5 aa, Artificial sequence
  DR4B117 HCDR1
SYSIH

SEQ ID NO: 40 - protein, 5 aa, Artificial sequence
  DR4B30 HCDR1
SDWIG

SEQ ID NO: 41 - protein, 5 aa, Artificial sequence
  DR4B127 and DR4B98 HCDR1
SYYIH

SEQ ID NO: 42 - protein, 17 aa, Artificial sequence
  DR4B117 HCDR2
YIIPEYGTANYAQKFQG

SEQ ID NO: 43 - protein, 17 aa, Artificial sequence
  DR4B30 HCDR2
IIRPGDSDTYYSPSFQG

SEQ ID NO: 44 - protein, 17 aa, Artificial sequence
  DR4B127  HCDR2
GIRPISGNAEYAQKFQG

SEQ ID NO: 45 - protein, 17 aa, Artificial sequence
  DR4B98 HCDR2
GIAPIYGTAYYAQKFQG

SEQ ID NO: 46 - protein, 17 aa, Artificial sequence
  DR4B117 HCDR3
GRYYIGNRRGSYYGFDY

SEQ ID NO: 47 - protein, 18 aa, Artificial sequence
  DR4B30 HCDR3
ESYYYVGVRYRPSYYFDY

SEQ ID NO: 48 - protein, 12 aa, Artificial sequence
  DR4B127  HCDR3
DASYYRNYGFDY

SEQ ID NO: 49 - protein, 12 aa, Artificial sequence
  DR4B98 HCDR3
DASWARAYGFDY

SEQ ID NO: 50 - protein, 12 aa, Artificial sequence
  DR4B117 LCDR1
RASQSVSSSYLA

SEQ ID NO: 51 - protein, 11 aa, Artificial sequence
  DR4B30, DR4B127, DR4B98 LCDR1
RASQSVSSYLA

SEQ ID NO: 52 - protein, 7 aa, Artificial sequence
  DR4B117 LCDR2
GASSRAT

SEQ ID NO: 53 - protein, 7 aa, Artificial sequence
  DR4B30, DR4B127, DR4B98 LCDR2
DASNRAT

SEQ ID NO: 54 - protein, 9 aa, Artificial sequence
  DR4B117 LCDR3
QQYGSSPLT

SEQ ID NO: 55 - protein, 9 aa, Artificial sequence
  DR4B30, DR4B127, DR4B98 LCDR3
QQRSNWPLT

SEQ ID NO: 56 - protein, 126 aa, Artificial sequence
  DR4B117 VH (DR4H4) amino acid
QVQLVQSGAEVKKPGSSVKVSCKASGGTFSSYSIHWVRQAPGQGLEWMGYIIPEYGTANYAQKFQGRVTITADESTSTAYMELSSLRSEDTAVYYCARGRYYIGNRRGSYYGFDYWGQGTLVTVSS

SEQ ID NO: 57 - protein, 127 aa, Artificial sequence
  DR4B30 VH (DR4H39) amino acid
EVQLVQSGAEVKKPGESLKISCKGSGYSFTSDWIGWVRQMPGKGLEWMGIIRPGDSDTYYSPSFQGQVTISADKSISTAYLQWSSLKASDTAVYYCARESYYYVGVRYRPSYYFDYWGQGTLVTVSS

SEQ ID NO: 58 - protein, 121 aa, Artificial sequence
  DR4B127 VH (DR4H7) amino acid
QVQLVQSGAEVKKPGSSVKVSCKASGGTFKSYYIHWVRQAPGQGLEWMGGIRPISGNAEYAQKFQGRVTITADESTSTAYMELSSLRSEDTAVYYCARDASYYRNYGFDYWGQGTLVTVSS

SEQ ID NO: 59 - protein, 121 aa, Artificial sequence
  DR4B98 VH (DR4H50) amino acid
QVQLVQSGAEVKKPGSSVKVSCKASGGTFKSYYIHWVRQAPGQGLEWMGGIAPIYGTAYYAQKFQGRVTITADESTSTAYMELSSLRSEDTAVYYCARDASWARAYGFDYWGQGTLVTVSS

SEQ ID NO: 60 - protein, 108 aa, Artificial sequence
  DR4B117 VL (PH9L1) amino acid
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIK

SEQ ID NO: 61 - protein, 107 aa, Artificial sequence
  DR4B30, DR4B127, DR4B98 VL
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGQGTKVEIK

SEQ ID NO: 62 - protein, 98 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCAR

SEQ ID NO: 63 - protein, 98 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCAR

SEQ ID NO: 64 - protein, 96 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSP

SEQ ID NO: 65 - protein, 95 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWP

SEQ ID NO: 66 - protein, 5 aa, Homo sapiens
QKRAA

SEQ ID NO: 67 - protein, 5 aa, Homo sapiens
QRRAA

SEQ ID NO: 68 - protein, 5 aa, Homo sapiens
RRRAA

SEQ ID NO: 69 - protein, 462 aa, Artificial sequence
  DR4G79 HC
QSVTQLGSHVSVSEGALVLLRCNYSSSVPPYLFWYVQYPNQGLQLLLKYTSAATLVKGINGFEAEFKKSETSFHLTKPSAHMSDAAEYFCAVSESPFGNEKLTFGTGTRLTIIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKGGGGSEDLYFQSGGGGSCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSHHHHHH

SEQ ID NO: 70 - protein, 508 aa, Artificial sequence
  DRG79 LC
GSVKVTQSSRYLVKRTGEKVFLECVQDMDHENMFWYRQDPGLGLRLIYFSYDVKMKEKGDIPEGYSVSREKKERFSLILESASTNQTSMYLCASSSTGLPYGYTFGSGTRLTVVEDLNKVFPPEVAVFEPSEAEISHTQKATLVCLATGFFPDHVELSWWVNGKEVHSGVSTDPQPLKEQPALNDSRYSLSSRLRVSATFWQNPRNHFRCQVQFYGLSENDEWTQDRAKPVTQIVSAEAWGRADCGFTGGGGSEDLYFQSGGGGSCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSDYKDDDDKWSHPQFEK

SEQ ID NO: 71 - protein, 13 aa, Artificial sequence
  Vimentin L70A mutant peptide
SAVRARSSVPGVR

SEQ ID NO: 72 - protein, 21 aa, Homo sapiens
EVVLLVATEGRVRVNSAYQDK

SEQ ID NO: 73 - protein, 5 aa, Artificial sequence
  HCDR1 genus
  MISC_FEATURE (2)..(2): Xaa may be Tyr or Asp
  MISC_FEATURE (3)..(3): Xaa may be Ser, Trp or Tyr
  MISC_FEATURE (5)..(5): Xaa may be His or Gly
SXXIX

SEQ ID NO: 74 - protein, 17 aa, Artificial sequence
  HCDR2 genus
  MISC_FEATURE (3)..(3): Xaa may be Arg or Ala
  MISC_FEATURE (6)..(6): Xaa may be Ser or Tyr
  MISC_FEATURE (8)..(8): Xaa may be Asn or Thr
  MISC_FEATURE (10)..(10): Xaa may be Glu or Tyr
GIXPIXGXAXYAQKFQG

SEQ ID NO: 75 - protein, 12 aa, Artificial sequence
  HCDR3 genus
  MISC_FEATURE (4)..(4): Xaa may be Tyr or Trp
  MISC_FEATURE (5)..(5): Xaa may be Tyr or Ala
  MISC_FEATURE (7)..(7): Xaa may be Asn or Ala
DASXXRXYGFDY

SEQ ID NO: 76 - protein, 12 aa, Artificial sequence
  LCDR1 genus
  MISC_FEATURE (9)..(9): Xaa may be Ser or deleted
RASQSVSSXYLA

SEQ ID NO: 77 - protein, 7 aa, Artificial sequence
  LCDR2 genus
  MISC_FEATURE (1)..(1): Xaa may be Gly or Asp
  MISC_FEATURE (4)..(): Xaa may be Ser or Asn
  misc_feature (4)..(4): Xaa can be any naturally occurring amino acid
XASXRAT

SEQ ID NO: 78 - protein, 9 aa, Artificial sequence
  LCDR3 genus
  MISC_FEATURE (3)..(3): Xaa may be Tyr or Arg
  MISC_FEATURE (4)..(4): Xaa may be  Gly or Ser
  MISC_FEATURE (5)..(5): Xaa may be Ser or Asn
  MISC_FEATURE (6)..(6): Xaa may be Ser or Trp
QQXXXXPLT

SEQ ID NO: 79 - DNA, 378 bp, Artificial sequence
  DR4B117 VH (DR4H4) polynucleotide
caggtgcagctggtgcagagcggcgcggaagtgaaaaaaccgggcagcagcgtgaaagtgagctgcaaagcgagcggcggcacctttagcagctattccattcactgggtgcgccaggcgccgggccagggcctggaatggatgggctacattattccggagtacgggactgccaattacgcgcagaaatttcagggccgcgtgaccattaccgctgatgaaagcaccagcaccgcgtatatggaactgagcagcctgcgcagcgaagataccgcggtgtattattgcgcgcgcggccgatactatatcggcaaccgtcgtggcagttattacggttttgactattggggccagggcaccctggtgaccgtctcgagt

SEQ ID NO: 80 - DNA, 324 bp, Artificial sequence
  DR4B117 VL (PH9L1) polynucleotide
gagatcgtgctgacccagagccccggcaccctgagcctgagccccggcgagcgggccaccctgagctgccgggccagccagagcgtgagcagcagctacctggcctggtaccagcagaagcccggccaggccccccggctgctgatctacggcgccagcagccgggccaccggcatccccgaccggttcagcggcagcggcagcggcaccgacttcaccctgaccatcagccggctggagcccgaggacttcgccgtgtactactgccagcagtacggcagcagccccctgaccttcggccagggcaccaaggtggagatcaag

SEQ ID NO: 81 - DNA, 381 bp, Artificial sequence
  DR4B30 VH (DR4H39) polynucleotide
gaagtgcagctggtgcagagcggcgcggaagtgaaaaaaccgggcgaaagcctgaaaattagctgcaaaggcagcggctatagctttaccagcgactggattggttgggtgcgccagatgccgggcaaaggcttggaatggatgggtatcattcgcccgggcgatagcgatacgtattacagcccgagctttcagggccaggtgaccattagcgcggataaaagcattagcaccgcgtatctgcagtggagcagcctgaaagcgagcgataccgcggtgtattattgcgcgcgtgaatcctattattacgttggcgtgcgttaccgtccaagctattatttcgattactggggccagggcaccctggtgaccgtctcgagt

SEQ ID NO: 82 - DNA, 321 bp, Artificial sequence
  DR4B30, DR4B127, DR4B98 VL
gagatcgtgctgacccagagccccgccaccctgagcctgagccccggcgagcgggccaccctgagctgccgggccagccagagcgtgagcagctacctggcctggtaccagcagaagcccggccaggccccccggctgctgatctacgacgccagcaaccgggccaccggcatccccgcccggttcagcggcagcggcagcggcaccgacttcaccctgaccatcagcagcctggagcccgaggacttcgccgtgtactactgccagcagcggagcaactggcccctgaccttcggccagggcaccaaggtggagatcaag

SEQ ID NO: 83 - DNA, 363 bp, Artificial sequence
  DR4B127 VH (DR4H7) polynucleotide
caggtgcagctggtgcagagcggcgcggaagtgaaaaaaccgggcagcagcgtgaaagtgagctgcaaagcgagcggcggcacctttaaatcctactacattcactgggtgcgccaggcgccgggccagggcctggaatggatgggtggtattcgtccgatcagcgggaatgctgagtacgcgcagaaatttcagggccgcgtgaccattaccgctgatgaaagcaccagcaccgcgtatatggaactgagcagcctgcgcagcgaagataccgcggtgtattattgcgcgcgcgatgcaagctattatcgtaattacggttttgactactggggccagggcaccctggtgaccgtctcgagt

SEQ ID NO: 84 - protein, 452 aa, Artificial sequence
  DR4B117 HC amino acid
QVQLVQSGAEVKKPGSSVKVSCKASGGTFSSYSIHWVRQAPGQGLEWMGYIIPEYGTANYAQKFQGRVTITADESTSTAYMELSSLRSEDTAVYYCARGRYYIGNRRGSYYGFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPAAASSVFLFPPKPKDTLMISRTPEVTCVVVDVSAEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 85 - protein, 453 aa, Artificial sequence
  DR4B30 HC amino acid
EVQLVQSGAEVKKPGESLKISCKGSGYSFTSDWIGWVRQMPGKGLEWMGIIRPGDSDTYYSPSFQGQVTISADKSISTAYLQWSSLKASDTAVYYCARESYYYVGVRYRPSYYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPAAASSVFLFPPKPKDTLMISRTPEVTCVVVDVSAEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 86 - protein, 447 aa, Artificial sequence
  DR4B127 HC amino acid
QVQLVQSGAEVKKPGSSVKVSCKASGGTFKSYYIHWVRQAPGQGLEWMGGIRPISGNAEYAQKFQGRVTITADESTSTAYMELSSLRSEDTAVYYCARDASYYRNYGFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPAAASSVFLFPPKPKDTLMISRTPEVTCVVVDVSAEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 87 - protein, 447 aa, Artificial sequence
  DR4B98 HC amino acid
QVQLVQSGAEVKKPGSSVKVSCKASGGTFKSYYIHWVRQAPGQGLEWMGGIAPIYGTAYYAQKFQGRVTITADESTSTAYMELSSLRSEDTAVYYCARDASWARAYGFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPAAASSVFLFPPKPKDTLMISRTPEVTCVVVDVSAEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 88 - protein, 215 aa, Artificial sequence
  DR4B117 LC amino acid
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 214 aa, Artificial sequence
  DR4B30, DR4B127, DR4B98 LC
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - DNA, 1356 bp, Artificial sequence
  DR4B117 HC
caggtgcagctggtgcagagcggcgcggaagtgaaaaaaccgggcagcagcgtgaaagtgagctgcaaagcgagcggcggcacctttagcagctattccattcactgggtgcgccaggcgccgggccagggcctggaatggatgggctacattattccggagtacgggactgccaattacgcgcagaaatttcagggccgcgtgaccattaccgctgatgaaagcaccagcaccgcgtatatggaactgagcagcctgcgcagcgaagataccgcggtgtattattgcgcgcgcggccgatactatatcggcaaccgtcgtggcagttattacggttttgactattggggccagggcaccctggtgaccgtctcgagtgcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgccgcagccagctcagtcttcctcttccccccaaaacccaaggacaccctcatgatctcccggacccctgaggtcacgtgcgtggtggtggacgtgagcgccgaagaccccgaggtccagttcaactggtacgtggacggcgtggaggtgcataatgccaagacaaagccacgggaggagcagttcaacagcacgttccgtgtggtcagcgtcctcaccgttctgcaccaggactggctgaacggcaaggagtacaagtgcaaggtctccaacaaaggcctcccatcctccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 91 - DNA, 1359 bp, Artificial sequence
  DR4B30 HC
gaagtgcagctggtgcagagcggcgcggaagtgaaaaaaccgggcgaaagcctgaaaattagctgcaaaggcagcggctatagctttaccagcgactggattggttgggtgcgccagatgccgggcaaaggcttggaatggatgggtatcattcgcccgggcgatagcgatacgtattacagcccgagctttcagggccaggtgaccattagcgcggataaaagcattagcaccgcgtatctgcagtggagcagcctgaaagcgagcgataccgcggtgtattattgcgcgcgtgaatcctattattacgttggcgtgcgttaccgtccaagctattatttcgattactggggccagggcaccctggtgaccgtctcgagtgcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgccgcagccagctcagtcttcctcttccccccaaaacccaaggacaccctcatgatctcccggacccctgaggtcacgtgcgtggtggtggacgtgagcgccgaagaccccgaggtccagttcaactggtacgtggacggcgtggaggtgcataatgccaagacaaagccacgggaggagcagttcaacagcacgttccgtgtggtcagcgtcctcaccgttctgcaccaggactggctgaacggcaaggagtacaagtgcaaggtctccaacaaaggcctcccatcctccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 92 - DNA, 1341 bp, Artificial sequence
  DR4B127  HC
caggtgcagctggtgcagagcggcgcggaagtgaaaaaaccgggcagcagcgtgaaagtgagctgcaaagcgagcggcggcacctttaaatcctactacattcactgggtgcgccaggcgccgggccagggcctggaatggatgggtggtattcgtccgatcagcgggaatgctgagtacgcgcagaaatttcagggccgcgtgaccattaccgctgatgaaagcaccagcaccgcgtatatggaactgagcagcctgcgcagcgaagataccgcggtgtattattgcgcgcgcgatgcaagctattatcgtaattacggttttgactactggggccagggcaccctggtgaccgtctcgagtgcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgccgcagccagctcagtcttcctcttccccccaaaacccaaggacaccctcatgatctcccggacccctgaggtcacgtgcgtggtggtggacgtgagcgccgaagaccccgaggtccagttcaactggtacgtggacggcgtggaggtgcataatgccaagacaaagccacgggaggagcagttcaacagcacgttccgtgtggtcagcgtcctcaccgttctgcaccaggactggctgaacggcaaggagtacaagtgcaaggtctccaacaaaggcctcccatcctccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 93 - DNA, 1341 bp, Artificial sequence
  DR4B98 HC
caggtgcagctggtgcagagcggcgcggaagtgaaaaaaccgggcagcagcgtgaaagtgagctgcaaagcgagcggcggcacctttaagtcctattatattcattgggtgcgccaggcgccgggccagggcctggaatggatgggcggtattgcaccaatttacggcaccgcttactacgcgcagaaatttcagggccgcgtgaccattaccgctgatgaaagcaccagcaccgcgtatatggaactgagcagcctgcgcagcgaagataccgcggtgtattattgcgcgcgtgatgcaagttgggcacgtgcatacggttttgattattggggccagggcaccctggtgaccgtctcgagtgcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgccgcagccagctcagtcttcctcttccccccaaaacccaaggacaccctcatgatctcccggacccctgaggtcacgtgcgtggtggtggacgtgagcgccgaagaccccgaggtccagttcaactggtacgtggacggcgtggaggtgcataatgccaagacaaagccacgggaggagcagttcaacagcacgttccgtgtggtcagcgtcctcaccgttctgcaccaggactggctgaacggcaaggagtacaagtgcaaggtctccaacaaaggcctcccatcctccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 94 - DNA, 645 bp, Artificial sequence
  DR4B117 LC
gagatcgtgctgacccagagccccggcaccctgagcctgagccccggcgagcgggccaccctgagctgccgggccagccagagcgtgagcagcagctacctggcctggtaccagcagaagcccggccaggccccccggctgctgatctacggcgccagcagccgggccaccggcatccccgaccggttcagcggcagcggcagcggcaccgacttcaccctgaccatcagccggctggagcccgaggacttcgccgtgtactactgccagcagtacggcagcagccccctgaccttcggccagggcaccaaggtggagatcaagcggaccgtggccgcccccagcgtgttcatcttcccccccagcgacgagcagctgaagagcggaaccgcaagcgtggtgtgcctgctgaacaacttctacccccgggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcttgcgaggtgacccaccagggcctgagcagccccgtgaccaagagcttcaaccggggcgagtgc

SEQ ID NO: 95 - DNA, 642 bp, Artificial sequence
  DR4B30, DR4B127, DR4B98 LC
gagatcgtgctgacccagagccccgccaccctgagcctgagccccggcgagcgggccaccctgagctgccgggccagccagagcgtgagcagctacctggcctggtaccagcagaagcccggccaggccccccggctgctgatctacgacgccagcaaccgggccaccggcatccccgcccggttcagcggcagcggcagcggcaccgacttcaccctgaccatcagcagcctggagcccgaggacttcgccgtgtactactgccagcagcggagcaactggcccctgaccttcggccagggcaccaaggtggagatcaagcggaccgtggccgcccccagcgtgttcatcttcccccccagcgacgagcagctgaagagcggaaccgcaagcgtggtgtgcctgctgaacaacttctacccccgggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcttgcgaggtgacccaccagggcctgagcagccccgtgaccaagagcttcaaccggggcgagtgc

SEQ ID NO: 96 - protein, 456 aa, Artificial sequence
  DR4B391 HC protein
QVQLVQSGAEVKKPGSSVKVSCKASGGTFSSYSIHWVRQAPGQGLEWMGYIIPEYGTANYAQKFQGRVTITADESTSTAYMELSSLRSEDTAVYYCARGRYYIGNRRGSYYGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 457 aa, Artificial sequence
  DR4B396 HC protein
EVQLVQSGAEVKKPGESLKISCKGSGYSFTSDWIGWVRQMPGKGLEWMGIIRPGDSDTYYSPSFQGQVTISADKSISTAYLQWSSLKASDTAVYYCARESYYYVGVRYRPSYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 451 aa, Artificial sequence
  DR4B392 HC protein
QVQLVQSGAEVKKPGSSVKVSCKASGGTFKSYYIHWVRQAPGQGLEWMGGIRPISGNAEYAQKFQGRVTITADESTSTAYMELSSLRSEDTAVYYCARDASYYRNYGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 451 aa, Artificial sequence
  DR4B401 HC protein
QVQLVQSGAEVKKPGSSVKVSCKASGGTFKSYYIHWVRQAPGQGLEWMGGIAPIYGTAYYAQKFQGRVTITADESTSTAYMELSSLRSEDTAVYYCARDASWARAYGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - DNA, 1368 bp, Artificial sequence
  DR4B391 HC polynucleotide
caggtgcagctggtgcagagcggcgcggaagtgaaaaaaccgggcagcagcgtgaaagtgagctgcaaagcgagcggcggcacctttagcagctattccattcactgggtgcgccaggcgccgggccagggcctggaatggatgggctacattattccggagtacgggactgccaattacgcgcagaaatttcagggccgcgtgaccattaccgctgatgaaagcaccagcaccgcgtatatggaactgagcagcctgcgcagcgaagataccgcggtgtattattgcgcgcgcggccgatactatatcggcaaccgtcgtggcagttattacggttttgactattggggccagggcaccctggtgaccgtctcgagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagcagcaggggcatcttcagtcttcctcttccccccaaaacccaaggacaccctcatgatctcccggacccctgaggtcacatgcgtggtggtggacgtgagcgccgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 101 - DNA, 1371 bp, Artificial sequence
  DR4B396 HC polynucleotide
gaagtgcagctggtgcagagcggcgcggaagtgaaaaaaccgggcgaaagcctgaaaattagctgcaaaggcagcggctatagctttaccagcgactggattggttgggtgcgccagatgccgggcaaaggcttggaatggatgggtatcattcgcccgggcgatagcgatacgtattacagcccgagctttcagggccaggtgaccattagcgcggataaaagcattagcaccgcgtatctgcagtggagcagcctgaaagcgagcgataccgcggtgtattattgcgcgcgtgaatcctattattacgttggcgtgcgttaccgtccaagctattatttcgattactggggccagggcaccctggtgaccgtctcgagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagcagcaggggcatcttcagtcttcctcttccccccaaaacccaaggacaccctcatgatctcccggacccctgaggtcacatgcgtggtggtggacgtgagcgccgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 102 - DNA, 1353 bp, Artificial sequence
  DR4B392 HC polynucleotide
caggtgcagctggtgcagagcggcgcggaagtgaaaaaaccgggcagcagcgtgaaagtgagctgcaaagcgagcggcggcacctttaaatcctactacattcactgggtgcgccaggcgccgggccagggcctggaatggatgggtggtattcgtccgatcagcgggaatgctgagtacgcgcagaaatttcagggccgcgtgaccattaccgctgatgaaagcaccagcaccgcgtatatggaactgagcagcctgcgcagcgaagataccgcggtgtattattgcgcgcgcgatgcaagctattatcgtaattacggttttgactactggggccagggcaccctggtgaccgtctcgagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagcagcaggggcatcttcagtcttcctcttccccccaaaacccaaggacaccctcatgatctcccggacccctgaggtcacatgcgtggtggtggacgtgagcgccgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 103 - DNA, 1353 bp, Artificial sequence
  DR4B401 polynucleotide
caggtgcagctggtgcagagcggcgcggaagtgaaaaaaccgggcagcagcgtgaaagtgagctgcaaagcgagcggcggcacctttaagtcctattatattcattgggtgcgccaggcgccgggccagggcctggaatggatgggcggtattgcaccaatttacggcaccgcttactacgcgcagaaatttcagggccgcgtgaccattaccgctgatgaaagcaccagcaccgcgtatatggaactgagcagcctgcgcagcgaagataccgcggtgtattattgcgcgcgtgatgcaagttgggcacgtgcatacggttttgattattggggccagggcaccctggtgaccgtctcgagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagcagcaggggcatcttcagtcttcctcttccccccaaaacccaaggacaccctcatgatctcccggacccctgaggtcacatgcgtggtggtggacgtgagcgccgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 104 - protein, 15 aa, Homo sapiens
KMRMATPLLMQALPM

SEQ ID NO: 105 - protein, 198 aa, Homo sapiens
GDTRPRFLEYSTSECHFFNGTERVRYLDRYFHNQEENVRFDSDVGEFRAVTELGRPDAEYWNSQKDLLEQKRGRVDNYCRHNYGVVESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSK

SEQ ID NO: 106 - protein, 198 aa, Homo sapiens
GDTRPRFLEQVKHECHFFNGTERVRFLDRYFYHQEEYVRFDSDVGEYRAVTELGRPDAEYWNSQKDILEDERAAVDTYCRHNYGVVESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSK

SEQ ID NO: 107 - protein, 198 aa, Homo sapiens
GDTRPRFLEEVKFECHFFNGTERVRLLERRVHNQEEYARYDSDVGEYRAVTELGRPDAEYWNSQKDLLERRRAAVDTYCRHNYGVGESFTVQRRVQPKVTVYPSKTQPLQHHNLLVCSVNGFYPGSIEVRWFRNGQEEKTGVVSTGLIQNGDWTFQTLVMLETVPQSGEVYTCQVEHPSVMSPLTVEWRARSESAQSK

SEQ ID NO: 108 - protein, 198 aa, Homo sapiens
GDTRPRFLWQPKRECHFFNGTERVRFLDRYFYNQEESVRFDSDVGEFRAVTELGRPDAEYWNSQKDILEQARAAVDTYCRHNYGVVESFTVQRRVQPKVTVYPSKTQPLQHHNLLVCSVSGFYPGSIEVRWFLNGQEEKAGMVSTGLIQNGDWTFQTLVMLETVPRSGEVYTCQVEHPSVTSPLTVEWRARSESAQSK

SEQ ID NO: 109 - protein, 472 aa, Artificial sequence
  DR4G143 beta chain
SAVRARSSVPGVRGSGSGSLEVLFQGPGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSKGGGGSEDLYFQSGGGGSCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKWSHPQFEK

SEQ ID NO: 110 - protein, 472 aa, Artificial sequence
  DR4G142 beta chain
SAVRARSSVPGVRGSGSGSLEVLFQGPGDTRPRFLEYSTSECHFFNGTERVRYLDRYFHNQEENVRFDSDVGEFRAVTELGRPDAEYWNSQKDLLEQKRGRVDNYCRHNYGVVESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSKGGGGSEDLYFQSGGGGSCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKWSHPQFEK

SEQ ID NO: 111 - protein, 474 aa, Artificial sequence
  DR4G119 beta chain
ACPKYVKQNTLKLATGSGSGSLEVLFQGPGDTRPRFLEEVKFECHFFNGTERVRLLERRVHNQEEYARYDSDVGEYRAVTELGRPDAEYWNSQKDLLERRRAAVDTYCRHNYGVGESFTVQRRVQPKVTVYPSKTQPLQHHNLLVCSVNGFYPGSIEVRWFRNGQEEKTGVVSTGLIQNGDWTFQTLVMLETVPQSGEVYTCQVEHPSVMSPLTVEWRARSESAQSKGGGGSEDLYFQSGGGGSCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKWSHPQFEK

SEQ ID NO: 112 - protein, 473 aa, Artificial sequence
  DR4G117 beta chain
LQYMRADQAAGGLRGSGSGSLEVLFQGPGDTRPRFLEYSTSECHFFNGTERVRYLDRYFHNQEENVRFDSDVGEFRAVTELGRPDAEYWNSQKDLLEQKRGRVDNYCRHNYGVVESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSKGGGGSEDLYFQSGGGGSCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKWSHPQFEK

SEQ ID NO: 113 - protein, 480 aa, Artificial sequence
  DR4G110 beta chain
EVVLLVATEGRVRVNSAYQDKGSGSGSLEVLFQGPGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSKGGGGSEDLYFQSGGGGSCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKWSHPQFEK

SEQ ID NO: 114 - protein, 474 aa, Artificial sequence
  DR4G104 beta chain
ACPKYVKQNTLKLATGSGSGSLEVLFQGPGDTRPRFLEQVKHECHFFNGTERVRFLDRYFYHQEEYVRFDSDVGEYRAVTELGRPDAEYWNSQKDILEDERAAVDTYCRHNYGVVESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSKGGGGSEDLYFQSGGGGSCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKWSHPQFEK

SEQ ID NO: 115 - protein, 474 aa, Artificial sequence
  DR4G103 beta chain
ACPKYVKQNTLKLATGSGSGSLEVLFQGPGDTRPRFLWQPKRECHFFNGTERVRFLDRYFYNQEESVRFDSDVGEFRAVTELGRPDAEYWNSQKDILEQARAAVDTYCRHNYGVVESFTVQRRVQPKVTVYPSKTQPLQHHNLLVCSVSGFYPGSIEVRWFLNGQEEKAGMVSTGLIQNGDWTFQTLVMLETVPRSGEVYTCQVEHPSVTSPLTVEWRARSESAQSKGGGGSEDLYFQSGGGGSCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKWSHPQFEK

SEQ ID NO: 116 - protein, 476 aa, Artificial sequence
  DR4G101 beta chain
EPGIAGFKGEQGPKGEPGSGSGSLEVLFQGPGDTRPRFLEQVKHECHFFNGTERVRFLDRYFYHQEEYVRFDSDVGEYRAVTELGRPDAEYWNSQKDILEDERAAVDTYCRHNYGVVESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSKGGGGSEDLYFQSGGGGSCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKWSHPQFEK

SEQ ID NO: 117 - protein, 473 aa, Artificial sequence
  DR4G101 beta chain
LQYMRADQAAGGLRGSGSGSLEVLFQGPGDTRPRFLEQVKHECHFFNGTERVRFLDRYFYHQEEYVRFDSDVGEYRAVTELGRPDAEYWNSQKDILEDERAAVDTYCRHNYGVVESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSKGGGGSEDLYFQSGGGGSCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKWSHPQFEK

SEQ ID NO: 118 - protein, 473 aa, Artificial sequence
  DR4G97 beta chain
LQYMRADQAAGGLRGSGSGSLEVLFQGPGDTRPRFLWQPKRECHFFNGTERVRFLDRYFYNQEESVRFDSDVGEFRAVTELGRPDAEYWNSQKDILEQARAAVDTYCRHNYGVVESFTVQRRVQPKVTVYPSKTQPLQHHNLLVCSVSGFYPGSIEVRWFLNGQEEKAGMVSTGLIQNGDWTFQTLVMLETVPRSGEVYTCQVEHPSVTSPLTVEWRARSESAQSKGGGGSEDLYFQSGGGGSCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKWSHPQFEK

SEQ ID NO: 119 - protein, 474 aa, Artificial sequence
  DR4G96 beta chain
KMRMATPLLMQALPMGSGSGSLEVLFQGPGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKGGGGSEDLYFQSGGGGSCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKWSHPQFEK

SEQ ID NO: 120 - protein, 474 aa, Artificial sequence
  DR4G86 beta chain
KMRMATPLLMQALPMGSGSGSLEVLFQGPGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSKGGGGSEDLYFQSGGGGSCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKWSHPQFEK

SEQ ID NO: 121 - DNA, 363 bp, Artificial sequence
  DR4B98 VH
caggtgcagctggtgcagagcggcgcggaagtgaaaaaaccgggcagcagcgtgaaagtgagctgcaaagcgagcggcggcacctttaagtcctattatattcattgggtgcgccaggcgccgggccagggcctggaatggatgggcggtattgcaccaatttacggcaccgcttactacgcgcagaaatttcagggccgcgtgaccattaccgctgatgaaagcaccagcaccgcgtatatggaactgagcagcctgcgcagcgaagataccgcggtgtattattgcgcgcgtgatgcaagttgggcacgtgcatacggttttgattattggggccagggcaccctggtgaccgtctcgagt

SEQ ID NO: 122 - protein, 15 aa, Homo sapiens
GIAGFKGEQGPKGEP

SEQ ID NO: 123 - protein, 5 aa, Artificial sequence
  DR4B78 , DR4B70, DR4B38 HCDR1
SYAMS

SEQ ID NO: 124 - protein, 5 aa, Artificial sequence
  DR4B33 HCDR1
SAYIN

SEQ ID NO: 125 - protein, 5 aa, Artificial sequence
  DR4B22 HCDR1
SYAMN

SEQ ID NO: 126 - protein, 17 aa, Artificial sequence
  DR4B78, DR4B70, DR4B38 HCDR2
AISGSGGSTYYADSVKG

SEQ ID NO: 127 - protein, 17 aa, Artificial sequence
  DR4B33 HCDR2
IIRPGDSRTRYSPSFQG

SEQ ID NO: 128 - protein, 17 aa, Artificial sequence
  DR4B22 HCDR2
AISGSGGYTNYADSVKG

SEQ ID NO: 129 - protein, 19 aa, Artificial sequence
  DR4B78  HCDR3
DGGYYRYVRTISGDYAFDY

SEQ ID NO: 130 - protein, 19 aa, Artificial sequence
  DR4B70 HCDR3
DSSYYRYIGRYLGDYAFDY

SEQ ID NO: 131 - protein, 19 aa, Artificial sequence
  DR4B38 HCDR3
DSGYYRLAAIGRSDYAFDY

SEQ ID NO: 132 - protein, 16 aa, Artificial sequence
  DR4B33 HCDR3
DGYYFVGSIIYYGMDV

SEQ ID NO: 133 - protein, 19 aa, Artificial sequence
  DR4B22 HCDR3
DGGYYRYVYRYPGDYAFGY

SEQ ID NO: 134 - protein, 11 aa, Artificial sequence
  DR4B33 LCDR1
RASQSISSYLN

SEQ ID NO: 135 - protein, 7 aa, Artificial sequence
  DR4B33 LCDR2
AASSLQS

SEQ ID NO: 136 - protein, 9 aa, Artificial sequence
  DR4B33 LCDR3
QQSYSTPLT

SEQ ID NO: 137 - protein, 128 aa, Artificial sequence
  DR4B78 VH (DR4H62)
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDGGYYRYVRTISGDYAFDYWGQGTLVTVSS

SEQ ID NO: 138 - protein, 128 aa, Artificial sequence
  DR4B70 VH (DR4H29)
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDSSYYRYIGRYLGDYAFDYWGQGTLVTVSS

SEQ ID NO: 139 - protein, 128 aa, Artificial sequence
  DR4B38 VH (DR4H56)
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDSGYYRLAAIGRSDYAFDYWGQGTLVTVSS

SEQ ID NO: 140 - protein, 125 aa, Artificial sequence
  DR4B33 VH (DR4H58)
EVQLVQSGAEVKKPGESLKISCKGSGYSFDSAYINWVRQMPGKGLEWMGIIRPGDSRTRYSPSFQGQVTISADKSISTAYLQWSSLKASDTAVYYCARDGYYFVGSIIYYGMDVWGQGTLVTVSS

SEQ ID NO: 141 - protein, 128 aa, Artificial sequence
  DR4B22 VH (DR4H16)
EVQLLESGGGLVQPGGSLRLSCAASGFTFSSYAMNWVRQAPGKGLEWVSAISGSGGYTNYADSVKGRFTISRDNSKNTLYLQMNSLRAEDTAVYYCARDGGYYRYVYRYPGDYAFGYWGQGTLVTVSS

SEQ ID NO: 142 - protein, 107 aa, Artificial sequence [sequence omitted]

SEQ ID NO: 143 - DNA, 384 bp, Artificial sequence
  DR4B78 VH (DR4H62)
gaagtgcagctgctggaaagcggcggcggcctggtgcagccgggcggcagcctgcgcctgagctgcgcggcgagcggctttacctttagcagctatgcgatgagctgggtgcgccaggcgccgggcaaaggcctggaatgggtgagcgcgatcagcggctccggtggctccacatattatgcggatagcgtgaaaggccgctttaccatttcacgagataacagcaaaaacaccctgtatctgcagatgaacagcctgcgcgcggaagataccgcggtgtattattgcgcgcgcgatggcggttattatcgttatgtgcgtacaatcagcggcgattatgcattcgactattggggccagggcaccctggtgaccgtctcgagt

SEQ ID NO: 144 - DNA, 384 bp, Artificial sequence
  DR4B70 VH (DR4H29)
gaagtgcagctgctggaaagcggcggcggcctggtgcagccgggcggcagcctgcgcctgagctgcgcggcgagcggctttacctttagcagctatgcgatgagctgggtgcgccaggcgccgggcaaaggcctggaatgggtgagcgcgatcagcggctccggtggctccacatattatgcggatagcgtgaaaggccgctttaccatttcacgagataacagcaaaaacaccctgtatctgcagatgaacagcctgcgcgcggaagataccgcggtgtattattgcgcgcgcgactccagctattatcgttacattggccgttatctgggcgactacgcattcgactactggggccagggcaccctggtgaccgtctcgagt

SEQ ID NO: 145 - DNA, 384 bp, Artificial sequence
  DR4B38 VH (DR4H56)
gaagtgcagctgctggaaagcggcggcggcctggtgcagccgggcggcagcctgcgcctgagctgcgcggcgagcggctttacctttagcagctatgcgatgagctgggtgcgccaggcgccgggcaaaggcctggaatgggtgagcgcgatcagcggctccggtggctccacatattatgcggatagcgtgaaaggccgctttaccatttcacgagataacagcaaaaacaccctgtatctgcagatgaacagcctgcgcgcggaagataccgcggtgtattattgcgcgcgtgactccggctattatcgtctggcagcaatcggccgttctgattacgcatttgattactggggccagggcaccctggtgaccgtctcgagt

SEQ ID NO: 146 - DNA, 375 bp, Artificial sequence
  DR4B33 VH (DR4H58)
gaagtgcagctggtgcagagcggcgcggaagtgaaaaaaccgggcgaaagcctgaaaattagctgcaaaggcagcggctatagcttcgatagcgcatacattaattgggtgcgccagatgccgggcaaaggcttggaatggatgggtattattcgtcctggcgattcccgcacgcgttacagcccgagctttcagggccaggtgaccattagcgcggataaaagcattagcaccgcgtatctgcagtggagcagcctgaaagcgagcgataccgcggtgtattattgcgcgcgtgacggctattattttgttggcagcatcatctattacggtatggacgtatggggccagggcaccctggtgaccgtctcgagt

SEQ ID NO: 147 - DNA, 384 bp, Artificial sequence
  DR4B22 VH (DR4H16)
gaagtgcagctgctggaaagcggcggcggcctggtgcagccgggcggcagcctgcgcctgagctgcgcggcgagcggctttaccttttcctcctatgcaatgaattgggtgcgccaggcgccgggcaaaggcctggaatgggtgagcgctattagcggttccggtgggtatacaaattatgcggatagcgtgaaaggccgctttaccatttcacgagataacagcaaaaacaccctgtatctgcagatgaacagcctgcgcgcggaagataccgcggtgtattattgcgcgcgtgacggtggttactaccggtatgtgtaccgttatccaggcgactatgcatttggctattggggccagggcaccctggtgaccgtctcgagt

SEQ ID NO: 148 - DNA, 321 bp, Artificial sequence
  DR4B33 VL (PH9L4)
gacatccagatgacccagagccccagcagcctgagcgccagcgtgggcgaccgggtgaccatcacctgccgggccagccagagcatcagcagctacctgaactggtaccagcagaagcccggcaaggcccccaagctgctgatctacgccgccagcagcctgcagagcggcgtgcccagccggttcagcggcagcggcagcggcaccgacttcaccctgaccatcagcagcctgcagcccgaggacttcgccacctactactgccagcagagctacagcacccccctgaccttcggccagggcaccaaggtggagatcaag

SEQ ID NO: 149 - protein, 454 aa, Artificial sequence
  DR4B78 HC
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDGGYYRYVRTISGDYAFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPAAASSVFLFPPKPKDTLMISRTPEVTCVVVDVSAEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 150 - protein, 454 aa, Artificial sequence
  DR4B70 HC
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDSSYYRYIGRYLGDYAFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPAAASSVFLFPPKPKDTLMISRTPEVTCVVVDVSAEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 151 - protein, 454 aa, Artificial sequence
  DR4B38 HC
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDSGYYRLAAIGRSDYAFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPAAASSVFLFPPKPKDTLMISRTPEVTCVVVDVSAEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 152 - protein, 451 aa, Artificial sequence
  DR4B33 HC
EVQLVQSGAEVKKPGESLKISCKGSGYSFDSAYINWVRQMPGKGLEWMGIIRPGDSRTRYSPSFQGQVTISADKSISTAYLQWSSLKASDTAVYYCARDGYYFVGSIIYYGMDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPAAASSVFLFPPKPKDTLMISRTPEVTCVVVDVSAEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 153 - protein, 454 aa, Artificial sequence
  DR4B22 HC
EVQLLESGGGLVQPGGSLRLSCAASGFTFSSYAMNWVRQAPGKGLEWVSAISGSGGYTNYADSVKGRFTISRDNSKNTLYLQMNSLRAEDTAVYYCARDGGYYRYVYRYPGDYAFGYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPAAASSVFLFPPKPKDTLMISRTPEVTCVVVDVSAEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 154 - protein, 214 aa, Artificial sequence
  DR4B33 LC
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 155 - DNA, 1362 bp, Artificial sequence
  DR4B78 HC
gaagtgcagctgctggaaagcggcggcggcctggtgcagccgggcggcagcctgcgcctgagctgcgcggcgagcggctttacctttagcagctatgcgatgagctgggtgcgccaggcgccgggcaaaggcctggaatgggtgagcgcgatcagcggctccggtggctccacatattatgcggatagcgtgaaaggccgctttaccatttcacgagataacagcaaaaacaccctgtatctgcagatgaacagcctgcgcgcggaagataccgcggtgtattattgcgcgcgcgatggcggttattatcgttatgtgcgtacaatcagcggcgattatgcattcgactattggggccagggcaccctggtgaccgtctcgagtgcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgccgcagccagctcagtcttcctcttccccccaaaacccaaggacaccctcatgatctcccggacccctgaggtcacgtgcgtggtggtggacgtgagcgccgaagaccccgaggtccagttcaactggtacgtggacggcgtggaggtgcataatgccaagacaaagccacgggaggagcagttcaacagcacgttccgtgtggtcagcgtcctcaccgttctgcaccaggactggctgaacggcaaggagtacaagtgcaaggtctccaacaaaggcctcccatcctccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 156 - DNA, 1362 bp, Artificial sequence
  DR4B70 HC
gaagtgcagctgctggaaagcggcggcggcctggtgcagccgggcggcagcctgcgcctgagctgcgcggcgagcggctttacctttagcagctatgcgatgagctgggtgcgccaggcgccgggcaaaggcctggaatgggtgagcgcgatcagcggctccggtggctccacatattatgcggatagcgtgaaaggccgctttaccatttcacgagataacagcaaaaacaccctgtatctgcagatgaacagcctgcgcgcggaagataccgcggtgtattattgcgcgcgcgactccagctattatcgttacattggccgttatctgggcgactacgcattcgactactggggccagggcaccctggtgaccgtctcgagtgcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgccgcagccagctcagtcttcctcttccccccaaaacccaaggacaccctcatgatctcccggacccctgaggtcacgtgcgtggtggtggacgtgagcgccgaagaccccgaggtccagttcaactggtacgtggacggcgtggaggtgcataatgccaagacaaagccacgggaggagcagttcaacagcacgttccgtgtggtcagcgtcctcaccgttctgcaccaggactggctgaacggcaaggagtacaagtgcaaggtctccaacaaaggcctcccatcctccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 157 - DNA, 1362 bp, Artificial sequence
  DR4B38 HC
gaagtgcagctgctggaaagcggcggcggcctggtgcagccgggcggcagcctgcgcctgagctgcgcggcgagcggctttacctttagcagctatgcgatgagctgggtgcgccaggcgccgggcaaaggcctggaatgggtgagcgcgatcagcggctccggtggctccacatattatgcggatagcgtgaaaggccgctttaccatttcacgagataacagcaaaaacaccctgtatctgcagatgaacagcctgcgcgcggaagataccgcggtgtattattgcgcgcgtgactccggctattatcgtctggcagcaatcggccgttctgattacgcatttgattactggggccagggcaccctggtgaccgtctcgagtgcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgccgcagccagctcagtcttcctcttccccccaaaacccaaggacaccctcatgatctcccggacccctgaggtcacgtgcgtggtggtggacgtgagcgccgaagaccccgaggtccagttcaactggtacgtggacggcgtggaggtgcataatgccaagacaaagccacgggaggagcagttcaacagcacgttccgtgtggtcagcgtcctcaccgttctgcaccaggactggctgaacggcaaggagtacaagtgcaaggtctccaacaaaggcctcccatcctccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 158 - DNA, 1353 bp, Artificial sequence
  DR4B33 HC
gaagtgcagctggtgcagagcggcgcggaagtgaaaaaaccgggcgaaagcctgaaaattagctgcaaaggcagcggctatagcttcgatagcgcatacattaattgggtgcgccagatgccgggcaaaggcttggaatggatgggtattattcgtcctggcgattcccgcacgcgttacagcccgagctttcagggccaggtgaccattagcgcggataaaagcattagcaccgcgtatctgcagtggagcagcctgaaagcgagcgataccgcggtgtattattgcgcgcgtgacggctattattttgttggcagcatcatctattacggtatggacgtatggggccagggcaccctggtgaccgtctcgagtgcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgccgcagccagctcagtcttcctcttccccccaaaacccaaggacaccctcatgatctcccggacccctgaggtcacgtgcgtggtggtggacgtgagcgccgaagaccccgaggtccagttcaactggtacgtggacggcgtggaggtgcataatgccaagacaaagccacgggaggagcagttcaacagcacgttccgtgtggtcagcgtcctcaccgttctgcaccaggactggctgaacggcaaggagtacaagtgcaaggtctccaacaaaggcctcccatcctccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 159 - DNA, 1362 bp, Artificial sequence
  DR4B22 HC
gaagtgcagctgctggaaagcggcggcggcctggtgcagccgggcggcagcctgcgcctgagctgcgcggcgagcggctttaccttttcctcctatgcaatgaattgggtgcgccaggcgccgggcaaaggcctggaatgggtgagcgctattagcggttccggtgggtatacaaattatgcggatagcgtgaaaggccgctttaccatttcacgagataacagcaaaaacaccctgtatctgcagatgaacagcctgcgcgcggaagataccgcggtgtattattgcgcgcgtgacggtggttactaccggtatgtgtaccgttatccaggcgactatgcatttggctattggggccagggcaccctggtgaccgtctcgagtgcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgccgcagccagctcagtcttcctcttccccccaaaacccaaggacaccctcatgatctcccggacccctgaggtcacgtgcgtggtggtggacgtgagcgccgaagaccccgaggtccagttcaactggtacgtggacggcgtggaggtgcataatgccaagacaaagccacgggaggagcagttcaacagcacgttccgtgtggtcagcgtcctcaccgttctgcaccaggactggctgaacggcaaggagtacaagtgcaaggtctccaacaaaggcctcccatcctccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 160 - DNA, 642 bp, Artificial sequence
  DR4B33 LC
gacatccagatgacccagagccccagcagcctgagcgccagcgtgggcgaccgggtgaccatcacctgccgggccagccagagcatcagcagctacctgaactggtaccagcagaagcccggcaaggcccccaagctgctgatctacgccgccagcagcctgcagagcggcgtgcccagccggttcagcggcagcggcagcggcaccgacttcaccctgaccatcagcagcctgcagcccgaggacttcgccacctactactgccagcagagctacagcacccccctgaccttcggccagggcaccaaggtggagatcaagcggaccgtggccgcccccagcgtgttcatcttcccccccagcgacgagcagctgaagagcggaaccgcaagcgtggtgtgcctgctgaacaacttctacccccgggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcttgcgaggtgacccaccagggcctgagcagccccgtgaccaagagcttcaaccggggcgagtgc

SEQ ID NO: 161 - protein, 109 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKWGQGTLVTVSS

SEQ ID NO: 162 - protein, 95 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTP